(19) United States
(12) Patent Application Publication
(10) Pub. No.: US 2013/0344553 A1
(43) Pub. Date: Dec. 26, 2013

(54) Designer Calvin-Cycle-Channeled and Hydrogenotrophic Production of Butanol and Related Higher Alcohols

(71) Applicant: James Weifu Lee, Cockeysville, MD (US)

(72) Inventor: James Weifu Lee, Cockeysville, MD (US)

(21) Appl. No.: 13/997,242

(22) PCT Filed: Dec. 20, 2011
(86) PCT No.: PCT/US11/66090

§ 371 Date: Jun. 22, 2013

Related U.S. Application Data
(63) Continuation-in-part of application No. 13/075,153, filed on Mar. 29, 2011.
Application No. 13/075,153 is a continuation-in-part of application No. 12/918,784, filed on Aug. 20, 2010.
(60) Provisional application No. 61/426,147, filed on Dec. 22, 2010.
(60) Provisional application No. 61/066,845, filed on Feb. 23, 2008.
(60) Provisional application No. 61/066,835, filed on Feb. 23, 2008.
(60) Provisional application No. 61/426,147, filed on Dec. 22, 2010.

(51) Int. Cl. C12P 7/16

(52) U.S. Cl. CPC C12P 7/16
USPC 435/160; 435/157

(57) ABSTRACT

Designer Calvin-cycle-channeled and hydrogenotrophic biofuel-production pathways, the associated designer genes and designer transgenic organisms for autotrophic production of butanol and related higher alcohols from carbon dioxide, hydrogen, and/or water are provided. The butanol and related higher alcohols include 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, and 6-methyl-1-heptanol. The designer autotrophic organisms such as designer transgenic oxyphotobacteria and algae comprise designer Calvin-cycle-channeled and hydrogenotrophic pathway gene(s) and biosafety-guarding technology for enhanced autotrophic production of butanol and related higher alcohols from carbon dioxide and water.

### CROSS-REFERENCE TO RELATED APPLICATIONS

This application claims the benefit of U.S. patent application Ser. No. 13/075,153 filed on Mar. 29, 2011, which is a continuation-in-part of co-pending U.S. patent application Ser. No. 12/918,784 filed on Aug. 20, 2010, which is the National Stage of International Application No. PCT/US2009/034801 filed on Feb. 21, 2009, which claims the benefit of U.S. Provisional Application No. 61/066,845 filed on Feb. 23, 2008, and U.S. Provisional Application No. 61/066,835 filed on Feb. 23, 2008. This application also claims the benefit of U.S. Provisional Application No. 61/426,147 filed on Dec. 22, 2010. The entire disclosures of all of these applications are incorporated herein by reference.

### FIELD OF THE INVENTION

The present invention generally relates to biosafety-guarded biofuel energy production technology. More specifically, the present invention provides an autotrophic advanced-biofuels production methodology based on designer transgenic plants, such as transgenic algae, blue-green algae (cyanobacteria and oxychlorobacteria), plant cells or bacterial cells that are created to use the reducing power (NADPH) or Hydrogen (H₂), and energy (ATP) acquired from the photosynthetic and/or hydrogenotrophic process for autotrophic synthesis of butanol and/or related higher alcohols from carbon dioxide (CO₂) and water (H₂O).

### REFERENCE TO SEQUENCE LISTING

The present invention contains references to amino acid sequences and/or nucleic acid sequences which have been submitted concurrently herewith as the sequence listing text file “JWL_{—}004_PCT_SeqListingFull_ST25.txt” updated on Dec. 18, 2911 from the efile of “JWL_{—}004_US1_SeqListingFull_ST25.txt”, file size 429 KB, created on Mar. 29, 2011, in electronic format using the Electronic Filing System of the U.S. Patent and Trademark Office. The aforementioned sequence listing was prepared with PatentIn 3.5, which complies with all format requirements specified in World Intellectual Property Organization Standard (WIPO) ST.25 and the related United States (US) final rule, and is incorporated herein by reference in its entirety including pursuant to 37 C.F.R. §1.52(e)(5) where applicable.

### BACKGROUND OF THE INVENTION

Butanol and/or related higher alcohols can be used as a liquid fuel to run engines such as cars. Butanol can replace gasoline and the energy contents of the two fuels are nearly the same (110,000 Btu per gallon for butanol; 115,000 Btu per gallon for gasoline). Butanol has many superior properties as an alternative fuel when compared to ethanol as well. These include: 1) Butanol has higher energy content (110,000 Btu per gallon butanol) than ethanol (84,000 Btu per gallon ethanol); 2) Butanol is six times less “evaporative” than ethanol and 13.5 times less evaporative than gasoline, making it safer to use as an oxygenate and thereby eliminating the need for very special blends during the summer and winter seasons; 3) Butanol can be transported through the existing fuel infrastructure including the gasoline pipelines whereas ethanol must be shipped via rail, barge or truck; and 4) Butanol can be used as replacement for gasoline gallon for gallon e.g. 100% or any other percentage, whereas ethanol can only be used as an additive to gasoline up to about 85% (E-85) and then only after significant modification to the engine (while butanol can work as a 100% replacement fuel without having to modify the current car engine).

A significant potential market for butanol and/or related higher alcohols as a liquid fuel already exists in the current transportation and energy systems. Butanol is also used as an industrial solvent. In the United States, currently, butanol is manufactured primarily from petroleum. Historically (1900s-1950s), biobutanol was manufactured from corn and molasses in a fermentation process that also produced acetone and ethanol and was known as an ABE (acetone, butanol, ethanol) fermentation typically with certain butanol-producing bacteria such as Clostridium acetobutylicum and Clostridium beijerinckii. When the USA lost its low-cost sugar supply from Cuba around 1954, however, butanol production by fermentation declined mainly because the price of petroleum dropped below that of sugar. Recently, there is renewed R&D interest in producing butanol and/or ethanol from biomass such as corn starch using Clostridia- and/or yeast-fermentation process. However, similarly to the situation of “cornstarch ethanol production,” the “cornstarch butanol production” process also requires a number of energy-consuming steps including agricultural corn-crop cultivation, corn-grain harvesting, corn-grain starch processing, and starch-to-sugar-to-butanol fermentation. The “cornstarch butanol production” process could also probably cost nearly as much energy as the energy value of its product butanol. This is not surprising, understandably because the cornstarch that the current technology can use represents only a small fraction of the corn crop biomass that includes the corn stalks, leaves and roots. The cornstovers are commonly discarded in the agricultural fields where they slowly decompose back to CO₂, because they represent largely lignocellulosic biomass materials that the current biorefinery industry cannot efficiently use for ethanol or butanol production. There are research efforts in trying to make ethanol or butanol from lignocellulosic plant biomass materials—a concept called “cellulosic ethanol” or “cellulosic butanol”. However, plant biomass has evolved effective mechanisms for resisting assault on its cell-wall structural sugars from the microbial and animal kingdoms. This property underlies a natural recalcitrance, creating roadblocks to the cost-effective transformation of lignocellulosic biomass to fermentable sugars. Therefore, one of its problems known as the “lignocellulosic recalcitrance” represents a formidable technical barrier to the cost-effective conversion of plant biomass to fermentable sugars. That is, because of the recalcitrance problem, lignocellulosic biomasses (such as cornstover, switchgrass, and woody plant materials) could not be readily converted to fermentable sugars to make ethanol or butanol without certain pretreatment, which is often associated with high processing cost. Despite more than 50 years of R&D efforts in lignocellulosic biomass pretreatment and fermentative butanol-production processing, the problem of recalcitrant lignocellulosics still remains as a formidable technical barrier that has not yet been eliminated so far. Furthermore, the steps of lignocellulosic biomass cultivation, harvesting, pretreatment processing, and cellulose-to-sugar-to-butanol fermentation all cost energy. Therefore, any new technology that could bypass these bottleneck problems of the biomass technology would be useful.

Oxyphotobacteria (also known as blue-green algae including cyanobacteria and oxychlorobacteria) and algae (such as Chlamydomonas reinhardtii, Platymonas subcordiformis, Chlorella fusca, Dunaliella salina, Ankistrodesmus braunii, and Scenedesmus obliquus), which can perform photosynthetic assimilation of CO₂ with O₂ evolution from water in a liquid culture medium with a maximal theoretical solar-to-biomass energy conversion of about 10%, have tremendous potential to be a clean and renewable energy resource. However, the wild-type oxygenic photosynthetic green plants, such as blue-green algae and eukaryotic algae, do not possess the ability to produce butanol directly from CO₂ and H₂O. The wild-type photosynthesis uses the reducing power (NADPH) and energy (ATP) from the photosynthetic water splitting and proton gradient-coupled electron transport process through the algal thylakoid membrane system to reduce CO₂ into carbohydrates (CH₂O)ₙ such as starch with a series of enzymes collectively called the “Calvin cycle” at the stroma region in an algal or green-plant chloroplast. The net result of the wild-type photosynthetic process is the conversion of CO₂ and H₂O into carbohydrates (CH₂O)ₙ and O₂ using sunlight energy according to the following process reaction:

nCO₂+nH₂O→(CH₂O)n+nO₂ [1]

The carbohydrates (CH₂O)n are then further converted to all kinds of complicated cellular (biomass) materials including proteins, lipids, and cellulose and other cell-wall materials during cell metabolism and growth.

In certain alga such as Chlamydomonas reinhardtii, some of the organic reserves such as starch could be slowly metabolized to ethanol (but not to butanol) through a secondary fermentative metabolic pathway. The algal fermentative metabolic pathway is similar to the yeast-fermentation process, by which starch is breakdown to smaller sugars such as glucose that is, in turn, transformed into pyruvate by a glycolysis process. Pyruvate may then be converted to formate, acetate, and ethanol by a number of additional metabolic steps (Gfeller and Gibbs (1984) “Fermentative metabolism of Chlamydomonas reinhardtii,” Plant Physiol. 75:212-218). The efficiency of this secondary metabolic process is quite limited, probably because it could use only a small fraction of the limited organic reserve such as starch in an algal cell. Furthermore, the native algal secondary metabolic process could not produce any butanol. As mentioned above, butanol (and/or related higher alcohols) has many superior physical properties to serve as a replacement for gasoline as a fuel. Therefore, a new photobiological and/or hydrogenotrophic butanol (and/or related higher alcohols)-producing mechanism with a high energy conversion efficiency is needed.

International Application No. PCT/US2009/034801 discloses a set of methods on designer photosynthetic organisms (such as designer transgenic plant, plant cells, algae and oxyphotobacteria) for photobiological production of butanol from carbon dioxide (CO₂) and water (H₂O).

### SUMMARY OF THE INVENTION

The present invention discloses designer Calvin-cycle-channeled and/or hydrogenotrophic pathways, the associated designer genes and designer transgenic photosynthetic organisms for autotrophic production of butanol and/or related higher alcohols that are selected from the group that consists of: 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, 6-methyl-1-heptanol, and combinations thereof.

The designer autotrophic organisms such as designer transgenic oxyphotobacteria and algae comprise designer Calvin-cycle-channeled and photosynthetic NADPH-enhanced pathway gene(s) and biosafety-guarding technology for enhanced photobiological production of butanol and related higher alcohols from carbon dioxide and water.

According to another embodiment, the transgenic autotrophic organism comprises a transgenic designer plant or plant cells selected from the group consisting of aquatic plants, plant cells, green algae, red algae, brown algae, blue-green algae (oxyphotobacteria including cyanobacteria and oxychlorobacteria), diatoms, marine algae, freshwater algae, salt-tolerant algal strains, cold-tolerant algal strains, heat-tolerant algal strains, antenna-pigment-deficient mutants, butanol-tolerant algal strains, higher-alcohols-tolerant algal strains, butanol-tolerant oxyphotobacteria, higher-alcohols-tolerant oxyphotobacteria, and combinations thereof.

According to one of the various embodiments, a designer Calvin-cycle-channeled photosynthetic NADPH-enhanced pathway that takes the Calvin-cycle intermediate product, 3-phosphoglycerate, and converts it into 1-butanol comprises a set of enzymes selected from the group consisting of: NADPH-dependent glyceraldehyde-3-phosphate dehydrogenase, NAD-dependent glyceraldehyde-3-phosphate dehydrogenase, phosphoglycerate mutase, enolase, pyruvate kinase, citramalate synthase, 2-methylmalate dehydratase, 3-isopropylmalate dehydratase, 3-isopropylmalate dehydrogenase, 2-isopropylmalate synthase, isopropylmalate isomerase, 2-keto acid decarboxylase, alcohol dehydrogenase, NADPH-dependent alcohol dehydrogenase, and butanol dehydrogenase.

According to one of the various embodiments, another designer Calvin-cycle-channeled photosynthetic NADPH-enhanced 1-butanol-production pathway comprises a set of enzymes selected from the group consisting of: NADPH-dependent glyceraldehyde-3-phosphate dehydrogenase, NAD-dependent glyceraldehyde-3-phosphate dehydrogenase, phosphoglycerate mutase, enolase, phosphoenolpyruvate carboxylase, aspartate aminotransferase, aspartokinase, aspartate-semialdehyde dehydrogenase, homoserine dehydrogenase, homoserine kinase, threonine synthase, threonine ammonia-lyase, 2-isopropylmalate synthase, isopropylmalate isomerase, 3-isopropylmalate dehydrogenase, 2-keto acid decarboxylase, and NAD-dependent alcohol dehydrogenase, NADPH-dependent alcohol dehydrogenase, and butanol dehydrogenase.

According to another embodiment, a designer Calvin-cycle-channeled photosynthetic NADPH-enhanced pathway that takes the Calvin-cycle intermediate product, 3-phosphoglycerate, and converts it into 2-methyl-1-butanol, comprises a set of enzymes selected from the group consisting of: NADPH-dependent glyceraldehyde-3-phosphate dehydrogenase, NAD-dependent glyceraldehyde-3-phosphate dehydrogenase, phosphoglycerate mutase, enolase, pyruvate kinase, citramalate synthase, 2-methylmalate dehydratase, 3-isopropylmalate dehydratase, 3-isopropylmalate dehydrogenase, acetolactate synthase, ketol-acid reductoisomerase, dihydroxy-acid dehydratase, 2-keto acid decarboxylase, NAD-dependent alcohol dehydrogenase, NADPH-dependent alcohol dehydrogenase, and 2-methylbutyraldehyde reductase.

According to another embodiment, a designer Calvin-cycle-channeled photosynthetic NADPH-enhanced pathway for photobiological production of 2-methyl-1-butanol production comprises a set of enzymes selected from the group consisting of: NADPH-dependent glyceraldehyde-3-phosphate dehydrogenase, NAD-dependent glyceraldehyde-3-phosphate dehydrogenase, phosphoglycerate mutase, enolase, phosphoenolpyruvate carboxylase, aspartate aminotransferase, aspartokinase, aspartate-semialdehyde dehydrogenase, homoserine dehydrogenase, homoserine kinase, threonine synthase, threonine ammonia-lyase, acetolactate synthase, ketol-acid reductoisomerase, dihydroxy-acid dehydratase, 2-keto acid decarboxylase, and NAD dependent alcohol dehydrogenase, NADPH dependent alcohol dehydrogenase, and 2-methylbutyraldehyde reductase.

According to another embodiment, a designer Calvin-cycle-channeled photosynthetic NADPH-enhanced pathway for photobiological production of isobutanol comprises a set of enzymes selected from the group consisting of: NADPH-dependent glyceraldehyde-3-phosphate dehydrogenase, NAD-dependent glyceraldehyde-3-phosphate dehydrogenase, phosphoglycerate mutase, enolase, pyruvate kinase, acetolactate synthase, ketol-acid reductoisomerase, dihydroxy-acid dehydratase, 2-keto acid decarboxylase, and NAD-dependent alcohol dehydrogenase, and NADPH-dependent alcohol dehydrogenase.

Likewise, a number of other designer Calvin-cycle-channeled photosynthetic NADPH-enhanced pathways are also disclosed according to one of the various embodiments for photobiological production of butanol and/or related higher alcohols such as 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, and/or 6-methyl-1-heptanol.

According to one of various embodiments, a method for photobiological production and harvesting of butanol and related higher alcohols comprises: a) introducing a transgenic photosynthetic organism into a photobiological reactor system, the transgenic photosynthetic organism comprising transgenes coding for a set of enzymes configured to act on an intermediate product of a Calvin cycle and to convert the intermediate product into butanol and/or related higher alcohols; b) using reducing power NADPH and energy ATP associated with the transgenic photosynthetic organism acquired from photosynthetic water splitting and proton gradient coupled electron transport process in the photobioreactor to synthesize butanol and/or related higher alcohols from carbon dioxide and water; and c) using a product separation process to harvest the synthesized butanol and/or related higher alcohols from the photobioreactor.

According to another embodiment, designer hydrogen-driven Calvin-cycle-channeled biofuel-production organisms for chemolithoautotrophic production of butanol and related higher alcohols comprises a set of oxygen-tolerant soluble hydrogenase and membrane-bound hydrogenases in combination with the designer Calvin-cycle-channeled biofuel-production pathways.

According to another embodiment, a designer organism comprises a designer anaerobic hydrogenotrophic system and a reductive-acetyl-CoA biofuel-production pathway(s) for hydrogen-driven chemolithoautotrophic production of 1-butanol(CH₃CH₂CH₂CH₂OH) from hydrogen (H₂) and carbon dioxide (CO₂) with its maximal H₂-to-butanol energy conversion efficiency as high as 91%. This designer autotrophic organism comprises a set of designer genes (e.g., designer DNA constructs) that express the designer anaerobic hydrogenotrophic butanol-production-pathway system comprising: energy converting hydrogenase (Ech), [NiFe]-hydrogenase (Mvh), Coenzyme F₄₂₀-reducing hydrogenase (Frh), native (or heterologous) soluble hydrogenase (SH), heterodissulfide reductase (Hdr), formylmethanofuran dehydroganse, formyl transferase, 10-methenyl-tetrahydromethanopterin cyclohydrolase, 10-methylene-H₄ methanopterin dehydrogenase, 10-methylene-H₄-methanopterin reductase, methyl-H₄-methanopterin: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, butanol dehydrogenase and/or alcohol dehydrogenase.

According to one of the various embodiments, a designer autotrophic organism comprises a designer methanogenic hydrogenotrophic system and a reductive-acetyl-CoA biofuel-production pathway(s) for anaerobic chemolithoautotrophic production of both 1-butanol (CH₃CH₂CH₂CH₂OH) and methane (CH₄) from hydrogen (H₂) and carbon dioxide (CO₂). This designer autotrophic organism comprises a set of designer genes that express a designer methanogenic hydrogenotrophic butanol-production-pathway system comprising: methyl-H4MPT: coenzyme-M methyltransferase Mtr, native (or heterologous) A₁Aₒ-ATP synthase, methyl-coenzyme M reductase Mcr, energy converting hydrogenase (Ech), [NiFe]-hydrogenase (Mvh), Coenzyme F₄₂₀-reducing hydrogenase (Frh), soluble hydrogenase (SH), heterodissulfide reductase (Hdr), formate dehydroganse, 10-formyl-H₄ folate synthetase, methenyltetrahydrofolate cyclohydrolase, 10-methylene-H₄ folate dehydrogenase, 10-methylene-H₄ folate reductase, methyl-H₄ folate: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, and butanol dehydrogenase and/or alcohol dehydrogenase.

### BRIEF DESCRIPTION OF THE DRAWINGS

FIG. 1 presents designer butanol-production pathways branched from the Calvin cycle using the reducing power (NADPH) and energy (ATP) from the photosynthetic water splitting and proton gradient-coupled electron transport process to reduce carbon dioxide (CO₂) into butanol CH₃CH₂CH₂CH₂OH with a series of enzymatic reactions.

FIG. 2A presents a DNA construct for designer butanol-production-pathway gene(s).

FIG. 2B presents a DNA construct for NADPH/NADH-conversion designer gene for NADPH/NADH inter-conversion.

FIG. 2C presents a DNA construct for a designer iRNA starch/glycogen-synthesis inhibitor(s) gene.

FIG. 2D presents a DNA construct for a designer starch-degradation-glycolysis gene(s).

FIG. 2E presents a DNA construct of a designer butanol-production-pathway gene(s) for cytosolic expression.

FIG. 2F presents a DNA construct of a designer butanol-production-pathway gene(s) with two recombination sites for integrative genetic transformation in oxyphotobacteria.

FIG. 2G presents a DNA construct of a designer biosafety-control gene(s).

FIG. 2H presents a DNA construct of a designer proton-channel gene(s).

FIG. 3A illustrates a cell-division-controllable designer organism that contains two key functions: designer biosafety mechanism(s) and designer biofuel-production pathway(s).

FIG. 3B illustrates a cell-division-controllable designer organism for photobiological production of butanol (CH₃CH₂CH₂CH₂OH) from carbon dioxide (CO₂) and water (H₂O) with designer biosafety mechanism(s).

FIG. 3C illustrates a cell-division-controllable designer organism for biosafety-guarded photobiological production of other biofuels such as ethanol (CH₃CH₂OH) from carbon dioxide (CO₂) and water (H₂O).

FIG. 4 presents designer Calvin-cycle-channeled and photosynthetic NADPH-enhanced pathways using the reducing power (NADPH) and energy (ATP) from the photosynthetic water splitting and proton gradient-coupled electron transport process to reduce carbon dioxide (CO₂) into 1-butanol(CH₃CH₂CH₂CH₂OH) with a series of enzymatic reactions.

FIG. 5 presents designer Calvin-cycle-channeled and photosynthetic NADPH-enhanced pathways using NADPH and ATP from the photosynthetic water splitting and proton gradient-coupled electron transport process to reduce carbon dioxide (CO₂) into 2-methyl-1-butanol (CH₃CH₂CH(CH₃)CH₂OH) with a series of enzymatic reactions.

FIG. 6 presents designer Calvin-cycle-channeled and photosynthetic NADPH-enhanced pathways using NADPH and ATP from the photosynthetic water splitting and proton gradient-coupled electron transport process to reduce carbon dioxide (CO₂) into isobutanol ((CH₃)₂CHCH₂OH) and 3-methyl-1-butanol(CH₃CH(CH₃)CH₂CH₂OH) with a series of enzymatic reactions.

FIG. 7 presents designer Calvin-cycle-channeled and photosynthetic NADPH-enhanced pathways using NADPH and ATP from the photosynthetic water splitting and proton gradient-coupled electron transport process to reduce carbon dioxide (CO₂) into 1-hexanol (CH₃CH₂CH₂CH₂CH₂CH₂OH) and 1-octanol (CH₃CH₂CH₂CH₂CH₂CH₂CH₂CH₂OH) with a series of enzymatic reactions.

FIG. 8 presents designer Calvin-cycle-channeled and photosynthetic NADPH-enhanced pathways using NADPH and ATP from the photosynthetic water splitting and proton gradient-coupled electron transport process to reduce carbon dioxide (CO₂) into 1-pentanol (CH₃CH₂CH₂CH₂CH₂OH), 1-hexanol (CH₃CH₂CH₂CH₂CH₂CH₂OH), and 1-heptanol (CH₃CH₂CH₂CH₂CH₂CH₂CH₂OH) with a series of enzymatic reactions.

FIG. 9 presents designer Calvin-cycle-channeled and photosynthetic NADPH-enhanced pathways using NADPH and ATP from the photosynthetic water splitting and proton gradient-coupled electron transport process to reduce carbon dioxide (CO₂) into 3-methyl-1-pentanol (CH₃CH₂CH(CH₃)CH₂CH₂OH), 4-methyl-1-hexanol (CH₃CH₂CH(CH₃)CH₂CH₂CH₂OH), and 5-methyl-1-heptanol (CH₃CH₂CH(CH₃)CH₂CH₂CH₂CH₂OH) with a series of enzymatic reactions.

FIG. 10 presents designer Calvin-cycle-channeled and photosynthetic NADPH-enhanced pathways using NADPH and ATP from the photosynthetic water splitting and proton gradient-coupled electron transport process to reduce carbon dioxide (CO₂) into 4-methyl-1-pentanol (CH₃CH(CH₃)CH₂CH₂CH₂OH), 5-methyl-1-hexanol (CH₃CH(CH₃)CH₂CH₂CH₂CH₂OH), and 6-methyl-1-heptanol (CH₃CH(CH₃)CH₂CH₂CH₂CH₂CH₂OH) with a series of enzymatic reactions.

FIG. 11 illustrates a designer organism with designer oxygen-tolerant hydrogenases and Calvin-cycle-channeled biofuel-production pathway(s) for aerobic chemolithoautotrophic production of biofuels such as butanol (CH₃CH₂CH₂CH₂OH) from hydrogen (H₂), carbon dioxide (CO₂), and oxygen (O₂).

FIG. 12 illustrates a designer organism that comprises a designer anaerobic hydrogenotrophic system with reductive-acetyl-CoA biofuel-production pathway(s) for anaerobic chemolithotrophic production of 1-butanol(CH₃CH₂CH₂CH₂OH) from hydrogen (H₂) and carbon dioxide (CO₂).

FIG. 13 presents a designer reductive-acetyl-CoA biofuel-production pathway for anaerobic hydrogenotrophic production of 1-butanol(CH₃CH₂ CH₂CH₂OH) from carbon dioxide (CO₂) with a series of enzymatic reactions.

FIG. 14 presents a designer ATP-required reductive-acetyl-CoA biofuel-production pathway for anaerobic hydrogenotrophic production of 1-butanol(CH₃ CH₂ CH₂CH₂OH) from carbon dioxide (CO₂) with a series of enzymatic reactions.

FIG. 15 illustrates a designer organism that comprises a designer methanogenic hydrogenotrophic system with reductive-acetyl-CoA biofuel-production pathway(s) for anaerobic chemolithotrophic production of both 1-butanol(CH₃CH₂CH₂CH₂OH) and methane (CH₄) from hydrogen (H₂) and carbon dioxide (CO₂).

FIG. 16 presents designer reductive-acetyl-CoA biofuel-production pathways for anaerobic hydrogenotrophic production of both 1-butanol(CH₃CH₂CH₂CH₂OH) and methane (CH₄) from carbon dioxide (CO₂) with a series of enzymatic reactions.

FIG. 17 presents designer ATP-required reductive-acetyl-CoA biofuel-production pathways for anaerobic hydrogenotrophic production of both 1-butanol(CH₃CH₂CH₂CH₂OH) and methane (CH₄) from carbon dioxide (CO₂) and with a series of enzymatic reactions.

### DETAILED DESCRIPTION OF THE INVENTION

The present invention is directed to an autotrophic butanol and related high alcohols production technology based on designer autotrophic organisms such as designer transgenic plants (e.g., algae and oxyphotobacteria), plant cells, or bacteria. In this context throughout this specification, a “higher alcohol” or “related higher alcohol” refers to an alcohol that comprises at least four carbon atoms, which includes both straight and branched alcohols such as 1-butanol and 2-methyl-1-butanol. The Calvin-cycle-channeled and photosynthetic-NADPH-enhanced pathways are constructed with designer enzymes expressed through use of designer genes in host photosynthetic organisms such as algae and oxyphotobacteria (including cyanobacteria and oxychlorobacteria) organisms for photobiological production of butanol and related higher alcohols. The said butanol and related higher alcohols are selected from the group consisting of: 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, and 6-methyl-1-heptanol. The designer plants and plant cells are created using genetic engineering techniques such that the endogenous photosynthesis regulation mechanism is tamed, and the reducing power (NADPH) and energy (ATP) acquired from the photosynthetic water splitting and proton gradient-coupled electron transport process can be used for immediate synthesis of higher alcohols, such as 1-butanol(CH₃CH₂CH₂CH₂OH) and 2-methyl-1-butanol(CH₃CH₂CH(CH₃)CH₂OH), from carbon dioxide (CO₂) and water (H₂O) according to the following generalized process reaction (where m, n, x and y are its molar coefficients) in accordance of the present invention:

m(CO₂)+n(H₂O)→x(higher alcohols)+y(O₂) [2]

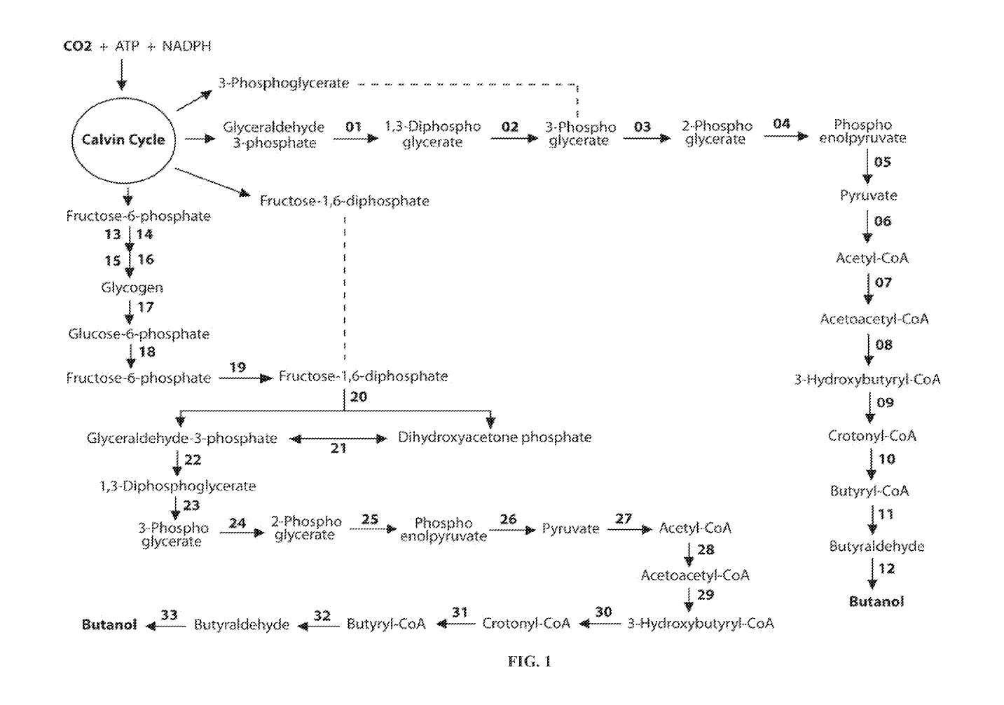

The photobiological higher-alcohols-production methods of the present invention completely eliminate the problem of recalcitrant lignocellulosics by bypassing the bottleneck problem of the biomass technology. As shown in FIG. 1, for example, the photosynthetic process in a designer organism effectively uses the reducing power (NADPH) and energy (ATP) from the photosynthetic water splitting and proton gradient-coupled electron transport process for immediate synthesis of butanol (CH₃CH₂CH₂CH₂OH) directly from carbon dioxide (CO₂) and water (H₂O) without being drained into the other pathway for synthesis of the undesirable lignocellulosic materials that are very hard and often inefficient for the biorefinery industry to use. This approach is also different from the existing “cornstarch butanol production” process. In accordance with this invention, butanol can be produced directly from carbon dioxide (CO₂) and water (H₂O) without having to go through many of the energy consuming steps that the cornstarch butanol-production process has to go through, including corn crop cultivation, corn-grain harvesting, corn-grain cornstarch processing, and starch-to-sugar-to-butanol fermentation. As a result, the photosynthetic butanol-production technology of the present invention is expected to have a much (more than 10-times) higher solar-to-butanol energy-conversion efficiency than the current technology. Assuming a 10% solar energy conversion efficiency for the proposed photosynthetic butanol production process, the maximal theoretical productivity (yield) could be about 72,700 kg of butanol per acre per year, which could support about 70 cars (per year per acre). Therefore, this invention could bring a significant capability to the society in helping to ensure energy security. The present invention could also help protect the Earth's environment from the dangerous accumulation of CO₂ in the atmosphere, because the present methods convert CO₂ directly into clean butanol energy.

A fundamental feature of the present methodology is utilizing a plant (e.g., an alga or oxyphotobacterium) or plant cells, introducing into the plant or plant cells nucleic acid molecules encoding for a set of enzymes that can act on an intermediate product of the Calvin cycle and convert the intermediate product into butanol as illustrated in FIG. 1, instead of making starch and other complicated cellular (biomass) materials as the end products by the wild-type photosynthetic pathway. Accordingly, the present invention provides, inter alia, methods for producing butanol and/or related higher alcohols based on a designer plant (such as a designer alga and a designer oxyphotobacterium), designer plant tissue, or designer plant cells, DNA constructs encoding genes of a designer butanol- and/or related higher alcohols-production pathway(s), as well as the designer algae, designer oxyphotobacteria (including designer cyanobacteria), designer plants, designer plant tissues, and designer plant cells created. The various aspects of the present invention are described in further detail hereinbelow.

### Host Photosynthetic Organisms

According to the present invention, a designer organism or cell for the photosynthetic butanol and/or related higher alcohols production of the invention can be created utilizing as host, any plant (including alga and oxyphotobacterium), plant tissue, or plant cells that have a photosynthetic capability, i.e., an active photosynthetic apparatus and enzymatic pathway that captures light energy through photosynthesis, using this energy to convert inorganic substances into organic matter. Preferably, the host organism should have an adequate photosynthetic CO₂ fixation rate, for example, to support photosynthetic butanol (and/or related higher alcohols) production from CO₂ and H₂O at least about 1,450 kg butanol per acre per year, more preferably, 7,270 kg butanol per acre per year, or even more preferably, 72,700 kg butanol per acre per year.

In a preferred embodiment, an aquatic plant is utilized to create a designer plant. Aquatic plants, also called hydrophytic plants, are plants that live in or on aquatic environments, such as in water (including on or under the water surface) or permanently saturated soil. As used herein, aquatic plants include, for example, algae, blue-green algae (cyanobacteria and oxychlorobacteria), submersed aquatic herbs (Hydrilla verticillate, Elodea densa, Hippuris vulgaris, Aponogeton Boivinianus Aponogeton Rigidifolius, Aponogeton Longiplumulosus, Didiplis Diandra, Vesicularia Dubyana, Hygrophilia Augustifolia, Micranthemum Umbrosum, Eichhornia Azurea, Saururus Cernuus, Cryptocoryne Lingua, Hydrotriche Hottoniiflora Eustralis Stellata, Vallisneria Rubra, Hygrophila Salicifolia, Cyperus Helferi, Cryptocoryne Petchii, Vallisneria americana, Vallisneria Torta, Hydrotriche Hottoniiflora, Crassula Helmsii, Limnophila Sessiliflora, Potamogeton Perfoliatus, Rotala Wallichii, Cryptocoryne Becketii, Blyxa Aubertii, Hygrophila Difformmis), duckweeds (Spirodela polyrrhiza, Wolffia globosa, Lemna trisulca, Lemna gibba, Lemna minor, Landoltia punctata), water cabbage (Pistia stratiotes), buttercups (Ranunculus), water caltrop (Trapa natans and Trapa bicornis), water lily (Nymphaea lotus, Nymphaeaceae and Nelumbonaceae), water hyacinth (Eichhornia crassipes), Bolbitis heudelotii, Cabomba sp., seagrasses (Heteranthera Zosterifolia, Posidoniaceae, Zosteraceae, Hydrocharitaceae, and Cymodoceaceae). Butanol (and/or related higher alcohols) produced from an aquatic plant can diffuse into water, permitting normal growth of the plants and more robust production of butanol from the plants. Liquid cultures of aquatic plant tissues (including, but not limited to, multicellular algae) or cells (including, but not limited to, unicellular algae) are also highly preferred for use, since the butanol (and/or related higher alcohols) molecules produced from a designer butanol (and/or related higher alcohols) production pathway(s) can readily diffuse out of the cells or tissues into the liquid water medium, which can serve as a large pool to store the product butanol (and/or related higher alcohols) that can be subsequently harvested by filtration and/or distillation/evaporation techniques.

Although aquatic plants or cells are preferred host organisms for use in the methods of the present invention, tissue and cells of non-aquatic plants, which are photosynthetic and can be cultured in a liquid culture medium, can also be used to create designer tissue or cells for photosynthetic butanol (and/or related higher alcohols) production. For example, the following tissue or cells of non-aquatic plants can also be selected for use as a host organism in this invention: the photoautotrophic shoot tissue culture of wood apple tree Feronia limonia, the chlorophyllous callus-cultures of corn plant Zea mays, the green root cultures of Asteraceae and Solanaceae species, the tissue culture of sugarcane stalk parenchyma, the tissue culture of bryophyte Physcomitrella patens, the photosynthetic cell suspension cultures of soybean plant (Glycine max), the photoautotrophic and photomixotrophic culture of green Tobacco (Nicofiana tabacum L.) cells, the cell suspension culture of Gisekia pharmaceoides (a C₄ plant), the photosynthetic suspension cultured lines of Amaranthus powellii Wats., Datura innoxia Mill., Gossypium hirsutum L., and Nicotiana tabacum×Nicotiana glutinosa L. fusion hybrid.

By “liquid medium” is meant liquid water plus relatively small amounts of inorganic nutrients (e.g., N, P, K etc, commonly in their salt forms) for photoautotrophic cultures; and sometimes also including certain organic substrates (e.g., sucrose, glucose, or acetate) for photomixotrophic and/or photoheterotrophic cultures.

In an especially preferred embodiment, the plant utilized in the butanol (and/or related higher alcohols) production method of the present invention is an alga or a blue-green alga. The use of algae and/or blue-green algae has several advantages. They can be grown in an open pond at large amounts and low costs. Harvest and purification of butanol (and/or related higher alcohols) from the water phase is also easily accomplished by distillation/evaporation or membrane separation.

Algae suitable for use in the present invention include both unicellular algae and multi-unicellular algae. Multicellular algae that can be selected for use in this invention include, but are not limited to, seaweeds such as Ulva latissima (sea lettuce), Ascophyllum nodosum, Codium fragile, Fucus vesiculosus, Eucheuma denticulatum, Gracilaria gracilis, Hydrodictyon reticulatum, Laminaria japonica, Undaria pinntifida, Saccharina japonica, Porphyra yezoensis, and Porphyra tenera. Suitable algae can also be chosen from the following divisions of algae: green algae (Chlorophyta), red algae (Rhodophyta), brown algae (Phaeophyta), diatoms (Bacillariophyta), and blue-green algae (Oxyphotobacteria including Cyanophyta and Prochlorophytes). Suitable orders of green algae include Ulvales, Ulotrichales, Volvocales, Chlorellales, Schizogoniales, Oedogoniales, Zygnematales, Cladophorales, Siphonales, and Dasycladales. Suitable genera of Rhodophyta are Porphyra, Chondrus, Cyanidioschyzon, Porphyridium, Gracilaria, Kappaphycus, Gelidium and Agardhiella. Suitable genera of Phaeophyta are Laminaria, Undaria, Macrocystis, Sargassum and Dictyosiphon. Suitable genera of Cyanophyta (also known as Cyanobacteria) include (but not limited to) Phoridium, Synechocystis, Syncechococcus, Oscillatoria, and Anabaena. Suitable genera of Prochlorophytes (also known as oxychlorobacteria) include (but not limited to) Prochloron, Prochlorothrix, and Prochlorococcus. Suitable genera of Bacillariophyta are Cyclotella, Cylindrotheca, Navicula, Thalassiosira, and Phaeodactylum. Preferred species of algae for use in the present invention include Chlamydomonas reinhardtii, Platymonas subcordiformis, Chlorella fusca, Chlorella sorokiniana, Chlorella vulgaris, ‘Chlorella’ ellipsoidea, Chlorella spp., Dunaliella salina, Dunaliella viridis, Dunaliella bardowil, Haematococcus pluvialis; Parachlorella kessleri, Betaphycus gelatinum, Chondrus crispus, Cyanidioschyzon merolae, Cyanidium caldarium, Galdieria sulphuraria, Gelidiella acerosa, Gracilaria changii, Kappaphycus alvarezii, Porphyra miniata, Ostreococcus tauri, Porphyra yezoensis, Porphyridium sp., Palmaria palmata, Gracilaria spp., Isochrysis galbana, Kappaphycus spp., Laminaria japonica, Laminaria spp., Monostroma spp., Nannochloropsis oculata, Porphyra spp., Porphyridium spp., Undaria pinnatifida, Ulva lactuca, Ulva spp., Undaria spp., Phaeodactylum Tricornutum, Navicula saprophila, Crypthecodinium cohnii, Cylindrotheca fusiformis, Cyclotella cryptica, Euglena gracilis, Amphidinium sp., Symbiodinium microadriaticum, Macrocystis pyrifera, Ankistrodesmus braunii, and Scenedesmus obliquus.

Preferred species of blue-green algae (oxyphotobacteria including cyanobacteria and oxychlorobacteria) for use in the present invention include Thermosynechococcus elongatus BP-1, Nostoc sp. PCC 7120, Synechococcus elongatus PCC 6301, Syncechococcus sp. strain PCC 7942, Syncechococcus sp. strain PCC 7002, Syncechocystis sp. strain PCC 6803, Prochlorococcus marinus MED4, Prochlorococcus marinus MIT 9313, Prochlorococcus marinus NATL1A, Prochlorococcus SS120, Spirulina platensis (Arthrospira platensis), Spirulina pacifica, Lyngbya majuscule, Anabaena sp., Synechocystis sp., Synechococcus elongates, Synechococcus (MC-A), Trichodesmium sp., Richelia intracellularis, Synechococcus WH7803, Synechococcus WH8102, Nostoc punctiforme, Syncechococcus sp. strain PCC 7943, Synechocyitis PCC 6714 phycocyanin-deficient mutant PD-1, Cyanothece strain 51142, Cyanothece sp. CCY0110, Oscillatoria limosa, Lyngbya majuscula, Symploca muscorum, Gloeobacter violaceus, Prochloron didemni, Prochlorothrix hollandica, Synechococcus (MC-A), Trichodesmium sp., Richelia intracellularis, Prochlorococcus marinus, Prochlorococcus SS120, Synechococcus WH8102, Lyngbya majuscula, Symploca muscorum, Synechococcus bigranulatus, cryophilic Oscillatoria sp., Phormidium sp., Nostoc sp.-1, Calothrix parietina, thermophilic Synechococcus bigranulatus, Synechococcus lividus, thermophilic Mastigocladus laminosus, Chlorogloeopsis fritschii PCC 6912, Synechococcus vulcanus, Synechococcus sp. strain MA4, Synechococcus sp. strain MA19, and Thermosynechococcus elongatus.

Proper selection of host photosynthetic organisms for their genetic backgrounds and certain special features is also beneficial. For example, a photosynthetic-butanol-producing designer alga created from cryophilic algae (psychrophiles) that can grow in snow and ice, and/or from cold-tolerant host strains such as Chlamydomonas cold strain CCMG1619, which has been characterized as capable of performing photosynthetic water splitting as cold as 4° C. (Lee, Blankinship and Greenbaum (1995), “Temperature effect on production of hydrogen and oxygen by Chlamydomonas cold strain CCMP1619 and wild type 137c,” Applied Biochemistry and Biotechnology 51/52:379-386), permits photobiological butanol production even in cold seasons or regions such as Canada. Meanwhile, a designer alga created from a thermophilic/thermotolerant photosynthetic organism such as thermophilic algae Cyanidium caldarium and Galdieria sulphuraria and/or thermophilic cyanobacteria (blue-green algae) such as Thermosynechococcus elongatus BP-1 and Synechococcus bigranulatus may permit the practice of this invention to be well extended into the hot seasons or areas such as Mexico and the Southwestern region of the United States including Nevada, California, Arizona, New Mexico and Texas, where the weather can often be hot. Furthermore, a photosynthetic-butanol-producing designer alga created from a marine alga, such as Platymonas subcordiformis, permits the practice of this invention using seawater, while the designer alga created from a freshwater alga such as Chlamydomonas reinhardtii can use freshwater. Additional optional features of a photosynthetic butanol (and/or related higher alcohols) producing designer alga include the benefits of reduced chlorophyll-antenna size, which has been demonstrated to provide higher photosynthetic productivity (Lee, Mets, and Greenbaum (2002). “Improvement of photosynthetic efficiency at high light intensity through reduction of chlorophyll antenna size,” Applied Biochemistry and Biotechnology, 98-100: 37-48) and butanol-tolerance (and/or related higher alcohols-tolerance) that allows for more robust and efficient photosynthetic production of butanol (and/or related higher alcohols) from CO₂ and H₂O. By use of a phycocyanin-deficient mutant of Synechocystis PCC 6714, it has been experimentally demonstrated that photoinhibition can be reduced also by reducing the content of light-harvesting pigments (Nakajima, Tsuzuki, and Ueda (1999) “Reduced photoinhibition of a phycocyanin-deficient mutant of Synechocystis PCC 6714”, Journal of Applied Phycology 10: 447-452). These optional features can be incorporated into a designer alga, for example, by use of a butanol-tolerant and/or chlorophyll antenna-deficient mutant (e.g., Chlamydomonas reinhardtii strain DS521) as a host organism, for gene transformation with the designer butanol-production-pathway genes. Therefore, in one of the various embodiments, a host alga is selected from the group consisting of green algae, red algae, brown algae, blue-green algae (oxyphotobacteria including cyanobacteria and prochlorophytes), diatoms, marine algae, freshwater algae, unicellular algae, multicellular algae, seaweeds, cold-tolerant algal strains, heat-tolerant algal strains, light-harvesting-antenna-pigment-deficient mutants, butanol-tolerant algal strains, higher alcohols-tolerant algal strains, and combinations thereof.

### Creating a Designer Butanol-Production Pathway in a Host

### Selecting Appropriate Designer Enzymes

One of the key features in the present invention is the creation of a designer butanol-production pathway to tame and work with the natural photosynthetic mechanisms to achieve the desirable synthesis of butanol directly from CO₂ and H₂O. The natural photosynthetic mechanisms include (1) the process of photosynthetic water splitting and proton gradient-coupled electron transport through the thylakoid membrane, which produces the reducing power (NADPH) and energy (ATP), and (2) the Calvin cycle, which reduces CO₂ by consumption of the reducing power (NADPH) and energy (ATP).

In accordance with the present invention, a series of enzymes are used to create a designer butanol-production pathway that takes an intermediate product of the Calvin cycle and converts the intermediate product into butanol as illustrated in FIG. 1. A “designer butanol-production-pathway enzyme” is hereby defined as an enzyme that serves as a catalyst for at least one of the steps in a designer butanol-production pathway. According to the present invention, a number of intermediate products of the Calvin cycle can be utilized to create designer butanol-production pathway(s); and the enzymes required for a designer butanol-production pathway are selected depending upon from which intermediate product of the Calvin cycle the designer butanol-production pathway branches off from the Calvin cycle.

In one example, a designer pathway is created that takes glyceraldehydes-3-phosphate and converts it into butanol by using, for example, a set of enzymes consisting of, as shown with the numerical labels 01-12 in FIG. 1, glyceraldehyde-3-phosphate dehydrogenase 01, phosphoglycerate kinase 02, phosphoglycerate mutase 03, enolase 04, pyruvate kinase 05, pyruvate-ferredoxin oxidoreductase 06, thiolase 07, 3-hydroxybutyryl-CoA dehydrogenase 08, crotonase 09, butyryl-CoA dehydrogenase 10, butyraldehyde dehydrogenase 11, and butanol dehydrogenase 12. In this glyceraldehydes-3-phosphate-branched designer pathway, for conversion of two molecules of glyceraldehyde-3-phosphate to butanol, two NADH molecules are generated from NAD⁺ at the step from glyceraldehyde-3-phosphate to 1,3-diphosphoglycerate catalyzed by glyceraldehyde-3-phosphate dehydrogenase 01; meanwhile two molecules of NADH are converted to NAD⁺: one at the step catalyzed by 3-hydroxybutyryl-CoA dehydrogenase 08 in reducing acetoacetyl-CoA to 3-hydroxybutyryl-CoA and another at the step catalyzed by butyryl-CoA dehydrogenase 10 in reducing crotonyl-CoA to butyryl-CoA. Consequently, in this glyceraldehydes-3-phosphate-branched designer pathway (01-12), the number of NADH molecules consumed is balanced with the number of NADH molecules generated. Furthermore, both the pathway step catalyzed by butyraldehyde dehydrogenase 11 (in reducing butyryl-CoA to butyraldehyde) and the terminal step catalyzed by butanol dehydrogenase 12 (in reducing butyraldehyde to butanol) can use NADPH, which can be regenerated by the photosynthetic water splitting and proton gradient-coupled electron transport process. Therefore, this glyceraldehydes-3-phosphate-branched designer butanol-production pathway can operate continuously.

In another example, a designer pathway is created that takes the intermediate product, 3-phosphoglycerate, and converts it into butanol by using, for example, a set of enzymes consisting of (as shown with the numerical labels 03-12 in FIG. 1) phosphoglycerate mutase 03, enolase 04, pyruvate kinase 05, pyruvate-ferredoxin oxidoreductase 06, thiolase 07, 3-hydroxybutyryl-CoA dehydrogenase 08, crotonase 09, butyryl-CoA dehydrogenase 10, butyraldehyde dehydrogenase 11, and butanol dehydrogenase 12. It is worthwhile to note that the last ten enzymes (03-12) of the glyceraldehydes-3-phosphate-branched designer butanol-producing pathway (01-12) are identical with those utilized in the 3-phosphoglycerate-branched designer pathway (03-12). In other words, the designer enzymes (01-12) of the glyceraldehydes-3-phosphate-branched pathway permit butanol production from both the point of 3-phosphoglycerate and the point glyceraldehydes 3-phosphate in the Calvin cycle. These two pathways, however, have different characteristics. Unlike the glyceraldehyde-3-phosphate-branched butanol-production pathway, the 3-phosphoglycerate-branched pathway which consists of the activities of only ten enzymes (03-12) could not itself generate any NADH that is required for use at two places: one at the step catalyzed by 3-hydroxybutyryl-CoA dehydrogenase 08 in reducing acetoacetyl-CoA to 3-hydroxybutyryl-CoA, and another at the step catalyzed by butyryl-CoA dehydrogenase 10 in reducing crotonyl-CoA to butyryl-CoA. That is, if (or when) a 3-hydroxybutyryl-CoA dehydrogenase and/or a butyryl-CoA dehydrogenase that can use strictly only NADH but not NADPH is employed, it would require a supply of NADH for the 3-phosphoglycerate-branched pathway (03-12) to operate. Consequently, in order for the 3-phosphoglycerate-branched butanol-production pathway to operate, it is important to use a 3-hydroxybutyryl-CoA dehydrogenase 08 and a butyryl-CoA dehydrogenase 10 that can use NADPH which can be supplied by the photo-driven electron transport process. Therefore, it is a preferred practice to use a 3-hydroxybutyryl-CoA dehydrogenase and a butyryl-CoA dehydrogenase that can use NADPH or both NADPH and NADH (i.e., NAD(P)H) for this 3-phosphoglycerate-branched designer butanol-production pathway (03-12 in FIG. 1). Alternatively, when a 3-hydroxybutyryl-CoA dehydrogenase and a butyryl-CoA dehydrogenase that can use only NADH are employed, it is preferably here to use an additional embodiment that can confer an NADPH/NADH conversion mechanism (to supply NADH by converting NADPH to NADH, see more detail later in the text) in the designer organism to facilitate photosynthetic production of butanol through the 3-phosphoglycerate-branched designer pathway.

In still another example, a designer pathway is created that takes fructose-1,6-diphosphate and converts it into butanol by using, as shown with the numerical labels 20-33 in FIG. 1, a set of enzymes consisting of aldolase 20, triose phosphate isomerase 21, glyceraldehyde-3-phosphate dehydrogenase 22, phosphoglycerate kinase 23, phosphoglycerate mutase 24, enolase 25, pyruvate kinase 26, pyruvate-NADP⁺ oxidoreductase (or pyruvate-ferredoxin oxidoreductase) 27, thiolase 28, 3-hydroxybutyryl-CoA dehydrogenase 29, crotonase 30, butyryl-CoA dehydrogenase 31, butyraldehyde dehydrogenase 32, and butanol dehydrogenase 33, with aldolase 20 and triose phosphate isomerase 21 being the only two additional enzymes relative to the glyceraldehydes-3-phosphate-branched designer pathway. The use of a pyruvate-NADP⁺ oxidoreductase 27 (instead of pyruvate-ferredoxin oxidoreductase) in catalyzing the conversion of a pyruvate molecule to acetyl-CoA enables production of an NADPH, which can be used in some other steps of the butanol-production pathway. The addition of yet one more enzyme in the designer organism, phosphofructose kinase 19, permits the creation of another designer pathway which branches off from the point of fructose-6-phosphate of the Calvin cycle for the production of butanol. Like the glyceraldehyde-3-phosphate-branched butanol-production pathway, both the fructose-1,6-diphosphate-branched pathway (20-33) and the fructose-6-phosphate-branched pathway (19-33) can themselves generate NADH for use in the pathway at the step catalyzed by 3-hydroxybutyryl-CoA dehydrogenase 29 to reduce acetoacetyl-CoA to 3-hydroxybutyryl-CoA and at the step catalyzed by butyryl-CoA dehydrogenase 31 to reduce crotonyl-CoA to butyryl-CoA. In each of these designer butanol-production pathways, the numbers of NADH molecules consumed are balanced with the numbers of NADH molecules generated; and both the butyraldehyde dehydrogenase 32 (catalyzing the step in reducing butyryl-CoA to butyraldehyde) and the butanol dehydrogenase 33 (catalyzing the terminal step in reducing butyraldehyde to butanol) can all use NADPH, which can be regenerated by the photosynthetic water splitting and proton gradient-coupled electron transport process. Therefore, these designer butanol-production pathways can operate continuously.

Table 1 lists examples of the enzymes including those identified above for construction of the designer butanol-production pathways. Throughout this specification, when reference is made to an enzyme, such as, for example, any of the enzymes listed in Table 1, it includes their isozymes, functional analogs, and designer modified enzymes and combinations thereof. These enzymes can be selected for use in construction of the designer butanol-production pathways (such as those illustrated in FIG. 1). The “isozymes or functional analogs” refer to certain enzymes that have the same catalytic function but may or may not have exactly the same protein structures. The most essential feature of an enzyme is its active site that catalyzes the enzymatic reaction. Therefore, certain enzyme-protein fragment(s) or subunit(s) that contains such an active catalytic site may also be selected for use in this invention. For various reasons, some of the natural enzymes contain not only the essential catalytic structure but also other structure components that may or may not be desirable for a given application. With techniques of bioinformatics-assisted molecular designing, it is possible to select the essential catalytic structure(s) for use in construction of a designer DNA construct encoding a desirable designer enzyme. Therefore, in one of the various embodiments, a designer enzyme gene is created by artificial synthesis of a DNA construct according to bioinformatics-assisted molecular sequence design. With the computer-assisted synthetic biology approach, any DNA sequence (thus its protein structure) of a designer enzyme may be selectively modified to achieve more desirable results by design. Therefore, the terms “designer modified sequences” and “designer modified enzymes” are hereby defined as the DNA sequences and the enzyme proteins that are modified with bioinformatics-assisted molecular design. For example, when a DNA construct for a designer chloroplast-targeted enzyme is designed from the sequence of a mitochondrial enzyme, it is a preferred practice to modify some of the protein structures, for example, by selectively cutting out certain structure component(s) such as its mitochondrial transit-peptide sequence that is not suitable for the given application, and/or by adding certain peptide structures such as an exogenous chloroplast transit-peptide sequence (e.g., a 135-bp Rubisco small-subunit transit peptide (RbcS2)) that is needed to confer the ability in the chloroplast-targeted insertion of the designer protein. Therefore, one of the various embodiments flexibly employs the enzymes, their isozymes, functional analogs, designer modified enzymes, and/or the combinations thereof in construction of the designer butanol-production pathway(s).

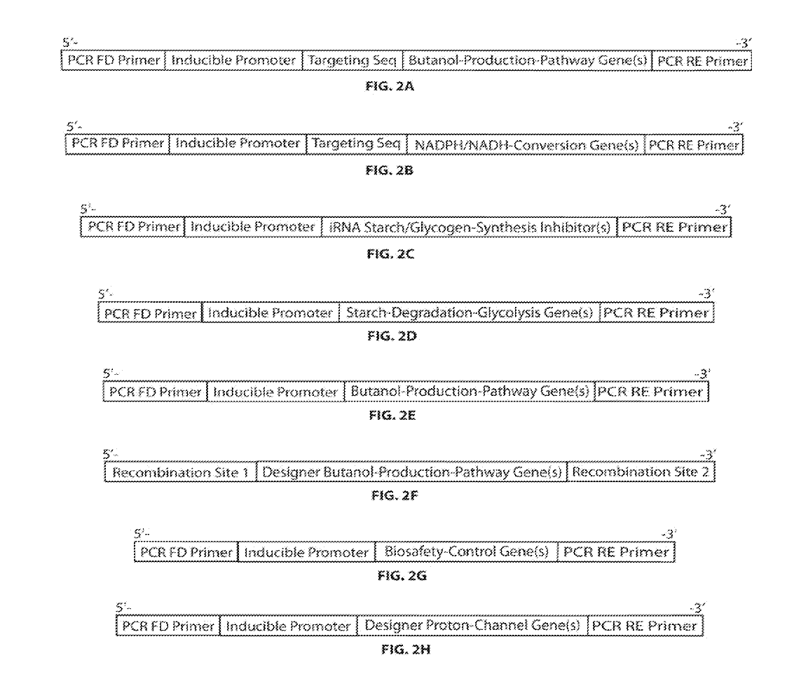

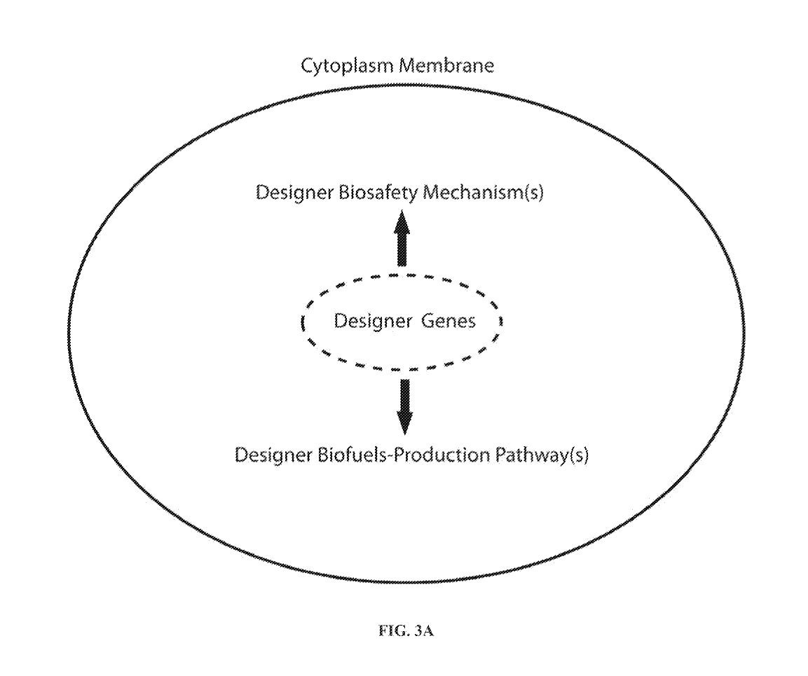

As shown in Table 1, many genes of the enzymes identified above have been cloned and/or sequenced from various organisms. Both genomic DNA and/or mRNA sequence data can be used in designing and synthesizing the designer DNA constructs for transformation of a host alga, oxyphotobacterium, plant, plant tissue or cells to create a designer organism for photobiological butanol production (FIG. 1). However, because of possible variations often associated with various source organisms and cellular compartments with respect to a specific host organism and its chloroplast/thylakoid environment where the butanol-production pathway(s) is designed to work with the Calvin cycle, certain molecular engineering art work in DNA construct design including codon-usage optimization and sequence modification is often necessary for a designer DNA construct (FIG. 2) to work well. For example, in creating a butanol-producing designer eukaryotic alga, if the source sequences are from cytosolic enzymes (sequences), a functional chloroplast-targeting sequence may be added to provide the capability for a designer unclear gene-encoded enzyme to insert into a host chloroplast to confer its function for a designer butanol-production pathway. Furthermore, to provide the switchability for a designer butanol-production pathway, it is also important to include a functional inducible promoter sequence such as the promoter of a hydrogenase (Hyd1) or nitrate reductase (Nia1) gene, or nitrite reductase (nirA) gene in certain designer DNA construct(s) as illustrated in FIG. 2A to control the expression of designer gene(s). In addition, as mentioned before, certain functional derivatives or fragments of these enzymes (sequences), chloroplast-targeting transit peptide sequences, and inducible promoter sequences can also be selected for use in full, in part or in combinations thereof, to create the designer organisms according to various embodiments of this invention. The arts in creating and using the designer organisms are further described hereinbelow.

### Targeting the Designer Enzymes to the Stroma Region of Chloroplasts

Some of the designer enzymes discussed above, such as, pyruvate-ferredoxin oxidoreductase, thiolase, 3-hydroxybutyryl-CoA dehydrogenase, crotonase, butyryl-CoA dehydrogenase, butyraldehyde dehydrogenase, and butanol dehydrogenase are known to function in certain special bacteria such as Clostridium; but wild-type plant chloroplasts generally do not possess these enzymes to function with the Calvin cycle. Therefore, in one of the various embodiments in creating a butanol-producing eukaryotic designer organism, designer nucleic acids encoding for these enzymes are expressed in the chloroplast(s) of a host cell. This can be accomplished by delivery of designer butanol-production-pathway gene(s) into the chloroplast genome of the eukaryotic host cell typically using a genegun. In certain extent, the molecular genetics of chloroplasts are similar to that of cyanobacteria. After being delivered into the chloroplast, a designer DNA construct that contains a pair of proper recombination sites as illustrated in FIG. 2F can be incorporated into the chloroplast genome through a natural process of homologous DNA double recombination.

In another embodiment, nucleic acids encoding for these enzymes are genetically engineered such that the enzymes expressed are inserted into the chloroplasts to operate with the Calvin cycle there. Depending on the genetic background of a particular host organism, some of the designer enzymes discussed above such as phosphoglycerate mutase and enolase may exist at some background levels in its native form in a wild-type chloroplast. For various reasons including often the lack of their controllability, however, some of the chloroplast background enzymes may or may not be sufficient to serve as a significant part of the designer butanol-production pathway(s). Furthermore, a number of useful inducible promoters happen to function in the nuclear genome. For example, both the hydrogenase (Hyd1) promoter and the nitrate reductase (Nia1) promoter that can be used to control the expression of the designer butanol-production pathways are located in the nuclear genome of Chlamydomonas reinhardtii, of which the genome has recently been sequenced. Therefore, in one of the various embodiments, it is preferred to use nuclear-genome-encodable designer genes to confer a switchable butanol-production pathway. Consequently, nucleic acids encoding for these enzymes also need to be genetically engineered with proper sequence modification such that the enzymes are controllably expressed and are inserted into the chloroplasts to create a designer butanol-production pathway.

According to one of the various embodiments, it is best to express the designer butanol-producing-pathway enzymes only into chloroplasts (at the stroma region), exactly where the action of the enzymes is needed to enable photosynthetic production of butanol. If expressed without a chloroplast-targeted insertion mechanism, the enzymes would just stay in the cytosol and not be able to directly interact with the Calvin cycle for butanol production. Therefore, in addition to the obvious distinctive features in pathway designs and associated approaches, another significant distinction is that one of the various embodiments innovatively employs a chloroplast-targeted mechanism for genetic insertion of many designer butanol-production-pathway enzymes into chloroplast to directly interact with the Calvin cycle for photobiological butanol production.

With a chloroplast stroma-targeted mechanism, the cells will not only be able to produce butanol but also to grow and regenerate themselves when they are returned to certain conditions under which the designer pathway is turned off, such as under aerobic conditions when designer hydrogenase promoter-controlled butanol-production-pathway genes are used. Designer algae, plants, or plant cells that contain normal mitochondria should be able to use the reducing power (NADH) from organic reserves (and/or some exogenous organic substrate such as acetate or sugar) to power the cells immediately after returning to aerobic conditions. Consequently, when the designer algae, plants, or plant cells are returned to aerobic conditions after use under anaerobic conditions for photosynthetic butanol production, the cells will stop making the butanol-producing-pathway enzymes and start to restore the normal photoautotrophic capability by synthesizing new and functional chloroplasts. Therefore, it is possible to use such genetically engineered designer alga/plant organisms for repeated cycles of photoautotrophic growth under normal aerobic conditions and efficient production of butanol directly from CO₂ and H₂O under certain specific designer butanol-producing conditions such as under anaerobic conditions and/or in the presence of nitrate when a Nia1 promoter-controlled butanol-production pathway is used.

The targeted insertion of designer butanol-production-pathway enzymes can be accomplished through use of a DNA sequence that encodes for a stroma “signal” peptide. A stroma-protein signal (transit) peptide directs the transport and insertion of a newly synthesized protein into stroma. In accordance with one of the various embodiments, a specific targeting DNA sequence is preferably placed in between the promoter and a designer butanol-production-pathway enzyme sequence, as shown in a designer DNA construct (FIG. 2A). This targeting sequence encodes for a signal (transit) peptide that is synthesized as part of the apoprotein of an enzyme in the cytosol. The transit peptide guides the insertion of an apoprotein of a designer butanol-production-pathway enzyme from cytosol into the chloroplast. After the apoprotein is inserted into the chloroplast, the transit peptide is cleaved off from the apoprotein, which then becomes an active enzyme.

A number of transit peptide sequences are suitable for use for the targeted insertion of the designer butanol-production-pathway enzymes into chloroplast, including but not limited to the transit peptide sequences of: the hydrogenase apoproteins (such as HydA1 (Hyd1) and HydA2, GenBank accession number AJ308413, AF289201, AY090770), ferredoxin apoprotein (Frx1, accession numbers L10349, P07839), thioredoxin m apoprotein (Trx2, X62335), glutamine synthase apoprotein (Gs2, Q42689), LhcII apoproteins (AB051210, AB051208, AB051205), PSII-T apoprotein (PsbT), PSII-S apoprotein (PsbS), PSII-W apoprotein (PsbW), CF₀CF₁ subunit-δapoprotein (AtpC), CF₀CF₁ subunit-6 apoprotein (AtpD, U41442), CFoCF₁ subunit-II apoprotein (AtpG), photosystem I (PSI) apoproteins (such as, of genes PsaD, PsaE, PsaF, PsaG, PsaH, and PsaK), Rubisco SSU apoproteins (such as RbcS2, X04472). Throughout this specification, when reference is made to a transit peptide sequence, such as, for example, any of the transit peptide sequence described above, it includes their functional analogs, modified designer sequences, and combinations thereof. A “functional analog” or “modified designer sequence” in this context refers to a peptide sequence derived or modified (by, e.g., conservative substitution, moderate deletion or addition of amino acids, or modification of side chains of amino acids) based on a native transit peptide sequence, such as those identified above, that has the same function as the native transit peptide sequence, i.e., effecting targeted insertion of a desired enzyme.

In certain specific embodiments, the following transit peptide sequences are used to guide the insertion of the designer butanol-production-pathway enzymes into the stroma region of the chloroplast: the Hyd1 transit peptide (having the amino acid sequence: msalvlkpca avsirgsscr arqvaprapl aastvrvala tleaparrlg nvacaa (SEQ ID NO: 54)), the RbcS2 transit peptides (having the amino acid sequence: maaviakssv saavarpars svrpmaalkp avkaapvaap aqanq (SEQ ID NO: 55)), ferredoxin transit peptide (having the amino acid sequence: mamamrs (SEQ ID NO: 56)), the CF₀CF₁ subunit-δ transit peptide (having the amino acid sequence: mlaaksiagp rafkasavra apkagrrtvv vma (SEQ ID NO: 57)), their analogs, functional derivatives, designer sequences, and combinations thereof.

### Use of a Genetic Switch to Control the Expression of a Designer Butanol-Producing Pathway.

Another key feature of the invention is the application of a genetic switch to control the expression of the designer butanol-producing pathway(s), as illustrated in FIG. 1. This switchability is accomplished through the use of an externally inducible promoter so that the designer transgenes are inducibly expressed under certain specific inducing conditions. Preferably, the promoter employed to control the expression of designer genes in a host is originated from the host itself or a closely related organism. The activities and inducibility of a promoter in a host cell can be tested by placing the promoter in front of a reporting gene, introducing this reporter construct into the host tissue or cells by any of the known DNA delivery techniques, and assessing the expression of the reporter gene.

In a preferred embodiment, the inducible promoter used to control the expression of designer genes is a promoter that is inducible by anaerobiosis, i.e., active under anaerobic conditions but inactive under aerobic conditions. A designer alga/plant organism can perform autotrophic photosynthesis using CO₂ as the carbon source under aerobic conditions, and when the designer organism culture is grown and ready for photosynthetic butanol production, anaerobic conditions will be applied to turn on the promoter and the designer genes that encode a designer butanol-production pathway(s).

A number of promoters that become active under anaerobic conditions are suitable for use in the present invention. For example, the promoters of the hydrogenase genes (HydA1 (Hyd1) and HydA2, GenBank accession number: AJ308413, AF289201, AY090770) of Chlamydomonas reinhardtii, which is active under anaerobic conditions but inactive under aerobic conditions, can be used as an effective genetic switch to control the expression of the designer genes in a host alga, such as Chlamydomonas reinhardtii. In fact, Chlamydomonas cells contain several nuclear genes that are coordinately induced under anaerobic conditions. These include the hydrogenase structural gene itself (Hyd1), the Cyc6 gene encoding the apoprotein of Cytochrome C₆, and the Cpx1 gene encoding coprogen oxidase. The regulatory regions for the latter two have been well characterized, and a region of about 100 bp proves sufficient to confer regulation by anaerobiosis in synthetic gene constructs (Quinn, Barraco, Ericksson and Merchant (2000). “Coordinate copper- and oxygen-responsive Cyc6 and Cpx1 expression in Chlamydomonas is mediated by the same element.” J Biol Chem 275: 6080-6089). Although the above inducible algal promoters may be suitable for use in other plant hosts, especially in plants closely related to algae, the promoters of the homologous genes from these other plants, including higher plants, can be obtained and employed to control the expression of designer genes in those plants.

In another embodiment, the inducible promoter used in the present invention is an algal nitrate reductase (Nia1) promoter, which is inducible by growth in a medium containing nitrate and repressed in a nitrate-deficient but ammonium-containing medium (Loppes and Radoux (2002) “Two short regions of the promoter are essential for activation and repression of the nitrate reductase gene in Chlamydomonas reinhardtii,” Mol Genet Genomics 268: 42-48). Therefore, the Nia1 (gene accession number AF203033) promoter can be selected for use to control the expression of the designer genes in an alga according to the concentration levels of nitrate and ammonium in a culture medium. Additional inducible promoters that can also be selected for use in the present invention include, for example, the heat-shock protein promoter HSP70A (accession number: DQ059999, AY456093, M98823; Schroda, Blocker, Beek (2000) The HSP70A promoter as a tool for the improved expression of transgenes in Chlamydomonas. Plant Journal 21:121-131), the promoter of CabII-1 gene (accession number M24072), the promoter of Ca1 gene (accession number P20507), and the promoter of Ca2 gene (accession number P24258).

In the case of blue-green algae (oxyphotobacteria including cyanobacteria and oxychlorobacteria), there are also a number of inducible promoters that can be selected for use in the present invention. For example, the promoters of the anaerobic-responsive bidirectional hydrogenase hox genes of Nostoc sp. PCC 7120 (GenBank: BA000019), Prochlorothrix hollandica (GenBank: U88400; hoxUYH operon promoter), Synechocystis sp. strain PCC 6803 (CyanoBase: sll1220 and sll1223), Synechococcus elongatus PCC 6301 (CyanoBase: syc1235_c), Arthrospira platensis (GenBank: ABC26906), Cyanothece sp. CCY0110 (GenBank: ZP_{—}01727419) and Synechococcus sp. PCC 7002 (GenBank: AAN03566), which are active under anaerobic conditions but inactive under aerobic conditions (Sjoholm, Oliveira, and Lindblad (2007) “Transcription and regulation of the bidirectional hydrogenase in the Cyanobacterium Nostoc sp. strain PCC 7120,” Applied and Environmental Microbiology, 73(17): 5435-5446), can be used as an effective genetic switch to control the expression of the designer genes in a host oxyphotobacterium, such as Nostoc sp. PCC 7120, Synechocystis sp. strain PCC 6803, Synechococcus elongatus PCC 6301, Cyanothece sp. CCY0110, Arthrospira platensis, or Synechococcus sp. PCC 7002.

In another embodiment in creating switchable butanol-production designer organisms such as switchable designer oxyphotobacteria, the inducible promoter selected for use is a nitrite reductase (nirA) promoter, which is inducible by growth in a medium containing nitrate and repressed in a nitrate-deficient but ammonium-containing medium (Qi, Hao, Ng, Slater, Baszis, Weiss, and Valentin (2005) “Application of the Synechococcus nirA promoter to establish an inducible expression system for engineering the Synechocystis tocopherol pathway,” Applied and Environmental Microbiology, 71(10): 5678-5684; Maeda, Kawaguchi, Ohe, and Omata (1998) “cis-Acting sequences required for NtcB-dependent, nitrite-responsive positive regulation of the nitrate assimilation operon in the Cyanobacterium Synechococcus sp. strain PCC 7942,” Journal of Bacteriology, 180(16):4080-4088). Therefore, the nirA promoter sequences can be selected for use to control the expression of the designer genes in a number of oxyphotobacteria according to the concentration levels of nitrate and ammonium in a culture medium. The nirA promoter sequences that can be selected and modified for use include (but not limited to) the nirA promoters of the following oxyphotobacteria: Synechococcus elongatus PCC 6301 (GenBank: AP008231, region 355890-255950), Synechococcus sp. (GenBank: X67680.1, D16303.1, D12723.1, and D00677), Synechocystis sp. PCC 6803 (GenBank: NP_{—}442378, BA000022, AB001339, D63999-D64006, D90899-D90917), Anabaena sp. (GenBank: X99708.1), Nostoc sp. PCC 7120 (GenBank: BA000019.2 and AJ319648), Plectonema boryanum (GenBank: D31732.1), Synechococcus elongatus PCC 7942 (GenBank: P39661, CP000100.1), Thermosynechococcus elongatus BP-1 (GenBank: BAC08901, NP_{—}682139), Phormidium laminosum (GenBank: CAA79655, Q51879), Mastigocladus laminosus (GenBank: ABD49353, ABD49351, ABD49349, ABD49347), Anabaena variabilis ATCC 29413 (GenBank: YP_{—}325032), Prochlorococcus marinus str. MIT 9303 (GenBank: YP_{—}001018981), Synechococcus sp. WH 8103 (GenBank: AAC17122), Synechococcus sp. WH 7805 (GenBank: ZP_{—}01124915), and Cyanothece sp. CCY0110 (GenBank: ZP_{—}01727861).

In yet another embodiment, an inducible promoter selected for use is the light- and heat-responsive chaperone gene groE promoter, which can be induced by heat and/or light [Kojima and Nakamoto (2007) “A novel light- and heat-responsive regulation of the groE transcription in the absence of HrcA or CIRCE in cyanobacteria,” FEBS Letters 581:1871-1880). A number of groE promoters such as the groES and groEL (chaperones) promoters are available for use as an inducible promoter in controlling the expression of the designer butanol-production-pathway enzymes. The groE promoter sequences that can be selected and modified for use in one of the various embodiments include (but not limited to) the groES and/or groEL promoters of the following oxyphotobacteria: Synechocystis sp. (GenBank: D12677.1), Synechocystis sp. PCC 6803 (GenBank: BA000022.2), Synechococcus elongatus PCC 6301 (GenBank: AP008231.1), Synechococcus sp (GenBank: M58751.1), Synechococcus elongatus PCC 7942 (GenBank: CP000100.1), Nostoc sp. PCC 7120 (GenBank: BA000019.2), Anabaena variabilis ATCC 29413 (GenBank: CP000117.1), Anabaena sp. L-31 (GenBank: AF324500); Thermosynechococcus elongatus BP-1 (CyanoBase: t110185, t110186), Synechococcus vulcanus (GenBank: D78139), Oscillatoria sp. NKBG091600 (GenBank: AF054630), Prochlorococcus marinus MIT9313 (GenBank: BX572099), Prochlorococcus marinus str. MIT 9303 (GenBank: CP000554), Prochlorococcus marinus str. MIT 9211 (GenBank: ZP_{—}01006613), Synechococcus sp. WH8102 (GenBank: BX569690), Synechococcus sp. CC9605 (GenBank: CP000110), Prochlorococcus marinus subsp. marinus str. CCMP1375 (GenBank: AE017126), and Prochlorococcus marinus MED4 (GenBank: BX548174).

Additional inducible promoters that can also be selected for use in the present invention include: for example, the metal (zinc)-inducible smt promoter of Synechococcus PCC 7942 (Erbe, Adams, Taylor and Hall (1996) “Cyanobacteria carrying an smt-lux transcriptional fusion as biosensors for the detection of heavy metal cations,” Journal of Industrial Microbiology, 17:80-83); the iron-responsive idiA promoter of Synechococcus elongatus PCC 7942 (Michel, Pistorius, and Golden (2001) “Unusual regulatory elements for iron deficiency induction of the idiA gene of Synechococcus elongatus PCC 7942” Journal of Bacteriology, 183(17):5015-5024); the redox-responsive cyanobacterial crhR promoter (Patterson-Fortin, Colvin and Owttrim (2006) “A LexA-related protein regulates redox-sensitive expression of the cyanobacterial RNA helicase, crhR”, Nucleic Acids Research, 34(12):3446-3454); the heat-shock gene hsp16.6 promoter of Synechocystis sp. PCC 6803 (Fang and Barnum (2004) “Expression of the heat shock gene hsp16.6 and promoter analysis in the Cyanobacterium, Synechocystis sp. PCC 6803,” Current Microbiology 49:192-198); the small heat-shock protein (Hsp) promoter such as Synechococcus vulcanus gene hspA promoter (Nakamoto, Suzuki, and Roy (2000) “Constitutive expression of a small heat-shock protein confers cellular thermotolerance and thermal protection to the photosynthetic apparatus in cyanobacteria,” FEBS Letters 483:169-174); the CO₂-responsive promoters of oxyphotobacterial carbonic-anhydrase genes (GenBank: EAZ90903, EAZ90685, ZP_{—}01624337, EAW33650, ABB17341, AAT41924, CAO89711, ZP_{—}00111671, YP_{—}400464, AAC44830; and CyanoBase: all2929, PMT1568 slr0051, slr1347, and syc0167_c); the nitrate-reductase-gene (narB) promoters (such as GenBank accession numbers: BAC08907, NP_{—}682145, AAO25121; ABI46326, YP_{—}732075, BAB72570, NP_{—}484656); the green/red light-responsive promoters such as the light-regulated cpcB2A2 promoter of Fremyella diplosiphon (Casey and Grossman (1994) “In vivo and in vitro characterization of the light-regulated cpcB2A2 promoter of Fremyella diplosiphont” Journal of Bacteriology, 176(20):6362-6374); and the UV-light responsive promoters of cyanobacterial genes lexA, recA and ruvB (Domain, Houot, Chauvat, and Cassier-Chauvat (2004) “Function and regulation of the cyanobacterial genes lexA, recA and ruvB: LexA is critical to the survival of cells facing inorganic carbon starvation,” Molecular Microbiology, 53(1):65-80).

Furthermore, in one of the various embodiments, certain “semi-inducible” or constitutive promoters can also be selected for use in combination of an inducible promoter(s) for construction of a designer butanol-production pathway(s) as well. For example, the promoters of oxyphotobacterial Rubisco operon such as the rbcL genes (GenBank: X65960, ZP_{—}01728542, Q3M674, BAF48766, NP_{—}895035, 0907262A; CyanoBase: PMT1205, PMM0550, Pro0551, tll1506, SYNW1718, glr2156, alr1524, slr0009), which have certain light-dependence but could be regarded almost as constitutive promoters, can also be selected for use in combination of an inducible promoter(s) such as the nirA, hox, and/or groE promoters for construction of the designer butanol-production pathway(s) as well.

Throughout this specification, when reference is made to inducible promoter, such as, for example, any of the inducible promoters described above, it includes their analogs, functional derivatives, designer sequences, and combinations thereof. A “functional analog” or “modified designer sequence” in this context refers to a promoter sequence derived or modified (by, e.g., substitution, moderate deletion or addition or modification of nucleotides) based on a native promoter sequence, such as those identified hereinabove, that retains the function of the native promoter sequence.

DNA Constructs and Transformation into Host Organisms

DNA constructs are generated in order to introduce designer butanol-production-pathway genes to a host alga, plant, plant tissue or plant cells. That is, a nucleotide sequence encoding a designer butanol-production-pathway enzyme is placed in a vector, in an operable linkage to a promoter, preferably an inducible promoter, and in an operable linkage to a nucleotide sequence coding for an appropriate chloroplast-targeting transit-peptide sequence. In a preferred embodiment, nucleic acid constructs are made to have the elements placed in the following 5′ (upstream) to 3′ (downstream) orientation: an externally inducible promoter, a transit targeting sequence, and a nucleic acid encoding a designer butanol-production-pathway enzyme, and preferably an appropriate transcription termination sequence. One or more designer genes (DNA constructs) can be placed into one genetic vector. An example of such a construct is depicted in FIG. 2A. As shown in the embodiment illustrated in FIG. 2A, a designer butanol-production-pathway transgene is a nucleic acid construct comprising: a) a PCR forward primer; b) an externally inducible promoter; c) a transit targeting sequence; d) a designer butanol-production-pathway-enzyme-encoding sequence with an appropriate transcription termination sequence; and e) a PCR reverse primer.

In accordance with various embodiments, any of the components a) through e) of this DNA construct are adjusted to suit for certain specific conditions. In practice, any of the components a) through e) of this DNA construct are applied in full or in part, and/or in any adjusted combination to achieve more desirable results. For example, when an algal hydrogenase promoter is used as an inducible promoter in the designer butanol-production-pathway DNA construct, a transgenic designer alga that contains this DNA construct will be able to perform autotrophic photosynthesis using ambient-air CO₂ as the carbon source and grows normally under aerobic conditions, such as in an open pond. When the algal culture is grown and ready for butanol production, the designer transgene(s) can then be expressed by induction under anaerobic conditions because of the use of the hydrogenase promoter. The expression of designer gene(s) produces a set of designer butanol-production-pathway enzymes to work with the Calvin cycle for photobiological butanol production (FIG. 1).

The two PCR primers are a PCR forward primer (PCR FD primer) located at the beginning (the 5′ end) of the DNA construct and a PCR reverse primer (PCR RE primer) located at the other end (the 3′ end) as shown in FIG. 2A. This pair of PCR primers is designed to provide certain convenience when needed for relatively easy PCR amplification of the designer DNA construct, which is helpful not only during and after the designer DNA construct is synthesized in preparation for gene transformation, but also after the designer DNA construct is delivered into the genome of a host alga for verification of the designer gene in the transformants. For example, after the transformation of the designer gene is accomplished in a Chlamydomonas reinhardtii-arg7 host cell using the techniques of electroporation and argininosuccinate lyase (arg7) complementation screening, the resulted transformants can be then analyzed by a PCR DNA assay of their nuclear DNA using this pair of PCR primers to verify whether the entire designer butanol-production-pathway gene (the DNA construct) is successfully incorporated into the genome of a given transformant. When the nuclear DNA PCR assay of a transformant can generate a PCR product that matches with the predicted DNA size and sequence according to the designer DNA construct, the successful incorporation of the designer gene(s) into the genome of the transformant is verified.

Therefore, the various embodiments also teach the associated method to effectively create the designer transgenic algae, plants, or plant cells for photobiological butanol production. This method, in one of embodiments, includes the following steps: a) Selecting an appropriate host alga, plant, plant tissue, or plant cells with respect to their genetic backgrounds and special features in relation to butanol production; b) Introducing the nucleic acid constructs of the designer genes into the genome of said host alga, plant, plant tissue, or plant cells; c) Verifying the incorporation of the designer genes in the transformed alga, plant, plant tissue, or plant cells with DNA PCR assays using the said PCR primers of the designer DNA construct; d) Measuring and verifying the designer organism features such as the inducible expression of the designer butanol-pathway genes for photosynthetic butanol production from carbon dioxide and water by assays of mRNA, protein, and butanol-production characteristics according to the specific designer features of the DNA construct(s) (FIG. 2A).

The above embodiment of the method for creating the designer transgenic organism for photobiological butanol production can also be repeatedly applied for a plurality of operational cycles to achieve more desirable results. In various embodiments, any of the steps a) through d) of this method described above are adjusted to suit for certain specific conditions. In various embodiments, any of the steps a) through d) of the method are applied in full or in part, and/or in any adjusted combination.

Examples of designer butanol-production-pathway genes (DNA constructs) are shown in the sequence listings. SEQ ID NO: 1 presents a detailed DNA construct of a designer Butanol Dehydrogenase gene (1809 bp) that includes a PCR FD primer (sequence 1-20), a 262-bp nitrate reductase Nia1 promoter (21-282), a 135-bp RbcS2 transit peptide (283-417), an enzyme-encoding sequence (418-1566) selected and modified from a Clostridium saccharoperbutylacetonicum Butanol Dehydrogenase sequence (AB257439), a 223-bp RbcS2 terminator (1567-1789), and a PCR RE primer (1790-1809). The 262-bp Nia1 promoter (DNA sequence 21-282) is used as an example of an inducible promoter to control the expression of a designer butanol-production-pathway Butanol Dehydrogenase gene (DNA sequence 418-1566). The 135-bp RbcS2 transit peptide (DNA sequence 283-417) is used as an example to guide the insertion of the designer enzyme (DNA sequence 418-1566) into the chloroplast of the host organism. The RbcS2 terminator (DNA sequence 1567-1789) is employed so that the transcription and translation of the designer gene is properly terminated to produce the designer apoprotein (RbcS2 transit peptide-Butanol Dehydrogenase) as desired. Because the Nia1 promoter is a nuclear DNA that can control the expression only for nuclear genes, the synthetic butanol-production-pathway gene in this example is designed according to the codon usage of Chlamydomonas nuclear genome. Therefore, in this case, the designer enzyme gene is transcribed in nucleus. Its mRNA is naturally translocated into cytosol, where the mRNA is translated to an apoprotein that consists of the RbcS2 transit peptide (corresponding to DNA sequence 283-417) with its C-terminal end linked together with the N-terminal end of the Butanol Dehydrogenase protein (corresponding to DNA sequence 418-1566). The transit peptide of the apoprotein guides its transportation across the chloroplast membranes and into the stroma area, where the transit peptide is cut off from the apoprotein. The resulting Butanol Dehydrogenase then resumes its function as an enzyme for the designer butanol-production pathway in chloroplast. The two PCR primers (sequences 1-20 and 1790-1809) are selected and modified from the sequence of a Human actin gene and can be paired with each other. Blasting the sequences against Chlamydomonas GenBank found no homologous sequences of them. Therefore, they can be used as appropriate PCR primers in DNA PCR assays for verification of the designer gene in the transformed alga.

SEQ ID NO: 2 presents example 2 for a designer Butyraldehyde Dehydrogenase DNA construct (2067 bp) that includes a PCR FD primer (sequence 1-20), a 262-bp nitrate reductase Nia1 promoter (21-282), a 135-bp RbcS2 transit peptide (283-417), a Butyraldehyde Dehydrogenase-encoding sequence (418-1824) selected and modified from a Clostridium saccharoperbutylacetonicum Butyraldehyde Dehydrogenase sequence (AY251646), a 223-bp RbcS2 terminator (1825-2047), and a PCR RE primer (2048-2067). This DNA construct is similar to example 1, SEQ ID NO: 1, except that a Butyraldehyde Dehydrogenase-encoding sequence (418-1824) selected and modified from a Clostridium saccharoperbutylacetonicum Butyraldehyde Dehydrogenase sequence (AY251646) is used.

SEQ ID NO: 3 presents example 3 for a designer Butyryl-CoA Dehydrogenase construct (1815 bp) that includes a PCR FD primer (sequence 1-20), a 262-bp nitrate reductase promoter (21-282), a 9-bp Xho I NdeI site (283-291), a 135-bp RbcS2 transit peptide (292-426), a Butyryl-CoA Dehydrogenase encoding sequence (427-1563) selected/modified from the sequences of a Clostridium beijerinckii Butyryl-CoA Dehydrogenase (AF494018), a 9-bp XbaI site (1564-1572), a 223-bp RbcS2 terminator (1573-1795), and a PCR RE primer (1796-1815) at the 3′ end. This DNA construct is similar to example 1, SEQ ID NO: 1, except that a Butyryl-CoA Dehydrogenase encoding sequence (427-1563) selected/modified from the sequences of a Clostridium beijerinckii Butyryl-CoA Dehydrogenase (AF494018) is used and restriction sites of Xho I NdeI and XbaI are added to make the key components such as the targeting sequence (292-426) and the designer enzyme sequence (427-1563) as a modular unit that can be flexible replaced when necessary to save cost of gene synthesis and enhance work productivity. Please note, the enzyme does not have to be Clostridium beijerinckii Butyryl-CoA Dehydrogenase; a number of butyryl-CoA dehydrogenase enzymes (such as those listed in Table 1) including their isozymes, designer modified enzymes, and functional analogs from other sources such as Butyrivibrio fibrisolvens, Butyrate producing bacterium L2-50, Thermoanaerobacterium thermosaccharolyticum, can also be selected for use.

SEQ ID NO: 4 presents example 4 for a designer Crotonase DNA construct (1482 bp) that includes a PCR FD primer (sequence 1-20), a 262-bp nitrate reductase promoter (21-282), a 9-bp Xho I NdeI site (283-291) a 135-bp RbcS2 transit peptide (292-426), a Crotonase-encoding sequence (427-1209) selected/modified from the sequences of a Clostridium beijerinckii Crotonase (Genbank: AF494018), a 21-bp Lumio-tag-encoding sequence (1210-1230), a 9-bp XbaI site (1231-1239) containing a stop codon, a 223-bp RbcS2 terminator (1240-1462), and a PCR RE primer (1463-1482) at the 3′ end. This DNA construct is similar to example 3, SEQ ID NO: 3, except that a Crotonase-encoding sequence (427-1209) selected/modified from the sequences of a Clostridium beijerinckii Crotonase (Genbank: AF494018) is used and a 21-bp Lumio-tag-encoding sequence (1210-1230) is added at the C-terminal end of the enolase sequence. The 21-bp Lumio-tag sequence (1210-1230) is employed here to encode a Lumio peptide sequence Gly-Cys-Cys-Pro-Gly-Cys-Cys, which can become fluorescent when treated with a Lumio reagent that is now commercially available from Invitrogen [https://catalog.invitrogen.com]. Lumio molecular tagging technology is based on an EDT (1,2-ethanedithiol) coupled biarsenical derivative (the Lumio reagent) of fluorescein that binds to an engineered tetracysteine sequence (Keppetipola, Coffman, and et al (2003). Rapid detection of in vitro expressed proteins using Lumio™ technology, Gene Expression, 25.3:7-11). The tetracysteine sequence consists of Cys-Cys-Xaa-Xaa-Cys-Cys, where Xaa is any non-cysteine amino acid such as Pro or Gly in this example. The EDT-linked Lumio reagent allows free rotation of the arsenic atoms that quenches the fluorescence of fluorescein. Covalent bond formation between the thiols of the Lumio's arsenic groups and the tetracysteines prevents free rotation of arsenic atoms that releases the fluorescence of fluorescein (Griffin, Adams, and Tsien (1998), “Specific covalent labeling of recombinant protein molecules inside live cells”, Science, 281:269-272). This also permits the visualization of the tetracysteine-tagged proteins by fluorescent molecular imaging. Therefore, use of the Lumio tag in this manner enables monitoring and/or tracking of the designer Crotonase when expressed to verify whether the designer butanol-production pathway enzyme is indeed delivered into the chloroplast of a host organism as designed. The Lumio tag (a short 7 amino acid peptide) that is linked to the C-terminal end of the Crotonase protein in this example should have minimal effect on the function of the designer enzyme, but enable the designer enzyme molecule to be visualized when treated with the Lumio reagent. Use of the Lumio tag is entirely optional. If the Lumio tag somehow affects the designer enzyme function, this tag can be deleted in the DNA sequence design.

SEQ ID NO: 5 presents example 5 for a designer 3-Hydroxybutyryl-CoA Dehydrogenase DNA construct (1367 bp) that includes a PCR FD primer (sequence 1-20), a 84-bp nitrate reductase promoter (21-104), a 9-bp Xho I NdeI site (105-113) a 135-bp RbcS2 transit peptide (114-248), a 3-Hydroxybutyryl-CoA Dehydrogenase-encoding sequence (249-1094) selected/modified from a Clostridium beijerinckii 3-Hydroxybutyryl-CoA Dehydrogenase sequence (Genbank: AF494018), a 21-bp Lumio-tag sequence (1095-1115), a 9-bp XbaI site (1116-1124), a 223-bp RbcS2 terminator (1125-1347), and a PCR RE primer (1348-1367). This DNA construct is similar to example 4, SEQ ID NO: 4, except that an 84-bp nitrate reductase promoter (21-104) and a 3-Hydroxybutyryl-CoA Dehydrogenase-encoding sequence (249-1094) selected/modified from a Clostridium beijerinckii 3-Hydroxybutyryl-CoA Dehydrogenase sequence (Genbank: AF494018) are used. The 84-bp nitrate-reductase promoter is artificially created by joining two partially homologous sequence regions (−231 to −201 and -77 to −25 with respect to the start site of transcription) of the native Chlamydomonas reinhardtii Nia1 promoter. Experimental studies have demonstrated that the 84-bp sequence is more active than the native Nia1 promoter (Loppes and Radoux (2002) “Two short regions of the promoter are essential for activation and repression of the nitrate reductase gene in Chlamydomonas reinhardtii,” Mol Genet Genomics 268: 42-48). Therefore, this is also an example where functional synthetic sequences, analogs, functional derivatives and/or designer modified sequences such as the synthetic 84-bp sequence can be selected for use according to various embodiments in this invention.

SEQ ID NO: 6 presents example 6 for a designer Thiolase DNA construct (1721 bp) that includes a PCR FD primer (sequence 1-20), a 84-bp nitrate reductase promoter (21-104), a 9-bp Xho I NdeI site (105-113) a 135-bp RbcS2 transit peptide (114-248), a Thiolase-encoding sequence (248-1448) selected/modified from a Butyrivibrio fibrisolvens Thiolase sequence (AB190764), a 21-bp Lumio-tag sequence (1449-1469), a 9-bp XbaI site (1470-1478), a 223-bp RbcS2 terminator (1479-1701), and a PCR RE primer (1702-1721). This DNA construct is also similar to example 4, SEQ ID NO: 4, except that a Thiolase-encoding-encoding sequence (249-1448) and an 84-bp synthetic Nia1 promoter (21-104) are used. This is another example that functional synthetic sequences can also be selected for use in designer DNA constructs.

SEQ ID NO: 7 presents example 7 for a designer Pyruvate-Ferredoxin Oxidoreductase DNA construct (4211 bp) that includes a PCR FD primer (sequence 1-20), a 2×84-bp nitrate reductase promoter (21-188), a 9-bp Xho I NdeI site (189-197) a 135-bp RbcS2 transit peptide (198-332), a Pyruvate-Ferredoxin Oxidoreductase-encoding sequence (333-3938) selected/modified from the sequences of a Mastigamoeba balamuthi Pyruvate-ferredoxin oxidoreductase (GenBank: AY101767), a 21-bp Lumio-tag sequence (3939-3959), a 9-bp XbaI site (3960-3968), a 223-bp RbcS2 terminator (3969-4191), and a PCR RE primer (4192-4211). This DNA construct is also similar to example 4, SEQ ID NO: 4, except a designer 2×84-bp Nia1 promoter and a Pyruvate-Ferredoxin Oxidoreductase-encoding sequence (333-3938) selected/modified from the sequences of a Mastigamoeba balamuthi Pyruvate-ferredoxin oxidoreductase (GenBank: AY101767) are used. The 2×84-bp Nia1 promoter is constructed as a tandem duplication of the 84-bp synthetic Nia1 promoter sequence presented in SEQ ID NO: 6 above. Experimental tests have shown that the 2×84-bp synthetic Nia1 promoter is even more powerful than the 84-bp sequence which is more active than the native Nia1 promoter (Loppes and Radoux (2002) “Two short regions of the promoter are essential for activation and repression of the nitrate reductase gene in Chlamydomonas reinhardtii,” Mol Genet Genomics 268: 42-48). Use of this type of inducible promoter sequences with various promoter strengths can also help in adjusting the expression levels of the designer enzymes for the butanol-production pathway(s).

SEQ ID NO: 8 presents example 8 for a designer Pyruvate Kinase DNA construct (2021 bp) that includes a PCR FD primer (sequence 1-20), a 84-bp nitrate reductase promoter (21-104), a 9-bp Xho I NdeI site (105-113) a 135-bp RbcS2 transit peptide (114-248), a pyruvate kinase-encoding sequence (249-1748) selected/modified from a Saccharomyces cerevisiae Pyruvate Kinase sequence (GenBank: AY949876), a 21-bp Lumio-tag sequence (1749-1769), a 9-bp XbaI site (1770-1778), a 223-bp RbcS2 terminator (1779-2001), and a PCR RE primer (2002-2021). This DNA construct is similar to example 6, SEQ ID NO: 6, except that a pyruvate kinase-encoding sequence (249-1748) is used.

SEQ ID NO: 9 presents example 9 for a designer Enolase gene (1815 bp) consisting of a PCR FD primer (sequence 1-20), a 262-bp nitrate reductase promoter (21-282), a 9-bp Xho I NdeI site (283-291) a 135-bp RbcS2 transit peptide (292-426), a enolase-encoding sequence (427-1542) selected/modified from the sequences of a Chlamydomonas reinhardtii cytosolic enolase (Genbank: X66412, P31683), a 21-bp Lumio-tag-encoding sequence (1507-1527), a 9-bp XbaI site (1543-1551) containing a stop codon, a 223-bp RbcS2 terminator (1552-1795), and a PCR RE primer (1796-1815) at the 3′ end. This DNA construct is similar to example 3, SEQ ID NO: 3, except that an enolase-encoding sequence (427-1542) selected/modified from the sequences of a Chlamydomonas reinhardtii cytosolic enolase is used.

SEQ ID NO: 10 presents example 10 for a designer Phosphoglycerate-Mutase DNA construct (2349 bp) that includes a PCR FD primer (sequence 1-20), a 262-bp nitrate reductase promoter (21-282), a 9-bp Xho I NdeI site (283-291), a 135-bp RbcS2 transit peptide (292-426), a phosphoglycerate-mutase encoding sequence (427-2097) selected/modified from the sequences of a Chlamydomonas reinhardtii cytosolic phosphoglycerate mutase (JGI Chlre2 protein ID 161689, Genbank: AF268078), a 9-bp XbaI site (2098-2106), a 223-bp RbcS2 terminator (2107-2329), and a PCR RE primer (2330-2349) at the 3′ end. This DNA construct is similar to example 3, SEQ ID NO: 3, except that a phosphoglycerate-mutase encoding sequence (427-2097) selected/modified from the sequences of a Chlamydomonas reinhardtii cytosolic phosphoglycerate mutase is used.

SEQ ID NO: 11 presents example 11 for a designer Phosphoglycerate Kinase DNA construct (1908 bp) that includes a PCR FD primer (sequence 1-20), a 262-bp nitrate reductase Nia1 promoter (21-282), a phosphoglycerate-kinase-encoding sequence (283-1665) selected from a Chlamydomonas reinhardtii chloroplast phosphoglycerate-kinase sequence including its chloroplast signal peptide and mature enzyme sequence (GenBank: U14912), a 223-bp RbcS2 terminator (1666-1888), and a PCR RE primer (1889-1908). This DNA construct is similar to example 1, SEQ ID NO: 1, except a phosphoglycerate-kinase-encoding sequence (283-1665) selected from a Chlamydomonas reinhardtii chloroplast phosphoglycerate-kinase sequence including its chloroplast signal peptide and mature enzyme sequence is used. Therefore, this is also an example where the sequence of a nuclear-encoded chloroplast enzyme such as the Chlamydomonas reinhardtii chloroplast phosphoglycerate kinase can also be used in design and construction of a designer butanol-production pathway gene when appropriate with a proper inducible promoter such as the Nia1 promoter (DNA sequence 21-282).

SEQ ID NO: 12 presents example 12 for a designer Glyceraldehyde-3-Phosphate Dehydrogenase gene (1677 bp) that includes a PCR FD primer (sequence 1-20), a 262-bp nitrate reductase Nia1 promoter (21-282), a 135-bp RbcS2 transit peptide (283-417), an enzyme-encoding sequence (418-1434) selected and modified from a Mesostigma viride cytosolic glyceraldehyde-3-phosphate dehydrogenase (mRNA) sequence (GenBank accession number DQ873404), a 223-bp RbcS2 terminator (1435-1657), and a PCR RE primer (1658-1677). This DNA construct is similar to example 1, SEQ ID NO: 1, except that an enzyme-encoding sequence (418-1434) selected and modified from a Mesostigma viride cytosolic glyceraldehyde-3-phosphate dehydrogenase (mRNA) sequence (GenBank accession number DQ873404) is used.

SEQ ID NO: 13 presents example 13 for a designer HydA1-promoter-linked Phosphoglycerate Mutase DNA construct (2351 bp) that includes a PCR FD primer (sequence 1-20), a 282-bp HydA1 promoter (21-302), a 135-bp RbcS2 transit peptide (303-437), a phosphoglycerate-mutase encoding sequence (438-2108) selected/modified from the sequences of a Chlamydomonas reinhardtii cytosolic phosphoglycerate mutase (JGI Chlre2 protein ID 161689, Genbank: AF268078), a 223-bp RbcS2 terminator (2109-2331), and a PCR RE primer (2332-2351). This designer DNA construct is quite similar to example 1, SEQ ID NO:1, except that a 282-bp HydA1 promoter (21-302) and a phosphoglycerate-mutase encoding sequence (438-2108) selected/modified from the sequences of a Chlamydomonas reinhardtii cytosolic phosphoglycerate mutase are used. The 282-bp HydA1 promoter (21-302) has been proven active by experimental assays at the inventor's laboratory. Use of the HydA1 promoter (21-302) enables activation of designer enzyme expression by using anaerobic culture-medium conditions.

With the same principle of using an inducible anaerobic promoter and a chloroplast-targeting sequence as that shown in SEQ ID NO: 13 (example 13), SEQ ID NOS: 14-23 show designer-gene examples 14-23. Briefly, SEQ ID NO: 14 presents example 14 for a designer HydA1-promoter-linked Enolase DNA construct (1796 bp) that includes a PCR FD primer (sequence 1-20), a 282-bp HydA1 promoter (21-302), a 135-bp RbcS2 transit peptide (303-437), a Enolase-encoding sequence (438-1553) selected/modified from the sequences of a Chlamydomonas reinhardtii cytosolic enolase (Genbank: X66412, P31683), a 223-bp RbcS2 terminator (1554-1776), and a PCR RE primer (1777-1796).

SEQ ID NO: 15 presents example 15 for a designer HydA1-promoter-controlled Pyruvate-Kinase DNA construct that includes a PCR FD primer (sequence 1-20), a 282-bp HydA1 promoter (21-302), a 135-bp RbcS2 transit peptide (303-437), a Pyruvate Kinase-encoding sequence (438-1589) selected/modified from a Chlamydomonas reinhardtii cytosolic pyruvate kinase sequence (JGI Chlre3 protein ID 138105), a 223-bp RbcS2 terminator (1590-1812), and a PCR RE primer (1813-1832).

SEQ ID NO:16 presents example 16 for a designer HydA1-promoter-linked Pyruvate-ferredoxin oxidoreductase DNA construct (4376 bp) that includes a PCR FD primer (sequence 1-20), a 282-bp HydA1 promoter (21-302), a 135-bp RbcS2 transit peptide (303-437), a Pyruvate-ferredoxin oxidoreductase-encoding sequence (438-4133) selected/modified from a Desulfovibrio africanus Pyruvate-ferredoxin oxidoreductase sequence (GenBank Accession Number Y09702), a 223-bp RbcS2 terminator (4134-4356), and a PCR RE primer (4357-4376).

SEQ ID NO:17 presents example 17 for a designer HydA1-promoter-linked Pyruvate-NADP⁺ oxidoreductase DNA construct (6092 bp) that includes a PCR FD primer (sequence 1-20), a 282-bp HydA1 promoter (21-302), a 135-bp RbcS2 transit peptide (303-437), a Pyruvate-NADP⁺ oxidoreductase-encoding sequence (438-5849) selected/modified from a Euglena gracilis Pyruvate-NADP⁺ oxidoreductase sequence (GenBank Accession Number AB021127), a 223-bp RbcS2 terminator (5850-6072), and a PCR RE primer (6073-6092).

SEQ ID NO:18 presents example 18 for a designer HydA1-promoter-linked Thiolase DNA construct (1856 bp) that includes a PCR FD primer (sequence 1-20), a 282-bp HydA1 promoter (21-302), a 135-bp RbcS2 transit peptide (303-437), a Thiolase-encoding sequence (438-1613) selected/modified from the sequences of a Thermoanaerobacterium thermosaccharolyticum Thiolase (GenBank Z92974), a 223-bp RbcS2 terminator (1614-1836), and a PCR RE primer (1837-1856).

SEQ ID NO:19 presents example 19 for a designer HydA1-promoter-linked 3-Hydroxybutyryl-CoA dehydrogenase DNA construct (1550 bp) that includes a PCR FD primer (sequence 1-20), a 282-bp HydA1 promoter (21-302), a 135-bp RbcS2 transit peptide (303-437), a 3-Hydroxybutyryl-CoA dehydrogenase-encoding sequence (438-1307) selected/modified from the sequences of a Thermoanaerobacterium thermosaccharolyticum 3-Hydroxybutyryl-CoA dehydrogenase (GenBank Z92974), a 223-bp RbcS2 terminator (1308-1530), and a PCR RE primer (1531-1550).

SEQ ID NO:20 presents example 20 for a designer HydA1-promoter-linked Crotonase DNA construct (1457 bp) that includes a PCR FD primer (sequence 1-20), a 282-bp HydA1 promoter (21-302), a 135-bp RbcS2 transit peptide (303-437), a Crotonase-encoding sequence (438-1214) selected/modified from the sequences of a Thermoanaerobacterium thermosaccharolyticum Crotonase (GenBank Z92974), a 223-bpRbcS2 terminator (1215-1437), and a PCR RE primer (1438-1457).

SEQ ID NO:21 presents example 21 for a designer HydA1-promoter-linked Butyryl-CoA dehydrogenase DNA construct (1817 bp) that includes a PCR FD primer (sequence 1-20), a 282-bp HydA1 promoter (21-302), a 135-bp RbcS2 transit peptide (303-437), a Butyryl-CoA dehydrogenase-encoding sequence (438-1574) selected/modified from the sequences of a Thermoanaerobacterium thermosaccharolyticum Butyryl-CoA dehydrogenase (GenBank Z92974), a 223-bp RbcS2 terminator (1575-1797), and a PCR RE primer (1798-1817).

SEQ ID NO: 22 presents example 22 for a designer HydA1-promoter-linked Butyraldehyde dehydrogenase DNA construct (2084 bp) that includes a PCR FD primer (sequence 1-20), a 282-bp HydA1 promoter (21-302), a 135-bp RbcS2 transit peptide (303-437), a Butyraldehyde dehydrogenase-encoding sequence (438-1841) selected/modified from the sequences of a Clostridium saccharoperbutylacetonicum Butyraldehyde dehydrogenase (GenBank AY251646), a 223-bp RbcS2 terminator (1842-2064), and a PCR RE primer (2065-2084).

SEQ ID NO: 23 presents example 23 for a designer HydA1-promoter-linked Butanol dehydrogenase DNA construct (1733 bp) that includes a PCR FD primer (sequence 1-20), a 282-bp HydA1 promoter (21-302), a 135-bp RbcS2 transit peptide (303-437), a Butanol dehydrogenase-encoding sequence (438-1490) selected/modified from the sequences of a Clostridium beijerinckii Butanol dehydrogenase (GenBank AF157307), a 223-bp RbcS2 terminator (1491-1713), and a PCR RE primer (1714-1733).

With the same principle of using a 2×84 synthetic Nia1 promoter and a chloroplast-targeting mechanism as mentioned previously, SEQ ID NOS:24-26 show more examples of designer-enzyme DNA-constructs. Briefly, SEQ ID NO: 24 presents example 24 for a designer Fructose-Diphosphate-Aldolase DNA construct that includes a PCR FD primer (sequence 1-20), a 2×84-bp NR promoter (21-188), a Fructose-Diphosphate Aldolase-encoding sequence (189-1313) selected/modified from a C. reinhardtii chloroplast fructose-1,6-bisphosphate aldolase sequence (GenBank: X69969), a 223-bpRbcS2 terminator (1314-1536), and a PCR RE primer (1537-1556).

SEQ ID NO: 25 presents example 24 for a designer Triose-Phosphate-Isomerase DNA construct that includes a PCR FD primer (sequence 1-20), a 2×84-bp NR promoter (21-188), a Triose-Phosphate Isomerase-encoding sequence (189-1136) selected and modified from a Arabidopsis thaliana chloroplast triosephosphate-isomerase sequence (GenBank: AF247559), a 223-bp RbcS2 terminator (1137-1359), and a PCR RE primer (1360-1379).

SEQ ID NO: 26 presents example 26 for a designer Phosphofructose-Kinase DNA construct that includes a PCR FD primer (sequence 1-20), a 2×84-bp NR promoter (21-188), a 135-bp RbcS2 transit peptide (189-323), a Phosphofructose Kinase-encoding sequence (324-1913) selected/modified from Arabidopsis thaliana 6-phosphofructokinase sequence (GenBank: NM_{—}001037043), a 223-bp RbcS2 terminator (1914-2136), and a PCR RE primer (2137-2156).

The nucleic acid constructs, such as those presented in the examples above, may include additional appropriate sequences, for example, a selection marker gene, and an optional biomolecular tag sequence (such as the Lumio tag described in example 4, SEQ ID NO: 4). Selectable markers that can be selected for use in the constructs include markers conferring resistances to kanamycin, hygromycin, spectinomycin, streptomycin, sulfonyl urea, gentamycin, chloramphenicol, among others, all of which have been cloned and are available to those skilled in the art. Alternatively, the selective marker is a nutrition marker gene that can complement a deficiency in the host organism. For example, the gene encoding argininosuccinate lyase (arg7) can be used as a selection marker gene in the designer construct, which permits identification of transformants when Chlamydomonas reinhardtii arg7-(minus) cells are used as host cells.

Nucleic acid constructs carrying designer genes can be delivered into a host alga, blue-green alga, plant, or plant tissue or cells using the available gene-transformation techniques, such as electroporation, PEG induced uptake, and ballistic delivery of DNA, and Agrobacterium-mediated transformation. For the purpose of delivering a designer construct into algal cells, the techniques of electroporation, glass bead, and biolistic genegun can be selected for use as preferred methods; and an alga with single cells or simple thallus structure is preferred for use in transformation. Transformants can be identified and tested based on routine techniques.

The various designer genes can be introduced into host cells sequentially in a step-wise manner, or simultaneously using one construct or in one transformation. For example, the ten DNA constructs shown in SEQ ID NO: 13-16 (or 17) and 18-23 for the ten-enzyme 3-phosphoglycerate-branched butanol-production pathway can be placed into a genetic vector such as p389-Arg7 with a single selection marker (Arg7). Therefore, by use of a plasmid in this manner, it is possible to deliver all the ten DNA constructs (designer genes) into an arginine-requiring Chlamydomonas reinhardtii-arg7 host (CC-48) in one transformation for expression of the 3-phosphoglycerate-branched butanol-production pathway (03-12 in FIG. 1). When necessary, a transformant containing the ten DNA constructs can be further transformed to get more designer genes into its genomic DNA with an additional selection marker such as streptomycin. By using combinations of various designer-enzymes DNA constructs such as those presented in SEQ ID NO: 1-26 in genetic transformation with an appropriate host organism, various butanol-production pathways such as those illustrated in FIG. 1 can be constructed. For example, the designer DNA constructs of SEQ ID NO: 1-12 can be selected for construction of the glyceraldehydes-3-phosphate-branched butanol-production pathway (01-12 in FIG. 1); The designer DNA constructs of SEQ ID NO: 1-12, 24, and 25 can be selected for construction of the fructose-1,6-diphosphate-branched butanol-production pathway (20-33); and the designer DNA constructs of SEQ ID NO: 1-12 and 24-26 can be selected for construction of the fructose-6-phosphate-branched butanol-production pathway (19-33).

### Additional Host Modifications to Enhance Photosynthetic Butanol Production

### An NADPH/NADH Conversion Mechanism

According to the photosynthetic butanol production pathway(s), to produce one molecule of butanol from 4CO₂ and 5H₂O is likely to require 14 ATP and 12 NADPH, both of which are generated by photosynthetic water splitting and photophosphorylation across the thylakoid membrane. In order for the 3-phosphoglycerate-branched butanol-production pathway (03-12 in FIG. 1) to operate, it is a preferred practice to use a butanol-production-pathway enzyme(s) that can use NADPH that is generated by the photo-driven electron transport process. Clostridium saccharoperbutylacetonicum butanol dehydrogenase (GenBank accession number: AB257439) and butyaldehyde dehydrogenase (GenBank: AY251646) are examples of a butanol-production-pathway enzyme that is capable of accepting either NADP(H) or NAD(H). Such a butanol-production-pathway enzyme that can use both NADPH and NADH (i.e., NAD(P)H) can also be selected for use in this 3-phosphoglycerate-branched and any of the other designer butanol-production pathway(s) (FIG. 1) as well. Clostridium beijerinckii Butyryl-CoA dehydrogenase (GenBank: AF494018) and 3-Hydroxybutyryl-CoA dehydrogenase (GenBank: AF494018) are examples of a butanol-production-pathway enzyme that can accept only NAD(H). When a butanol-production-pathway enzyme that can only use NADH is employed, it may require an NADPH/NADH conversion mechanism in order for this 3-phosphoglycerate-branched butanol-production pathway to operate well. However, depending on the genetic backgrounds of a host organism, a conversion mechanism between NADPH and NADH may exist in the host so that NADPH and NADH may be interchangeably used in the organism. In addition, it is known that NADPH could be converted into NADH by a NADPH-phosphatase activity (Pattanayak and Chatterjee (1998) “Nicotinamide adenine dinucleotide phosphate phosphatase facilitates dark reduction of nitrate: regulation by nitrate and ammonia,” Biologia Plantarium 41(1):75-84) and that NAD can be converted to NADP by a NAD kinase activity (Muto, Miyachi, Usuda, Edwards and Bassham (1981) “Light-induced conversion of nicotinamide adenine dinucleotide to nicotinamide adenine dinucleotide phosphate in higher plant leaves,” Plant Physiology 68(2):324-328; Matsumura-Kadota, Muto, Miyachi (1982) “Light-induced conversion of NAD⁺ to NADP⁺ in Chlorella cells,” Biochimica Biophysica Acta 679(2):300-300). Therefore, when enhanced NADPH/NADH conversion is desirable, the host may be genetically modified to enhance the NADPH phosphatase and NAD kinase activities. Thus, in one of the various embodiments, the photosynthetic butanol-producing designer plant, designer alga or plant cell further contains additional designer transgenes (FIG. 2B) to inducibly express one or more enzymes to facilitate the NADPH/NADH inter-conversion, such as the NADPH phosphatase and NAD kinase (GenBank: XM_{—}001609395, XM_{—}001324239), in the stroma of algal chloroplast.

Another embodiment that can provide an NADPH/NADH conversion mechanism is by properly selecting an appropriate branching point at the Calvin cycle for a designer butanol-production pathway to branch from. To confer this NADPH/NADH conversion mechanism by pathway design according to this embodiment, it is a preferred practice to branch a designer butanol-production pathway at or after the point of glyceraldehydes-3-phosphate of the Calvin cycle as shown in FIG. 1. In these pathway designs, the NADPH/NADH conversion is achieved essentially by a two-step mechanism: 1) Use of the step with the Calvin-cycle's glyceraldehyde-3-phosphate dehydrogenase, which uses NADPH in reducing1,3-diphosphoglycerate to glyceraldehydes-3-phosphate; and 2) use of the step with the designer pathway's NAD⁺-dependent glyceraldehyde-3-phosphate dehydrogenase 01, which produces NADH in oxidizing glyceraldehyde-3-phosphate to 1,3-diphosphoglycerate. The net result of the two steps described above is the conversion of NADPH to NADH, which can supply the needed reducing power in the form of NADH for the designer butanol-production pathway(s). For step 1), use of the Calvin-cycle's NADPH-dependent glyceraldehyde-3-phosphate dehydrogenase naturally in the host organism is usually sufficient. Consequently, introduction of a designer NAD⁺-dependent glyceraldehyde-3-phosphate dehydrogenase 01 to work with the Calvin-cycle's NADPH-dependent glyceraldehyde-3-phosphate dehydrogenase may confer the function of an NADPH/NADH conversion mechanism, which is needed for the 3-phosphoglycerate-branched butanol-production pathway (03-12 in FIG. 1) to operate well. For this reason, the designer NAD⁺-dependent glyceraldehyde-3-phosphate-dehydrogenase DNA construct (example 12, SEQ ID NO:12) is used also as an NADPH/NADH-conversion designer gene (FIG. 2B) to support the 3-phosphoglycerate-branched butanol-production pathway (03-12 in FIG. 1) in one of the various embodiments. This also explains why it is important to use a NAD⁺-dependent glyceraldehyde-3-phosphate dehydrogenase 01 to confer this two-step NADPH/NADH conversion mechanism for the designer butanol-production pathway(s). Therefore, in one of the various embodiments, it is also a preferred practice to use a NAD⁺-dependent glyceraldehyde-3-phosphate dehydrogenase, its isozymes, functional derivatives, analogs, designer modified enzymes and/or combinations thereof in the designer butanol-production pathway(s) as illustrated in FIG. 1.

iRNA Techniques to Further Tame Photosynthesis Regulation Mechanism

In another embodiment of the present invention, the host plant or cell is further modified to tame the Calvin cycle so that the host can directly produce liquid fuel butanol instead of synthesizing starch (glycogen in the case of oxyphotobacteria), celluloses and lignocelluloses that are often inefficient and hard for the biorefinery industry to use. According to the one of the various embodiments, inactivation of starch-synthesis activity is achieved by suppressing the expression of any of the key enzymes, such as, starch synthase (glycogen synthase in the case of oxyphotobacteria) 13, glucose-1-phosphate (G-1-P) adenylyltransferase 14, phosphoglucomutase 15, and hexose-phosphate-isomerase 16 of the starch-synthesis pathway which connects with the Calvin cycle (FIG. 1).

Introduction of a genetically transmittable factor that can inhibit the starch-synthesis activity that is in competition with designer butanol-production pathway(s) for the Calvin-cycle products can further enhance photosynthetic butanol production. In a specific embodiment, a genetically encoded-able inhibitor (FIG. 2C) to the competitive starch-synthesis pathway is an interfering RNA (iRNA) molecule that specifically inhibits the synthesis of a starch-synthesis-pathway enzyme, for example, starch synthase 16, glucose-1-phosphate (G-1-P) adenylyltransferase 15, phosphoglucomutase 14, and/or hexose-phosphate-isomerase 13 as shown with numerical labels 13-16 in FIG. 1. The DNA sequences encoding starch synthase iRNA, glucose-1-phosphate (G-1-P) adenylyltransferase iRNA, a phosphoglucomutase iRNA and/or a G-P-isomerase iRNA, respectively, can be designed and synthesized based on RNA interference techniques known to those skilled in the art (Liszewski (Jun. 1, 2003) Progress in RNA interference, Genetic Engineering News, Vol. 23, number 11, pp. 1-59). Generally speaking, an interfering RNA (iRNA) molecule is anti-sense but complementary to a normal mRNA of a particular protein (gene) so that such iRNA molecule can specifically bind with the normal mRNA of the particular gene, thus inhibiting (blocking) the translation of the gene-specific mRNA to protein (Fire, Xu, Montgomery, Kostas, Driver, Mello (1998) “Potent and specific genetic interference by double-stranded RNA in Caenorhabditis elegans”. Nature 391(6669):806-11; Dykxhoorn, Novina, Sharp (2003) “Killing the messenger: short RNAs that silence gene expression”, Nat Rev Mol Cell Biol. 4(6):457-67).

Examples of a designer starch-synthesis iRNA DNA construct (FIG. 2C) are shown in SEQ ID NO: 27 and 28 listed. Briefly, SEQ ID NO: 27 presents example 27 for a designer Nia1-promoter-controlled Starch-Synthase-iRNA DNA construct (860 bp) that includes a PCR FD primer (sequence 1-20), a 262-bp Nia1 promoter (21-282), a Starch-Synthase iRNA sequence (283-617) consisting of start codon atg and a reverse complement sequence of two unique sequence fragments of a Chlamydomonas reinhardtii starch-synthase-mRNA sequence (GenBank: AF026422), a 223-bp RbcS2 terminator (618-850), and a PCR RE primer (851-860). Because of the use of a Nia1 promoter (21-282), this designer starch-synthesis iRNA gene is designed to be expressed only when needed to enhance photobiological butanol production in the presence of its specific inducer, nitrate (NO₃⁻), which can be added into the culture medium as a fertilizer for induction of the designer organisms. The Starch-Synthase iRNA sequence (283-617) is designed to bind with the normal mRNA of the starch synthase gene, thus blocking its translation into a functional starch synthase. The inhibition of the starch/glycogen synthase activity at 16 in this manner is to channel more photosynthetic products of the Calvin cycle into the Calvin-cycle-branched butanol-production pathway(s) such as the glyceraldehydes-3-phosphate-branched butanol-production pathway 01-12 as illustrated in FIG. 1.

SEQ ID NO: 28 presents example 28 for a designer HydA1-promoter-controlled Starch-Synthase-iRNA DNA construct (1328 bp) that includes a PCR FD primer (sequence 1-20), a 282-bp HydA1 promoter (21-302), a designer Starch-Synthase iRNA sequence (303-1085), a 223-bp RbcS2 terminator (1086-1308), and a PCR RE primer (1309-1328). The designer Starch-Synthase-iRNA sequence (303-1085) comprises of: a 300-bp sense fragment (303-602) selected from the first 300-bp unique coding sequence of a Chlamydomonas reinhardtii starch synthase mRNA sequence (GenBank: AF026422), a 183-bp designer intron-like loop (603-785), and a 300-bp antisense sequence (786-1085) complement to the first 300-bp coding sequence of a Chlamydomonas reinhardtii starch-synthase-mRNA sequence (GenBank: AF026422). This designer Starch-Synthase-iRNA sequence (303-1085) is designed to inhibit the synthesis of starch synthase by the following two mechanisms. First, the 300-bp antisense complement iRNA sequence (corresponding to DNA sequence 786-1085) binds with the normal mRNA of the starch synthase gene, thus blocking its translation into a functional starch synthase. Second, the 300-bp antisense complement iRNA sequence (corresponding to DNA sequence 786-1085) can also bind with the 300-bp sense counterpart (corresponding to DNA sequence 303-602) in the same designer iRNA molecule, forming a hairpin-like double-stranded RNA structure with the 183-bp designer intron-like sequence (603-785) as a loop. Experimental studies have shown that this type of hairpin-like double-stranded RNA can also trigger post-transcriptional gene silencing (Fuhrmann, Stahlberg, Govorunova, Rank and Hegemann (2001) Journal of Cell Science 114:3857-3863). Because of the use of a HydA1 promoter (21-302), this designer starch-synthesis-iRNA gene is designed to be expressed only under anaerobic conditions when needed to enhance photobiological butanol production by channeling more photosynthetic products of the Calvin cycle into the butanol-production pathway(s) such as 01-12, 03-12, and/or 20-33 as illustrated in FIG. 1.

### Designer Starch-Degradation and Glycolysis Genes

In yet another embodiment of the present invention, the photobiological butanol production is enhanced by incorporating an additional set of designer genes (FIG. 2D) that can facilitate starch/glycogen degradation and glycolysis in combination with the designer butanol-production gene(s) (FIG. 2A). Such additional designer genes for starch degradation include, for example, genes coding for 17: amylase, starch phosphorylase, hexokinase, phosphoglucomutase, and for 18: glucose-phosphate-isomerase (G-P-isomerase) as illustrated in FIG. 1. The designer glycolysis genes encode chloroplast-targeted glycolysis enzymes: glucosephosphate isomerase 18, phosphofructose kinase 19, aldolase 20, triose phosphate isomerase 21, glyceraldehyde-3-phosphate dehydrogenase 22, phosphoglycerate kinase 23, phosphoglycerate mutase 24, enolase 25, and pyruvate kinase 26. The designer starch-degradation and glycolysis genes in combination with any of the butanol-production pathways shown in FIG. 1 can form additional pathway(s) from starch/glycogen to butanol (17-33). Consequently, co-expression of the designer starch-degradation and glycolysis genes with the butanol-production-pathway genes can enhance photobiological production of butanol as well. Therefore, this embodiment represents another approach to tame the Calvin cycle for enhanced photobiological production of butanol. In this case, some of the Calvin-cycle products flow through the starch synthesis pathway (13-16) followed by the starch/glycogen-to-butanol pathway (17-33) as shown in FIG. 1. In this case, starch/glycogen acts as a transient storage pool of the Calvin-cycle products before they can be converted to butanol. This mechanism can be quite useful in maximizing the butanol-production yield in certain cases. For example, at high sunlight intensity such as around noon, the rate of Calvin-cycle photosynthetic CO₂ fixation can be so high that may exceed the maximal rate capacity of a butanol-production pathway(s); use of the starch-synthesis mechanism allows temporary storage of the excess photosynthetic products to be used later for butanol production as well.

FIG. 1 also illustrates the use of a designer starch/glycogen-to-butanol pathway with designer enzymes (as labeled from 17 to 33) in combination with a Calvin-cycle-branched designer butanol-production pathway(s) such as the glyceraldehydes-3-phosphate-branched butanol-production pathway 01-12 for enhanced photobiological butanol production. Similar to the benefits of using the Calvin-cycle-branched designer butanol-production pathways, the use of the designer starch/glycogen-to-butanol pathway (17-33) can also help to convert the photosynthetic products to butanol before the sugars could be converted into other complicated biomolecules such as lignocellulosic biomasses which cannot be readily used by the biorefinery industries. Therefore, appropriate use of the Calvin-cycle-branched designer butanol-production pathway(s) (such as 01-12, 03-12, and/or 20-33) and/or the designer starch/glycogen-to-butanol pathway (17-33) may represent revolutionary inter alia technologies that can effectively bypass the bottleneck problems of the current biomass technology including the “lignocellulosic recalcitrance” problem.

Another feature is that a Calvin-cycle-branched designer butanol-production pathway activity (such as 01-12, 03-12, and/or 20-33) can occur predominantly during the days when there is light because it uses an intermediate product of the Calvin cycle which requires supplies of reducing power (NADPH) and energy (ATP) generated by the photosynthetic water splitting and the light-driven proton-translocation-coupled electron transport process through the thylakoid membrane system. The designer starch/glycogen-to-butanol pathway (17-33) which can use the surplus sugar that has been stored as starch/glycogen during photosynthesis can operate not only during the days, but also at nights. Consequently, the use of a Calvin-cycle-branched designer butanol-production pathway (such as 01-12, 03-12, and/or 20-33) together with a designer starch/glycogen-to-butanol pathway(s) (17-33) as illustrated in FIG. 1 enables production of butanol both during the days and at nights.

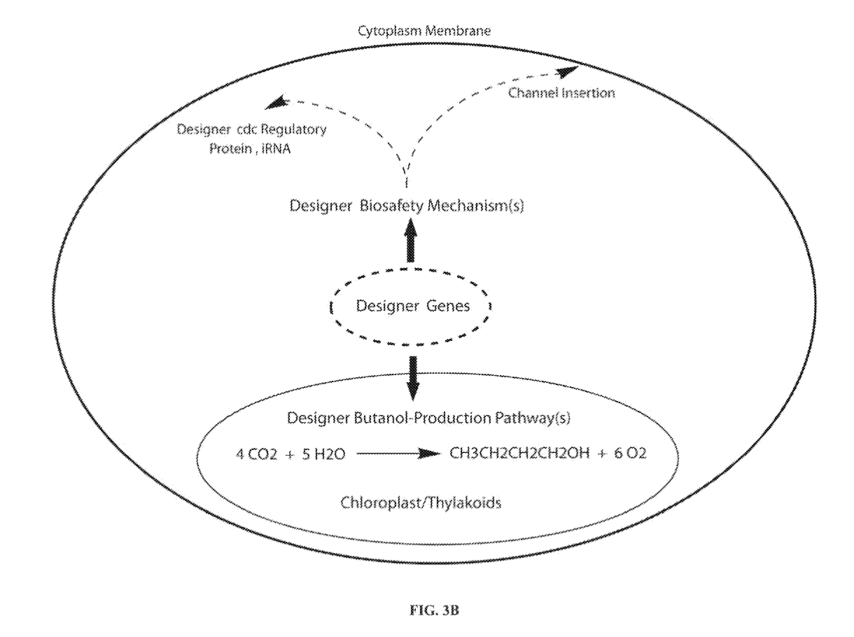

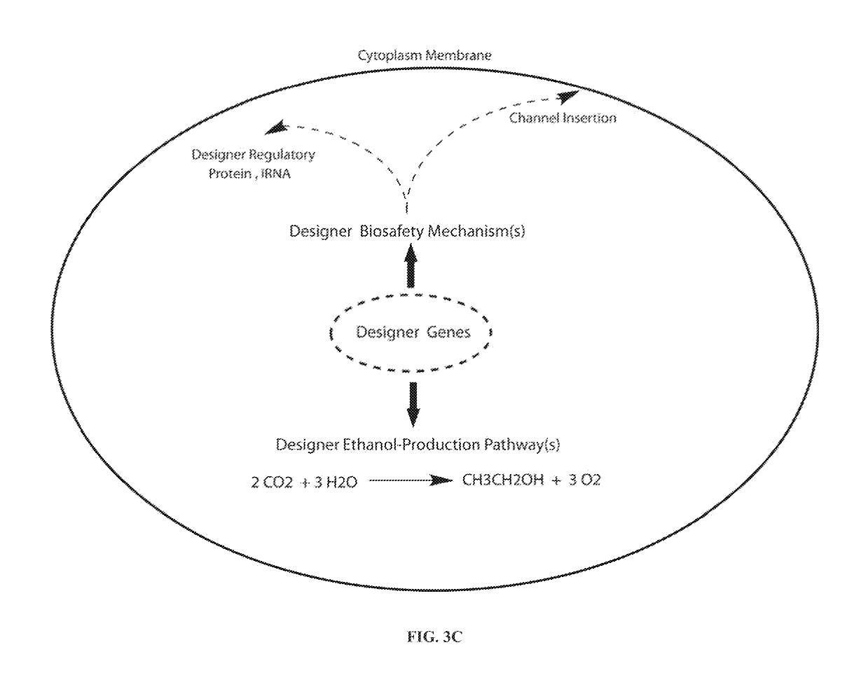

Because the expression for both the designer starch/glycogen-to-butanol pathway(s) and the Calvin-cycle-branched designer butanol-production pathway(s) is controlled by the use of an inducible promoter such as an anaerobic hydrogenase promoter, this type of designer organisms is also able to grow photoautotrophically under aerobic (normal) conditions. When the designer photosynthetic organisms are grown and ready for photobiological butanol production, the cells are then placed under the specific inducing conditions such as under anaerobic conditions [or an ammonium-to-nitrate fertilizer use shift, if designer Nia1/nirA promoter-controlled butanol-production pathway(s) is used] for enhanced butanol production, as shown in FIGS. 1 and 3.

Examples of designer starch (glycogen)-degradation genes are shown in SEQ ID NO: 29-33 listed. Briefly, SEQ ID NO:29 presents example 29 for a designer Amylase DNA construct (1889 bp) that includes a PCR FD primer (sequence 1-20), a 2×84-bp NR promoter (21-188), a 9-bp Xho I NdeI site (189-197), a 135-bp RbcS2 transit peptide (198-332), an Amylase-encoding sequence (333-1616) selected and modified from a Barley alpha-amylase (GenBank: J04202A my46 expression tested in aleurone cells), a 21-bp Lumio-tag sequence (1617-1637), a 9-bp XbaI site (1638-1646), a 223-bp RbcS2 terminator (1647-1869), and a PCR RE primer (1870-1889).

SEQ ID NO: 30 presents example 30 for a designer Starch-Phosphorylase DNA construct (3089 bp) that includes a PCR FD primer (sequence 1-20), a 2×84-bp NR promoter (21-188), a 135-bp RbcS2 transit peptide (189-323), a Starch Phosphorylase-encoding sequence (324-2846) selected and modified from a Citrus root starch-phosphorylase sequence (GenBank: AY098895, expression tested in citrus root), a 223-bp RbcS2 terminator (2847-3069), and a PCR RE primer (3070-3089).

SEQ ID NO: 31 presents example 31 for a designer Hexose-Kinase DNA construct (1949 bp) that includes a PCR FD primer (sequence 1-20), a 2×84-bp NR promoter (21-188), a 135-bp RbcS2 transit peptide (189-323), a Hexose Kinase-encoding sequence (324-1706) selected and modified from Ajellomyces capsulatus hexokinase mRNA sequence (Genbank: XM_{—}001541513), a 223-bp RbcS2 terminator (1707-1929), and a PCR RE primer (1930-1949).

SEQ ID NO: 32 presents example 32 for a designer Phosphoglucomutase DNA construct (2249 bp) that includes a PCR FD primer (sequence 1-20), a 2×84-bp NR promoter (21-188), a 135-bp RbcS2 transit peptide (189-323), a Phosphoglucomutase-encoding sequence (324-2006) selected and modified from Pichia stipitis phosphoglucomutase sequence (GenBank: XM_{—}001383281), a 223-bp RbcS2 terminator (2007-2229), and a PCR RE primer (2230-2249).

SEQ ID NO: 33 presents example 33 for a designer Glucosephosphate-Isomerase DNA construct (2231 bp) that includes a PCR FD primer (sequence 1-20), a 2×84-bp NR promoter (21-188), a 135-bp RbcS2 transit peptide (189-323), a Glucosephosphate Isomerase-encoding sequence (324-1988) selected and modified from a S. cerevisiae phosphoglucoisomerase sequence (GenBank: M21696), a 223-bp RbcS2 terminator (1989-2211), and a PCR RE primer (2212-2231).

The designer starch-degradation genes such as those shown in SEQ ID NO: 29-33 can be selected for use in combination with various designer butanol-production-pathway genes for construction of various designer starch-degradation butanol-production pathways such as the pathways shown in FIG. 1. For example, the designer genes shown in SEQ ID NOS: 1-12, 24-26, and 29-33 can be selected for construction of a Nia1 promoter-controlled starch-to-butanol production pathway that comprises of the following designer enzymes: amylase, starch phosphorylase, hexokinase, phosphoglucomutase, glucosephosphate isomerase, phosphofructose kinase, fructose diphosphate aldolase, triose phosphate isomerase, glyceraldehyde-3-phosphate dehydrogenase, phosphoglycerate kinase, phosphoglycerate mutase, enolase, pyruvate kinase, pyruvate-NADP⁺ oxidoreductase (or pyruvate-ferredoxin oxidoreductase), thiolase, 3-hydroxybutyryl-CoA dehydrogenase, crotonase, butyryl-CoA dehydrogenase, butyraldehyde dehydrogenase, and butanol dehydrogenase. This starch/glycogen-to-butanol pathway 17-33 may be used alone and/or in combinations with other butanol-production pathway(s) such as the 3-phosphoglycerate-branched butanol-production pathway 03-12 as illustrated in FIG. 1.

### Distribution of Designer Butanol-Production Pathways Between Chloroplast and Cytoplasm

In yet another embodiment of the present invention, photobiological butanol productivity is enhanced by a selected distribution of the designer butanol-production pathway(s) between chloroplast and cytoplasm in a eukaryotic plant cell. That is, not all the designer butanol-production pathway(s) (FIG. 1) have to operate in the chloroplast; when needed, part of the designer butanol-production pathway(s) can operate in cytoplasm as well. For example, in one of the various embodiments, a significant part of the designer starch-to-butanol pathway activity from dihydroxyacetone phosphate to butanol (21-33) is designed to occur at the cytoplasm while the steps from starch to dihydroxyacetone phosphate (17-20) are in the chloroplast. In this example, the linkage between the chloroplast and cytoplasm parts of the designer pathway is accomplished by use of the triose phosphate-phosphate translocator, which facilitates translocation of dihydroxyacetone across the chloroplast membrane. By use of the triose phosphate-phosphate translocator, it also enables the glyceraldehyde-3-phospahte-branched designer butanol-production pathway to operate not only in chloroplast, but also in cytoplasm as well. The cytoplasm part of the designer butanol-production pathway can be constructed by use of designer butanol-production pathway genes (DNA constructs of FIG. 2A) with their chloroplast-targeting sequence omitted as shown in FIG. 2E.

Designer Oxyphotobacteria with Designer Butanol-Production Pathways in Cytoplasm

In prokaryotic photosynthetic organisms such as blue-green algae (oxyphotobacteria including cyanobacteria and oxychlorobacteria), which typically contain photosynthetic thylakoid membrane but no chloroplast structure, the Calvin cycle is located in the cytoplasm. In this special case, the entire designer butanol-production pathway(s) (FIG. 1) including (but not limited to) the glyceraldehyde-3-phosphate branched butanol-production pathway (01-12), the 3-phosphpglycerate-branched butanol-production pathway (03-12), the fructose-1,6-diphosphate-branched pathway (20-33), the fructose-6-phosphate-branched pathway (19-33), and the starch (or glycogen)-to-butanol pathways (17-33) are adjusted in design to operate with the Calvin cycle in the cytoplasm of a blue-green alga. The construction of the cytoplasm designer butanol-production pathways can be accomplished by use of designer butanol-production pathway genes (DNA construct of FIG. 2A) with their chloroplast-targeting sequence all omitted. When the chloroplast-targeting sequence is omitted in the designer DNA construct(s) as illustrated in FIG. 2E, the designer gene(s) is transcribed and translated into designer enzymes in the cytoplasm whereby conferring the designer butanol-production pathway(s). The designer gene(s) can be incorporated into the chromosomal and/or plasmid DNA in host blue-green algae (oxyphotobacteria including cyanobacteria and oxychlorobacteria) by using the techniques of gene transformation known to those skilled in the art. It is a preferred practice to integrate the designer genes through an integrative transformation into the chromosomal DNA that can usually provide better genetic stability for the designer genes. In oxyphotobacteria such as cyanobacteria, integrative transformation can be achieved through a process of homologous DNA double recombination into the host's chromosomal DNA using a designer DNA construct as illustrated in FIG. 2F, which typically, from the 5′ upstream to the 3′ downstream, consists of: recombination site 1, a designer butanol-production-pathway gene(s), and recombination site 2. This type of DNA constructs (FIG. 2F) can be delivered into oxyphotobacteria (blue-green algae) with a number of available genetic transformation techniques including electroporation, natural transformation, and/or conjugation. The transgenic designer organisms created from blue-green algae are also called designer blue-green algae (designer oxyphotobacteria including designer cyanobacteria and designer oxychlorobacteria).

Examples of designer oxyphotobacterial butanol-production-pathway genes are shown in SEQ ID NO: 34-45 listed. Briefly, SEQ ID NO:34 presents example 34 for a designer oxyphotobacterial Butanol Dehydrogenase DNA construct (1709 bp) that includes a PCR FD primer (sequence 1-20), a 400-bp nitrite reductase (nirA) promoter from Thermosynechococcus elongatus BP-1 (21-420), an enzyme-encoding sequence (421-1569) selected and modified from a Clostridium saccharoperbutylacetonicum Butanol Dehydrogenase sequence (AB257439), a 120-bp rbcS terminator from Thermosynechococcus elongatus BP-1 (1570-1689), and a PCR RE primer (1690-1709) at the 3′ end.

SEQ ID NO:35 presents example 35 for a designer oxyphotobacterial Butyraldehyde Dehydrogenase DNA construct (1967 bp) that includes a PCR FD primer (sequence 1-20), a 400-bp Thermosynechococcus elongatus BP-1 nitrite reductase nirA promoter (21-420), an enzyme-encoding sequence (421-1827) selected and modified from a Clostridium saccharoperbutylacetonicum Butyraldehyde Dehydrogenase sequence (AY251646), a 120-bp rbcS terminator from Thermosynechococcus (1828-1947), and a PCR RE primer (1948-1967).

SEQ ID NO:36 presents example 36 for a designer oxyphotobacterial Butyryl-CoA Dehydrogenase DNA construct (1602 bp) that includes a PCR FD primer (sequence 1-20), a 305-bp Thermosynechococcus elongatus BP-1 nitrate reductase promoter (21-325), a Butyryl-CoA Dehydrogenase encoding sequence (326-1422) selected/modified from the sequences of a Clostridium beijerinckii Butyryl-CoA Dehydrogenase (AF494018), a 120-bp Thermosynechococcus rbcS terminator (1423-1582), and a PCR RE primer (1583-1602).

SEQ ID NO:37 presents example 37 for a designer oxyphotobacterial Crotonase DNA construct (1248 bp) that includes a PCR FD primer (sequence 1-20), a 305-bp Thermosynechococcus elongatus BP-1 nitrate reductase promoter (21-325), a Crotonase-encoding sequence (326-1108) selected/modified from the sequences of a Clostridium beijerinckii Crotonase (GenBank: AF494018), 120-bp Thermosynechococcus elongatus BP-1 rbcS terminator (1109-1228), and a PCR RE primer (1229-1248).

SEQ ID NO:38 presents example 38 for a designer oxyphotobacterial 3-Hydroxybutyryl-CoA Dehydrogenase DNA construct (1311 bp) that include of a PCR FD primer (sequence 1-20), a 305-bp nirA promoter from (21-325), a 3-Hydroxybutyryl-CoA Dehydrogenase-encoding sequence (326-1171) selected/modified from a Clostridium beijerinckii 3-Hydroxybutyryl-CoA Dehydrogenase sequence Crotonase (GenBank: AF494018), a 120-bp Thermosynechococcus rbcS terminator (1172-1291), and a PCR RE primer (1292-1311).

SEQ ID NO:39 presents example 39 for a designer oxyphotobacterial Thiolase DNA construct (1665 bp) that includes a PCR FD primer (sequence 1-20), a 305-bp Thermosynechococcus nirA promoter (21-325), a Thiolase-encoding sequence (326-1525) selected from a Butyrivibrio fibrisolvens Thiolase sequence (AB190764), a 120-bp Thermosynechococcus rbcS terminator (1526-1645), and a PCR RE primer (1646-1665).

SEQ ID NO:40 presents example 40 for a designer oxyphotobacterial Pyruvate-Ferredoxin Oxidoreductase DNA construct (4071 bp) that includes a PCR FD primer (sequence 1-20), a 305-bp nirA promoter from Thermosynechococcus elongatus BP-1 (21-325), a Pyruvate-Ferredoxin Oxidoreductase-encoding sequence (326-3931) selected/modified from the sequences of a Mastigamoeba balamuthi Pyruvate-ferredoxin oxidoreductase (GenBank: AY101767), a 120-bp rbcS terminator from Thermosynechococcus elongatus BP-1 (3932-4051), and a PCR RE primer (4052-4071).

SEQ ID NO:41 presents example 41 for a designer oxyphotobacterial Pyruvate Kinase DNA construct (1806 bp) that includes a PCR FD primer (sequence 1-20), a 305-bp nirA promoter from Thermosynechococcus (21-325), a pyruvate kinase-encoding sequence (326-1666) selected/modified from a Thermoproteus tenax pyruvate kinase (GenBank: AF065890), a 120-bp Thermosynechococcus rbcS terminator (1667-1786), and a PCR RE primer (1787-1806).

SEQ ID NO:42 presents example 42 for a designer oxyphotobacterial Enolase DNA construct (1696 bp) that includes a PCR FD primer (sequence 1-20), a 231-bp nirA promoter from Thermosynechococcus (21-251), a enolase-encoding sequence (252-1556) selected/modified from the sequences of a Chlamydomonas cytosolic enolase (GenBank: X66412, P31683), a 120-bp rbcS terminator from Thermosynechococcus (1557-1676), and a PCR RE primer (1677-1696).

SEQ ID NO:43 presents example 43 for a designer oxyphotobacterial Phosphoglycerate-Mutase DNA construct (2029 bp) that includes a PCR FD primer (sequence 1-20), a 231-bp nirA promoter from Thermosynechococcus elongatus BP-1 (21-251), a phosphoglycerate-mutase encoding sequence (252-1889) selected/modified from the sequences of a Pelotomaculum thermopropionicum SI phosphoglycerate mutase (GenBank: YP_{—}001213270), a 120-bp Thermosynechococcus rbcS terminator (1890-2009), and a PCR RE primer (2010-2029).

SEQ ID NO:44 presents example 44 for a designer oxyphotobacterial Phosphoglycerate Kinase DNA construct (1687 bp) that includes a PCR FD primer (sequence 1-20), a 231-bp nirA promoter from Thermosynechococcus elongatus BP-1 (21-251), a phosphoglycerate-kinase-encoding sequence (252-1433) selected from Pelotomaculum thermopropionicum SI phosphoglycerate kinase (BAF60903), a 234-bp Thermosynechococcus elongatus BP-1 rbcS terminator (1434-1667), and a PCR RE primer (1668-1687).

SEQ ID NO:45 presents example 45 for a designer oxyphotobacterial Glyceraldehyde-3-Phosphate Dehydrogenase DNA construct (1514 bp) that includes a PCR FD primer (sequence 1-20), a 305-bp Thermosynechococcus elongatus BP-1 nirA promoter (21-325), an enzyme-encoding sequence (326-1260) selected and modified from Blastochloris viridis NAD-dependent Glyceraldehyde-3-phosphate dehydrogenase (CAC80993), a 234-bp rbcS terminator from Thermosynechococcus elongatus BP-1 (1261-1494), and a PCR RE primer (1495-1514).

The designer oxyphotobacterial genes such as those shown in SEQ ID NO: 34-45 can be selected for use in full or in part, and/or in combination with various other designer butanol-production-pathway genes for construction of various designer oxyphotobacterial butanol-production pathways such as the pathways shown in FIG. 1. For example, the designer genes shown in SEQ ID NOS: 34-45 can be selected for construction of an oxyphotobacterial nirA promoter-controlled and glyceraldehyde-3-phosphate-branched butanol-production pathway (01-12) that comprises of the following designer enzymes: NAD-dependent glyceraldehyde-3-phosphate dehydrogenase 01, phosphoglycerate kinase 02, phosphoglycerate mutase 03, enolase 04, pyruvate kinase 05, pyruvate-ferredoxin oxidoreductase (or pyruvate-NADP⁺ oxidoreductase) 06, thiolase 07, 3-hydroxybutyryl-CoA dehydrogenase 08, crotonase 09, butyryl-CoA dehydrogenase 10, butyraldehyde dehydrogenase 11, and butanol dehydrogenase 12. Use of these designer oxyphotobacterial butanol-production-pathway genes (SEQ ID NOS: 34-45) in a thermophilic and/or thermotolerant cyanobacterium may represent a thermophilic and/or thermotolerant butanol-producing oxyphotobacterium. Fox example, use of these designer genes (SEQ ID NOS: 34-45) in a thermophilic/thermotolerant cyanobacterium such as Thermosynechococcus elongatus BP-1 may represent a designer thermophilic/thermotolerant butanol-producing cyanobacterium such as a designer butanol-producing Thermosynechococcus.

### Further Host Modifications to Help Ensure Biosafety

The present invention also provides biosafety-guarded photosynthetic biofuel (e.g., butanol and/or related higher alcohols) production methods based on cell-division-controllable designer transgenic plants (such as algae and oxyphotobacteria) or plant cells. For example, the cell-division-controllable designer photosynthetic organisms (FIG. 3) are created through use of a designer biosafety-control gene(s) (FIG. 2G) in conjunction with the designer butanol-production-pathway gene(s) (FIGS. 2A-2F) such that their cell division and mating function can be controllably stopped to provide better biosafety features.

In one of the various embodiments, a fundamental feature is that a designer cell-division-controllable photosynthetic organism (such as an alga, plant cell, or oxyphotobacterium) contains two key functions (FIG. 3A): a designer biosafety mechanism(s) and a designer biofuel-production pathway(s). As shown in FIG. 3B, the designer biosafety feature(s) is conferred by a number of mechanisms including: (1) the inducible insertion of designer proton-channels into cytoplasm membrane to permanently disable any cell division and mating capability, (2) the selective application of designer cell-division-cycle regulatory protein or interference RNA (iRNA) to permanently inhibit the cell division cycle and preferably keep the cell at the G₁ phase or G₀ state, and (3) the innovative use of a high-CO₂-requiring host photosynthetic organism for expression of the designer biofuel-production pathway(s). Examples of the designer biofuel-production pathway(s) include the designer butanol-production pathway(s), which work with the Calvin cycle to synthesize biofuel such as butanol directly from carbon dioxide (CO₂) and water (H₂O). The designer cell-division-control technology can help ensure biosafety in using the designer organisms for photosynthetic biofuel production. Accordingly, this embodiment provides, inter alia, biosafety-guarded methods for producing biofuel (e.g., butanol and/or related higher alcohols) based on a cell-division-controllable designer biofuel-producing alga, cyanobacterium, oxychlorobacterium, plant or plant cells.

In one of the various embodiments, a cell-division-controllable designer butanol-producing eukaryotic alga or plant cell is created by introducing a designer proton-channel gene (FIG. 2H) into a host alga or plant cell (FIG. 3B). SEQ ID NO: 46 presents example 46 for a detailed DNA construct of a designer Nia1-promoter-controlled proton-channel gene (609 bp) that includes a PCR FD primer (sequence 1-20), a 262-bp nitrate reductase Nia1 promoter (21-282), a Melittin proton-channel encoding sequence (283-366), a 223-bp RbcS2 terminator (367-589), and a PCR RE primer (590-609).

The expression of the designer proton-channel gene (FIG. 2H) is controlled by an inducible promoter such as the nitrate reductase (Nia1) promoter, which can also be used to control the expression of a designer biofuel-production-pathway gene(s). Therefore, before the expression of the designer gene(s) is induced, the designer organism can grow photoautotrophically using CO₂ as the carbon source and H₂O as the source of electrons just like wild-type organism. When the designer organism culture is grown and ready for photobiological production of biofuels, the cell culture is then placed under a specific inducing condition (such as by adding nitrate into the culture medium if the nitrate reductase (Nia1) promoter is used as an inducible promoter) to induce the expression of both the designer proton-channel gene and the designer biofuel-production-pathway gene(s). The expression of the proton-channel gene is designed to occur through its transcription in the nucleus and its translation in the cytosol. Because of the specific molecular design, the expressed proton channels are automatically inserted into the cytoplasm membrane, but leave the photosynthetic thylakoid membrane intact. The insertion of the designer proton channels into cytoplasm membrane collapses the proton gradient across the cytoplasm membrane so that the cell division and mating function are permanently disabled. However, the photosynthetic thylakoid membrane inside the chloroplast is kept intact (functional) so that the designer biofuel-production-pathway enzymes expressed into the stroma region can work with the Calvin cycle for photobiological production of biofuels from CO₂ and H₂O. That is, when both the designer proton-channel gene and the designer biofuel-production-pathway gene(s) are turned on, the designer organism becomes a non-reproducible cell for dedicated photosynthetic production of biofuels. Because the cell division and mating function are permanently disabled (killed) at this stage, the designer-organism culture is no longer a living matter except its catalytic function for photochemical conversion of CO₂ and H₂O into a biofuel. It will no longer be able to mate or exchange any genetic materials with any other cells, even if it somehow comes in contact with a wild-type cell as it would be the case of an accidental release into the environments.

According to one of the various embodiments, the nitrate reductase (Nia1) promoter or nitrite reductase (nirA) promoter is a preferred inducible promoter for use to control the expression of the designer genes. In the presence of ammonium (but not nitrate) in culture medium, for example, a designer organism with Nia1-promoter-controlled designer proton-channel gene and biofuel-production-pathway gene(s) can grow photoauotrophically using CO₂ as the carbon source and H₂O as the source of electrons just like a wild-type organism. When the designer organism culture is grown and ready for photobiological production of biofuels, the expression of both the designer proton-channel gene and the designer biofuel-production-pathway gene(s) can then be induced by adding some nitrate fertilizer into the culture medium. Nitrate is widely present in soils and nearly all surface water on Earth. Therefore, even if a Nia1-promoter-controlled designer organism is accidentally released into the natural environment, it will soon die since the nitrate in the environment will trig the expression of a Nia1-promoter-controlled designer proton-channel gene which inserts proton-channels into the cytoplasm membrane thereby killing the cell. That is, a designer photosynthetic organism with Nia1-promoter-controlled proton-channel gene is programmed to die as soon as it sees nitrate in the environment. This characteristic of cell-division-controllable designer organisms with Nia1-promoter-controlled proton-channel gene provides an added biosafety feature.

The art in constructing proton-channel gene (FIG. 2H) with a thylakoid-membrane targeting sequence has recently been disclosed [James W. Lee (2007). Designer proton-channel transgenic algae for photobiological hydrogen production, PCT International Publication Number: WO 2007/134340 A2]. In the present invention of creating a cell-division-controllable designer organism, the thylakoid-membrane-targeting sequence must be omitted in the proton-channel gene design. For example, the essential components of a Nia1-promoter-controlled designer proton-channel gene can simply be a Nia1 promoter linked with a proton-channel-encoding sequence (without any thylakoid-membrane-targeting sequence) so that the proton channel will insert into the cytoplasm membrane but not into the photosynthetic thylakoid membrane.

According to one of the various embodiments, it is a preferred practice to use the same inducible promoter such as the Nia1 promoter to control the expression of both the designer proton-channel gene and the designer biofuel-production pathway genes. In this way, the designer biofuel-production pathway(s) can be inducibly expressed simultaneously with the expression of the designer proton-channel gene that terminates certain cellular functions including cell division and mating.

In one of the various embodiments, an inducible promoter that can be used in this designer biosafety embodiment is selected from the group consisting of the hydrogenase promoters [HydA1 (Hyd1) and HydA2, accession number: AJ308413, AF289201, AY090770], the Cyc6 gene promoter, the Cpx1 gene promoter, the heat-shock protein promoter HSP70A, the CabII-1 gene (accession number M24072) promoter, the Ca1 gene (accession number P20507) promoter, the Ca2 gene (accession number P24258) promoter, the nitrate reductase (Nia1) promoter, the nitrite-reductase-gene (nirA) promoters, the bidirectional-hydrogenase-gene hox promoters, the light- and heat-responsive groE promoters, the Rubisco-operon rbcL promoters, the metal (zinc)-inducible smt promoter, the iron-responsive idiA promoter, the redox-responsive crhR promoter, the heat-shock-gene hsp16.6 promoter, the small heat-shock protein (Hsp) promoter, the CO₂-responsive carbonic-anhydrase-gene promoters, the green/red light responsive cpcB2A2 promoter, the UV-light responsive lexA, recA and ruvB promoters, the nitrate-reductase-gene (narB) promoters, and combinations thereof.

In another embodiment, a cell-division-controllable designer photosynthetic organism is created by use of a carbonic anhydrase deficient mutant or a high-CO₂-requiring mutant as a host organism to create the designer biofuel-production organism. High-CO₂-requiring mutants that can be selected for use in this invention include (but not limited to): Chlamydomonas reinhardtii carbonic-anhydrase-deficient mutant12-1C(CC-1219 ca1 mt-), Chlamydomonas reinhardtii cia3 mutant (Plant Physiology 2003, 132:2267-2275), the high-CO₂-requiring mutant M3 of Synechococcus sp. Strain PCC 7942, or the carboxysome-deficient cells of Synechocystis sp. PCC 6803 (Plant biol (Stuttg) 2005, 7:342-347) that lacks the CO₂-concentrating mechanism can grow photoautotrophically only under elevated CO₂ concentration level such as 0.2-3% CO₂.

Under atmospheric CO₂ concentration level (380 ppm), the carbonic anhydrase deficient or high-CO₂-requiring mutants commonly cannot survive. Therefore, the key concept here is that a high-CO₂-requiring designer biofuel-production organism that lacks the CO₂ concentrating mechanism will be grown and used for photobiological production of biofuels always under an elevated CO₂ concentration level (0.2-5% CO₂) in a sealed bioreactor with CO₂ feeding. Such a designer transgenic organism cannot survive when it is exposed to an atmospheric CO₂ concentration level (380 ppm=0.038% CO₂) because its CO₂-concetrating mechanism (CCM) for effective photosynthetic CO₂ fixation has been impaired by the mutation. Even if such a designer organism is accidentally released into the natural environment, its cell will soon not be able to divide or mate, but die quickly of carbon starvation since it cannot effectively perform photosynthetic CO₂ fixation at the atmospheric CO₂ concentration (380 ppm). Therefore, use of such a high-CO₂-requiring mutant as a host organism for the genetic transformation of the designer biofuel-production-pathway gene(s) represents another way in creating the envisioned cell-division-controllable designer organisms for biosafety-guarded photobiological production of biofuels from CO₂ and H₂O. No designer proton-channel gene is required here.

In another embodiment, a cell-division-controllable designer organism (FIG. 3B) is created by use of a designer cell-division-cycle regulatory gene as a biosafety-control gene (FIG. 2G) that can control the expression of the cell-division-cycle (cdc) genes in the host organism so that it can inducibly turn off its reproductive functions such as permanently shutting off the cell division and mating capability upon specific induction of the designer gene.

Biologically, it is the expression of the natural cdc genes that controls the cell growth and cell division cycle in cyanobacteria, algae, and higher plant cells. The most basic function of the cell cycle is to duplicate accurately the vast amount of DNA in the chromosomes during the S phase (S for synthesis) and then segregate the copies precisely into two genetically identical daughter cells during the M phase (M for mitosis). Mitosis begins typically with chromosome condensation: the duplicated DNA strands, packaged into elongated chromosomes, condense into the much-more compact chromosomes required for their segregation. The nuclear envelope then breaks down, and the replicated chromosomes, each consisting of a pair of sister chromatids, become attached to the microtubules of the mitotic spindle. As mitosis proceeds, the cell pauses briefly in a state called metaphase, when the chromosomes are aligned at the equator of the mitotic spindle, poised for segregation. The sudden segregation of sister chromatids marks the beginning of anaphase during which the chromosomes move to opposite poles of the spindle, where they decondense and reform intact nuclei. The cell is then pinched into two by cytoplasmic division (cytokinesis) and the cell division is then complete. Note, most cells require much more time to grow and double their mass of proteins and organelles than they require to replicate their DNA (the S phase) and divide (the M phase). Therefore, there are two gap phases: a G₁ phase between M phase and S phase, and a G2 phase between S phase and mitosis. As a result, the eukaryotic cell cycle is traditionally divided into four sequential phases: G₁, S, G₂, and M. Physiologically, the two gap phases also provide time for the cell to monitor the internal and external environment to ensure that conditions are suitable and preparation are complete before the cell commits itself to the major upheavals of S phase and mitosis. The G₁ phase is especially important in this aspect. Its length can vary greatly depending on external conditions and extracellular signals from other cells. If extracellular conditions are unfavorable, for example, cells delay progress through G₁ and may even enter a specialized resting state known as G₀ (G zero), in which they remain for days, weeks, or even for years before resuming proliferation. Indeed, many cells remain permanently in G₀ state until they die.

In one of the various embodiments, a designer gene(s) that encodes a designer cdc-regulatory protein or a specific cdc-iRNA is used to inducibly inhibit the expression of certain cdc gene(s) to stop cell division and disable the mating capability when the designer gene(s) is trigged by a specific inducing condition. When the cell-division-controllable designer culture is grown and ready for photosynthetic production of biofuels, for example, it is a preferred practice to induce the expression of a specific designer cdc-iRNA gene(s) along with induction of the designer biofuel-production-pathway gene(s) so that the cells will permanently halt at the G₁ phase or G₀ state. In this way, the grown designer-organism cells become perfect catalysts for photosynthetic production of biofuels from CO₂ and H₂O while their functions of cell division and mating are permanently shut off at the G₁ phase or G₀ state to help ensure biosafety.

Use of the biosafety embodiments with various designer biofuel-production-pathways genes listed in SEQ ID NOS: 1-45 (and 58-165) can create various biosafety-guarded photobiological biofuel producers (FIGS. 3A, 3B, and 3C). Note, SEQ ID NOS: 46 and 1-12 (examples 1-12) represent an example for a cell-division-controllable designer eukaryotic organism such as a cell-division-controllable designer alga (e.g., Chlamydomonas) that contains a designer Nia1-promoter-controlled proton-channel gene (SEQ ID NO: 46) and a set of designer Nia1-promoter-controlled butanol-production-pathway genes (SEQ ID NOS: 1-12). Because the designer proton-channel gene and the designer biofuel-production-pathway gene(s) are all controlled by the same Nia1-promoter sequences, they can be simultaneously expressed upon induction by adding nitrate fertilizer into the culture medium to provide the biosafety-guarded photosynthetic biofuel-producing capability as illustrated in FIG. 3B. Use of the designer Nia1-promoter-controlled butanol-production-pathway genes (SEQ ID NOS: 1-12) in a high CO₂-requiring host photosynthetic organism, such as Chlamydomonas reinhardtii carbonic-anhydrase-deficient mutant12-1C(CC-1219 ca1 mt-) or Chlamydomonas reinhardtii cia3 mutant, represents another example in creating a designer cell-division-controllable photosynthetic organism to help ensure biosafety.

This designer biosafety feature may be useful to the production of other biofuels such as biooils, biohydrogen, ethanol, and intermediate products as well. For example, this biosafety embodiment in combination with a set of designer ethanol-production-pathway genes such as those shown SEQ ID NOS: 47-53 can represent a cell-division-controllable ethanol producer (FIG. 3C). Briefly, SEQ ID NO: 47 presents example 47 for a detailed DNA construct (1360 base pairs (bp)) of a nirA-promoter-controlled designer NAD-dependent Glyceraldehyde-3-Phosphate-Dehydrogenase gene including: a PCR FD primer (sequence 1-20), a 88-bp nirA promoter (21-108) selected from the Synechococcus sp. (freshwater cyanobacterium) nitrite-reductase-gene promoter sequence, an enzyme-encoding sequence (109-1032) selected and modified from a Cyanidium caldarium cytosolic NAD-dependent glyceraldehyde-3-phosphate-dehydrogenase sequence (GenBank accession number: CAC85917), a 308-bp Synechococcus rbcS terminator (1033-1340), and a PCR RE primer (1341-1360) at the 3′ end.

SEQ ID NO: 48 presents example 48 for a designer nirA-promoter-controlled Phosphoglycerate Kinase DNA construct (1621 bp) that includes a PCR FD primer (sequence 1-20), a 88-bp Synechococcus sp. strain PCC 7942 nitrite-reductase nirA promoter (21-108), a phosphoglycerate-kinase-encoding sequence (109-1293) selected from a Geobacillus kaustophilus phosphoglycerate-kinase sequence (GenBank: BAD77342), a 308-bp Synechococcus rbcS terminator (1294-1601), and a PCR RE primer (1602-1621).

SEQ ID NO: 49 presents example 49 for a designer nirA-promoter-controlled Phosphoglycerate-Mutase DNA construct (1990 bp) that includes a PCR FD primer (sequence 1-20), a 88-bp Synechococcus sp. strain PCC 7942 nitrite-reductase nirA promoter (21-108), a 9-bp Xho I NdeI site (109-117), a phosphoglycerate-mutase encoding sequence (118-1653) selected from the sequences of a Caldicellulosiruptor saccharolyticus DSM 8903 phosphoglycerate mutase (GenBank: ABP67536), a 9-bp XbaI site (1654-1662), a 308-bp Synechococcus sp. strain PCC 7942 rbcS terminator (1663-1970), and a PCR RE primer (1971-1990).

SEQ ID NO: 50 presents example 50 for a designer nirA-promoter-controlled Enolase DNA construct (1765 bp) that includes a PCR FD primer (sequence 1-20), a 88-bp Synechococcus sp. strain PCC 7942 nitrite reductase nirA promoter (21-108), a 9-bp Xho I NdeI site (109-117), an enolase-encoding sequence (118-1407) selected from the sequence of a Cyanothece sp. CCY0110 enolase (GenBank: ZP_{—}01727912), a 21-bp Lumio-tag-encoding sequence (1408-1428), a 9-bp XbaI site (1429-1437) containing a stop codon, a 308-bp Synechococcus rbcS terminator (1438-1745), and a PCR RE primer (1746-1765) at the 3′ end.

SEQ ID NO: 51 presents example 51 for a designer nirA-promoter-controlled Pyruvate Kinase DNA construct (1888 bp) that includes a PCR FD primer (sequence 1-20), a 88-bp Synechococcus nitrite reductase nirA promoter (21-108), a 9-bp Xho I NdeI site (109-117), a Pyruvate-Kinase-encoding sequence (118-1530) selected from a Selenomonas ruminantium Pyruvate Kinase sequence (GenBank: AB037182), a 21-bp Lumio-tag sequence (1531-1551), a 9-bp XbaI site (1552-1560), a 308-bp Synechococcus rbcS terminator (1561-1868), and a PCR RE primer (1869-1888).

SEQ ID NO: 52 presents example 52 for a designer nirA-promoter-controlled Pyruvate Decarboxylase DNA construct (2188 bp) that includes a PCR FD primer (sequence 1-20), a 88-bp Synechococcus nitrite reductase nirA promoter (21-108), a 9-bp Xho I NdeI site (109-117), a Pyruvate-Decarboxylase-encoding sequence (118-1830) selected from the sequences of a Pichia stipitis pyruvate-decarboxylase sequence (GenBank: XM_{—}001387668), a 21-bp Lumio-tag sequence (1831-1851), a 9-bp XbaI site (1852-1860), a 308-bp Synechococcus rbcS terminator (1861-2168), and a PCR RE primer (2169-2188) at the 3′ end.

SEQ ID NO: 53 presents example 53 for a nirA-promoter-controlled designer NAD(P)H-dependent Alcohol Dehydrogenase DNA construct (1510 bp) that includes a PCR FD primer (sequence 1-20), a 88-bp Synechococcus nitrite-reductase nirA promoter (21-108), a NAD(P)H dependent Alcohol-Dehydrogenase-encoding sequence (109-1161) selected/modified (its mitochondrial signal peptide sequence removed) from the sequence of a Kluyveromyces lactis alcohol dehydrogenase (ADH3) gene (GenBank: X62766), a 21-bp Lumio-tag sequence (1162-1182), a 308-bp Synechococcus rbcS terminator (1183-1490), and a PCR RE primer (1491-1510) at the 3′ end.

Note, SEQ ID NOS: 47-53 (DNA-construct examples 47-53) represent a set of designer nirA-promoter-controlled ethanol-production-pathway genes that can be used in oxyphotobacteria such as Synechococcus sp. strain PCC 7942. Use of this set of designer ethanol-production-pathway genes in a high-CO₂-requiring cyanobacterium such as the Synechococcus sp. Strain PCC 7942 mutant M3 represents another example of cell-division-controllable designer cyanobacterium for biosafety-guarded photosynthetic production of biofuels from CO₂ and H₂O.

### More on Designer Calvin-Cycle-Channeled Production of Butanol and Related Higher Alcohols

The present invention further discloses designer Calvin-cycle-channeled and photosynthetic-NADPH (reduced nicotinamide adenine dinucleotide phosphate)-enhanced pathways, associated designer DNA constructs (designer genes) and designer transgenic photosynthetic organisms for photobiological production of butanol and related higher alcohols from carbon dioxide and water. In this context throughout this specification as mentioned before, a “higher alcohol” or “related higher alcohol” refers to an alcohol that comprises at least four carbon atoms, including both straight and branched higher alcohols such as 1-butanol and 2-methyl-1-butanol. The Calvin-cycle-channeled and photosynthetic-NADPH-enhanced pathways are constructed with designer enzymes expressed through use of designer genes in host photosynthetic organisms such as algae and oxyphotobacteria (including cyanobacteria and oxychlorobacteria) organisms for photobiological production of butanol and related higher alcohols. The said butanol and related higher alcohols are selected from the group consisting of: 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, and 6-methyl-1-heptanol. The designer photosynthetic organisms such as designer transgenic algae and oxyphotobacteria (including cyanobacteria and oxychlorobacteria) comprise designer Calvin-cycle-channeled and photosynthetic NADPH-enhanced pathway gene(s) and biosafety-guarding technology for enhanced photobiological production of butanol and related higher alcohols from carbon dioxide and water.

Photosynthetic water splitting and its associated proton gradient-coupled electron transport process generates chemical energy intermediate in the form of adenosine triphosphate (ATP) and reducing power in the form of reduced nicotinamide adenine dinucleotide phosphate (NADPH). However, certain butanol-related metabolic pathway enzymes such as the NADH-dependent butanol dehydrogenase (GenBank accession numbers: YP_{—}148778, NP_{—}561774, AAG23613, ZP_{—}05082669, ADO12118, ADC48983) can use only reduced nicotinamide adenine dinucleotide (NADH) but not NADPH. Therefore, to achieve a true coupling of a designer pathway with the Calvin cycle for photosynthetic production of butanol and related higher alcohols, it is a preferred practice to use an effective NADPH/NADH conversion mechanism and/or NADPH-using enzyme(s) (such as NADPH-dependent enzymes) in construction of a compatible designer pathway(s) to couple with the photosynthesis/Calvin-cycle process in accordance with the present invention.

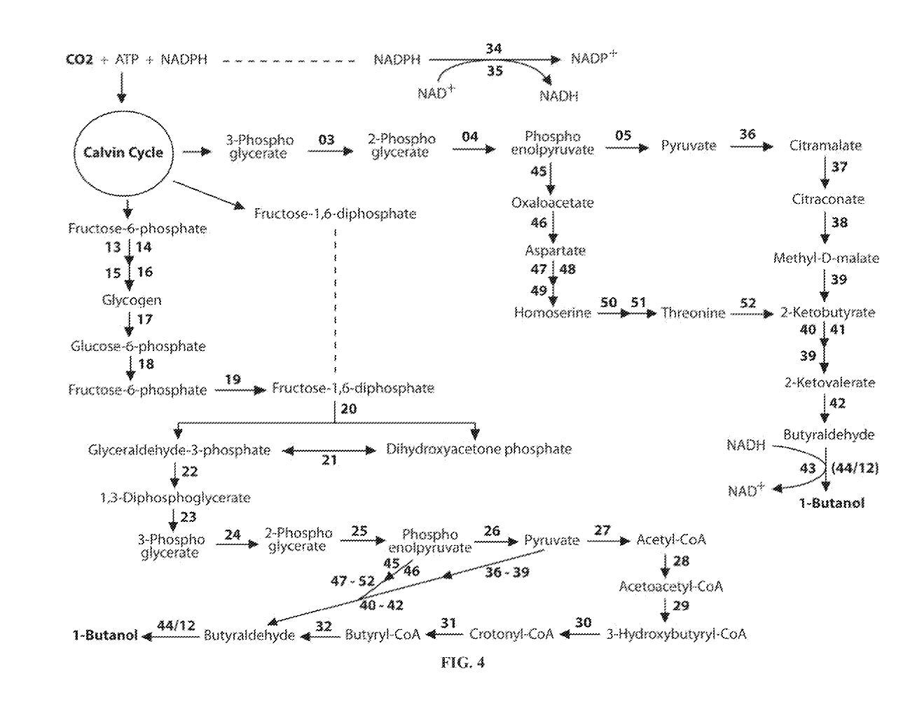

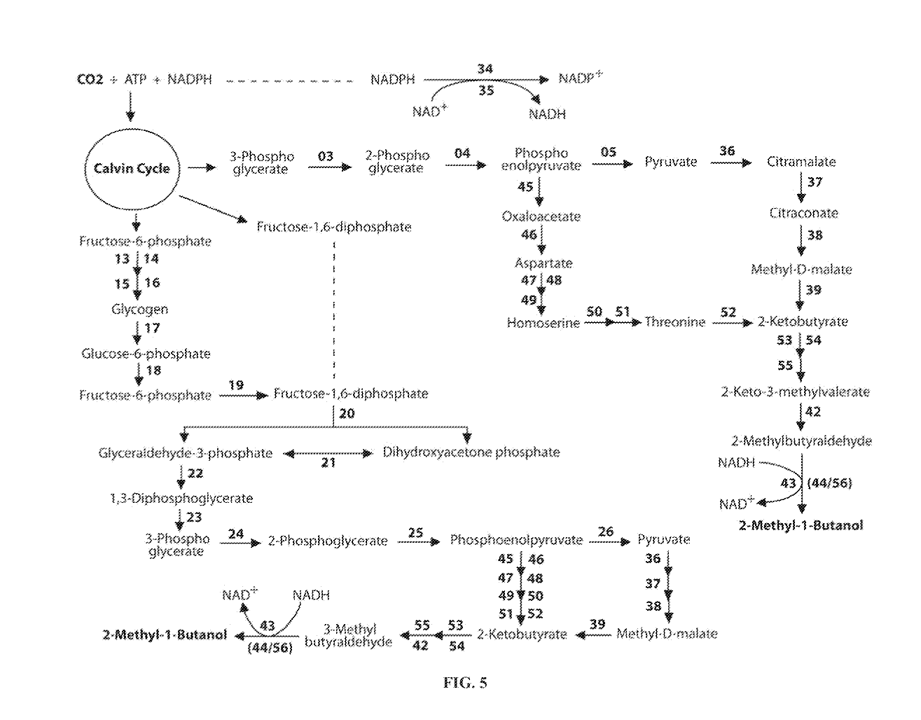

According to one of the various embodiments, a number of various designer Calvin-cycle-channeled pathways can be created by use of an NADPH/NADH conversion mechanism in combination with certain amino-acids-metabolic pathways for production of butanol and higher alcohols from carbon dioxide and water. The Calvin-cycle-channeled and photosynthetic-NADPH-enhanced pathways are constructed typically with designer enzymes that are selectively expressed through use of designer genes in a host photosynthetic organism such as a host alga or oxyphotobacterium for production of butanol and higher alcohols. A list of exemplary enzymes that can be selected for use in construction of the Calvin-cycle-channeled and photosynthetic-NADPH-enhanced pathways are presented in Table 1. As shown in FIGS. 4-10, the net results of the designer Calvin-cycle-channeled and photosynthetic NADPH-enhanced pathways in working with the Calvin cycle are production of butanol and related higher alcohols from carbon dioxide (CO₂) and water (H₂O) using photosynthetically generated ATP (Adenosine triphosphate) and NADPH (reduced nicotinamide adenine dinucleotide phosphate). A significant feature is the innovative utilization of an NADPH-dependent glyceraldehyde-3-phosphate dehydrogenase 34 and a nicotinamide adenine dinucleotide (NAD)-dependent glyceraldehyde-3-phosphate dehydrogenase 35 to serve as a NADPH/NADH conversion mechanism that can convert certain amount of photosynthetically generated NADPH to NADH which can then be used by NADH-requiring pathway enzymes such as an NADH-dependent alcohol dehydrogenase 43 (examples of its encoding gene with GenBank accession numbers are: BAB59540, CAA89136, NP_{—}148480) for production of butanol and higher alcohols.

More specifically, an NADPH-dependent glyceraldehyde-3-phosphate dehydrogenase 34 (e.g., GenBank accession numbers: ADC37857, ADC87332, YP_{—}003471459, ZP_{—}04395517, YP_{—}003287699, ZP_{—}07004478, ZP_{—}04399616) catalyzes the following reaction that uses NADPH in reducing 1,3-Diphosphoglycerate (1,3-DiPGA) to 3-Phosphoglyaldehyde (3-PGAld) and inorganic phosphate (Pi):

1,3-DiPGA+NADPH+H⁺→3-PGAld+NADP⁺+Pi [3]

Meanwhile, an NAD-dependent glyceraldehyde-3-phosphate dehydrogenase 35 (e.g., GenBank: ADM41489, YP_{—}003095198, ADC36961, ZP_{—}07003925, ACQ61431, YP_{—}002285269, ADN80469, ACI60574) catalyzes the oxidation of 3-PGAld by oxidized nicotinamide adenine dinucleotide (NAD⁺) back to 1,3-DiPGA:

3-PGAld+NAD⁺+Pi→1,3-DiPGA+NADH+H⁺ [4]

The net result of the enzymatic reactions [3] and [4] is the conversion of photosynthetically generated NADPH to NADH, which various NADH-requiring designer pathway enzymes such as NADH-dependent alcohol dehydrogenase 43 can use in producing butanol and related higher alcohols. When there is too much NADH, this NADPH/NADH conversion system can run also reversely to balance the supply of NADH and NADPH. Therefore, it is a preferred practice to innovatively utilize this NADPH/NADH conversion system under control of a designer switchable promoter such as nirA (or Nia1 for eukaryotic system) promoter when/if needed to achieve robust production of butanol and related higher alcohols. Various designer Calvin-cycle-channeled pathways in combination of a NADPH/NADH conversion mechanism with certain amino-acids-metabolism-related pathways for photobiological production of butanol and related higher alcohols are further described hereinbelow.

TABLE 1
lists examples of enzymes for construction of designer Calvin-cycle-linked pathways for
production of butanol and related higher alcohols.

| | | GenBank Accession |
|---|---|---|
| | | Number, JGI Protein ID or |
| Enzyme/callout number | Source (Organism) | Citation |
| 03: | Oceanithermus profundus DSM 14977; | ADR35708; |
| Phosphoglycerate mutase | ‘Nostoc azollae’ 0708; | ADI65627, YP_003722750; |
| (phosphoglyceromutase) | Thermotoga lettingae TMO; | YP_001470593, ABV33529; |
| | Syntrophothermus lipocalidus DSM | ADI02216, YP_003702781; |
| | 12680; | |
| | Pelotomaculum thermopropionicum SI; | YP_001212148; |
| | Fervidobacterium nodosum Rt17-B1; | YP_001409891; |
| | Caldicellulosiruptor bescii DSM 6725; | YP_002573254, YP_002573195; |
| | Fervidobacterium nodosum Rt17-B1; | ABS60234; |
| | Thermotoga petrophila RKU-1; | ABQ47079, YP_001244998; |
| | Deferribacter desulfuricans SSM1; | YP_003496402, BAI80646; |
| | Cyanobium sp. PCC 7001; | ZP_05046421; |
| | Cyanothece sp. PCC 8802; | YP_003138980, YP_003138979; |
| | Chlamydomonas reinhardtii cytoplasm; | JGI Chlre2 protein ID 161689, |
| | Aspergillus fumigatus; Coccidioides | GenBank: AF268078; |
| | immitis; Leishmania braziliensis; | XM_747847; XM_749597; |
| | Ajellomyces capsulatus; | XM_001248115; XM_001569263; |
| | Monocercomonoides sp.; Aspergillus | XM_001539892; DQ665859; |
| | clavatus; Arabidopsis thaliana; Zea | XM_001270940; NM_117020; |
| | mays | M80912 |
| 04: | Syntrophothermus lipocalidus DSM | ADI02602, YP_003703167; |
| Enolase | 12680; ‘Nostoc azollae’ 0708; | ADI63801; |
| | Thermotoga petrophila RKU-1; | ABQ46079; |
| | Spirochaeta thermophila DSM 6192; | YP_003875216, ADN02943; |
| | Cyanothece sp. PCC 7822; | YP_003886899, ADN13624; |
| | Hydrogenobacter thermophilus TK-6; | YP_003432637, BAI69436; |
| | Thermosynechococcus elongatus BP-1, | BAC08209; |
| | Prochlorococcus marinus str. MIT | ABO16851; |
| | 9301; Synechococcus sp. WH 5701; | ZP_01083626; |
| | Trichodesmium erythraeum IMS101; | ABG51970; |
| | Anabaena variabilis ATCC 29413; | ABA23124; |
| | Nostoc sp. PCC 7120; | BAB75237; |
| | Chlamydomonas reinhardtii cytoplasm; | GenBank: X66412, P31683; |
| | Arabidopsis thaliana; Leishmania | AK222035; DQ221745; |
| | Mexicana; Lodderomyces elongisporus; | XM_001528071; XM_001611873; |
| | Babesia bovis; Sclerotinia sclerotiorum; | XM_001594215; XM_001483612; |
| | Pichia guilliermondii; | AB221057; EF122486, U09450; |
| | Spirotrichonympha leidyi; Oryza sativa; | DQ845796; AB088633; U82438; |
| | Trimastix pyriformis; Leuconostoc | D64113; U13799; AY307449; |
| | mesenteroides; Davidiella tassiana; | U17973 |
| | Aspergillus oryzae; | |
| | Schizosaccharomyces pombe; Brassica | |
| | napus; Zea mays | |
| 05: | Syntrophothermus lipocalidus DSM | ADI02459, YP_003703024; |
| Pyruvate kinase | 12680; Cyanothece sp. PCC 8802; | YP_002372431; |
| | Thermotoga lettingae TMO; | YP_001471580, ABV34516; |
| | Caldicellulosiruptor bescii DSM 6725; | YP_002573139; |
| | Geobacillus kaustophilus HTA426; | YP_148872; |
| | Thermosynechococcus elongatus BP-1; | NP_681306, BAC08068; |
| | Thermosipho melanesiensis BI429; | YP_001306168, ABR30783; |
| | Thermotoga petrophila RKU-1; | YP_001244312, ABQ46736; |
| | Caldicellulosiruptor saccharolyticus | ABP67416, YP_001180607; |
| | DSM 8903; Cyanothece sp. PCC 7425; | ACL43749, YP_002482578; |
| | Acaryochloris marina MBIC11017; | YP_001514814; |
| | Cyanothece sp. PCC 8801; | YP_003138017; |
| | Microcystis aeruginosa NIES-843; | YP_001655408; |
| | Cyanothece sp. PCC 7822; | YP_003890281; |
| | cyanobacterium UCYN-A; | YP_003422225; |
| | Arthrospira maxima CS-328; | ZP_03273505; |
| | Synechococcus sp. PCC 7335; | ZP_05035056; |
| | Chlamydomonas reinhardtii cytoplasm; | JGI Chlre3 protein ID 138105; |
| | Arabidopsis thaliana; Saccharomyces | GenBank: AK229638; AY949876, |
| | cerevisiae; Babesia bovis; Sclerotinia | AY949890, AY949888; |
| | sclerotiorum; Trichomonas vaginalis; | XM_001612087; XM_001594710; |
| | Pichia guilliermondii; Pichia stipitis; | XM_001329865; XM_001487289; |
| | Lodderomyces elongisporus; | XM_001384591; XM_001528210; |
| | Coccidioides immitis; Trimastix | XM_001240868; DQ845797; |
| | pyriformis; Glycine max (soybean) | L08632 |
| 06a: | Peranema trichophorum; Euglena | GenBank: EF114757; AB021127, |
| Pyruvate-NADP⁺ | gracilis | AJ278425 |
| oxidoreductase | | |
| 06b: | Mastigamoeba balamuthi; Desulfovibrio | GenBank: AY101767; Y09702; |
| Pyruvate-ferredoxin | africanus; Entamoeba histolytica; | U30149; XM_001582310, |
| oxidoreductase | Trichomonas vaginalis; | XM_001313670, XM_001321286, |
| | Cryptosporidium parvum; | XM_001307087, |
| | Cryptosporidium baileyi; Giardia | XM_001311860, XM_001314776, |
| | lamblia; Entamoeba histolytica; | XM_001307250; EF030517; |
| | Hydrogenobacter thermophilus; | EF030516; XM_764947; |
| | Clostridium pasteurianum; | XM_651927; AB042412; Y17727 |
| 07: | Butyrivibrio fibrisolvens; butyrate- | GenBank: AB190764; DQ987697; |
| Thiolase | producing bacterium L2-50; | Z92974; |
| | Thermoanaerobacterium | |
| | thermosaccharolyticum; | |
| 08: | Clostridium beijerinckii; Butyrivibrio | GenBank: AF494018; AB190764; |
| 3-Hydroxybutyryl-CoA | fibrisolvens; Ajellomyces capsulatus; | XM_001537366; XM_741533; |
| dehydrogenase | Aspergillus fumigatus; Aspergillus | XM_001274776; XM_001262361; |
| | clavatus; Neosartorya fischeri; | DQ987697; BT001208; Z92974; |
| | Butyrate-producing bacterium L2-50; | |
| | Arabidopsis thaliana; | |
| | Thermoanaerobacterium | |
| | thermosaccharolyticum; | |
| 09: | Clostridium beijerinckii; Butyrivibrio | GenBank: AF494018; AB190764; |
| Crotonase | fibrisolvens; Butyrate-producing | DQ987697; Z92974 |
| | bacterium L2-50; | |
| | Thermoanaerobacterium | |
| | thermosaccharolyticum; | |
| 10: | Clostridium beijerinckii; Butyrivibrio | GenBank: AF494018; AB190764; |
| Butyryl-CoA dehydrogenase | fibrisolvens; Butyrate-producing | DQ987697; Z92974 |
| | bacterium L2-50; | |
| | Thermoanaerobacterium | |
| | thermosaccharolyticum; | |
| 11: | Clostridium | GenBank: AY251646 |
| Butyraldehyde | saccharoperbutylacetonicum | |
| dehydrogenase | | |
| 12a: | Geobacillus kaustophilus HTA426; | YP_148778, BAD77210; |
| NADH-dependent Butanol | Clostridium perfringens str. 13; | NP_561774, BAB80564; |
| dehydrogenase | Carboxydothermus hydrogenoformans; | AAG23613; |
| | Pseudovibrio sp. JE062; | ZP_05082669, EEA96294; |
| | Clostridium carboxidivorans P7; | ADO12118; |
| | Bacillus pseudofirmus OF4; | ADC48983, YP_003425875; |
| | Oceanobacillus iheyensis HTE831; | NP_693981, BAC15015; |
| | Slackia exigua ATCC 700122; | ZP_06159969, EEZ61452; |
| | Fusobacterium ulcerans ATCC 49185; | ZP_05633940; |
| | Listeria monocytogenes FSL J1-175; | ZP_05388801; |
| | Chlorobium chlorochromatii CaD3; | ABB28961; |
| | Clostridium perfringens D str. JGS1721; | ZP_02952811; |
| | Clostridium perfringens NCTC | ZP_02641897; |
| | 8239; Clostridium perfringens CPE str. | ZP_02638128; |
| | F4969; Clostridium perfringens B str. | ZP_02634798; |
| | ATCC 3626; | EDT24774; |
| | Clostridium botulinum NCTC 2916; | ZP_02614964, ZP_02614746; |
| | Nostoc sp. PCC 7120; | NP_488606, BAB76265; |
| 12b: | Clostridium perfringens str. 13; | NP_562172, BAB80962; |
| NADPH-dependent Butanol | Clostridium saccharobutylicum; | AAA83520; |
| dehydrogenase | Subdoligranulum variabile DSM 15176; | EFB77036; |
| | Butyrivibrio crossotus DSM 2876; | EFF67629, ZP_05792927; |
| | Oribacterium sp. oral taxon 078 str. | ZP_06597730, EFE92592; |
| | F0262; Clostridium sp. M62/1; | EFE12215, ZP_06346636; |
| | Clostridium hathewayi DSM 13479; | EFC98086, ZP_06115415; |
| | Subdoligranulum variabile DSM 15176; | ZP_05979561; |
| | Faecalibacterium prausnitzii A2-165; | ZP_05615704, EEU95840; |
| | Blautia hansenii DSM 20583; | ZP_05853889, EEX22072; |
| | Roseburia intestinalis L1-82, | ZP_04745071, EEU99657; |
| | Bacillus cereus Rock3-28; | ZP_04236939, EEL31374; |
| | Eubacterium rectale ATCC 33656; | YP_002938098, ACR75964; |
| | Clostridium sp. HGF2; | EFR36834; |
| | Atopobium rimae ATCC 49626; | ZP_03568088; |
| | Clostridium perfringens D str. JGS1721; | ZP_02952006; |
| | Clostridium perfringens NCTC 8239; | ZP_02642725; |
| | Clostridium butyricum 5521; | ZP_02950013, ZP_02950012; |
| | Clostridium carboxidivorans P7; | ZP_06856327; |
| | Clostridium botulinum E3 str. Alaska | YP_001922606, YP_001922335, |
| | E43; Clostridium novyi NT; | ACD52989; YP_878939; |
| | Clostridium botulinum B str. Eklund | YP_001887401; |
| | 17B; Thermococcus sp. AM4; | EEB74113; |
| | Fusobacterium sp. D11; | EFD81183; |
| | Anaerococcus vaginalis ATCC 51170; | ZP_05473100, EEU12061; |
| | Clostridium perfringens CPE str. | EDT27639; |
| | F4969; Clostridium perfringens B str. | EDT24389; |
| | ATCC 3626; | |
| 13: | Chlamydomonas reinhardtii; Phaseolus | GenBank: AF026422, AF026421, |
| Starch synthase | vulgaris; Oryza sativa; Arabidopsis | DQ019314, AF433156; |
| | thaliana; Colocasia esculenta; | AB293998; D16202, AB115917, |
| | Amaranthus cruentus; Parachlorella | AY299404; AF121673, |
| | kessleri; Triticum aestivum; Sorghum | AK226881; NM_101044; |
| | bicolor; Astragalus membranaceus; | AY225862, AY142712; |
| | Perilla frutescens; Zea mays; Ipomoea | DQ178026; AB232549; Y16340; |
| | batatas | AF168786; AF097922; |
| | | AF210699; AF019297; AF068834 |
| 14: | Arabidopsis thaliana; Zea mays; | GenBank: NM_127730, |
| Glucose-1-phosphate | Chlamydia trachomatis; Solanum | NM_124205, NM_121927, |
| adenylyltransferase | tuberosum (potato); Shigella flexneri; | AY059862; EF694839, |
| | Lycopersicon esculentum | EF694838; AF087165; P55242; |
| | | NP_709206; T07674 |
| 15: | Oryza sativa plastid; Ajellomyces | GenBank: AC105932, AF455812; |
| Phosphoglucomutase | capsulatus; Pichia stipitis; | XM_001536436; XM_001383281; |
| | Lodderomyces elongisporus; Aspergillus | XM_001527445; XM_749345; |
| | fumigatus; Arabidopsis thaliana; | NM_124561, NM_180508, |
| | Populus tomentosa; Oryza sativa; Zea | AY128901; AY479974; |
| | mays | AF455812; U89342, U89341 |
| 16: | Staphylococcus carnosus subsp. | YP_002633806, CAL27621; |
| Hexose-phosphate-isomerase | carnosus TM300; | |
| 17: | Hordeum vulgare aleuron cells; | GenBank: J04202; |
| Alpha-amylase; | Trichomonas vaginalis; Phanerochaete | XM_001319100; EF143986; |
| | chrysosporium; Chlamydomonas | AY324649; NM_129551; |
| | reinhardtii; Arabidopsis thaliana; | X07896; |
| | Dictyoglomus thermophilum heat-stable | |
| | amylase gene; | |
| Beta-amylase; | Arabidopsis thaliana; Hordeum vulgare; | GenBank: NM_113297; D21349; |
| | Musa acuminate; | DQ166026; |
| Starch phosphorylase; | Citrus hybrid cultivar root; Solanum | GenBank: AY098895; P53535; |
| | tuberosum chloroplast; Arabidopsis | NM_113857, NM_114564; |
| | thaliana; Triticum aestivum; Ipomoea | AF275551; M64362 |
| | batatas; | |
| 18: | Chlamydomonas reinhardtii; | JGI Chlre3 protein ID 135202; |
| Glucose-phosphate (glucose- | Saccharomyces cerevisiae; Pichia | GenBank: M21696; |
| 6-phosphate) isomerase | stipitis; Ajellomyces capsulatus; | XM_001385873; |
| | Spinacia oleracea cytosol; Oryza sativa | XM_001537043; T09154; |
| | cytoplasm; Arabidopsis thaliana; Zea | P42862; NM_123638, |
| | mays | NM_118595; U17225 |
| 19: | Chlamydomonas reinhardtii; | JGI Chlre2 protein ID 159495; |
| Phosphofructose kinase | Arabidopsis thaliana; Ajellomyces | GenBank: NM_001037043, |
| | capsulatus; Yarrowia lipolytica; Pichia | NM_179694, NM_119066, |
| | stipitis; Dictyostelium discoideum; | NM_125551; XM_001537193; |
| | Tetrahymena thermophila; | AY142710; XM_001382359, |
| | Trypanosoma brucei; Plasmodium | XM_001383014; XM_639070; |
| | falciparum; Spinacia oleracea; | XM_001017610; XM_838827; |
| | | XM_001347929; DQ437575; |
| 20: | Chlamydomonas reinhardtii chloroplast; | GenBank: X69969; AF308587; |
| Fructose-diphosphate | Fragaria x ananassa cytoplasm; Homo | NM_005165; XM_001609195; |
| aldolase | sapiens; Babesia bovis; Trichomonas | XM_001312327, XM_001312338; |
| | vaginalis; Pichia stipitis; Arabidopsis | XM_001387466; NM_120057, |
| | thaliana | NM_001036644 |
| 21: | Arabidopsis thaliana; Chlamydomonas | GenBank: NM_127687, |
| Triose phosphate isomerase | reinhardtii; Sclerotinia sclerotiorum; | AF247559; AY742323; |
| | Chlorella pyrenoidosa; Pichia | XM_001587391; AB240149; |
| | guilliermondii; Euglena intermedia; | XM_001485684; DQ459379; |
| | Euglena longa; Spinacia oleracea; | AY742325; L36387; AY438596; |
| | Solanum chacoense; Hordeum vulgare; | U83414; EF575877; |
| | Oryza sativa | |
| 34: | Staphylococcus aureus 04-02981; | ADC37857; |
| NADPH-dependent | Staphylococcus lugdunensis; | ADC87332; |
| Glyceraldehyde-3-phosphate | Staphylococcus lugdunensis HKU09; | YP_003471459; |
| dehydrogenase | Vibrio cholerae BX 330286; | ZP_04395517; |
| | Vibrio sp. Ex25; | YP_003287699; |
| | Pseudomonas savastanoi pv.; | ZP_07004478, EFI00105; |
| | Vibrio cholerae B33; | ZP_04399616 |
| | Grimontia hollisae CIP 101886; | ZP_06052988, EEY71738; |
| | Vibrio mimicus MB-451, | ZP_06041160; |
| | Vibrio coralliilyticus ATCC BAA-450; | ZP_05886203; |
| | Vibrio cholerae MJ-1236; | YP_002876243; |
| | Zea mays cytosolic NADP dependent; | NP_001105589; |
| | Apium graveolens; | AAF08296; |
| | Vibrio cholerae B33; | EEO17521; |
| | Vibrio cholerae TMA 21; | EEO13209; |
| | Vibrio cholerae bv. albensis VL426; | EEO01829; |
| | Vibrio orientalis CIP 102891; | ZP_05943395; |
| | Vibrio cholerae MJ-1236; | ACQ62447; |
| | Vibrio cholerae CT 5369-93; | ZP_06049761; |
| | Vibrio sp. RC586; | ZP_06079970; |
| | Vibrio furnissii CIP 102972; | ZP_05878983; |
| | Vibrio metschnikovii CIP 69.14; | ZP_05883187; |
| 35: | Edwardsiella tarda FL6-60; | ADM41489; |
| NAD-dependent | Flavobacteriaceae bacterium 3519-10; | YP_003095198; |
| Glyceraldehyde-3-phosphate | Staphylococcus aureus 04-02981; | ADC36961; |
| dehydrogenase | Pseudomonas savastanoi pv. savastanoi | ZP_07003925; |
| | NCPPB 3335; | |
| | Vibrio cholerae MJ-1236; | ACQ61431, YP_002878104; |
| | Streptococcus pyogenes NZ131; | YP_002285269; |
| | Helicobacter pylori 908; | ADN80469; |
| | Streptococcus pyogenes NZ131; | ACI60574; |
| | Staphylococcus lugdunensis HKU09; | ADC88142; |
| | Vibrio sp. Ex25; | ACY51070; |
| | Stenotrophomonas chelatiphaga; | ADK67090; |
| | Pseudoxanthomonas dokdonensis; | ADK67075; |
| | Stenotrophomonas maltophilia; | ADK67085, ACH90636; |
| | Vibrio cholerae B33; Photobacterium | ZP_04401333; |
| | damselae subsp. damselae CIP 102761; | ZP_06155532; |
| | Vibrio sp. RC586; | ZP_06080908; |
| | Grimontia hollisae CIP 101886; | ZP_06052393; |
| | Vibrio furnissii CIP 102972; | EEX42220; |
| | Acidithiobacillus caldus ATCC 51756; | ZP_05292346; |
| | Nostoc sp. PCC 7120; | CAC41000; |
| | Vibrio cholerae BX 330286; | EEO22474; |
| | Vibrio cholerae TMA 21; | EEO13042; |
| | Nostoc sp. PCC 7120; | CAC41000; |
| | Pinus sylvestris; | CAA04942; |
| | Cheilanthes yavapensis; | ACO58643, ACO58642; |
| | Cheilanthes wootonii; | ACO58624, ACO58623; |
| | Astrolepis laevis; | CBH41484, CBH41483; |
| 36: | Hydrogenobacter thermophilus TK-6; | YP_003433013, ADO45737, |
| (R)-Citramalate synthase | Geobacter bemidjiensis Bem; | BAI69812; |
| (EC 2.3.1.182) | Geobacter sulfurreducens KN400; | ACH38284; |
| | Methanobrevibacter ruminantium M1; | ADI84633; |
| | Leptospira biflexa serovar Patoc strain | CP001719; |
| | ‘Patoc 1 (Paris)’; Leptospira biflexa | ABK13757; |
| | serovar Monteralerio; Leptospira | ABK13756; |
| | interrogans serovar Australis; | ABK13755; |
| | Leptospira interrogans serovar | ABK13753; |
| | Pomona; Leptospira interrogans | ABK13754; |
| | serovar Autumnalis; Leptospira | ABK13752; |
| | interrogans serovar Pyrogenes; | ABK13751; |
| | Leptospira interrogans serovar | ABK13750; |
| | Canicola; Leptospira interrogans | ABK13749; |
| | serovar Lai; Acetohalobium arabaticum | ADL11763, |
| | DSM 5501; Leadbetterella byssophila | YP_003998693; |
| | DSM 17132; Bacteroides xylanisolvens | CBK66631; |
| | XB1A; Mucilaginibacter paludis DSM | EFQ72644; |
| | 18603; Prevotella ruminicola 23; | ADE82919; |
| | Flavobacterium johnsoniae UW101; | ABQ04337; |
| | Victivallis vadensis ATCC BAA-548; | ZP_06244204, |
| | Prevotella copri DSM 18205; | EFA99692; |
| | Alistipes shahii WAL 8301; | EFB36404, ZP_06251228; |
| | Methylobacter tundripaludum SV96; | CBK64953; |
| | Methanosarcina mazei Go1; | ZP_07654184; |
| | | NP_632695; |
| 37: | Eubacterium eligens ATCC 27750 | YP_002930810, YP_002930809; |
| (R)-2-Methylmalate | Methanocaldococcus jannaschii; | P81291; |
| dehydratase (large and small | Sebaldella termitidis ATCC 33386; | ACZ06998; |
| subunits) | Eubacterium eligens ATCC 27750; | ACR72362, ACR72361, |
| (EC 4.2.1.35) | | ACR72363, YP_002930808; |
| 38: | Thermotoga petrophila RKU-1; | ABQ46641, ABQ46640; |
| 3-Isopropylmalate | Cyanothece sp. PCC 7822; | YP_003886427, YP_003889452; |
| dehydratase (large + small | Syntrophothermus lipocalidus DSM | ADI02900, ADI02899, |
| subunits) | 12680; | YP_003703465, ADI01294; |
| (EC 4.2.1.33) | Caldicellulosiruptor saccharolyticus | ABP66933, ABP66934; |
| | DSM 8903; | |
| | Pelotomaculum thermopropionicum SI, | YP_001211082, YP_001211083; |
| | Caldicellulosiruptor bescii DSM 6725; | YP_002573950, YP_002573949; |
| | Caldicellulosiruptor saccharolyticus | YP_001180124, YP_001180125; |
| | DSM 8903; | leuC, ECK0074, JW0071; |
| | E. coli; | leuD, ECK0073, JW0070; |
| | Spirochaeta thermophila DSM 6192; | YP_003875294, YP_003873373; |
| | Pelotomaculum thermopropionicum SI; | YP_001213069, YP_001213068; |
| | Hydrogenobacter thermophilus TK-6; | YP_003433547, YP_003432351; |
| | Deferribacter desulfuricans SSM1; | YP_003495505, YP_003495504; |
| | Anoxybacillus flavithermus WK1; | ACJ32977, ACJ32978; |
| | Thermosynechococcus elongatus BP-1; | BAC08461, BAC08786; |
| | Geobacillus kaustophilus HTA426; | BAD76941, BAD76940; |
| | Synechocystis sp. PCC 6803; | BAA18738, BAA18298; |
| | Chlamydomonas reinhardtii; | XP_001702135, XP_001696402; |
| 39: | Thermotoga petrophila RKU-1; | ABQ46392, YP_001243968; |
| 3-Isopropylmalate | Cyanothece sp. PCC 7822; | YP_003888480, ADN15205; |
| dehydrogenase | Thermosynechococcus elongatus BP-1; | BAC09152, NP_682390; |
| (EC 1.1.1.85) | Syntrophothermus lipocalidus DSM | ADI02898, YP_003703463; |
| | 12680; | |
| | Caldicellulosiruptor bescii DSM 6725; | ADQ78220; |
| | Paludibacter propionicigenes WB4; | YP_002573948; |
| | Leadbetterella byssophila DSM 17132; | YP_003998692; |
| | Caldicellulosiruptor saccharolyticus | ABP66935; |
| | DSM 8903; Thermus thermophilus; | AAA16706, YP_001180126; |
| | Pelotomaculum thermopropionicum SI; | YP_001211084; |
| | Geobacillus kaustophilus HTA426; | YP_148510, BAD76942; |
| | Hydrogenobacter thermophilus TK-6; | YP_003433176; |
| | Spirochaeta thermophila DSM 6192; | YP_003873639; |
| | Deferribacter desulfuricans SSM1; | YP_003495917; |
| | Anoxybacillus flavithermus WK1; | YP_002314961; |
| | Volvox carteri f. nagariensis; | XP_002955062, EFJ43816; |
| | Chlamydomonas reinhardtii; | XP_001701074, XP_001701073; |
| | Ostreococcus tauri; | XP_003083133; |
| 40: | Thermotoga petrophila RKU-1; | ABQ46395, YP_001243971; |
| 2-Isopropylmalate synthase | Cyanothece sp. PCC 7822; | YP_003890122, ADN16847; |
| (EC 2.3.3.13) | Cyanothece sp. PCC 8802; | ACU99797; |
| | Nostoc punctiforme PCC 73102; | ACC82459; |
| | Pelotomaculum thermopropionicum SI; | YP_001211081; |
| | Hydrogenobacter thermophilus TK-6; | YP_003432474, BAI69273; |
| | E. coli; Caldicellulosiruptor | NP_414616, AAC73185; |
| | saccharolyticus DSM 8903; | ABP66753, YP_001179944; |
| | Syntrophothermus lipocalidus DSM | YP_003703466, ADI02901; |
| | 12680; Geobacillus kaustophilus | YP_148511, BAD76943; |
| | HTA426; Caldicellulosiruptor bescii | YP_002572404; |
| | DSM 6725; Anoxybacillus flavithermus | YP_002314960, ACJ32975; |
| | WK1; Deferribacter desulfuricans | YP_003496874, BAI81118; |
| | SSM1; Thermosynechococcus elongatus | NP_682187, BAC08949; |
| | BP-1; Spirochaeta thermophila DSM | ADN03009, YP_003875282; |
| | 6192; Thermotoga lettingae TMO; | YP_001469896, ABV32832; |
| | Volvox carteri f. nagariensis; | XP_002945733, |
| | Micromonas sp. RCC299; | EFJ52728; |
| | Micromonas pusilla CCMP1545; | ACO69978, XP_002508720; |
| | Chlamydomonas reinhardtii; | XP_003063010, EEH52949; |
| | | XP_001696603, EDP08580; |
| 41: | Geobacillus kaustophilus HTA426; | YP_148509, YP_148508; |
| isopropylmalate isomerase | Anabaena variabilis ATCC 29413; | YP_324467, YP_324466; |
| large/small subunits | Synechocystis sp. PCC 6803; | NP_442926, NP_441618; |
| (EC 4.2.1.33) | Anoxybacillus flavithermus WK1; | YP_002314962, YP_002314963; |
| | Thermosynechococcus elongatus BP-1; | NP_682024, NP_681699; |
| | Spirochaeta thermophila DSM 6192; | YP_003873372; |
| | Salmonella enterica subsp. enterica | CBG23133, CBG23132; |
| | serovar Typhimurium str. D23580; | |
| | Staphylococcus aureus A5937; | ZP_05702396; |
| | Francisella philomiragia subsp. | EET20545; |
| | philomiragia ATCC 25015; | AAA53236; |
| | Neisseria lactamica; Francisella | ABK88972; |
| | novicida U112; Staphylococcus aureus | EEV86047; |
| | A5937; Staphylococcus aureus subsp. | ZP_05607839; |
| | aureus 68-397; Fusobacterium sp. | EEO38992; |
| | 2_1_31; Francisella novicida GA99- | EDN35429; |
| | 3549; marine bacterium HP15; | ADP98363, ADP98362; |
| | Bacillus licheniformis ATCC 14580; | YP_092517, YP_092516; |
| | Rhodobacter sphaeroides 2.4.1; | YP_353947, YP_353945; |
| | Bordetella petrii DSM 12804; | YP_001631647, YP_001631646; |
| | Agrobacterium vitis S4; | YP_002551071, YP_002551071; |
| 42: | Lactococcus lactis; | AAS49166; |
| 2-keto acid decarboxylase | Lactococcus lactis subsp. lactis KF147; | ADA65057, YP_003353820; |
| (EC 4.1.1.72, etc) | Lactococcus lactis subsp. Lactis; | |
| | Kluyveromyces marxianus; | CAG34226; |
| | Kluyveromyces lactis; | AAA35267; |
| | Mycobacterium avium 104; | CAA59953; |
| | Mycobacterium ulcerans Agy99; | A0QBE6; |
| | Mycobacterium bovis; | A0PL16; |
| | Mycobacterium leprae; | Q7U140; |
| | Proteus mirabilis HI4320; | Q9CBD6; |
| | Staphylococcus aureus 04-02981; | YP_002150004; |
| | Acetobacter pasteurianus; | ADC36400; |
| | Saccharomyces cerevisiae; | AAM21208; |
| | Zymomonas mobilis subsp. mobilis CP4; | CAA39398; |
| | Mycobacterium tuberculosis; | AAA27696; |
| | Mycobacterium smegmatis str. MC2 | O53865; |
| | 155; Mycobacterium bovis BCG str. | A0R480; |
| | Pasteur 1173P2; | A1KGY5; |
| 43: | Thermoplasma volcanium GSS1; | BAB59540 |
| Alcohol dehydrogenase | Gluconacetobacter hansenii ATCC | ZP_06834544; |
| (NAD dependent) | 23769; Saccharomyces cerevisiae; | CAA89136; |
| (EC 1.1.1.1); | Aeropyrum pernix K1; | NP_148480; |
| | Rhodobacterales bacterium HTCC2083; | ZP_05073895; |
| | Bradyrhizobium japonicum USDA 110; | NP_769420; |
| | Syntrophothermus lipocalidus DSM | ADI01021; |
| | 12680; Fervidobacterium nodosum | YP_001411173; |
| | Rt17-B1; Desulfotalea psychrophila | YP_065604; |
| | LSv54; Acetobacter pasteurianus IFO | BAI03878; |
| | 3283-03; Gluconobacter oxydans 621H; | YP_192500; |
| | Aeromonas hydrophila subsp. | ABK38651; |
| | hydrophila ATCC 7966; Acetobacter | BAI00830; |
| | pasteurianus IFO 3283-01; | EFL29096; |
| | Streptomyces hygroscopicus ATCC | |
| | 53653; | |
| 44: | Pelotomaculum thermopropionicum SI; | YP_001211038, BAF58669; |
| Alcohol dehydrogenase | Fusobacterium sp. 7_1; | ZP_04573952, EEO43462; |
| (NADPH dependent) (EC | Pichia pastoris GS115; | XP_002494014, XP_002490014; |
| 1.1.1.2); | Pichia pastoris GS115; | CAY71835, XP_002492217, |
| | Escherichia coli str. K-12 substr. | CAY67733; |
| | MG1655; | yqhD, NP_417484, AAC76047; |
| | Clostridium hathewayi DSM 13479; | EFC99049; |
| | Clostridium butyricum 5521; | ZP_02948287 |
| | Fusobacterium ulcerans ATCC 49185; | ZP_05632371; |
| | Fusobacterium sp. D11; Desulfovibrio | ZP_05440863; |
| | desulfuricans subsp. desulfuricans str. | YP_389756; |
| | G20; Clostridium novyi NT; | YP_878957; |
| | Clostridium tetani E88; | NP_782735; |
| | Aureobasidium pullulans; | ADG56699; |
| | Scheffersomyces stipitis CBS 6054, | ABN66271, XP_001384300; |
| | Thermotoga lettingae TMO; | YP_001471424; |
| | Thermotoga petrophila RKU-1; | YP_001244106; |
| | Coprinopsis cinerea okayama7#130; | XP_001834460; |
| | Saccharomyces cerevisiae EC1118; | CAY82157; |
| | Saccharomyces cerevisiae JAY291; | EEU07174; |
| 45: | Thermaerobacter subterraneus DSM | EFR61439; |
| Phosphoenolpyruvate | 13965; Cyanothece sp. PCC 7822; | YP_003887888; |
| carboxylase | Thermus sp.; Rhodothermus marinus; | BAA07723; CAA67760; |
| (EC 4.1.1.31) | Thermosynechococcus elongatus BP-1; | NP_682702, BAC09464; |
| | Leadbetterella byssophila DSM 17132; | YP_003998059, ADQ17706; |
| | Riemerella anatipestifer DSM 15868; | ADQ81501, YP_004045007; |
| | Mucilaginibacter paludis DSM 18603; | EFQ77722; |
| | Truepera radiovictrix DSM 17093; | YP_003706036; |
| | Ferrimonas balearica DSM 9799; | YP_003911597, ADN74523; |
| | Meiothermus silvanus DSM 9946; | YP_003685046; |
| | Nocardiopsis dassonvillei subsp. | YP_003681843; |
| | dassonvillei DSM 43111; E. coli, | ZP_07594313, ZP_07565817; |
| | Meiothermus ruber DSM 1279; | ADD27759; |
| | Olsenella uli DSM 7084; | YP_003801346, ADK68466; |
| | Ktedonobacter racemifer DSM 44963; | ZP_06967036, EFH90147; |
| | Rhodopirellula baltica SH 1; | NP_866412, CAD78193; |
| | Oceanithermus profundus DSM 14977; | ADR36285; |
| | marine bacterium HP15; | ADP96559; |
| | Marivirga tractuosa DSM 4126; | ADR23252; |
| | Mucilaginibacter paludis DSM 18603; | ZP_07746438; |
| | Streptomyces coelicolor A3(2); | NP_627344; |
| | Delftia acidovorans SPH-1; | ABX34873; |
| | Actinobacillus pleuropneumoniae | ZP_07544559; |
| | serovar 13 str. N273; Prochlorococcus | ABO18389; |
| | marinus str. MIT 9301; | |
| | Prochlorococcus marinus str. NATL1A | ABM76577; |
| | Prochlorococcus marinus str. MIT | ABM72969; |
| | 9515; Clostridium cellulovorans 743B; | YP_003842669, ADL50905; |
| | Neisseria meningitidis Z2491; | CAM07667; |
| | Deinococcus geothermalis DSM 11300; | ABF44963; |
| | Micromonospora sp. L5; | ZP_06399624; |
| | Chlorobium phaeobacteroides DSM | ABL64615; |
| | 266; Arthrobacter sp. FB24; | YP_830113; |
| | Rhodomicrobium vannielii ATCC | YP_004010507; |
| | 17100; Gordonia bronchialis DSM | YP_003273502; |
| | 43247; Thermus aquaticus Y51MC23; | ZP_03496338; |
| | Burkholderia ambifaria IOP40-10; | ZP_02894226; |
| 46: | Thermotoga lettingae TMO; | YP_001470126; |
| Aspartate aminotransferase | Synechococcus elongatus PCC 6301; | YP_172275; |
| (EC 2.6.1.1) | Synechococcus elongatus PCC 7942; | YP_401562; |
| | Thermosipho melanesiensis BI429; | YP_001306480; |
| | Thermotoga petrophila RKU-1; | YP_001244588; |
| | Thermus thermophilus; | BAA07487; |
| | Anoxybacillus flavithermus WK1; | YP_002315494; |
| | Bacillus sp.; E. coli, | AAA22250; aspC: BAB34434; |
| | Pelotomaculum thermopropionicum SI; | YP_001211971; |
| | Phormidium lapideum; | BAB86290; |
| | Fervidobacterium nodosum Rt17-B1; | YP_001410686, YP_001409589; |
| | Geobacillus kaustophilus HTA426; | YP_148025, YP_147632, |
| | Thermosynechococcus elongatus BP-1; | YP_146225; NP_683147; |
| | Anoxybacillus flavithermus WK1; | ACJ34747; |
| | Geobacillus kaustophilus HTA426; | BAD77213, BAD76064; |
| | Spirochaeta thermophila DSM 6192; | YP_003874653; |
| | Caldicellulosiruptor bescii DSM 6725; | YP_002572445; |
| | Caldicellulosiruptor saccharolyticus | YP_001179582; |
| | DSM 8903; | |
| | Arabidopsis thaliana; | AAA79371; |
| | Glycine max; | AAA33942; |
| | Lupinus angustifolius; | CAA42430; |
| | Chlamydomonas reinhardtii; | XP_001696609; |
| | Micromonas pusilla CCMP1545; | XP_003060871; |
| 47: | Thermotoga lettingae TMO; | YP_001470361, ABV33297; |
| Aspartokinase (EC = 2.7.2.4) | Cyanothece sp. PCC 8802; | YP_003136939; |
| | Thermotoga petrophila RKU-1 | YP_001244864, YP_001243977; |
| | Hydrogenobacter thermophilus TK-6; | YP_003432105, BAI68904; |
| | Anoxybacillus flavithermus WK1; | ACJ35001; |
| | Bacillus sp.; | AAA22251; |
| | Spirochaeta thermophila DSM 6192; | YP_003873788, ADN01515; |
| | Anoxybacillus flavithermus WK1; | ACJ34043, YP_002316986; |
| | Geobacillus kaustophilus HTA426; | BAD77480, YP_149048; |
| | Syntrophothermus lipocalidus DSM | ADI02230, YP_003702795; |
| | 12680; E. coli; | ZP_07594328, ZP_07565832; |
| | Thermosynechococcus elongatus BP-1; | NP_682623, BAC09385; |
| | Fervidobacterium nodosum Rt17-B1; | ABS59942, YP_001410786; |
| | Spirochaeta thermophila DSM 6192; | YP_003873302, ADN01029; |
| | Pelotomaculum thermopropionicum SI; | YP_001212149, YP_001211837; |
| | Caldicellulosiruptor saccharolyticus | ABP66605; |
| | DSM 8903; Caldicellulosiruptor bescii | YP_002573821; |
| | DSM 6725; Thermosipho melanesiensis | YP_001307097, ABR31712; |
| | BI429; Thermotoga lettingae TMO; | YP_001470985, ABV33921; |
| | Arabidopsis thaliana; | CAA67376; |
| | Chlamydomonas reinhardtii; | XP_001698576, EDP08069, |
| | | XP_001695256; |
| 48: | Thermotoga lettingae TMO; | YP_001470981, ABV33917; |
| Aspartate-semialdehyde | Trichodesmium erythraeum IMS101; | ABG50031; |
| dehydrogenase | Prochlorococcus marinus str. MIT | ABM76828; |
| | 9303; | ABQ47283, YP_001244859; |
| | Thermotoga petrophila RKU-1; | ABP67176, YP_001180367; |
| | Caldicellulosiruptor saccharolyticus | ADI01804, YP_003702369; |
| | DSM 8903; Syntrophothermus | YP_001460230, YP_001464895; |
| | lipocalidus DSM 12680; E. coli; | YP_001409594, ABS59937; |
| | Fervidobacterium nodosum Rt17-B1 | YP_002573009; |
| | Caldicellulosiruptor bescii DSM 6725; | YP_001307092, ABR31707; |
| | Thermosipho melanesiensis BI429; | YP_003875128, ADN02855; |
| | Spirochaeta thermophila DSM 6192; | YP_001211836, BAF59467; |
| | Pelotomaculum thermopropionicum SI; | YP_003432252, BAI69051; |
| | Hydrogenobacter thermophilus TK-6; | YP_002316029, ACJ34044; |
| | Anoxybacillus flavithermus WK1; | YP_147128, BAD75560; |
| | Geobacillus kaustophilus HTA426; | YP_003496635, BAI80879; |
| | Deferribacter desulfuricans SSM1; | NP_680860, BAC07622; |
| | Thermosynechococcus elongatus BP-1; | AAG23574, AAG23573; |
| | Carboxydothermus hydrogenoformans; | XP_001695059, EDP02211; |
| | Chlamydomonas reinhardtii; | ABH11018; |
| | Polytomella parva; | ACU30050; |
| | Glycine max; | ACG41594; |
| | Zea mays; | ABR26065; |
| | Oryza sativa Indica Group; | |
| 49: | Syntrophothermus lipocalidus DSM | ADI02231, YP_003702796; |
| Homoserine dehydrogenase | 12680; Cyanothece sp. PCC 7822; | YP_003887242; |
| | Caldicellulosiruptor bescii DSM 6725; | YP_002573819; |
| | Caldicellulosiruptor saccharolyticus | ABP66607, YP_001179798; |
| | DSM 8903; E. coli; | EFJ98002; |
| | Spirochaeta thermophila DSM 6192; | YP_003873441, ADN01168; |
| | Pelotomaculum thermopropionicum SI; | YP_001212151, BAF59782; |
| | Hydrogenobacter thermophilus TK-6; | YP_003431981, BAI68780; |
| | Anoxybacillus flavithermus WK1; | YP_002316756, ACJ34771; |
| | Geobacillus kaustophilus HTA426; | YP_148817, BAD77249; |
| | Deferribacter desulfuricans SSM1; | YP_003496401, BAI80645; |
| | Thermosynechococcus elongatus BP-1; | NP_681068, BAC07830; |
| | Glycine max; | ABG78600, AAZ98830; |
| | Chlamydomonas reinhardtii; | XP_001699712, EDP07408; |
| | Micromonas sp. RCC299; | ACO69662, XP_002508404; |
| 50: | Thermotoga petrophila RKU-1; | YP_001243979, ABQ46403; |
| Homoserine kinase | Cyanothece sp. PCC 7822; | YP_003886645; |
| (EC 2.7.1.39) | Caldicellulosiruptor bescii DSM 6725; | YP_002573820; |
| | Caldicellulosiruptor saccharolyticus | ABP66606, YP_001179797; |
| | DSM 8903; E. coli; | AP_000667, BAB96580; |
| | Anoxybacillus flavithermus WK1; | YP_002316754, ACJ34769; |
| | Geobacillus kaustophilus HTA426; | YP_148815, BAD77247; |
| | Thermosynechococcus elongatus BP-1; | NP_682555, BAC09317; |
| | Pelotomaculum thermopropionicum SI; | YP_001212150, BAF59781; |
| | Hydrogenobacter thermophilus TK-6; | YP_003433124, BAI69923; |
| | Chlamydomonas reinhardtii; | XP_001701899, EDP06874; |
| | Prototheca wickerhamii; | ABC24954; |
| | Arabidopsis thaliana; | NP_179318, AAD33097; |
| | Glycine max; | ACU26535; |
| | Zea mays; | ACG46592; |
| 51: | Thermotoga petrophila RKU-1; | YP_001243978, ABQ46402; |
| Threonine synthase | Cyanothece sp. PCC 7425; | YP_002485009; |
| (EC 4.2.99.2) | Thermosipho melanesiensis BI429; | YP_001306558, ABR31173; |
| | Syntrophothermus lipocalidus DSM | ADI02519, YP_003703084; |
| | 12680; E. coli; | AP_000668, NP_414545; |
| | Pelotomaculum thermopropionicum SI; | YP_001213220; |
| | Anoxybacillus flavithermus WK1; | YP_002316755, ACJ34770; |
| | Caldicellulosiruptor bescii DSM 6725; | YP_002572552; |
| | Caldicellulosiruptor saccharolyticus | YP_001180015, ABP66824; |
| | DSM 8903; Hydrogenobacter | YP_003433070, YP_003433019, |
| | thermophilus TK-6; Geobacillus | BAI69869, BAI69818; |
| | kaustophilus HTA426; | YP_148816, YP_147614; |
| | Thermosynechococcus elongatus BP-1; | NP_682017, NP_681772, |
| | Spirochaeta thermophila DSM 6192; | BAC08534, BAC08779; |
| | Deferribacter desulfuricans SSM1; | YP_003873303, ADN01030; |
| | Geobacillus kaustophilus HTA426; | YP_003495358, BAI79602; |
| 52: | Geobacillus kaustophilus HTA426; | BAD76058, BAD75876, |
| Threonine ammonia-lyase | Prochlorococcus marinus str. MIT | YP_147626, YP_147444; |
| (EC 4.3.1.19) | 9202; Synechococcus sp. PCC 7335; | ZP_05137562; ZP_05035047; |
| | Thermotoga petrophila RKU-1; | ABQ46585, YP_001244161; |
| | Pelotomaculum thermopropionicum SI; | YP_001210652, BAF58283; |
| | Anoxybacillus flavithermus WK1; | YP_002315804, YP_002315746; |
| | Deferribacter desulfuricans SSM1; | YP_003497384, BAI81628; |
| | E. coli; | YP_001746093, ZP_07690697; |
| | Neisseria lactamica ATCC 23970; | EEZ76650, ZP_05986317; |
| | Citrobacter youngae ATCC 29220; | EFE07783, ZP_06571237; |
| | Neisseria polysaccharea ATCC 43768; | EFH23894, ZP_06863451; |
| | Providencia rettgeri DSM 1131; | EFE52186, ZP_06127162; |
| | Neisseria subflava NJ9703; | EFC51529, ZP_05985502; |
| | Mannheimia haemolytica PHL213; | ZP_04978734; |
| | Achromobacter piechaudii ATCC | ZP_06687730, ZP_06684811; |
| | 43553; Neisseria meningitidis ATCC | ZP_07369980, EFM04207; |
| | 13091; Synechococcus sp. CC9902; | |
| | Synechococcus sp. PCC 7002; | ABB26032; |
| | Synechococcus sp. WH 8109; | ACA99606; |
| | Cyanobium sp. PCC 7001; | ZP_05790446, EEX07646; |
| | Anabaena variabilis ATCC 29413; | EDY39077, ZP_05045768; |
| | Microcoleus chthonoplastes PCC 7420; | ABA20300; |
| | Chlamydomonas reinhardtii; | ZP_05029756; |
| | | XP_001701816, EDP06791; |
| 53: | Caldicellulosiruptor saccharolyticus | ABP66750, ABP66751, |
| Acetolactate synthase | DSM 8903; | YP_001179942, ABP66455, |
| (EC 2.2.1.6) | | YP_001179941, YP_001179646; |
| | Thermotoga petrophila RKU-1; | YP_001243976, YP_003345845, |
| | | ADA66432, ADA66431, |
| | | ABQ46399, YP_001243975, |
| | | ABQ46400, YP_003345846; |
| | Thermosynechococcus elongatus BP-1; | NP_682614, BAC09376, |
| | | NP_681670, BAC08432, |
| | | NP_682086; |
| | Syntrophothermus lipocalidus DSM | ADI02904, YP_003703469, |
| | 12680; | ADI02903, YP_003703468; |
| | Pelotomaculum thermopropionicum SI; | BAF58709, BAF58917, |
| | | YP_001211286, YP_001211078; |
| | Geobacillus kaustophilus HTA426; | BAD76946, YP_148514, |
| | | BAD76945, YP_148513: |
| | Caldicellulosiruptor bescii DSM 6725; | ACM59790, ACM59628, |
| | | ACM59629, YP_002572563, |
| | | YP_002572401, YP_002572402; |
| | | YP_003432299, YP_003432300, |
| | Hydrogenobacter thermophilus TK-6; | BAI69099, BAI69098; |
| | | YP_003874926, YP_003874927, |
| | Spirochaeta thermophila DSM 6192; | ADN02654, ADN02653, |
| | | ACJ33615, YP_002314957, |
| | Anoxybacillus flavithermus WK1; | ACJ32972, ACJ32973, |
| | | YP_002314958; |
| | | YP_003496879, BAI81123, |
| | Deferribacter desulfuricans SSM1; | YP_003496878, BAI81122; |
| | | AP_004121, BAE77622, |
| | Escherichia coli str. K-12 substr. | AP_004122, BAE77623, |
| | W3110; | BAE77528, AP_004027, |
| | | BAB96646, AP_000741; |
| | | BAA12700; |
| | Saccharomyces cerevisiae, | EDN64495, CAA89744, |
| | | EDV09697; |
| | Thermus aquaticus; | YP_001735999, ACB00744; |
| | Synechococcus sp. PCC 7002; | YP_002376012; |
| | Cyanothece sp. PCC 7424; | YP_324035; |
| | Anabaena variabilis ATCC 29413; | NP_487595, BAB75254; |
| | Nostoc sp. PCC 7120; | YP_001655615; |
| | Microcystis aeruginosa NIES-843; | NP_441297, BAA17984, |
| | Synechocystis sp. PCC 6803; | CAA66718, NP_441304, |
| | | NP_442206, BAA10276 ; |
| | Synechococcus sp. JA-2-3B′a(2-13); | YP_478353; |
| | Synechococcus sp. JA-3-3Ab; | YP_475372, ABD00213, |
| | | ABD00270, YP_475476, |
| | | YP_475533; |
| | Chlamydomonas reinhardtii; | AAC03784, AAB88292, |
| | | XP_001700185, EDO98300, |
| | | XP_001695168, EDP01876; |
| | Volvox carteri; | AAC04854, AAB88296; |
| | Bacillus subtilis subsp. subtilis str. 168; | CAB07802 (AlsS); |
| | Bacillus licheniformis ATCC 14580; | AAU42663 (AlsS); |
| 54: | Syntrophothermus lipocalidus DSM | ADI02902, YP_003703467; |
| Ketol-acid reductoisomerase | 12680; Caldicellulosiruptor | ABP66752, YP_001179943; |
| (EC 1.1.1.86) | saccharolyticus DSM 8903; E. coli; | AAA67577, YP_001460567; |
| | Thermotoga petrophila RKU-1; | ABQ46398, YP_001243974; |
| | Calditerrivibrio nitroreducens DSM | YP_004050904; |
| | 19672; | |
| | Spirochaeta thermophila DSM 6192; | YP_003874858, ADN02585; |
| | Pelotomaculum thermopropionicum SI; | YP_001211079, BAF58710; |
| | Cyanothece sp. PCC 7822; | YP_003885458; |
| | Hydrogenobacter thermophilus TK-6; | YP_003433279, BAI70078; |
| | Anoxybacillus flavithermus WK1; | YP_002314959, ACJ32974; |
| | Caldicellulosiruptor bescii DSM 6725; | YP_002572403; |
| | Geobacillus kaustophilus HTA426; | YP_148512, BAD76944; |
| | Deferribacter desulfuricans SSM1; | YP_003496877, BAI81121; |
| | Thermosynechococcus elongatus BP-1; | NP_683044, BAC09806; |
| | Cyanothece sp. PCC 7425; | YP_002482078; |
| | Nostoc punctiforme PCC 73102; | ACC82013; |
| | Trichodesmium erythraeum IMS101; | ABG53327; |
| | Synechococcus sp. PCC 7335; | ZP_05036558; |
| | Microcoleus chthonoplastes PCC 7420; | ZP_05026584; |
| | Prochlorococcus marinus str. MIT | ABO18124; |
| | 9301; Cyanobium sp. PCC 7001; | EDY39000; |
| | Arthrospira sp. PCC 8005; | ZP_07166132; |
| | Arabidopsis thaliana; | CAA48253, NP_001078309; |
| | Pisum sativum (pea); | CAA76854; |
| | Zea mays; | ACG35752; |
| | Chlamydomonas reinhardtii; | XP_001702649, EDP06428; |
| | Polytomella parva; | ABH11013; |
| 55: | Thermotoga petrophila RKU-1; | YP_001243973, ABQ46397; |
| Dihydroxy-acid dehydratase | Cyanothece sp. PCC 7822; | YP_003887466; |
| (EC 4.2.1.9) | Marivirga tractuosa DSM 4126; | YP_004053736; |
| | Geobacillus kaustophilus HTA426; | YP_147899, BAD76331, |
| | Syntrophothermus lipocalidus DSM | YP_147822, BAD76254; |
| | 12680; | ADI02905, YP_003703470; |
| | Spirochaeta thermophila DSM 6192; | YP_003874669, ADN02396; |
| | Anoxybacillus flavithermus WK1; | YP_002315593; |
| | Caldicellulosiruptor bescii DSM 6725; | YP_002572562; |
| | Caldicellulosiruptor saccharolyticus | YP_001179645, ABP66454; |
| | DSM 8903; E. coli; | ADR29155, YP_001460564; |
| | Deferribacter desulfuricans SSM1; | YP_003496880, BAI81124; |
| | Thermosynechococcus elongatus BP-1; | NP_681848, BAC08610; |
| | Hydrogenobacter thermophilus TK-6; | YP_003431766, BAI68565; |
| | Nostoc punctiforme PCC 73102; | ACC82168, ADN14191; |
| | ‘Nostoc azollae’ 0708; | ADI62939; |
| | Arthrospira maxima CS-328; | EDZ97146; |
| | Prochlorococcus marinus str. MIT | AB017457; |
| | 9301; Cyanobium sp. PCC 7001; | ZP_05044537, EDY37846; |
| | Synechococcus sp. PCC 7335; | ZP_05037932; |
| | Arthrospira platensis str. Paraca; | ZP_06383646; |
| | Microcystis aeruginosa NIES-843; | BAG02689; |
| | Chlamydomonas reinhardtii; | XP_001693179, EDP03205; |
| | Arabidopsis thaliana; | BAB03011; |
| | Oryza sativa Indica Group; | ABR25557; |
| | Glycine max; | ACU26534; |
| 56: | Schizosaccharomyces japonicus | XP_002173231, EEB06938; |
| 2-Methylbutyraldehyde | yFS275; | |
| reductase | Pichia pastoris GS115; | XP_002490018, CAY67737, |
| (EC 1.1.1.265) | | XM_002489973; |
| | Saccharomyces cerevisiae S288c; | DAA12209, NP_010656 , |
| | | NM_001180676 ; |
| | Aspergillus fumigatus Af293; | XP_752003; |
| | Debaryomyces hansenii CBS767; | XP_002770138; |
| | Debaryomyces hansenii | CAR65507; |
| | Kluyveromyces lactis; | CAH02579; |
| | Lachancea thermotolerans CBS 6340; | XP_002554884; |
| | Lachancea thermotolerans; | CAR24447, CAR23718; |
| | Saccharomyces cerevisiae EC1118; | CAY78868; |
| | Saccharomyces cerevisiae JAY291; | EEU08013; |
| 57: | Saccharomyces cerevisiae S288c; | DAA10635, NM_001183405, |
| 3-Methylbutanal reductase | | NP_014490; |
| (EC 1.1.1.265) | Saccharomyces cerevisiae EC1118; | CAY86141; |
| | Saccharomyces cerevisiae JAY291; | EEU07090; |
| 07′: | Geobacillus kaustophilus HTA426; | YP_147173, BAD75605; |
| 3-Ketothiolase (reversible) | Azohydromonas lata; | |
| | Rhodoferax ferrireducens T118; | YP_523526; |
| | Allochromatium vinosum; | CAA01849, CAA01846; |
| | Dechloromonas aromatica RCB; | YP_286222; |
| | Rhodobacter sphaeroides ATCC 17029; | YP_001041914; |
| | Rhodobacter sphaeroides ATCC 17025; | YP_001166229; |
| | Bacillus sp. 256; | ABX11181; |
| | Silicibacter lacuscaerulensis ITI-1157; | ZP_05785678; |
| | Aspergillus fumigatus Af293; | XP_752635; |
| | Rhizobium etli; | AAK21958; |
| | Citreicella sp. SE45; | ZP_05784120, ZP_05781517; |
| | Silicibacter sp. TrichCH4B; | ZP_05742998; |
| | Azohydromonas lata; | AAC83659, AAD10275; |
| | Chromobacterium violaceum; | AAC69616; |
| | Dinoroseobacter shibae DFL 12; | ABV95064; |
| | Alcaligenes sp. SH-69; | AAP41838; |
| | Candida dubliniensis CD36; | CAX43351, XP_002418052; |
| | Pseudomonas sp. 14-3; | CAK18903; |
| | Aspergillus flavus NRRL3357; | XP_002375989; |
| | Aedes aegypti; | EAT37298, EAT37297, |
| | | XP_001654752, XP_001654751; |
| | Scheffersomyces stipitis CBS 6054; | ABN68380, XP_001386409; |
| | Cyanothece sp. PCC 7424; | YP_002375827, ACK68959; |
| | Cyanothece sp. PCC 7822; | YP_003886602, ADN13327; |
| | Microcystis aeruginosa NIES-843; | BAG04828; |
| 08′: | Syntrophothermus lipocalidus DSM | YP_003702743, ADI02178, |
| 3-Hydroxyacyl-CoA | 12680; | ADI01287, ADI01071; |
| dehydrogenase | Oceanithermus profundus DSM 14977; | ADR36325; |
| | Anoxybacillus flavithermus WK1; | YP_002317076, YP_002315864; |
| | Pelotomaculum thermopropionicum SI; | YP_001210823, BAF58454; |
| | Geobacillus kaustophilus HTA426; | YP_149248, YP_147889; |
| | Deferribacter desulfuricans SSM1; | YP_003497047, BAI81291; |
| | Glomerella graminicola M1.001; | EFQ32520, EFQ35765; |
| | Legionella pneumophila str. Corby; | YP_001250712, ABQ55366; |
| | Aspergillus fumigatus Af293; | XP_748706, XP_748351; |
| | Coprinopsis cinerea okayama7#130; | EAU80763; |
| | Botryotinia fuckeliana B05.10; | XP_001559519; |
| | Coccidioides posadasii; E. coli; | ABH10642; YP_001462756; |
| | Chelativorans sp. BNC1; | YP_675197; |
| | Nostoc punctiforme PCC 73102; | ACC81853, YP_001866796; |
| | Oscillatoria sp. PCC 6506; | ZP_07114022, CBN59220; |
| 09′: | Bordetella petrii; | CAP41574; |
| Enoyl-CoA dehydratase | Bordetella petrii DSM 12804; | YP_001629844; |
| | Anoxybacillus flavithermus WK1; | YP_002315700, YP_002314932; |
| | Geobacillus kaustophilus HTA426; | YP_148541, YP_147845, |
| | Geobacillus kaustophilus; | BAD76199; BAD18341; |
| | Syntrophothermus lipocalidus DSM | ADI02939, ADI02740, |
| | 12680; | ADI02007, ADI01364; |
| | Acinetobacter sp. SE19; | AAG10018; |
| | Scheffersomyces stipitis CBS 6054; | ABN64617, XP_001382646; |
| | Laccaria bicolor S238N-H82; | EDR09131, XP_001888157; |
| | Alternaria alternate; | BAH83503, |
| | Ajellomyces dermatitidis ER-3; | EEQ91989; |
| | Aspergillus fumigatus Af293; | EAL93360, XP_755398; |
| | Cryptococcus neoformans var. | XP_572730; |
| | neoformans JEC21; E. Coli; | ADN73405, YP_001458194; |
| | Aspergillus flavus NRRL3357; | XP_002377859; |
| | Laccaria bicolor S238N-H82; | EDR01115; |
| | Neosartorya fischeri NRRL 181; | EAW18645; |
| | Nostoc sp. ‘Peltigera membranacea | ADA69246; |
| | cyanobiont’; | |
| 10′: | Xanthomonas campestris pv. | CAP53709; |
| 2-Enoyl-CoA reductase | Campestris; Xanthomonas campestris | YP_001905744; |
| | pv. campestris str. B100; Xanthomonas | ZP_06489037; |
| | campestris pv. musacearum | |
| | NCPPB4381; Xanthomonas campestris | ZP_06487845; |
| | pv. vasculorum NCPPB702; | |
| | Aeromicrobium marinum DSM 15272; | ZP_07718056, EFQ82338; |
| | Rhodobacterales bacterium HTCC2083; | ZP_05074461, EDZ42121; |
| | Lysinibacillus fusiformis ZC1; | |
| | Mycobacterium smegmatis str. MC2 | ZP_07049092, EFI69525; |
| | 155; | YP_886510, ABK76225; |
| | Lysinibacillus sphaericus C3-41; | |
| | Coprinopsis cinerea okayama7#130; | YP_001699417, ACA41287; |
| | Arthroderma gypseum CBS 118893; | XP_002910885, EFI27391; |
| | Paracoccidioides brasiliensis Pb01; | EFR05506; |
| | Paracoccidioides brasiliensis Pb18; | XP_002796528, EEH39074; |
| | Ajellomyces capsulatus G186AR; | EEH43955; |
| | Ostreococcus tauri; | EEH03439; |
| | Jatropha curcas; | XP_003083795, CAL57762; |
| | | ACS32302; |
| 11′: | Clostridium cellulovorans 743B; | YP_003845606, ADL53842; |
| Acyl-CoA reductase (EC | Thermosphaera aggregans DSM 11486; | YP_003649571, ADG90619; |
| 1.2.1.50) | Delftia acidovorans SPH-1; | |
| | Comamonas testosteroni KF-1; | YP_001565543, ABX37158; |
| | Bifidobacterium longum subsp. infantis | ZP_03543536; |
| | ATCC 15697; | YP_002321654, ACJ51276; |
| | Clostridium papyrosolvens DSM 2782; | |
| | Acidovorax avenae subsp. avenae ATCC | ZP_05497968, EEU57047; |
| | 19860; | ZP_06211782, EFA39209; |
| | Comamonas testosteroni KF-1; | |
| | Aminomonas paucivorans DSM 12260; | EED67822; |
| | Herpetosiphon aurantiacus ATCC | ZP_07740542, EFQ24431 ; |
| | 23779; | ABX07240, YP_001547368; |
| | Clostridium beijerinckii NCIMB 8052; | |
| | Geobacillus sp. G11MC16; | ABR34265, YP_001309221; |
| | Clostridium lentocellum DSM 5427; | ZP_03148237, EDY05596; |
| | Leadbetterella byssophila DSM 17132; | ZP_06885967, EFG96716; |
| | Actinosynnema mirum DSM 43827; | YP_003997212, ADQ16859; |
| | Haliangium ochraceum DSM 14365; | YP_003101455, ACU37609 ; |
| | Photobacterium phosphoreum; | ACY16972, YP_003268865; |
| | Simmondsia chinensis; | AAT00788; |
| | Hevea brasiliensis; | AAD38039; |
| | Arabidopsis thaliana; | AAR88762; |
| | | ABE65991; |
| 12′: | Mycobacterium chubuense NBB4; | ACZ56328; |
| Hexanol dehydrogenase | | |
| 12″: | Drosophila subobscura; | ABO61862, ABO65263, |
| Octanol dehydrogenase | | CAD43362, CAD43361, |
| EC 1.1.1.73 | | CAD54410, CAD43360, |
| | | CAD43359, CAD43358 |
| | | CAD43357, CAD43356; |
| 43′: | Pyrococcus furiosus DSM 3638; | AAC25556; |
| Short chain alcohol | Burkholderia vietnamiensis G4; | ABO56626; |
| dehydrogenase | Geobacillus thermoleovorans; | BAA94092; |
| | Geobacillus kaustophilus HTA426; | YP_146837, BAD75269; |
| | Anoxybacillus flavithermus WK1; | YP_002314715, ACJ32730; |
| | Helicobacter pylori PeCan4; | YP_003927327, ADO07277; |
| | Mycobacterium chubuense NBB4; | ACZ56328; |
| | Mycobacterium avium subsp. avium | ZP_05215778; |
| | ATCC 25291; Aspergillus oryzae; | BAE71320; |
| | cyanobacterium UCYN-A; | YP_003421738, ADB95357; |
| | Anabaena circinalis AWQC131C; | ABI75134; |
| | Cylindrospermopsis raciborskii T3; | ABI75108; |
| | Helicobacter pylori Sat464; | ADO05766; |
| | Helicobacter pylori Cuz20; | ADO04259; |
| | Mycobacterium intracellulare ATCC | ZP_05228059, ZP_05228058; |
| | 13950; Mycobacterium avium subsp. | ZP_05215779; |
| | avium ATCC 25291; Gluconacetobacter | |
| | hansenii ATCC 23769; Helicobacter | ZP_06834730, EFG83978; |
| | pylori Shi470; | YP_001910563, ACD48533; |
| | Mycobacterium avium 104; | YP_880627, ABK67217; |
| | Citrus sinensis; | ADH82118; |
| | Gossypium hirsutum; | ABD65462; |
| | Arabidopsis halleri; | ABZ02361, ABZ02360; |
| | Paracoccidioides brasiliensis Pb01; | XP_002792148, EEH34889; |
| | Pyrenophora tritici-repentis Pt-1C-BFP; | XP_001940779, EDU43498; |
| | Ajellomyces capsulatus H143; | EER38733; |
| | Scheffersomyces stipitis CBS 6054; | XP_001382930, ABN64901; |
| 70: | Ralstonia eutropha H16; | NP_942643 (hoxK), NP_942644 |
| Membrane-bound | | (hoxG), YP_015633 (hoxZ); |
| hydrogenase (MBH) | | AAP85757 (hoxK), AAP85758 |
| | Ralstonia eutropha H16; | (hoxG), AAA16463 (hoxZ); |
| | | ABF08183 (hoxK), YP_583451 |
| | Cupriavidus metallidurans CH34; | (hoxG), ABF08182 (hoxG); |
| | | ADK12981, ADK12980; |
| | Thiocapsa roseopersicina, | ACJ15972; |
| | Thermococcus onnurineus NA1; | YP_004763067; |
| | Thermococcus sp. 4557; | YP_004763083; |
| | Thermococcus sp. 4557; | YP_004763081; |
| | Thermococcus sp. 4557; | AEK73406; |
| | Thermococcus sp. 4557; | AEK73404; |
| | Thermococcus sp. 4557; | NP_579163; |
| | Pyrococcus furiosus DSM 3638; | NP_579162; |
| | Pyrococcus furiosus DSM 3638; | YP_004624085; |
| | Pyrococcus yayanosii CH1; | YP_004624086; |
| | Pyrococcus yayanosii CH1; | YP_004624087; |
| | Pyrococcus yayanosii CH1; | NP_142896; |
| | Pyrococcus horikoshii OT3; | BAK19334; |
| | Hydrogenovibrio marinus; | CAA63615; |
| | Alcaligenes sp.; | CAA63616; |
| | Rubrivivax sp.; | BAF73677; |
| | Hydrogenobacter thermophilus TK-6; | ACS32538; |
| | Thermococcus gammatolerans EJ3; | ADN36337; |
| | Methanoplanus petrolearius DSM | YP_002958402; |
| | 11571; Thermococcus gammatolerans | YP_004638463 (hoxZ); |
| | EJ3; Oligotropha carboxidovorans | AEI08136 (hoxZ); |
| | OM5; Aquifex aeolicus VF5; | NP_213456 (hoxZ); |
| | Centipeda periodontii DSM 2778; | ZP_08500995 (hoxZ); |
| | Selenomonas noxia ATCC 43541; | ZP_06602778 (hoxZ); |
| | Allochromatium vinosum DSM 180; | ADC63224 (hoxZ); |
| | Thiomonas intermedia K12; | ADG32404 (hoxZ); |
| | Aquifex aeolicus VF5; | AAC06857 (hoxZ); |
| 71: | Ralstonia eutropha H16; | AAP85843 (hoxY), AAP85844 |
| Soluble hydrogenase (SH) | Ralstonia eutropha H16; | (HoxH); NP_942730 (hoxH), |
| (NAD(P)-reducing) | Ralstonia eutropha H16; | NP_942729 (hoxY); |
| | Ralstonia eutropha H16; | NP_942727 (hoxF), NP_942728 |
| | Ralstonia eutropha H16; | (hoxU); AAP85841 (hoxF), |
| | Ralstonia eutropha H16; | AAP85842 (hoxU); AAC06140 |
| | Ralstonia eutropha H16; | (hoxF), AAC06141 (hoxU), |
| | Ralstonia eutropha H16; | AAC06142 (hoxY), |
| | Ralstonia eutropha H16; | AAC06143 (hoxH); |
| | Rhodobacter capsulatus; | AAD38065 (hoxH); |
| | Azotobacter vinelandii DJ; | YP_002797671 (hoxH); |
| | Microcystis aeruginosa NIES-843; | BAG01243 (hoxH); |
| | Acaryochloris marina MBIC11017; | ABW32682 (hoxH); |
| | Synechococcus sp. PCC 7002; | AAN03569 (hoxH); |
| | Synechococcus elongatus PCC 6301; | CAA66383 (hoxH); |
| | Synechococcus elongatus PCC 6301; | CAA66382 (hoxY); |
| | Allochromatium vinosum; | AAX89151 (hoxY); |
| | Microcystis aeruginosa PCC 7806; | CAO88137 (hoxY); |
| | Azotobacter vinelandii DJ; | YP_002797670 (hoxY); |
| | Synechococcus elongatus PCC 6301; | CAA66381 (hoxU); |
| | Allochromatium vinosum; | AAX89150 (hoxU); |
| | Arthrospira platensis FACHB341; | ABC26909 (hoxU); |
| | Microcystis aeruginosa PCC 7806; | CAO88140 (hoxU); |
| | Lyngbya majuscula CCAP 1446/4; | AAY57574 (hoxU); |
| | Synechococcus elongatus PCC 6301; | YP_172263 (hoxU); |
| | Cyanothece sp. ATCC 51142; | YP_001803733 (hoxU); |
| | Synechococcus elongatus PCC 6301; | CAA73873 (hoxF); |
| | Allochromatium vinosum; | AAX89149 (hoxF); |
| | Arthrospira platensis FACHB341; | ABC26907 (hoxF); |
| | Synechococcus sp. PCC 7002; | YP_001733465 (hoxF); |
| | Anaerolinea thermophila UNI-1; | BAJ63286 (hoxH); |
| | Caloramator australicus RC3; | CCC57856 (hoxF); |
| 72: | Ralstonia eutropha H16; | NP_942649 (hoxO), AAP85763 |
| Hydrogenase accessary | Ralstonia eutropha H16; | (hoxO), AAA16467 (hoxO); |
| proteins | Cupriavidus metallidurans CH34; | ABF08176 (hoxO); YP_583445 |
| | Cupriavidus metallidurans CH34; | (hoxO); |
| | Ralstonia eutropha H16; | NP_942650 (hoxQ), AAP85764 |
| | | (hoxQ), AAA16468 (hoxQ); |
| | Cupriavidus metallidurans CH34; | ABF08175 (hoxQ), YP_583444 |
| | | (hoxQ); |
| | Azotobacter vinelandii; | AAA19504 (hoxQ); |
| | Salmonella enterica subsp.; | EHC91928 (hoxQ/hoxR), |
| | | EFX49216 (hoxQ/hoxR), |
| | Escherichia coli B354; | ZP_06652932 (hoxQ); |
| | Methyloversatilis universalis FAM5; | ZP_08506135 (hoxQ); |
| | Shigella flexneri CDC 796-83; | EFW61888 (hoxQ); |
| | Ralstonia eutropha H16; | AAA16469 (hoxR), |
| | | NP_942651(hoxR); |
| | Azotobacter vinelandii; | AAA19505 (hoxR); |
| | Ralstonia eutropha H16; | NP_942652 (hoxT), AAP85766 |
| | | (hoxT), AAA16470 (hoxT); |
| | Cupriavidus metallidurans CH34; | ABF08173 (hoxT); |
| | Azotobacter vinelandii DJ; | YP_002802114 (hoxT), |
| | | ACO1139 (hoxT); |
| | Ralstonia eutropha H16; | NP_942648 (hoxL), AAP85762 |
| | | (hoxL), AAA16466 (hoxL); |
| | Azotobacter vinelandii; | AAA19502 (hoxL); |
| | Oligotropha carboxidovorans OM5; | YP_015634 (hoxL); |
| | Cupriavidus metallidurans CH34; | ABF08177 (hoxL), YP_583446 |
| | Salmonella enterica subsp. enterica | (hoxL); |
| | serovar Weltevreden str. 2007-60-3289- | CBY95754 (hoxL); |
| | 1; Oligotropha carboxidovorans OM5; | YP_004638464 (hoxL); |
| | Oligotropha carboxidovorans OM4; | AEI04509 (hoxL); |
| | Azotobacter vinelandii DJ; | YP_002802118 (hoxL), |
| | | ACO81143 (hoxL); |
| | Methyloversatilis universalis FAM5; | ZP_08506137 (hoxL), EGK70316 |
| | | (hoxL); |
| | Ralstonia eutropha H16; | NP_942653 (hoxV), AAP85767 |
| | | (hoxV), AAA16471 (hoxV); |
| | Azotobacter vinelandii; | AAA19507 (hoxV); |
| | Oligotropha carboxidovorans OM5; | YP_015636 (HoxV); |
| | Cupriavidus metallidurans CH34; | ABF08172 (hoxV); |
| | Azotobacter vinelandii DJ; | YP_002802113 (hoxV); |
| | Cupriavidus metallidurans CH34; | YP_583441 (hoxV); |
| | Methyloversatilis universalis FAM5; | ZP_08506132 (hoxV); |
| | Methyloversatilis universalis FAM5; | EGK70311 (hoxV); |
| | Ralstonia eutropha H16 | NP_942647 (hoxM); |
| | Oligotropha carboxidovorans OM5, | YP_004638462 (hoxM); |
| | Oligotropha carboxidovorans OM4; | AEI04507 (hoxM); |
| | Azotobacter vinelandii; | AAA19501 (hoxM); |
| | Azotobacter vinelandii DJ; | YP_002802119 (hoxM); |
| | Cupriavidus metallidurans CH34; | YP_583447 (hoxM); |
| | Hydrogenobacter thermophilus TK-6; | BAF73673 (hoxM); |
| | Hydrogenobacter thermophilus TK-6; | YP_003432119 (hoxM); |
| | Thermoproteus tenax Kra 1; | CCC80713 (hoxM); |
| | Acidithiobacillus sp. GGI-221; | EGQ60729 (hoxM); |
| | Methyloversatilis universalis FAM5; | ZP_08506138 (hoxM); |
| | Burkholderiales bacterium 1_1_47; | ZP_07342912 (hoxM); |
| | Thiomonas intermedia K12; | YP_003644737 (hoxM); |
| | Thermococcus gammatolerans EJ3; | YP_002958602 |
| | | (hybD/hycI/hoxM); |
| | Ralstonia eutropha H16; | NP_942661 (hoxA), AAP85775; |
| | Azorhizobium caulinodans ORS 571; | AAS91037 (hoxA); |
| | Bradyrhizobium japonicum; | CAA78991 (hoxA); |
| | Hyphomicrobium sp. MC1; | YP_004674255 (hoxA); |
| | Azoarcus sp. BH72; | YP_935307 (hoxA); |
| | Methyloversatilis universalis FAM5; | ZP_08506123 (hoxA); |
| | Grimontia hollisae CIP 101886; | ZP_06053565 (hoxA); |
| | Oxalobacteraceae bacterium; | ZP_08276168 (hoxA); |
| | Ralstonia eutropha H16; | NP_942662 (hoxB), AAP85776; |
| | Azoarcus sp. BH72; | YP_935309 (hoxB); |
| | Oligotropha carboxidovorans OM5; | YP_004638467 (hoxB); |
| | Ralstonia eutropha H16; | AAP85777 (hoxC), NP_942663; |
| | Azoarcus sp. BH72; | YP_935310 (hoxC); |
| | Oligotropha carboxidovorans OM4; | AEI04502 (hoxC); |
| | Oligotropha carboxidovorans OM5; | YP_004638457 (hoxC); |
| | Oxalobacteraceae bacterium | ZP_08276171 (hoxJ), EGF30361 |
| | IMCC9480; | (hoxJ); |
| | Alcaligenes hydrogenophilus; | AAB49362 (hoxJ); |
| | Synechocystis sp. PCC 6803; | BAA18357 (hypA); |
| | Ralstonia eutropha H16; | NP_942654 (hypA1); |
| | Ralstonia eutropha H16; | NP_942733 (hypA2); |
| | Ralstonia eutropha H16; | NP_942716 (hypA3); |
| | Cupriavidus metallidurans CH34; | YP_583440 (hypA); |
| | Ralstonia eutropha H16; | NP_942655 (hypB1); |
| | Ralstonia eutropha H16; | AAP85769 (hypB1); |
| | Butyrivibrio proteoclasticus B316; | YP_003830670 (hypB1); |
| | Oligotropha carboxidovorans OM5; | YP_004638455 (hypB); |
| | Oligotropha carboxidovorans OM4; | AEI04500 (hypB); |
| | Desulfitobacterium metallireducens | ZP_08976390 (hypB), |
| | DSM 15288; | EHC20145 (hypB); |
| | Synechocystis sp. PCC 6803; | BAA18180 (hypC); |
| | Cyanothece sp. CCY0110; | EAZ91066 (hypC); |
| | Cupriavidus metallidurans CH34, | ABF08421(hypC); |
| | Ralstonia eutropha H16; | NP_942657 (hypC1); |
| | Ralstonia eutropha H16; | AAP85826 (hypC2); |
| | Ralstonia eutropha H16; | CAA49734 (hypD); |
| | Cupriavidus metallidurans CH34; | YP_583436 (hypD); |
| | Cupriavidus metallidurans CH34; | ABF08422 (hypD); |
| | Escherichia coli BL21(DE3); | ACT44398 (hypD); |
| | Synechocystis sp. PCC 6803; | BAA17478 (hypE); |
| | Ralstonia eutropha H16; | CAA49735 (hypE); |
| | Ralstonia eutropha H16; | NP_942659 (hypE1); |
| | Ralstonia eutropha H16; | AAP85829 (hypE2); |
| | Rhizobium leguminosarum; | CAA37164 (hypE); |
| | Azotobacter vinelandii; | AAA19513 (hypE); |
| | Aeropyrum pernix K1; | NP_148343 (hypE); |
| | Sulfolobus solfataricus P2; | NP_341628 (hypE); |
| | Hydrogenobacter thermophilus TK-6; | YP_003432665 (hypE); |
| | Pelotomaculum thermopropionicum SI; | YP_001212249 (hypE); |
| | Syntrophothermus lipocalidus DSM | ADI01176 (hypE), |
| | 12680; | YP_003701741 (hypE); |
| | Hydrogenobacter thermophilus TK-6; | YP_003432667 (hypF); |
| | Pelotomaculum thermopropionicum SI; | YP_001212246 (hypF); |
| | Syntrophothermus lipocalidus DSM | ADI01173 (hypF), |
| | 12680; Caldicellulosiruptor bescii DSM | YP_003701738 (hypF); |
| | 6725; | YP_002572964 (hypF); |
| | Ralstonia eutropha H16; | CAA49731 (hypF); |
| | Ralstonia eutropha H16; | NP_942660 (hypX); |
| | Ralstonia eutropha H16; | AAP85774 (hypX) |
| | Hydrogenobacter thermophilus TK-6; | YP_003433460 (hypX); |
| | Rhizobium leguminosarum; | CAA37165 (hypX); |
| | Methyloversatilis universalis FAM5; | ZP_08506124 (hoxX); |
| | Cupriavidus metallidurans CH34; | ABF08424 (hoxX); |
| | Ralstonia eutropha H16; | CAA52735 (hoxX); |
| 73: | Desulfobulbus propionicus DSM 2032; | ADY56959, YP_004195043; |
| NAD(P)-dependent | Acetohalobium arabaticum DSM 5501; | YP_003826884; |
| hydrogenase | Ilyobacter polyt; ropus DSM 2926; beta | ADO82414; |
| | proteobacterium KB13 | EDZ65062, ZP_05082375; |
| | Acetohalobium arabaticum DSM 5501; | ADL11819 |
| 74: | Moorella thermoacetica ATCC 39073; | YP_429324, ABC18781; |
| Formate dehydrogenase | Moorella thermoacetica ATCC 39073; | YP_431142, ABC20599; |
| using NAD(P)H | Moorella thermoacetica; | AAB18330 (α), AAB18329 (β); |
| | Methanosaeta harundinacea 6Ac; | AET63712, AET63711, |
| | Methanoculleus marisnigri JR1; | YP_001047290; |
| | Methanocorpusculum labreanum Z; | YP_001029904, YP_001029903; |
| | Helicobacter bilis ATCC 43879; | ZP_04582064 (NADPH); |
| | Helicobacter bilis ATCC 43879; | EEO23341 (NADPH); |
| | Pelotomaculum thermopropionicum SI; | YP_001213196; |
| | Hydrogenobacter thermophilus TK-6; | YP_003432807; |
| | Hydrogenobacter thermophilus TK-6; | YP_003433330 (NDA dependent); |
| | Klebsiella variicola At-22; | ADC58081, YP_003439113; |
| | Azospirillum sp. B510; | YP_003451652, YP_003450092; |
| | Thermococcus gammatolerans EJ3; | YP_002958615; |
| | Yersinia pestis Antiqua; | ABG15899; |
| | Thermofilum pendens Hrk 5; | YP_919603; |
| | Ferrimonas balearica DSM 9799; | YP_003913071; |
| | Thermodesulfatator indicus DSM | AEH46025; |
| | 15286; Shewanella baltica BA175; | AEG12633; |
| | Methanocella paludicola SANAE; | YP_003357462, YP_003357461; |
| | Methanosaeta harundinacea 6Ac; | AET64643, AET64987, |
| | | AET65705; |
| 75: | Moorella thermoacetica ATCC 39073; | YP_428991; |
| 10-Formyl-H₄ folate | Methanocorpusculum labreanum Z; | YP_001030445; |
| synthetase (ADP | Sphingomonas paucimobilis; | BAD61061; |
| forming, 10- | Desulfatibacillum alkenivorans AK-01; | ACL05327; |
| Formyltetrahydrofolate | Corynebacterium aurimucosum; | YP_002834788; |
| Synthetase) | Clostridium acidurici; | AAA53187; |
| | Sphingobium sp. SYK-6; | YP_004834408; |
| | Listeria monocytogenes serotype 4b str. | YP_002758587; |
| | CLIP 80459; Vibrio fischeri MJ11; | YP_002156619; |
| | Anoxybacillus flavithermus WK1; | YP_002315932; |
| | Thermotoga lettingae TMO; | YP_001471133; |
| | Fervidobacterium nodosum Rt17-B1; | YP_001410584; |
| | Thermosipho melanesiensis BI429; | YP_001305561; |
| | Thermotoga petrophila RKU-1 | YP_001244647 |
| | Pelotomaculum thermopropionicum SI; | YP_001210750; |
| 76: | Moorella thermoacetica ATCC 39073; | YP_430368, ABC19825; |
| 5,10-Methenyl-H₄ folate | Thermotoga lettingae TMO; | ABV34070; |
| cyclohydrolase | Caldicellulosiruptor bescii DSM 6725; | YP_002572856; |
| (Methenyltetrahydrofolate | Thermotoga petrophila RKU-1; | ABQ47072; |
| cyclohydrolase) | Anoxybacillus flavithermus WK1; | YP_002315305; |
| | Geobacillus kaustophilus HTA426; | BAD76681; |
| | Geobacillus kaustophilus HTA426; | YP_148249; |
| | Synechococcus sp. JA-2-3B′a(2-13); | YP_476354; |
| | Synechococcus sp. JA-3-3Ab; | YP_475381; |
| | Exiguobacterium sp. AT1b; | YP_002884899; |
| | Thermotoga lettingae TMO; | YP_001471134; |
| 77: | Moorella thermoacetica ATCC 39073; | ABC19825, YP_430368; |
| 5,10-Methylene-H₄ folate | Geobacillus kaustophilus HTA426; | BAD76681; |
| dehydrogenase | Syntrophothermus lipocalidus; | ADI01214; |
| | Caldicellulosiruptor kronotskyensis; | ADQ46551; |
| | Caldicellulosiruptor kristjanssonii; | ADQ40482; |
| | Caldicellulosiruptor hydrothermalis; | ADQ07463; |
| | Caldicellulosiruptor owensensis OL; | ADQ04336; |
| | Caldicellulosiruptor hydrothermalis; | YP_003992832; |
| | Kosmotoga olearia TBF 19.5.1; | ACR80790; |
| | Exiguobacterium sp. AT1b; | ACQ69454; |
| | Komagataella pastoris CBS 7435; | CCA37557; |
| | Homo sapiens; | AAH09806; |
| | Taeniopygia guttata; | XP_002200380; |
| | Syntrophobotulus glycolicus DSM 8271; | ADY56189; |
| | Olsenella uli DSM 7084; | ADK67906; |
| 78: | Moorella thermoacetica ATCC 39073; | YP_430048, ABC19505; |
| 5,10-Methylene-H₄ | Syntrophothermus lipocalidus; | ADI02156; |
| folate reductase | Fervidobacterium nodosum Rt17-B1; | ABS61421; |
| (Methylenetetrahydrofolate | Thermotoga petrophila RKU-1; | ABQ46674; |
| reductase) | Fervidobacterium nodosum Rt17-B1; | ABS61126; |
| | Thermotoga lettingae TMO; | ABV33918; |
| | Thermosipho melanesiensis BI429; | YP_001305980; |
| | Synechococcus sp. JA-2-3B′a(2-13); | YP_477166; |
| | Hippea maritima DSM 10411; | YP_004340445; |
| | Spirochaeta thermophila DSM 6192; | YP_003875363; |
| | Deferribacter desulfuricans SSM1; | YP_003496368; |
| | Hydrogenobacter thermophilus TK-6; | YP_003432279; |
| | Pelotomaculum thermopropionicum SI; | BAF59187, YP_001211556; |
| 79: | Moorella thermoacetica ATCC 39073; | YP_430950, YP_430174; |
| Methyl-H₄ folate: corrinoid | Pelotomaculum thermopropionicum SI; | YP_001211554; |
| iron-sulfur protein | Clostridium carboxidivorans P7; | ADO12092; |
| Methyltransferase | Desulfitobacterium hafniense DCB-2; | YP_002461301; |
| (Methyltetrahydrofolate:corrinoid/ | Dinoroseobacter shibae DFL 12; | YP_001533020; |
| iron-sulfur protein | Ammonifex degensii KC4; | YP_003238352; |
| Methyltransferase) | Desulfotomaculum acetoxidans; | YP_003190781; |
| | Rhodobacter sphaeroides KD131; | YP_002525435; |
| | Carboxydothermus hydrogenoformans; | YP_360065; |
| | Rhodobacter sphaeroides 2.4.1; | YP_352826; |
| | Heliobacterium modesticaldum Ice1; | YP_001680302; |
| | Sinorhizobium meliloti 1021; Acetonema | NP_386092; |
| | longum DSM 6540 | ZP_08625620; |
| 80: | Moorella thermoacetica; | AAA23255; |
| Corrinoid iron-sulfur protein | Carboxydothermus hydrogenoformans | 2H9A_A, 2H9A_B; |
| (CFeSP) | Clostridium ragsdalei; | AEI90763, AEI90762; |
| | Clostridium autoethanogenum; | AEI90746, AEI90745; |
| | Clostridium sticklandii DSM 519; | YP_003936194; |
| | Clostridium sticklandii; | CBH21289; |
| 81: | Moorella thermoacetica ATCC 39073; | ABC19516, YP_430059; |
| CO dehydrogenase/acetyl- | Moorella thermoacetica ATCC 39073; | YP_430813 (CODH); |
| CoA synthase (Fd²⁻) | Moorella thermoacetica; | AAA23229, AAA23228; |
| | Caldicellulosiruptor kristjanssonii; | ADQ39747; |
| | Caldicellulosiruptor saccharolyticus; | YP_001179230; |
| | Clostridium ragsdalei; | AEI90761; |
| | Clostridium autoethanogenum; | AEI90744; |
| | Desulfosporosinus orientis DSM 765; | AET68776; |
| | Methanococcus aeolicus Nankai-3; | ABR56750; |
| | Desulfobacca acetoxidans DSM 11109; | YP_004370981; |
| | Thermodesulfatator indicus; | AEH46031; |
| | Acetohalobium arabaticum DSM 5501; | ADL12817; |
| | Desulfarculus baarsii DSM 2075; | YP_003806211; |
| | Archaeoglobus veneficus SNP6; | YP_004341848; |
| | Methanosalsum zhilinae DSM 4017; | AEH60991; |
| | Thermosediminibacter oceani; | ADL07576; |
| | Desulfotomaculum kuznetsovii; | YP_004517493, YP_004516875; |
| | Methanosalsum zhilinae DSM 4017; | AEH60989, AEH60993; |
| 82: | Thermodesulfobium narugense; | YP_004437266; |
| Pyruvate synthase (Fd²⁻) | Desulfobacca acetoxidans; | YP_004370392; |
| | Archaeoglobus veneficus SNP6; | YP_004341929; |
| | Hippea maritima DSM 10411; | YP_004339618; |
| | Desulfurobacterium | YP_004281767, YP_004281766, |
| | thermolithotrophum; | ADY73708; |
| | Archaeoglobus veneficus; | AEA47214; |
| | Thermodesulfobium narugense; | AEE14134; |
| | Archaeoglobus veneficus SNP6; | YP_004341930; |
| | Thermobacillus composti KWC4; | ZP_08918406; |
| | Desulfobacca acetoxidans; | AEB09210; |
| | Methanolinea tarda NOBI-1; | EHF09898; |
| | Methanobacterium sp. AL-21; | YP_004289712, ADZ08740; |
| | Methanocella paludicola SANAE; | YP_003356312, YP_003356313; |
| 83: | Methanothermobacter marburgensis str. | ADL58895, ADL58894, |
| Formylmethanofuran | Marburg; | ADL58283, ADL58893, |
| dehydrogenase (Fmd) (Fd²⁻) | | ADL57751, ADL57749, |
| | | ADL57750, ADL57748; |
| | Methanothermobacter | CAA66401, CAA61212, |
| | thermautotrophicus; | CAA66400, CAA66402; |
| | Methanothermobacter | CAA61213, CAA61214, |
| | thermautotrophicus; | CAA61210, CAA61211, |
| | | CAA61209; |
| | Agrobacterium sp. H13-3; | YP_004444030; |
| | Agrobacterium vitis S4; | YP_002547540; |
| | Methylomonas methanica MC09; | YP_004511613; |
| | Desulfobacca acetoxidans DSM 11109; | YP_004370144, AEB08963; |
| | Methylovorus glucosetrophus SIP3-4; | YP_003051278; |
| | Methylotenera mobilis JLW8; | YP_003048298; |
| | Methylotenera versatilis 301; | ADI29297; |
| | Methanoculleus marisnigri JR1; | YP_001046285, YP_001046287, |
| | | YP_001046533; |
| | Methanosaeta harundinacea 6Ac; | AET63761, AET64650, |
| | | AET65189, AET64652; |
| | Methanosphaera stadtmanae; | ABC56660, ABC56659, |
| | | YP_447302, ABC56661, |
| | | ABC56658, ABC56657; |
| 84: | Methanothermobacter marburgensis str. | ADL59225, |
| Formyl transferase | Marburg; | YP_003850538; |
| | Methanosaeta harundinacea 6Ac; | AET65566; |
| | Methanosarcina barkeri; | CAA62582; |
| | Methanopyrus kandleri AV19; | NP_614099; |
| | Thermosipho melanesiensis BI429; | YP_001305762; |
| | Desulfobacca acetoxidans DSM 11109; | YP_004369335; |
| | Methylobacterium chloromethanicum; | YP_002421530; |
| | Methylomicrobium alcaliphilum; | YP_004917963; |
| | Methanopyrus kandleri AV19; | NP_613403; |
| | Methanoculleus marisnigri JR1; | YP_001046543; |
| | Methanocorpusculum labreanum Z; | YP_001029658, YP_001029834; |
| | Methanopyrus kandleri AV19; | AAM02029, AAM01333; |
| | Methanocella paludicola SANAE; | YP_003356088, BAI61105; |
| 85: | Methanosphaera stadtmanae; | ABC57615, YP_448258; |
| 5,10-Methenyl- | Methanothermus fervidus DSM 2088; | YP_004003819; |
| tetrahydromethanopterin (H4 | Methanosalsum zhilinae DSM 4017; | AEH61193; |
| methanopterin) | Methanohalophilus mahii DSM 5219; | ADE36644; |
| cyclohydrolase | Methanoplanus petrolearius; | ADN34846; |
| | Archaeoglobus veneficus SNP6; | YP_004342719; |
| | Planctomyces brasiliensis DSM 5305; | YP_004269775; |
| | Methylobacillus flagellates; | AAD55893; |
| | Xanthobacter autotrophicus; | AAD55896; |
| | Methylosinus trichosporium OB3b; | AAD56174; |
| | Methylobacterium organophilum; | AAD55900; |
| | Methylococcus capsulatus; | AAD55899; |
| | Methylomicrobium kenyense; | AAS88982; |
| | Methylomonas sp. LW13; | AAS88987; |
| | Methylosinus sp. LW2; | AAS88975; |
| | Methylomicrobium kenyense; | AAS86344; |
| | Methanohalophilus mahii DSM 5219; | YP_003542289; |
| | Methanolinea tarda NOBI-1; | EHF09908; |
| | Methanothermococcus okinawensis IH1; | YP_004577331; |
| | Methanobacterium sp. SWAN-1; | YP_004519292; |
| | Methylomonas methanica MC09; | YP_004513168; |
| 86: | Methanothermobacter marburgensis; | ADL57660, YP_003848973; |
| 5,10-Methylene-H₄- | Methanosphaera stadtmanae; | YP_447224; |
| methanopterin | Methanococcus maripaludis X1; | AEK19019; |
| dehydrogenase (F₄₂₀H₂) | Methanothermobacter | CAA63376; |
| | thermautotrophicus; | |
| | Methanopyrus kandleri; | CAA43127; |
| | Methylobacterium extorquens AM1; | AAC27020; |
| | Methylobacillus flagellatus KT; | ABE49928; |
| | Xanthobacter autotrophicus; | AAD55895; |
| | Methyloversatilis universalis FAM5; | ZP_08504846; |
| | Methylobacterium chloromethanicum; | ACK83011; |
| | Methylobacterium populi BJ001; | YP_001924478; |
| | Methylobacterium extorquens PA1; | YP_001639299; |
| | Burkholderia sp. CCGE1001; | YP_004230417; |
| | Methylovorus sp. MP688; | YP_004039958; |
| | Methanocaldococcus fervens AG86; | YP_003128308; |
| | Methanocaldococcus jannaschii; | NP_247770; |
| | Methanobrevibacter smithii; | YP_001273145; |
| 87: | Methanoplanus petrolearius; | ADN36752; |
| 5,10-Methylene-H₄- | Methanocaldococcus sp. FS406-22; | YP_003458803; |
| methanopterin reductase | Methanocaldococcus infernus ME; | ADG13507; |
| (F₄₂₀H₂) | Methanocaldococcus fervens AG86; | ACV24808; |
| | anococcus maripaludis C6; | ABX01642; |
| | Stenotrophomonas sp. SKA14; | EED39154, ZP_05135093; |
| | Amycolatopsis mediterranei S699; | AEK43785; |
| | Corynebacterium glutamicum; | EHE83474; |
| | Acinetobacter sp. DR1; | ADI90167; |
| | Acinetobacter baumannii ABNIH4; | EGU03459; |
| | Acinetobacter sp. DR1; | YP_003731540; |
| | Paenibacillus terrae HPL-003; | AET61191; |
| | Acinetobacter baumannii ABNIH3; | EGT94264; |
| | Cupriavidus necator N-1; | AEI79563; |
| | Herbaspirillum seropedicae SmR1; | YP_003777169; |
| | Burkholderia cenocepacia HI2424; | YP_840196; |
| | Methanobrevibacter ruminantium M1; | YP_003423269, ADC46377; |
| | Methanococcus voltae A3; | ADI37005; |
| | Methanococcus aeolicus Nankai-3; | ABR56603; |
| | Methanocaldococcus vulcanius M7; | ACX71899; |
| 88: | Methanothermobacter marburgensis; | MTBMA_c02920; |
| Methyl-H4-methanopterin: | Methanothermobacter marburgensis str. | ADL57900; |
| corrinoid iron-sulfur protein | Marburg; | |
| methyltransferase | | |
| 89: | Methanothermobacter marburgensis; | MTBMA_c02910; |
| Corrinoid iron-sulfur protein | Methanothermobacter marburgensis str. | ADL57899; |
| (MTBMA_c02910) | Marburg; | |
| 90: | Methanothermobacter marburgensis; | αMTBMA_c02870/14220/14210/ |
| CO dehydrogenase/acetyl- | | 14200; |
| CoA synthase (Fd²⁻ _{red}) | | ε MTBMA_c14190/02880; |
| | | βMTBMA_c02890; |
| | Methanothermobacter marburgensis str. | ADL57895; |
| | Marburg; | ADL59006; |
| | | ADL57897; |
| 91: | Methanosphaera stadtmanae; | ABC57827 (ehbA); |
| Energy converting | Methanosphaera stadtmanae; | ABC57826 (ehbB); |
| hydrogenase (Ech) | Methanosphaera stadtmanae; | ABC57825 (ehbC); |
| | Methanosphaera stadtmanae; | ABC57824 (ehbD); |
| | Methanosphaera stadtmanae; | ABC57823 (ehbE); |
| | Methanosphaera stadtmanae; | ABC57822 (ehbF); |
| | Methanosphaera stadtmanae; | ABC57821 (ehbG); |
| | Methanosphaera stadtmanae; | ABC57820 (ehbH); |
| | Methanosphaera stadtmanae; | ABC57819 (ehbI); |
| | Methanosphaera stadtmanae; | ABC57818 (ehbJ); |
| | Methanosphaera stadtmanae; | ABC57817 (ehbK); |
| | Methanosphaera stadtmanae; | ABC57816 (ehbL); |
| | Methanosphaera stadtmanae; | ABC57815 (ehbM); |
| | Methanosphaera stadtmanae; | ABC57814 (ehbN); |
| | Methanosphaera stadtmanae; | ABC57813 (ehbO); |
| | Methanosphaera stadtmanae; | ABC57812(ehbP); |
| | Methanosphaera stadtmanae; | ABC57807 (ehbQ); |
| | Methanothermobacter marburgensis; | ADL59203, YP_003850516; |
| | Methanobacterium sp. SWAN-1; | YP_004520980; |
| | Methanobrevibacter ruminantium M1; | YP_003424741, ADC47849; |
| 92: | Methanosphaera stadtmanae; | ABC56714 (mtrA); |
| Methyl-H4MPT: coenzyme | Methanosphaera stadtmanae; | ABC56713 (mtrB); |
| M methyltransferase (MtrA-H) | Methanosphaera stadtmanae; | YP_447355 (mrtC); |
| | Methanosphaera stadtmanae; | YP_447354 (mtrD); |
| | Methanosaeta harundinacea 6Ac; | AET65445 (mtrE); |
| | Methanopyrus kandleri AV19; | AAM01871 (mtrE); |
| | Methanoculleus marisnigri JR1; | YP_001046527 (mtrE); |
| | Methanoculleus marisnigri JR1 | YP_001046522 (mtrF); |
| | Methanopyrus kandleri AV19; | NP_614768 (mtrF); |
| | Methanosphaera stadtmanae; | YP_447359 (mtrG); |
| | Methanosphaera stadtmanae; | YP_447360 (mtrH); |
| | Archaeoglobus fulgidus DSM 4304; | NP_068850 (mtrH); |
| | Methanopyrus kandleri AV19; | AAM01874 (mtrB); |
| | Methanocella paludicola SANAE; | BAI60614 (mtrB); |
| | Methanosaeta harundinacea 6Ac; | AET65448 (mtrB); |
| | Methanoculleus marisnigri JR1; | YP_001046524 (mtrB); |
| | Methanocella paludicola SANAE; | YP_003355598 (mtrA); |
| | Methanocella paludicola SANAE; | YP_003355597 (mtrB); |
| | Methanocella paludicola SANAE; | YP_003355596 (mtrC); |
| | Methanocella paludicola SANAE; | YP_003355595 (mtrD); |
| | Methanocella paludicola SANAE; | YP_003355594 (mtrE); |
| | Methanocella paludicola SANAE; | BAI60616 (mtrF); |
| | Methanocella paludicola SANAE; | YP_003355600 (mtrG); |
| | Methanocella paludicola SANAE; | YP_003355601 (mtrH); |
| 93: | Methanobacterium aarhusense; | AAR27839 (mcrA); |
| Methyl-coenzyme M | Methanobacterium sp. MB4; | ABG78755 (mcrA); |
| reductase (Mcr) | Methanosphaera stadtmanae; | CAE48306 (mcrA) |
| | Methanosphaera stadtmanae; | CAE48303 (mcrB) |
| | Methanosphaera stadtmanae; | ABC56709 (mcrC); |
| | Methanosphaera stadtmanae; | CAE48305 (McrG) |
| | Methanosphaera stadtmanae; | ABC56731, ABC56728; |
| | Methanosphaera stadtmanae; | YP_447371, ABC56730 (mrtG); |
| | Methanosphaera stadtmanae; | ABC56794; |
| 94: | Methanocella paludicola SANAE; | YP_003357823 (hdrA); |
| Heterodisulfide reductases | Methanocella paludicola SANAE; | YP_003357824 (hdrB); |
| (HdrABC, HdrDE) | Methanocella paludicola SANAE; | YP_003357825 (hdrC) |
| | Methanosaeta harundinacea 6Ac; | AET63985 (hdrA); |
| | Methanosaeta harundinacea 6Ac; | AET63982 (hdrB); |
| | Methanosaeta harundinacea 6Ac; | AET63983 (C); |
| | Methanosaeta harundinacea 6Ac; | AET64166 (D); |
| | Methanosaeta harundinacea 6Ac; | AET64165 (E); |
| | Methanopyrus kandleri AV19; | NP_613552 (hdrA); |
| | Methanopyrus kandleri AV19; | NP_613857 (hdrB); |
| | Methanopyrus kandleri AV19; | NP_613858 (hdrC); |
| 95: | Methanosphaera stadtmanae; | ABC56726 (mvhA); |
| [NiFe]-hydrogenase | Cyanobium sp. PCC 7001; | EDY38497 (mvhA); |
| MvhADG (non-F420 | Methanothermobacter marburgensis; | ADL59096 (mvhA) |
| reducing hydrogenase; | Methanobrevibacter ruminantium M1 | YP_003424648 (mvhA); |
| methyl viologen-reducing | Desulfobacterium autotrophicum HRM2 | YP_002602450 (mvhA) |
| hydrogenase) | Desulfatibacillum alkenivorans AK-01 | ACL06634 (mvhA); |
| | Methanothermobacter marburgensis; | ADL59095 (mvhB); |
| | Desulfatibacillum alkenivorans AK-01; | ACL06636 (mvhB); |
| | Methanobrevibacter smithii DSM 2374; | ZP_05975561 (mvhB); |
| | Methanothermobacter marburgensis; | ADL59098 (mvhD); |
| | Methanothermobacter marburgensis; | YP_003850411 (mvhD); |
| | Methanobrevibacter smithii; | YP_001273574 (mvhD); |
| | Methanobrevibacter smithii; | ABQ87206 (mvhD); |
| | Methanothermobacter | AAB02349 (mvhD); |
| | thermautotrophicus; | |
| | Methanothermobacter marburgensis; | ADL59097 (mvhG); |
| | Desulfatibacillum alkenivorans AK-01; | ACL06635 (mvhG); |
| | Cyanobium sp. PCC 7001; | EDY38425 (mvhG); |
| | Methanosphaera stadtmanae; | ABC56725 (mvhG); |
| | Methanobrevibacter smithii DSM 2374; | EFC93226 (mvhG); |
| | Desulfatibacillum alkenivorans AK-01; | ACL06638; |
| | Desulfatibacillum alkenivorans AK-01; | ACL03322; |
| | Methanoculleus marisnigri JR1; | YP_001046332 (hypF); |
| 96: | Methanocella paludicola SANAE; | YP_003357229 (frhB-1); |
| Coenzyme F420-reducing | Methanocella paludicola SANAE; | YP_003357467 (frhB-2); |
| hydrogenase (Frh) | Methanocella paludicola SANAE; | YP_003357509 (frhB-3); |
| | Synechococcus elongatus PCC 7942; | ABB57389 (frhB); |
| | Synechocystis sp. PCC 6803; | BAA18574, YP_001735870; |
| | Synechococcus sp. WH 7803; | YP_001225273; |
| | Synechococcus sp. RCC307; | YP_001227030; |
| | Cyanothece sp. PCC 8802; | ACV00312 (frhB); |
| | Cyanobium sp. PCC 7001; | EDY39891 (fehB); |
| | Synechococcus sp. RS9916; | EAU74116 (frhB); |
| | Synechococcus sp. JA-2-3B′a(2-13); | YP_477499; |
| | Pelotomaculum thermopropionicum SI; | YP_001212042, YP_001211959; |
| | Methanothermus fervidus DSM 2088; | YP_004004590; |
| | Methanococcus maripaludis S2; | CAF30376 (A), NP_988502 (A); |
| | Methanococcus maripaludis S2; | NP_988505 (B); |
| | Methanococcus maripaludis S2; | NP_988503 (D); |
| | Methanococcus maripaludis S2; | NP_988504 (G); |
| 97: | Methanobrevibacter ruminantium M1; | YP_003423444 (ahaA); |
| A₁Aₒ-ATP synthase (AhaA-IK) | Methanobrevibacter ruminantium M1; | YP_003423445 (ahaB); |
| | Methanobrevibacter ruminantium M1; | YP_003423442 (ahaC); |
| | Methanobrevibacter ruminantium M1 | ADC46554 (ahaD); |
| | Methanobrevibacter ruminantium M1; | ADC46549 (ahaE); |
| | Methanobrevibacter ruminantium M1; | YP_003423443 (ahaF); |
| | Methanobrevibacter ruminantium M1; | YP_003423438 (ahaH) |
| | Methanobrevibacter ruminantium M1; | ADC46547 (ahaI); |
| | Methanobrevibacter ruminantium M1; | YP_003423440 (ahaK); |
| | Ferroplasma acidarmanus fer1; | ZP_05570724; |
| | Thermococcus sibiricus MM 739; | YP_002995194; |
| | Thermoproteus tenax Kra 1; | CCC82573; |
| | Thermoproteus tenax Kra 1; | CCC82176; |
| | Methanosarcina mazei Go1; | AAC06375 (ahaA); |
| | Methanosarcina mazei Go1; | AAC06376 (ahaB); |
| | Methanosarcina mazei Go1; | AAC06373 (ahaC); |
| | Methanosarcina mazei Go1; | AAC06377 (ahaD) |
| | Methanosarcina mazei Go1; | AAC06372 (ahaE); |
| | Methanosarcina mazei Go1; | AAC06374 (ahaF); |
| | Methanosarcina mazei Go1; | AAC06378 (ahaG); |
| 98: | Methanosarcina mazei Go1; | CAA58177 (mhtA); |
| Membrane bound | Methanosarcina acetivorans C2A; | NP_616088 (mhtA); |
| cytochrome-containing F420- | Archaeoglobus fulgidus DSM 4304; | NP_070209 (mhtA); |
| nonreducing hydrogenase | Ferroglobus placidus DSM 10642; | ADC65001 (mhtA); |
| (VhtGAC, VhtD) | Methanosarcina acetivorans C2A; | NP_616088 (mhtB); |
| | Archaeoglobus fulgidus DSM 4304; | NP_070209 (mhtB); |
| | Methanosarcina mazei Go1; | CAA58178 (mhtB); |
| | Methanocella paludicola SANAE; | YP_003357991 (mhtC); |
| | Methanosarcina acetivorans C2A; | NP_616084(mhtC); |
| | Methanosarcina mazei Go1; | CAA58178 (nhtC); |
| | Methanosarcina mazei Go1; | NP_634195 (mhtC); |
| | Methanosarcina acetivorans C2A; | AAM04564 (mhtC); |
| | Methanosarcina mazei Go1; | CAA62962 (nhtD); |
| | Methanocella paludicola SANAE; | YP_003355429 (mhtD); |
| | Methanosarcina acetivorans C2A; | NP_616085 (mhtD); |
| | Methanosarcina acetivorans C2A; | NP_616087 (mhtG); |
| | Methanosarcina mazei Go1; | CAA581769 (mhtG); |
| | Methanocella paludicola SANAE; | YP_003357989 (mhtG); |
| | Methanosarcina acetivorans C2A; | AAM04562 (mhtG); |
| | Archaeoglobus fulgidus DSM 4304; | AAB89863 (mhtG); |
| 99a: | Methanobrevibacter ruminantium M1; | YP_003423415 (cofA); |
| CofA:Lactaldehyde | Methanobrevibacter ruminantium M1; | ADC46523 (cofA); |
| dehydrogenase (for F₄₂₀ | Methanothermococcus okinawensis IH1; | YP_004576675; |
| synthesis) | Methanotorris igneus Kol 5; | YP_004484309; |
| | Methanolinea tarda NOBI-1; | EHF10591; |
| | Methanobacterium sp. SWAN-1; | YP_004520759; |
| | Methanobacterium sp. AL-21; | YP_004289639; |
| | Methanolinea tarda NOBI-1; | ZP_09042363; |
| 99b: | Methanothermobacter marburgensis; | cofB; |
| CofB: L-Lactate kinase (for | Methanothermobacter | cofB; |
| F₄₂₀ synthesis) | thermautotrophicus | |
| 99c: | Methanothermobacter marburgensis; | ADL58588; |
| CofC: 2-phospho-L-lactate | Haloquadratum walsbyi C23; | CCC41432; |
| guanylyltransferase (for F₄₂₀ | Methanobrevibacter ruminantium M1; | YP_003423696; |
| synthesis) | Archaeoglobus veneficus SNP6; | YP_004342334; |
| | Natronobacterium gregoryi SP2; | ZP_08967286; |
| | Methanosalsum zhilinae DSM 4017; | AEH61444; |
| | Methanoplanus petrolearius; | ADN35493; |
| | Methanolinea tarda NOBI-1; | EHF10295; |
| 99d: | Methanococcus maripaludis S2; | NP987524; |
| CofD: LPPG:Fo 2-phospho- | Archaeoglobus veneficus SNP6; | YP_004341066; |
| L-lactate transferase (for F₄₂₀ | Methanospirillum hungatei JF-1; | YP_503864; |
| synthesis) | Methanococcus maripaludis XI; | YP_004742044; |
| | Methanocella paludicola SANAE; | YP_003356970; |
| | Methanosphaera stadtmanae; | YP_448417; |
| | Methanopyrus kandleri AV19; | NP_614772; |
| | Methanoculleus marisnigri JR1; | YP_001048050; |
| | Methanosaeta harundinacea 6Ac; | AET64321; |
| | Methanocorpusculum labreanum Z; | YP_001029596; |
| | Methanococcus maripaludis S2; | CAF29960; |
| 99e: | Methanothermobacter | NP_276154; |
| CofE: F₄₂₀-0: gamma- | thermautotrophicus; | |
| glutamyl ligase | Methanocorpusculum labreanum Z; | YP_001030766; |
| (for F₄₂₀ synthesis) | Methanothermus fervidus DSM 2088; | YP_004003885; |
| | Methanohalophilus mahii DSM 5219; | ADE37403; |
| | Mycobacterium sp. Spyr1; | YP_004078486; |
| | Halogeometricum borinquense; | YP_004035572; |
| | Methanococcus maripaludis C5; | ABO35054; |
| | Methanosarcina barkeri str. Fusaro; | YP_305815; |
| | Methanocorpusculum labreanum Z; | YP_001030766; |
| | Methanococcoides burtonii DSM 6242; | YP_566482; |
| | Methanoculleus marisnigri JR1; | ABN57125; |
| | Methanosaeta thermophila PT; | ABK13958; |
| | Acidothermus cellulolyticus 11B | ABK53734; |
| 99f: | Methanobrevibacter ruminantium M1; | YP_003424716 (cofG); |
| CofGH: Fo synthase (for F₄₂₀ | Methanococcus maripaludis S2; | CAF30432 (cofG); |
| synthesis) | Methanosphaera stadtmanae; | YP_447349 (cofG) |
| | Methanocella paludicola SANAE; | YP_003357513 (cofG); |
| | Methanopyrus kandleri AV19; | NP_614181 (cofG); |
| | Synechococcus sp. PCC 7002; | YP_001734664 (cofG); |
| | Cyanothece sp. PCC 7425; | YP_002481576 (cofG); |
| | Synechococcus elongatus PCC 7942; | ABB56922 (cofG); |
| | Synechocystis sp. PCC 6803 | NP_440537 (cofG) |
| | Synechococcus elongatus PCC 7942; | YP_399705 (cofH); |
| | Synechocystis sp. PCC 6803; | NP_440146 (cofH); |
| | Thermosynechococcus elongatus BP-1; | NP_682387 (cofH); |
| | Cyanothece sp. ATCC 51472; | EHC24992 (cofH); |
| | Methanosphaera stadtmanae; | ABC56793 (cofH); |
| | Methanococcus maripaludis S2; | NP_987177 (cofH); |
| | Methanobrevibacter ruminantium M1, | YP_003424008 (cofH); |
| | Methanosarcina mazei Go1; | NP_634520 (cofH); |
| | Methanocella paludicola SANAE; | YP_003357511 (cofH); |
| 100: | Methanocella paludicola SANAE; | YP_003355454; |
| Pyridoxal phosphate- | Methanobrevibacter ruminantium M1; | YP_003424638; |
| dependent L-tyrosine | Thermococcus gammatolerans EJ3; | YP_002960503; |
| decarboxylase (mfnA for | Halobacterium salinarum R1; | YP_001688512; |
| methanofuran synthesis) | Methanothermobacter marburgensis; | ADL59079; |
| | Thermococcus gammatolerans EJ3; | ACS34639; |
| | Haloferax vokanii DS2; | YP_003534871; |
| 101a: | Methanosphaera stadtmanae; | YP_447347; |
| MptA: GTP cyclohydrolase | Methanobrevibacter ruminantium M1; | YP_003424704; |
| (for Methanopterin synthesis) | Methanococcus maripaludis S2; | NP_987154; |
| | Pyrococcus horikoshii OT3; | NP_143623; |
| | Thermococcus gammatolerans EJ3; | YP_002959796; |
| | Methanosarcina mazei Go1; | NP_633246; |
| | Methanospirillum hungatei JF-1; | YP_503757; |
| | Thermococcus kodakarensis KOD1; | YP_183206; |
| | Methanopyrus kandleri AV19; | NP_613770; |
| | Methanosarcina acetivorans C2A; | NP_619377; |
| | Methanocaldococcus fervens AG86; | YP_003128348; |
| | Methanoregula boonei 6A8; | YP_001403641; |
| | Methanothermobacter | NP_276324; |
| | thermautotrophicus; | |
| | Methanosarcina barkeri str. Fusaro; | YP_304731; |
| | Methanocaldococcus jannaschii; | NP_247760; |
| 101b: | Methanococcus maripaludis C5; | ABO35741; |
| MptB: Cyclic | Roseobacter denitrificans OCh 114; | YP_683148; |
| phosphodiesterase | Arabidopsis thaliana; | AEE84108; |
| (for Methanopterin synthesis) | Zea mays; | NP_001151923; |
| | Medicago truncatula; | XP_003629873; |
| 101c: | Methanothermus fervidus DSM 2088; | YP_004003771; |
| RFAP: | Methanocella paludicola SANAE; | YP_003356610; |
| Ribofuranosylaminobenzene | Methanoplanus petrolearius; | ADN37264; |
| 5′-phosphate synthase (for | Methanobrevibacter ruminantium M1; | YP_003424432; |
| Methanopterin synthesis) | Archaeoglobus veneficus SNP6; | YP_004342012; |
| | Thermococcus sp. AM4; | YP_002582695; |
| | Methanococcus maripaludis S2; | NP_987399; |
| | Methanothermus fervidus DSM 2088; | ADP77009; |
| | Methanocella paludicola SANAE; | BAI61627; |
| 102a: | Methanothermobacter marburgensis; | ADL57861; |
| ComA: Phosphosulfolactate | Methanococcus maripaludis S2; | NP_987393; |
| synthase (for Coenzyme M | Methanosphaera stadtmanae; | ABC57647; |
| synthesis) | Methanothermus fervidus DSM 2088; | YP_004004617; |
| | Methanothermococcus okinawensis IH1; | YP_004575938; |
| | Methanobacterium sp. SWAN-1; | YP_004519242; |
| | Methanocaldococcus fervens AG86; | YP_003127444; |
| | Methanococcus voltae A3; | ADI36986; |
| | Methanococcus maripaludis C6; | YP_001548728; |
| | Methanobacterium sp. AL-21; | YP_004291430; |
| | Methanococcus aeolicus Nankai-3; | YP_001324357; |
| | Methanotorris igneus Kol 5; | AEF96400; |
| | Methanobacterium sp. AL-21 | ADZ10458; |
| | Methanococcus maripaludis X1; | AEK19167; |
| | Methanocaldococcus infernus ME; | ADG13665; |
| | Methanocaldococcus sp. FS406-22; | YP_003457919; |
| 102b: | Methanococcus maripaludis S2; | NP_987281; |
| ComB: 2- | Methanopyrus kandleri AV19; | AAM01355; |
| Phosphosulfolactate | Methanothermobacter marburgensis; | YP_003850451; |
| phosphatase (for Coenzyme | Methanococcus maripaludis S2; | CAF29717; |
| M synthesis) | Methanocella paludicola SANAE; | YP_003357619 |
| | Methanothermus fervidus DSM 2088; | YP_004004784; |
| | Methanothermus fervidus DSM 2088; | ADP78022; |
| | Methanobacterium sp. AL-21; | YP_004289567; |
| | Methanobrevibacter ruminantium M1; | YP_003424691; |
| | Synechocystis sp. PCC 6803; | BAK50080; |
| | Synechococcus sp. JA-2-3B′a(2-13); | YP_476548; |
| | Synechococcus sp. PCC 7002; | YP_001735079; |
| | Synechococcus sp. WH 7803; | YP_001224757; |
| | Cyanothece sp. ATCC 51472; | EHC21417; |
| | Synechococcus sp. WH 8016; | ZP_08955317; |
| 102c: | Methanothermobacter marburgensis; | ADL59162; |
| ComC: Sulfolactate | Methanosphaera stadtmanae; | ABC56689; |
| | Methanothermobacter marburgensis; | YP_003850475; |
| dehydrogenase (for | Methanothermus fervidus DSM 2088; | YP_004003953; |
| Coenzyme M synthesis) | Roseobacter litoralis Och 149; | YP_004689622; |
| | Methanococcus maripaludis C5; | ABO34766; |
| | Methanothermus fervidus DSM 2088; | ADP77191; |
| 102d: | Methanosarcina acetivorans C2A; | NP_618188; |
| ComDE: Sulfopyruvate | Methanocella paludicola SANAE; | YP_003357048; |
| decarboxylase (for | Methanocorpusculum labreanum Z; | YP_001029945; |
| Coenzyme M synthesis) | Methanoculleus marisnigri JR1; | ABN56047; |
| | Methanosarcina barkeri str. Fusaro; | YP_306991; |
| | Methanocella paludicola SANAE; | BAI62065; |
| | Methanosphaera stadtmanae; | ABC56687; |
| | Methanococcus maripaludis S2; | NP_988809; |
| 102e: | Methanothermobacter marburgensis; | comF; |
| ComF: Sulfoacetaldehyde | Methanothermobacter | comF; |
| dehydrogenase (for | thermautotrophicus | |
| Coenzyme M synthesis) | | |
| 103a: | Methanopyrus kandleri AV19; | AAM01606; |
| LeuA homolog: | Methanothermobacter | AAB85956; |
| Isopropylmalate synthase | thermautotrophicus; | |
| (for Coenzyme B synthesis) | Thermoproteus tenax; | CAF18516; |
| | Thermoplasma volcanium GSS1; | NP_111428; |
| | Methanobrevibacter smithii; | ABQ87451; |
| | Methanosphaera stadtmanae; | YP_447259; |
| | Methanobrevibacter ruminantium M1; | YP_003424897; |
| | Methanococcus maripaludis S2; | NP_988183; |
| | Synechocystis sp. PCC 6803 | NP_442009; |
| | Synechococcus elongatus PCC 7942; | ABB56460; |
| | Cyanothece sp. ATCC 51472; | EHC25498; |
| | Synechococcus sp. WH 8016; | ZP_08954784; |
| | Synechococcus sp. JA-2-3B′a(2-13) | YP_477672; |
| | Thermosynechococcus elongatus BP-1; | NP_682187; |
| 103b: | Methanopyrus kandleri AV19; | NP_614498; |
| LeuB homolog: | Methanothermobacter marburgensis; | ADL58232; |
| Isopropylmalate | Methanothermus fervidus DSM 2088; | YP_004004146; |
| dehydrogenase (for | Methanocella paludicola SANAE; | YP_003358048; |
| Coenzyme B synthesis) | Methanosphaera stadtmanae; | YP_447715; |
| | Methanocella paludicola SANAE; | BAI63065; |
| | Methanococcus maripaludis S2; | CAF30095; |
| | Synechocystis sp. PCC 6803; | NP_441348; |
| | Synechococcus elongatus PCC 7942; | ABB57535; |
| | Cyanothece sp. ATCC 51472; | EHC23198; |
| | Synechococcus sp. JA-2-3B′a(2-13; | YP_477855; |
| | Thermosynechococcus elongatus BP-1; | NP_682390; |
| 103c: | Marinobacter adhaerens HP15; | ADP98363, ADP98362; |
| LeuCD homolog: | Halorhabdus tiamatea SARL4B; | ZP_08559069; |
| Isopropylmalate isomerase | Haloarcula marismortui ATCC 43049; | YP_135090; |
| (for Coenzyme B synthesis) | Halomicrobium mukohataei; | YP_003178469; |
| | Haladaptatus paucihalophilus DX253; | ZP_08045715; |
| | Escherichia coli O103:H2 str. 12009; | YP_003220086, YP_003220085; |
| | Synechocystis sp. PCC 6803; | NP_442926, NP_441618; |
| | Cyanothece sp. PCC 8801; | YP_002370476, YP_002373868; |
| | Nostoc sp. PCC 7120; | NP_485460, NP_485459; |
| | Synechococcus sp. JA-2-3B′a(2-13); | YP_478232, YP_476588; |
| | Thermosynechococcus elongatus BP-1; | NP_681699, NP_682024; |

### Designer Calvin-Cycle-Channeled 1-Butanol Producing Pathways

According to one of the various embodiments, a designer Calvin-cycle-channeled pathway is created that takes the Calvin-cycle intermediate product, 3-phosphoglycerate, and converts it into 1-butanol by using, for example, a set of enzymes consisting of (as shown with the numerical labels 34, 35, 03-05, 36-43 in FIG. 4): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, citramalate synthase 36, 2-methylmalate dehydratase 37, 3-isopropylmalate dehydratase 38, 3-isopropylmalate dehydrogenase 39, 2-isopropylmalate synthase 40, isopropylmalate isomerase 41, 2-keto acid decarboxylase 42, and alcohol dehydrogenase (NAD dependent) 43. In this pathway design, as mentioned above, the NADPH-dependent glyceraldehyde-3-phosphate dehydrogenase 34 and NAD-dependent glyceraldehyde-3-phosphate dehydrogenase 35 serve as a NADPH/NADH conversion mechanism that can covert certain amount of photosynthetically generated NADPH to NADH which can be used by the NADH-requiring alcohol dehydrogenase 43 (examples of its encoding gene with the following GenBank accession numbers: BAB59540, CAA89136, NP_{—}148480) for production of 1-butanol by reduction of butyraldehyde.

According to one of the various embodiments, it is a preferred practice to also use an NADPH-dependent alcohol dehydrogenase 44 that can use NADPH as the source of reductant so that it can help alleviate the requirement of NADH supply for enhanced photobiological production of butanol and other alcohols. As listed in Table 1, examples of NADPH-dependent alcohol dehydrogenase 44 include (but not limited to) the enzyme with any of the following GenBank accession numbers: YP_{—}001211038, ZP_{—}04573952, XP_{—}002494014, CAY71835, NP_{—}417484, EFC99049, and ZP_{—}02948287.

Note, the 2-keto acid decarboxylase 42 (e.g., AAS49166, ADA65057, CAG34226, AAA35267, CAA59953, A0QBE6, A0PL16) and alcohol dehydrogenase 43 (and/or 44) have quite broad substrate specificity. Consequently, their use can result in production of not only 1-butanol but also other alcohols such as propanol depending on the genetic and metabolic background of the host photosynthetic organisms. This is because all 2-keto acids can be converted to alcohols by the 2-keto acid decarboxylase 42 and alcohol dehydrogenase 43 (and/or 44) owning to their broad substrate specificity. Therefore, according to another embodiment, it is a preferred practice to use a substrate-specific enzyme such as butanol dehydrogenase 12 when/if production of 1-butanol is desirable. As listed in Table 1, examples of butanol dehydrogenase 12 are NADH-dependent butanol dehydrogenase (e.g., GenBank: YP_{—}148778, NP_{—}561774, AAG23613, ZP_{—}05082669, ADO12118) and/or NAD(P)H-dependent butanol dehydrogenase (e.g., NP_{—}562172, AAA83520, EFB77036, EFF67629, ZP_{—}06597730, EFE12215, EFC98086, ZP_{—}05979561).

In one of the various embodiments, another designer Calvin-cycle-channeled 1-butanol production pathway is created that takes the Calvin-cycle intermediate product, 3-phosphoglycerate, and converts it into 1-butanol by using, for example, a set of enzymes consisting of (as shown with the numerical labels 34, 35, 03, 04, 45-52 and 40-43 (44/12) in FIG. 4): NADPH-dependent glyceraldehyde-3-phosphate dehydrogenase 34, NAD-dependent glyceraldehyde-3-phosphate dehydrogenase 35, phosphoglycerate mutase 03, enolase 04, phosphoenolpyruvate carboxylase 45, aspartate aminotransferase 46, aspartokinase 47, aspartate-semialdehyde dehydrogenase 48, homoserine dehydrogenase 49, homoserine kinase 50, threonine synthase 51, threonine ammonia-lyase 52, 2-isopropylmalate synthase 40, isopropylmalate isomerase 41, 3-isopropylmalate dehydrogenase 39, 2-keto acid decarboxylase 42, and NAD-dependent alcohol dehydrogenase 43 (and/or NADPH-dependent alcohol dehydrogenase 44, or butanol dehydrogenase 12).

According to another embodiment, the amino-acids-metabolism-related 1-butanol production pathways [numerical labels 03-05, 36-43; and/or 03, 04, 45-52 and 39-43 (44/12)] can operate in combination and/or in parallel with other photobiological butanol production pathways. For example, as shown also in FIG. 4, the Frctose-6-photophate-branched 1-butanol production pathway (numerical labels 13-32 and 44/12) can operate with the parts of amino-acids-metabolism-related pathways [numerical labels 36-42, and/or 45-52 and 40-42) with pyruvate and/or phosphoenolpyruvate as their joining points.

Examples of designer Calvin-cycle-channeled 1-butanol production pathway genes (DNA constructs) are shown in the DNA sequence listings. SEQ ID NOS: 58-70 represent a set of designer genes for a designer nirA-promoter-controlled Calvin-cycle-channeled 1-butanol production pathway (as shown with numerical labels 34, 35, 03-05, and 36-43 in FIG. 4) in a host oxyphotobacterium such as Thermosynechococcus elongatus BP1. Briefly, SEQ ID NO: 58 presents example 58 of a designer nirA-promoter-controlled NADPH-dependent Glyceraldehyde-3-Phosphate Dehydrogenase (34) DNA construct (1417 bp) that comprises: a PCR FD primer (sequence 1-20), a 231-bp nirA promoter from Thermosynechococcus elongatus BP1 (21-251), an enzyme-encoding sequence (252-1277) selected/modified from the sequences of a Staphylococcus aureus 04-02981 NADPH-dependent glyceraldehyde-3-phosphate dehydrogenase (GenBank: ADC37857), a 120-bp rbcS terminator from BP1 (1278-1397), and a PCR RE primer (1398-1417) at the 3′ end.

SEQ ID NO: 59 presents example 59 of a designer nirA-promoter-controlled NAD-dependent glyceraldehyde-3-phosphate dehydrogenase (35) DNA construct (1387 bp) that comprises: a PCR FD primer (sequence 1-20), a 231-bp nirA promoter from Thermosynechococcus elongatus BP1 (21-251), an enzyme-encoding sequence (252-1247) selected/modified from the sequences of an Edwardsiella tarda FL6-60 NAD-dependent glyceraldehyde-3-phosphate dehydrogenase (GenBank: ADM41489), a 120-bp rbcS terminator from BP1 (1248-1367), and a PCR RE primer (1368-1387) at the 3′ end.

SEQ ID NO: 60 presents example 60 of a designer nirA-promoter-controlled Phosphoglycerate Mutase (03) DNA construct (1627 bp) that includes a PCR FD primer (sequence 1-20), a 231-bp nirA promoter from Thermosynechococcus elongatus BP1 (21-251), an enzyme-encoding sequence (252-1487) selected/modified from the sequences of a Oceanithermus profundus DSM 14977 phosphoglycerate mutase (GenBank: ADR35708), a 120-bp rbcS terminator from BP1 (1488-1607), and a PCR RE primer (1608-1627).

SEQ ID NO: 61 presents example 61 of a designer nirA-promoter-controlled Enolase (04) DNA construct (1678 bp) that includes a PCR FD primer (sequence 1-20), a 231-bp nirA promoter from Thermosynechococcus elongatus BP1 (21-251), an enzyme-encoding sequence (252-1538) selected from the sequences of a Syntrophothermus Enolase (GenBank: ADI02602), a 120-bp rbcS terminator from BP1 (1539-1658), and a PCR RE primer (1659-1678).

SEQ ID NO: 62 presents example 62 of a designer nirA-promoter-controlled Pyruvate Kinase (05) DNA construct (2137 bp) that includes a PCR FD primer (sequence 1-20), a 231-bp nirA promoter from Thermosynechococcus elongatus BP1 (21-251), an enzyme-encoding sequence (252-1997) selected from the sequences of a Syntrophothermus lipocalidus pyruvate kinase (GenBank: ADI02459), a 120-bp rbcS terminator from BP1 (1998-2117), and a PCR RE primer (2118-2137).

SEQ ID NO: 63 presents example 63 of a designer nirA-promoter-controlled Citramalate Synthase (36) DNA construct (2163 bp) that includes a PCR FD primer (sequence 1-20), a 305-bp nirA promoter (21-325), an enzyme-encoding sequence (326-1909) selected and modified from Hydrogenobacter thermophilus TK-6 citramalate synthase (YP_{—}003433013), a 234-bp rbcS terminator from BP1 (1910-2143), and a PCR RE primer (2144-2163).

SEQ ID NO: 64 presents example 64 of a designer nirA-promoter-controlled 3-Isopropylmalate/(R)-2-Methylmalate Dehydratase (37) DNA construct (2878 bp) consisting of a PCR FD primer (sequence 1-20), a 231-bp nirA promoter from Thermosynechococcus elongatus BP1 (21-251), a 3-isopropylmalate/(R)-2-methylmalate dehydratase large subunit-encoding sequence (252-2012) selected/modified from the sequences of an Eubacterium 3-isopropylmalate/(R)-2-methylmalate dehydratase large subunit (YP_{—}002930810), a 231-bp nirA promoter from Thermosynechococcus (2013-2243), a 3-isopropylmalate/(R)-2-methylmalate dehydratase small subunit-encoding sequence (2244-2738) selected/modified from the sequences of an Eubacterium 3-isopropylmalate/(R)-2-methylmalate dehydratase small subunit (YP_{—}002930809), a 120-bp rbcS terminator from BP1 (2739-2858), and a PCR RE primer (2859-2878).

SEQ ID NO: 65 presents example 65 of a designer nirA-promoter-controlled 3-Isopropylmalate Dehydratase (38) DNA construct (2380 bp) comprises: a PCR FD primer (sequence 1-20), a 231-bp nirA promoter from Thermosynechococcus elongatus BP1 (21-251), a 3-isopropylmalate dehydratase large subunit-encoding sequence (252-1508) selected/modified from the sequences of a Thermotoga petrophila 3-isopropylmalate dehydratase large subunit (ABQ46641), a 231-bp nirA promoter from Thermosynechococcus elongatus BP1 (1509-1739), a 3-isopropylmalate dehydratase small subunit-encoding sequence (1740-2240) selected/modified from the sequences of a Thermotoga 3-isopropylmalate dehydratase small subunit (ABQ46640), a 120-bp rbcS terminator from BP1 (2241-2360), and a PCR RE primer (2361-2380).

SEQ ID NO: 66 presents example 66 of a designer nirA-promoter-controlled 3-Isopropylmalate Dehydrogenase (39) DNA construct (1456 bp) consisting of: a PCR FD primer (1-20), a 231-bp nirA promoter from Thermosynechococcus elongatus BP1 (21-251), a 3-isopropylmalate dehydrogenase-encoding sequence (252-1316) selected from the sequences of a Thermotoga 3-isopropylmalate dehydrogenase (GenBank: CP000702 Region 349983 . . . 351047), a 120-bp rbcS terminator from BP1 (1317-1436), and a PCR RE primer (1437-1456).

SEQ ID NO: 67 presents example 67 of a designer nirA-promoter-controlled 2-Isopropylmalate Synthase (40, EC 4.1.3.12) DNA construct (1933 bp) consisting of: a PCR FD primer (sequence 1-20), a 231-bp nirA promoter from Thermosynechococcus elongatus (21-251), an enzyme-encoding sequence (252-1793) selected/modified from the sequences of a Thermotoga petrophila 3-isopropylmalate dehydrogenase (CP000702 Region: 352811 . . . 354352), a 120-bp rbcS terminator from BP1 (1794-1913), and a PCR RE primer (1914-1933).

SEQ ID NO: 68 presents example 68 of a designer nirA-promoter-controlled Isopropylmalate Isomerase (41) DNA construct (2632 bp) comprises: a PCR FD primer (sequence 1-20), a 231-bp nirA promoter from Thermosynechococcus elongatus BP1 (21-251), a isopropylmalate isomerase large subunit-encoding sequence (252-1667) selected/modified from the sequences of a Geobacillus kaustophilus 3-isopropylmalate isomerase large subunit (YP_{—}148509), a 231-bp nirA promoter from Thermosynechococcus (1668-1898), a isopropylmalate isomerase small subunit-encoding sequence (1899-2492) selected from the sequences of a Geobacillus kaustophilus isopropylmalate isomerase small subunit (YP_{—}148508), a 120-bp rbcS terminator from BP1 (2493-2612), and a PCR RE primer (2613-2632).

SEQ ID NO: 69 presents example 69 of a designer nirA-promoter-controlled 2-Keto Acid Decarboxylase (42) DNA construct (2035 bp) consisting of: a PCR FD primer (sequence 1-20), a 231-bp nirA promoter from Thermosynechococcus elongatus BP1 (21-251), a 2-keto acid decarboxylase-encoding sequence (252-1895) selected/modified from the sequences of a Lactococcus lactis branched-chain alpha-ketoacid decarboxylase (AAS49166), a 120-bp rbcS terminator from BP1 (1896-2015), and a PCR RE primer (2016-2035) at the 3′ end.

SEQ ID NO: 70 presents example 70 of a designer nirA-promoter-controlled NAD-dependent Alcohol Dehydrogenase (43) DNA construct (1426 bp) consisting of: a PCR FD primer (sequence 1-20), a 231-bp nirA promoter from Thermosynechococcus elongatus BP1 (21-251), an enzyme-encoding sequence (252-1286) selected/modified from the sequences of an Aeropyrum pernix K1 NAD-dependent alcohol dehydrogenase (NP_{—}148480), a 120-bp rbcS terminator from BP1 (1287-1406), and a PCR RE primer (1407-1426).

As mentioned before, use of an NADPH-dependent alcohol dehydrogenase 44 that can use NADPH as the source of reductant can help alleviate the requirement of NADH supply for enhanced photobiological production of butanol and other alcohols. SEQ ID NO: 71 presents example 71 of a designer nirA-promoter-controlled NADPH-dependent Alcohol Dehydrogenase (44) DNA construct (1468 bp) that comprises: a PCR FD primer (sequence 1-20), a 231-bp nirA promoter from Thermosynechococcus elongatus BP1 (21-251), an enzyme-encoding sequence (252-1328) selected from the sequences of a Pichia pastoris NADPH-dependent medium chain alcohol dehydrogenase with broad substrate specificity (XP_{—}002494014), a 120-bp rbcS terminator from BP1 (1329-1458), and a PCR RE primer (1459-1468) at the 3′ end. In one of the examples, this type of NADPH-dependent alcohol dehydrogenase gene (SEQ ID NO: 71) is also used in construction of Calvin-cycle-channeled butanol production pathway.

However, because of the broad substrate specificity of the 2-keto acid decarboxylase (42, SEQ ID NO: 69) and the alcohol dehydrogenase (43, SEQ ID NO: 70; or 44, SEQ ID NO: 71), the pathway expressed with designer genes of SEQ ID NO: 69 and SEQ ID NO: 71 (and/or SEQ ID NO: 70) can result in the production of alcohol mixtures rather than single alcohols since all 2-keto acids can be converted to alcohols by the two broad substrate specificity enzymes. Therefore, to improve the specificity for 1-butanol production, it is a preferred practice to use a more substrate-specific butanol dehydrogenase 12. SEQ ID NO: 72 presents example 72 of a designer nirA-promoter-controlled NADH-dependent Butanol Dehydrogenase (12a) DNA construct (1555 bp) that includes a PCR FD primer (sequence 1-20), a 231-bp nirA promoter from Thermosynechococcus elongatus BP1 (21-251), an enzyme-encoding sequence (252-1415) selected/modified from the sequences of a Geobacillus kaustophilus NADH-dependent butanol dehydrogenase (YP_{—}148778), a 120-bp rbcS terminator from BP1 (1416-1535), and a PCR RE primer (1536-1555) at the 3′ end.

SEQ ID NO: 73 presents example 73 of a designer nirA-promoter-controlled NADPH-dependent Butanol Dehydrogenase (12b) DNA construct (1558 bp) consisting of a PCR FD primer (sequence 1-20), a 231-bp nirA promoter from Thermosynechococcus elongatus BP1 (21-251), a NADPH-dependent butanol dehydrogenase-encoding sequence (252-1418) selected/modified from the sequences of a Clostridium perfringens NADPH-dependent butanol dehydrogenase (NP_{—}562172), a 120-bp rbcS terminator from BP1 (1419-1528), and a PCR RE primer (1529-1558) at the 3′ end.

Use of SEQ ID NOS: 72 and/or 73 (12a and/or 12b) along with SEQ ID NOS: 58-69 represents a specific Calvin-cycle-channeled 1-butanol production pathway numerically labeled as 34, 35, 03-05, 36-42 and 12 in FIG. 4.

SEQ ID NOS: 74-81 represent an alternative (amino acids metabolism-related) pathway (45-52 in FIG. 4) that branches from the point of phosphoenolpyruvate and merges at the point of 2-ketobutyrate in the Calvin-cycle-channeled 1-butanol production pathway. Briefly, SEQ ID NO: 74 presents example 74 of a designer nirA-promoter-controlled Phosphoenolpyruvate Carboxylase (45) DNA construct (3646 bp) consisting of: a PCR FD primer (sequence 1-20), a 231-bp nirA promoter from Thermosynechococcus elongatus BP1 (21-251), an enzyme-encoding sequence (252-3506) selected/modified from the sequences of a Thermaerobacter subterraneus DSM 13965 Phosphoenolpyruvate carboxylase (EFR61439), a 120-bp rbcS terminator from BP1 (3507-3626), and a PCR RE primer (3627-3646) at the 3′ end.

SEQ ID NO: 75 presents example 75 of a designer nirA-promoter-controlled Aspartate Aminotransferase (46) DNA construct (1591 bp) that includes a PCR FD primer (sequence 1-20), a 231-bp nirA promoter from Thermosynechococcus elongatus BP1 (21-251), an enzyme-encoding sequence (252-1451) selected/modified from the sequences of a Thermotoga lettingae aspartate aminotransferase (YP_{—}001470126), a 120-bp rbcS terminator from BP1 (1452-1471), and a PCR RE primer (1472-1591).

SEQ ID NO: 76 presents example 76 of a designer nirA-promoter-controlled Aspartate

Kinase (47) DNA construct (1588 bp) that includes a PCR FD primer (sequence 1-20), a 231-bp nirA promoter from Thermosynechococcus elongatus BP1 (21-251), an enzyme-encoding sequence (252-1448) selected/modified from the sequences of a Thermotoga lettingae TMO aspartate kinase (YP_{—}001470361), a 120-bp rbcS terminator from BP1 (1449-1568), and a PCR RE primer (1569-1588).

SEQ ID NO: 77 presents example 77 of a designer nirA-promoter-controlled Aspartate-Semialdehyde Dehydrogenase (48) DNA construct (1411 bp) that includes a PCR FD primer (sequence 1-20), a 231-bp nirA promoter from Thermosynechococcus elongatus BP1 (21-251), an enzyme-encoding sequence (252-1271) selected/modified from the sequences of a Thermotoga lettingae TMO aspartate-semialdehyde dehydrogenase (YP_{—}001470981), a 120-bp rbcS terminator from BP1 (1272-1391), and a PCR RE primer (1392-1411) at the 3′ end.

SEQ ID NO: 78 presents example 78 of a designer nirA-promoter-controlled Homoserine Dehydrogenase (49) DNA construct (1684 bp) that includes a PCR FD primer (sequence 1-20), a 231-bp nirA promoter from Thermosynechococcus elongatus BP1 (21-251), an enzyme-encoding sequence (252-1544) selected/modified from the sequences of a Syntrophothermus lipocalidus DSM 12680 homoserine dehydrogenase (ADI02231), a 120-bp rbcS terminator from BP1 (1545-1664), and a PCR RE primer (1665-1684) at the 3′ end.

SEQ ID NO: 79 presents example 79 of a designer nirA-promoter-controlled Homoserine Kinase (50) DNA construct (1237 bp) that includes a PCR FD primer (sequence 1-20), a 231-bp nirA promoter from Thermosynechococcus elongatus BP1 (21-251), an enzyme-encoding sequence (252-1097) selected/modified from the sequences of a Thermotoga petrophila RKU-1 Homoserine Kinase (YP_{—}001243979), a 120-bp rbcS terminator from BP1 (1098-1217), and a PCR RE primer (1218-1237) at the 3′ end.

SEQ ID NO: 80 presents example 80 of a designer nirA-promoter-controlled Threonine Synthase (51) DNA construct (1438 bp) that includes a PCR FD primer (sequence 1-20), a 231-bp nirA promoter from Thermosynechococcus (21-251), an enzyme-encoding sequence (252-1298) selected from the sequences of a Thermotoga Threonine Synthase (YP_{—}001243978), a 120-bp rbcS terminator from BP1 (1299-1418), and a PCR RE primer (1419-1438).

SEQ ID NO: 81 presents example 81 of a designer nirA-promoter-controlled Threonine Ammonia-Lyase (52) DNA construct (1600 bp) consisting of a PCR FD primer (sequence 1-20), a 231-bp nirA promoter from Thermosynechococcus elongatus BP1 (21-251), an enzyme-encoding sequence (252-1460) selected/modified from the sequences of a Geobacillus kaustophilus threonine ammonia-lyase (BAD75876), a 120-bp rbcS terminator from BP1 (1461-1580), and a PCR RE primer (1581-1600) at the 3′ end.

Note, SEQ ID NOS: 58-61, 74-81, 66-69, and 72 (and/or 73) represent a set of sample designer genes that can express a Calvin-cycle 3-phophoglycerate-branched photosynthetic NADPH-enhanced 1-butanol production pathway of 34, 35, 03, 04, 45-52 40, 41, 39, 42, and 12 while SEQ ID NOS: 58-69 and 72 (and/or 73) represent another set of sample designer genes that can express another Calvin-cycle 3-phophoglycerate-branched photosynthetic NADPH-enhanced 1-butanol production pathway as numerically labeled as 34, 35, 03-05, 36-42, and 12 in FIG. 4. The net results of the designer photosynthetic NADPH-enhanced pathways in working with the Calvin cycle are photobiological production of 1-butanol (CH₃CH₂CH₂CH₂OH) from carbon dioxide (CO₂) and water (H₂O) using photosynthetically generated ATP (Adenosine triphosphate) and NADPH (reduced nicotinamide adenine dinucleotide phosphate) according to the following process reaction:

4CO₂+5H₂O→CH₃CH₂CH₂CH₂OH+6O₂ [5]

### Designer Calvin-Cycle-Channeled 2-Methyl-1-Butanol Producing Pathways

According to one of the various embodiments, a designer Calvin-cycle-channeled 2-Methyl-1-Butanol production pathway is created that takes the Calvin-cycle intermediate product, 3-phosphoglycerate, and converts it into 2-methyl-1-butanol by using, for example, a set of enzymes consisting of (as shown with the numerical labels 34, 35, 03-05, 36-39, 53-55, 42, 43 or 44/56 in FIG. 5): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, citramalate synthase 36, 2-methylmalate dehydratase 37, 3-isopropylmalate dehydratase 38, 3-isopropylmalate dehydrogenase 39, acetolactate synthase 53, ketol-acid reductoisomerase 54, dihydroxy-acid dehydratase 55, 2-keto acid decarboxylase 42, and NAD-dependent alcohol dehydrogenase 43 (or NADPH-dependent alcohol dehydrogenase 44; more preferably, 2-methylbutyraldehyde reductase 56).

In another embodiment, a designer Calvin-cycle-channeled 2-methyl-1-butanol production pathway is created that takes the intermediate product, 3-phosphoglycerate, and converts it into 2-methyl-1-butanol by using, for example, a set of enzymes consisting of (as shown with the numerical labels 34, 35, 03, 04, 45-55, 42, 43 or 44/56 in FIG. 5): NADPH-dependent glyceraldehyde-3-phosphate dehydrogenase 34, NAD-dependent glyceraldehyde-3-phosphate dehydrogenase 35, phosphoglycerate mutase 03, enolase 04, phosphoenolpyruvate carboxylase 45, aspartate aminotransferase 46, aspartokinase 47, aspartate-semialdehyde dehydrogenase 48, homoserine dehydrogenase 49, homoserine kinase 50, threonine synthase 51, threonine ammonia-lyase 52, acetolactate synthase 53, ketol-acid reductoisomerase 54, dihydroxy-acid dehydratase 55, 2-keto acid decarboxylase 42, and NAD dependent alcohol dehydrogenase 43 (or NADPH dependent alcohol dehydrogenase 44; more preferably, 2-methylbutyraldehyde reductase 56).

These pathways (FIG. 5) are quite similar to those of FIG. 4, except that acetolactate synthase 53, ketol-acid reductoisomerase 54, dihydroxy-acid dehydratase 55, and 2-methylbutyraldehyde reductase 56 are used to produce 2-Methyl-1-Butanol.

SEQ ID NO: 82 presents example 82 of a designer nirA-promoter-controlled Acetolactate Synthase (53) DNA construct (2107 bp) that includes a PCR FD primer (sequence 1-20), a 231-bp nirA promoter from Thermosynechococcus elongatus BP1 (21-251), an acetolactate synthase-encoding sequence (252-1967) selected/modified from the sequences of a Bacillus subtilis subsp. subtilis str. 168 acetolactate synthase (CAB07802), a 120-bp rbcS terminator from BP1 (1968-2087), and a PCR RE primer (2088-2107) at the 3′ end.

SEQ ID NO: 83 presents example 83 of a designer nirA-promoter-controlled Ketol-Acid Reductoisomerase (54) DNA construct (1405 bp) that includes a PCR FD primer (sequence 1-20), a 231-bp nirA promoter from Thermosynechococcus elongatus BP1 (21-251), a ketol-acid reductoisomerase-encoding sequence (252-1265) selected/modified from the sequences of a Syntrophothermus lipocalidus DSM 12680 ketol-acid reductoisomerase (ADI02902), a 120-bp rbcS terminator from BP1 (1266-1385), and a PCR RE primer (1386-1405) at the 3′ end.

SEQ ID NO: 84 presents example 84 of a designer nirA-promoter-controlled Dihydroxy-Acid Dehydratase (55) DNA construct (2056 bp) that includes a PCR FD primer (1-20), a 231-bp nirA promoter from Thermosynechococcus elongatus BP1 (21-251), an enzyme-encoding sequence (252-1916) selected from the sequences of a Thermotoga dihydroxy-acid dehydratase (YP_{—}001243973), a 120-bp rbcS terminator from BP1 (1917-2036), and a PCR RE primer (2037-2056).

SEQ ID NO: 85 presents example 85 of a designer nirA-promoter-controlled 2-Methylbutyraldehyde Reductase (56) DNA construct (1360 bp) that includes a PCR FD primer (sequence 1-20), a 231-bp nirA promoter from Thermosynechococcus elongatus BP1 (21-251), an enzyme-encoding sequence (252-1220) selected/modified from the sequences of a Schizosaccharomyces japonicus 2-methylbutyraldehyde reductase (XP_{—}002173231), a 120-bp rbcS terminator from BP1 (1221-1340), and a PCR RE primer (1341-1360) at the 3′ end.

Note, SEQ ID NOS: 58-66, 82-84, 69 and 85 represent another set of sample designer genes that can express a Calvin-cycle 3-phophoglycerate-branched photosynthetic NADPH-enhanced 2-methyl-1-butanol production pathway numerically labeled as 34, 35, 03-05, 36-39, 53-55, 42 and 56; while SEQ ID NOS: 58-61, 74-84, 69 and 85 represent a set of sample designer genes that can express another Calvin-cycle 3-phophoglycerate-branched photosynthetic NADPH-enhanced 2-methyl-1-butanol production pathway of 34, 35, 03, 04, 45-55, 42 and 56 in FIG. 5. These designer genes can be used in combination with other pathway gene(s) to express certain other pathways such as a Calvin-cycle Fructose-6-phosphate branched 2-methyl-1-butanol production pathway numerically labeled as 13-26, 36-39, 53-55, 42 and 56 (and/or, as 13-25, 45-55, 42 and 56) in FIG. 5 as well. The net results of the designer photosynthetic NADPH-enhanced pathways in working with the Calvin cycle are production of 2-methyl-1-butanol [CH₃CH₂CH(CH₃)CH₂OH] from carbon dioxide (CO₂) and water (H₂O) using photosynthetically generated ATP and NADPH according to the following process reaction:

10CO₂+12H₂O→2CH₃CH₂CH(CH₃)CH₂OH+15O₂ [6]

### Calvin-Cycle-Channeled Pathways for Production of Isobutanol and 3-Methyl-1-Butanol

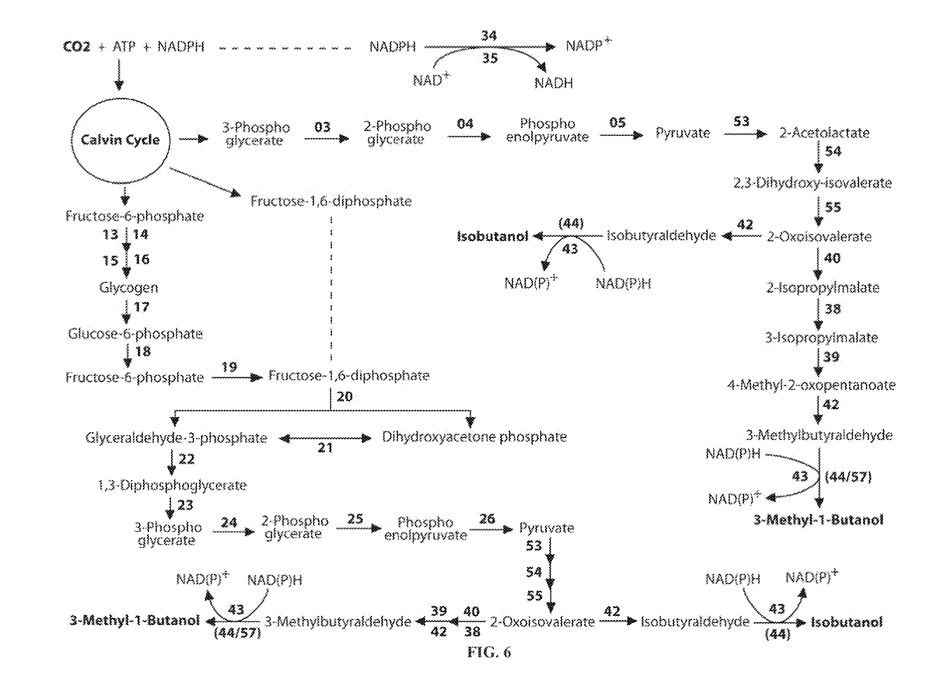

According to one of the various embodiments, a designer Calvin-cycle-channeled pathway is created that takes the Calvin-cycle intermediate product, 3-phosphoglycerate, and converts it into isobutanol by using, for example, a set of enzymes consisting of (as shown with numerical labels 34, 35, 03-05, 53-55, 42, 43 (or 44) in FIG. 6): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, acetolactate synthase 53, ketol-acid reductoisomerase 54, dihydroxy-acid dehydratase 55, 2-keto acid decarboxylase 42, and NAD-dependent alcohol dehydrogenase 43 (or NADPH-dependent alcohol dehydrogenase 44). The net result of this pathway in working with the Calvin cycle is photobiological production of isobutanol ((CH₃)₂CHCH₂OH) from carbon dioxide (CO₂) and water (H₂O) using photosynthetically generated ATP and NADPH according to the following process reaction:

4CO₂+5H₂O→(CH₃)₂CHCH₂OH+6I₂ [7]

According to another embodiment, a designer Calvin-cycle-channeled pathway is created that takes the intermediate product, 3-phosphoglycerate, and converts it into 3-methyl-1-butanol by using, for example, a set of enzymes consisting of (as shown with the numerical labels 34, 35, 03-05, 53-55, 40, 38, 39, 42, 43 (or 44/57) in FIG. 6): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, acetolactate synthase 53, ketol-acid reductoisomerase 54, dihydroxy-acid dehydratase 55, 2-isopropylmalate synthase 40, 3-isopropylmalate dehydratase 38, 3-isopropylmalate dehydrogenase 39, 2-keto acid decarboxylase 42, and NAD-dependent alcohol dehydrogenase 43 (or NADPH-dependent alcohol dehydrogenase 44; or more preferably, 3-methylbutanal reductase 57). The net result of this pathway in working with the Calvin cycle is photobiological production of 3-methyl-1-butanol(CH₃CH(CH₃)CH₂CH₂OH) from carbon dioxide (CO₂) and water (H₂O) using photosynthetically generated ATP and NADPH according to the following process reaction:

10CO₂+12H₂O→4CH₃CH(CH₃)CH₂CH₂OH+15O₂ [8]

These designer pathways (FIG. 6) share a number of designer pathway enzymes with those of FIGS. 4 and 5, except that a 3-methylbutanal reductase 57 is preferably used for production of 3-methyl-1-butanol; they all have a common feature of using an NADPH-dependent glyceraldehyde-3-phosphate dehydrogenase 34 and an NAD-dependent glyceraldehyde-3-phosphate dehydrogenase 35 as an NADPH/NADH conversion mechanism to covert certain amount of photosynthetically generated NADPH to NADH which can be used by NADH-requiring pathway enzymes such as an NADH-requiring alcohol dehydrogenase 43.

SEQ ID NO: 86 presents example 86 of a designer nirA-promoter-controlled 3-Methylbutanal Reductase (57) DNA construct (1420 bp) that includes a PCR FD primer (sequence 1-20), a 231-bp nirA promoter from Thermosynechococcus elongatus BP1 (21-251), an enzyme-encoding sequence (252-1280) selected/modified from the sequences of a Saccharomyces cerevisiae S288c 3-Methylbutanal reductase (DAA10635), a 120-bp rbcS terminator from BP1 (1281-1400), and a PCR RE primer (1401-1420) at the 3′ end.

SEQ ID NOS: 58-62, 82-84, 69, 70 (or 71) represent a set of sample designer genes that can express a Calvin-cycle 3-phosphoglycerate-branched photosynthetic NADPH-enhanced isobutanol production pathway (34, 35, 03-05, 53-55, 42, 43 or 44); while SEQ ID NOS: 58-62, 82-84, 65-67, 69 and 86 represent another set of sample designer genes that can express a Calvin-cycle 3-phosphoglycerate-branched photosynthetic NADPH-enhanced 3-methyl-1-butanol production pathway (34, 35, 03-05, 53-55, 40, 38, 39, 42, and 57 in FIG. 6).

These designer genes can be used with certain other designer genes to express certain other pathways such as a Calvin-cycle Fructose-6-phosphate-branched 3-methyl-1-butanol production pathway shown as 13-26, 53-54, 39-40, 42 and 57 (or 43/44) in FIG. 6 as well. The net results of the designer photosynthetic NADPH-enhanced pathways in working with the Calvin cycle are also production of isobutanol ((CH₃)₂CHCH₂OH) and/or 3-methyl-1-butanol (CH₃CH(CH₃)CH₂CH₂OH) from carbon dioxide (CO₂) and water (H₂O) using photosynthetically generated ATP and NADPH.

### Designer Calvin-Cycle-Channeled Pathways for Production of 1-Hexanol and 1-Octanol

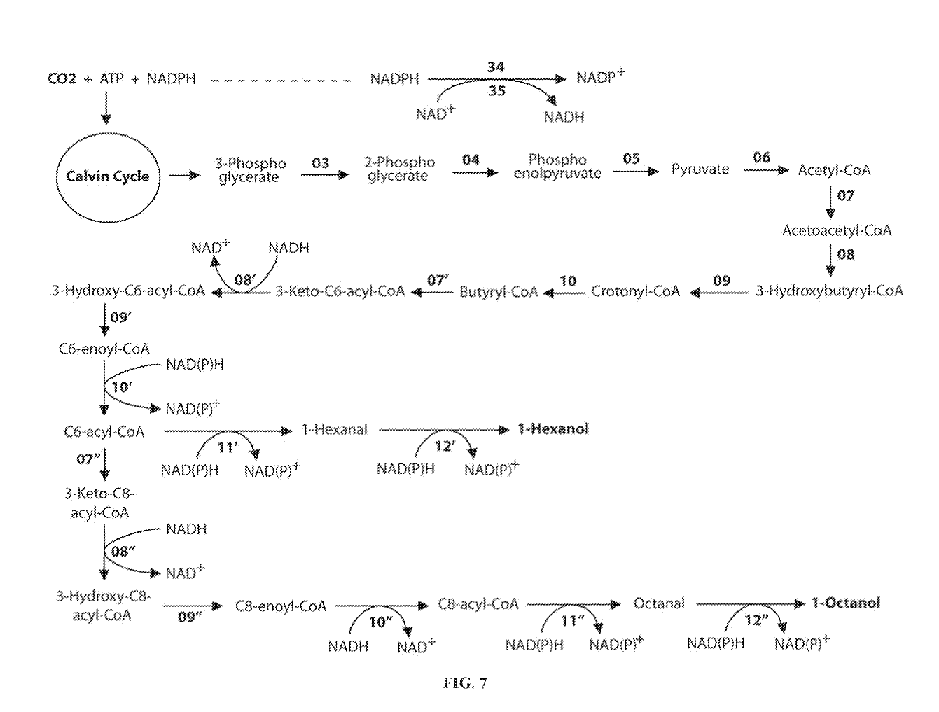

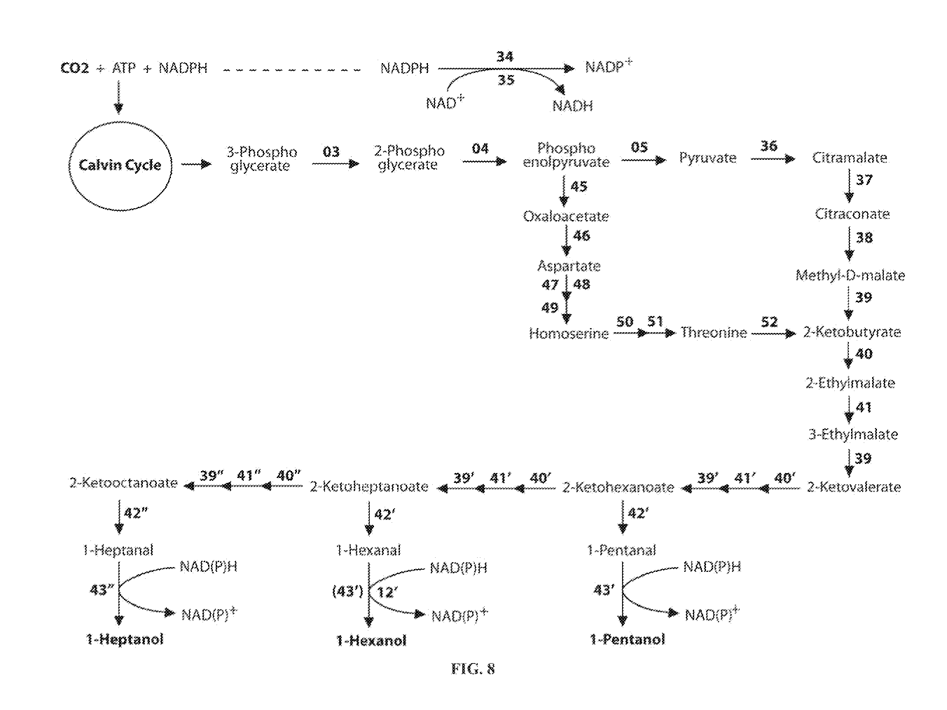

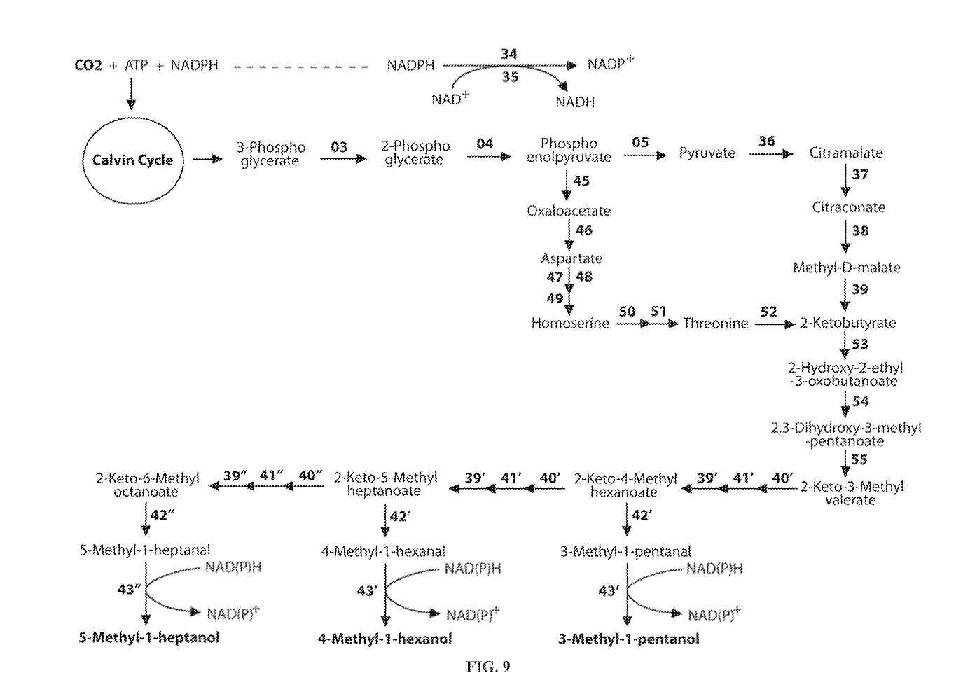

According to one of the various embodiments, a designer Calvin-cycle-channeled pathway is created that takes the Calvin-cycle intermediate product, 3-phosphoglycerate, and converts it into 1-hexanol by using, for example, a set of enzymes consisting of (as shown with the numerical labels 34, 35, 03-10, 07′-12′ in FIG. 7): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, pyruvate-ferredoxin oxidoreductase 06, thiolase 07, 3-hydroxybutyryl-CoA dehydrogenase 08, crotonase 09, butyryl-CoA dehydrogenase 10, designer 3-ketothiolase 07′, designer 3-hydroxyacyl-CoA dehydrogenase 08′, designer enoyl-CoA dehydratase 09′, designer 2-enoyl-CoA reductase 10′, designer acyl-CoA reductase 11′, and hexanol dehydrogenase 12′. The net result of this designer pathway in working with the Calvin cycle is photobiological production of 1-hexanol (CH₃CH₂CH₂CH₂CH₂CH₂OH) from carbon dioxide (CO₂) and water (H₂O) using photosynthetically generated ATP and NADPH according to the following process reaction:

6CO₂+7H₂O→CH₃CH₂CH₂CH₂CH₂CH₂OH+9O₂ [9]

According to another embodiment, a designer Calvin-cycle-channeled pathway is created that takes the intermediate product, 3-phosphoglycerate, and converts it into 1-octanol by using, for example, a set of enzymes consisting of (as shown with the numerical labels 34, 35, 03-10, 07′-10′, and 07″-12″ in FIG. 7): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, pyruvate-ferredoxin oxidoreductase 06, thiolase 07, 3-hydroxybutyryl-CoA dehydrogenase 08, crotonase 09, butyryl-CoA dehydrogenase 10, designer 3-ketothiolase 07′, designer 3-hydroxyacyl-CoA dehydrogenase 08′, designer enoyl-CoA dehydratase 09′, designer 2-enoyl-CoA reductase 10′, designer 3-ketothiolase 07″, designer 3-hydroxyacyl-CoA dehydrogenase 08″, designer enoyl-CoA dehydratase 09″, designer 2-enoyl-CoA reductase 10″, designer acyl-CoA reductase 11″, and octanol dehydrogenase 12″.

These pathways represent a significant upgrade in the pathway designs with part of a previously disclosed 1-butanol production pathway (03-10). The key feature is the utilization of an NADPH-dependent glyceraldehyde-3-phosphate dehydrogenase 34 and an NAD-dependent glyceraldehyde-3-phosphate dehydrogenase 35 as a mechanism for NADPH/NADH conversion to drive an NADH-requiring designer hydrocarbon chain elongation pathway (07′-10′) for 1-hexanol production (07′-12′ as shown in FIG. 7).

SEQ ID NOS: 87-92 represent a set of designer genes that can express the designer hydrocarbon chain elongation pathway for 1-hexanol production (07′-12′ as shown in FIG. 7). Briefly, SEQ ID NO: 87 presents example 87 of a designer nirA-promoter-controlled 3-Ketothiolase (07′) DNA construct (1540 bp) that includes a PCR FD primer (sequence 1-20), a 231-bp nirA promoter from Thermosynechococcus elongatus BP1 (21-251), an enzyme-encoding sequence (252-1400) selected/modified from the sequences of a Geobacillus kaustophilus 3-Ketothiolase (YP_{—}147173), a 120-bp rbcS terminator from BP1 (1401-1520), and a PCR RE primer (1521-1540).

SEQ ID NO: 88 presents example 88 of a designer nirA-promoter-controlled 3-Hydroxyacyl-CoA Dehydrogenase (08′) DNA construct (1231 bp) that includes a PCR FD primer (sequence 1-20), a 231-bp nirA promoter from Thermosynechococcus elongatus BP1 (21-251), an enzyme-encoding sequence (252-1091) selected/modified from the sequences of a Syntrophothermus lipocalidus 3-Hydroxyacyl-CoA dehydrogenase (YP_{—}003702743), a 120-bp rbcS terminator from BP1 (1092-1211), and a PCR RE primer (1212-1231).

SEQ ID NO: 89 presents example 89 of a designer nirA-promoter-controlled Enoyl-CoA Dehydratase (09′) DNA construct (1162 bp) that includes a PCR FD primer (sequence 1-20), a 231-bp nirA promoter from Thermosynechococcus elongatus BP1 (21-251), an enzyme-encoding sequence (252-1022) selected/modified from the sequences of a Bordetella petrii Enoyl-CoA dehydratase (CAP41574), a 120-bp rbcS terminator from BP1 (1023-1442), and a PCR RE primer (1443-1162) at the 3′ end.

SEQ ID NO: 90 presents example 90 of a designer nirA-promoter-controlled 2-Enoyl-CoA Reductase (10′) DNA construct (1561 bp) that includes a PCR FD primer (sequence 1-20), a 231-bp nirA promoter from Thermosynechococcus elongatus BP1 (21-251), an enzyme-encoding sequence (252-1421) selected/modified from the sequences of a Xanthomonas campestris 2-Enoyl-CoA Reductase (CAP53709), a 120-bp rbcS terminator from BP1 (1422-1541), and a PCR RE primer (1542-1561).

SEQ ID NO: 91 presents example 91 of a designer nirA-promoter-controlled Acyl-CoA Reductase (11′) DNA construct (1747 bp) that includes a PCR FD primer (sequence 1-20), a 231-bp nirA promoter from Thermosynechococcus elongatus BP1 (21-251), an enzyme-encoding sequence (252-1607) selected/modified from the sequences of a Clostridium cellulovorans Acyl-CoA reductase (YP_{—}003845606), a 120-bp rbcS terminator from BP1 (1608-1727), and a PCR RE primer (1728-1747).

SEQ ID NO: 92 presents example 92 of a designer nirA-promoter-controlled Hexanol Dehydrogenase (12′) DNA construct (1450 bp) that includes a PCR FD primer (sequence 1-20), a 231-bp nirA promoter from Thermosynechococcus elongatus BP1 (21-251), an enzyme-encoding sequence (252-1310) selected/modified from the sequences of a Mycobacterium chubuense hexanol dehydrogenase (ACZ56328), a 120-bp rbcS terminator from BP1 (1311-1430), and a PCR RE primer (1431-1450).

SEQ ID NO: 93 presents example 93 of a designer nirA-promoter-controlled Octanol Dehydrogenase (12″) DNA construct (1074 bp) that includes a PCR FD primer (sequence 1-20), a 231-bp nirA promoter from Thermosynechococcus elongatus BP1 (21-251), an enzyme-encoding sequence (252-934) selected/modified from the sequences of a Drosophila subobscura octanol dehydrogenase (ABO65263), a 120-bp rbcS terminator from BP1 (935-1054), and a PCR RE primer (1055-1074) at the 3′ end.

Note, the designer enzymes of SEQ ID NOS: 87-91 have certain broad substrate specificity. Consequently, they can also be used as designer 3-ketothiolase 07″, designer 3-hydroxyacyl-CoA dehydrogenase 08″, designer enoyl-CoA dehydratase 09″, designer 2-enoyl-CoA reductase 10″, and designer acyl-CoA reductase 11″. Therefore, SEQ ID NOS: 87-91 and 93 represent a set of designer genes that can express another designer hydrocarbon chain elongation pathway for 1-octanol production (07′40′ and 07″-12″ as shown in FIG. 7). SEQ ID NO: 93 (encoding for octanol dehydrogenase 12″) is one of the key designer genes that enable production of 1-octanol production in this pathway. The net result of this pathway in working with the Calvin cycle are photobiological production of 1-octanol (CH₃CH₂CH₂CH₂CH₂CH₂CH₂CH₂OH) from carbon dioxide (CO₂) and water (H₂O) using photosynthetically generated ATP and NADPH according to the following process reaction:

8CO₂+9H₂O→CH₃CH₂CH₂CH₂CH₂CH₂CH₂CH₂OH+12O₂ [10]

### Calvin-Cycle-Channeled Pathways for Production of 1-Pentanol, 1-Hexanol and 1-Heptanol

According to one of the various embodiments, a designer Calvin-cycle-channeled pathway is created that takes the Calvin-cycle intermediate product, 3-phosphoglycerate, and converts it into 1-pentanol, 1-hexanol, and/or 1-heptanol by using, for example, a set of enzymes consisting of (as shown with the numerical labels 34, 35, 03-05, 36-41, 39, 39′-43′, 39′-43′, 12′, and 39″-43″ in FIG. 8): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, citramalate synthase 36, 2-methylmalate dehydratase 37, 3-isopropylmalate dehydratase 38, 3-isopropylmalate dehydrogenase 39, 2-isopropylmalate synthase 40, isopropylmalate isomerase 41, 3-isopropylmalate dehydrogenase 39, designer isopropylmalate synthase 40′, designer isopropylmalate isomerase 41′, designer 3-isopropylmalate dehydrogenase 39′, designer 2-keto acid decarboxylase 42′, short-chain alcohol dehydrogenase 43′, hexanol dehydrogenase 12′, designer isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″. This designer pathway works with the Calvin cycle using photosynthetically generated ATP and NADPH for photobiological production of 1-pentanol (CH₃CH₂CH₂CH₂CH₂OH), 1-hexanol (CH₃CH₂CH₂CH₂CH₂CH₂OH), and/or 1-heptanol (CH₃CH₂CH₂CH₂CH₂CH₂CH₂OH) from carbon dioxide (CO₂) and water (H₂O) according to the following process reactions:

10CO₂+12H₂O→2CH₃CH₂CH₂CH₂CH₂OH+15O₂ [11]

6CO₂+7H₂O→CH₃CH₂CH₂CH₂CH₂CH₂OH+9O₂ [12]

14CO₂+16H₂O→2CH₃CH₂CH₂CH₂CH₂CH₂CH₂OH+21O₂ [13]

According to another embodiment, a designer Calvin-cycle-channeled pathway is created that takes the intermediate product, 3-phosphoglycerate, and converts it into 1-pentanol, 1-hexanol, and/or 1-heptanol by using, for example, a set of enzymes consisting of (as shown with the numerical labels 34, 35, 03, 04, 45-52, 40, 41, 39, 39′-43′, 39′-43′, 12′, and 39″-43″ in FIG. 8): NADPH-dependent glyceraldehyde-3-phosphate dehydrogenase 34, NAD-dependent glyceraldehyde-3-phosphate dehydrogenase 35, phosphoglycerate mutase 03, enolase 04, phosphoenolpyruvate carboxylase 45, aspartate aminotransferase 46, aspartokinase 47, aspartate-semialdehyde dehydrogenase 48, homoserine dehydrogenase 49, homoserine kinase 50, threonine synthase 51, threonine ammonia-lyase 52, 2-isopropylmalate synthase 40, isopropylmalate isomerase 41, 3-isopropylmalate dehydrogenase 39, designer isopropylmalate synthase 40′, designer isopropylmalate isomerase 41′, designer 3-isopropylmalate dehydrogenase 39′, designer 2-keto acid decarboxylase 42′, short-chain alcohol dehydrogenase 43′, hexanol dehydrogenase 12′, designer isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″.

These pathways (FIG. 8) share a common feature of using an NADPH-dependent glyceraldehyde-3-phosphate dehydrogenase 34 and an NAD-dependent glyceraldehyde-3-phosphate dehydrogenase 35 as a mechanism for NADPH/NADH conversion to drive production of 1-pentanol, 1-hexanol, and/or 1-heptanol through a designer Calvin-cycle-channeled pathway in combination with a designer hydrocarbon chain elongation pathway (40′, 41′, 39′). This embodiment also takes the advantage of the broad substrate specificity (promiscuity) of 2-isopropylmalate synthase 40, isopropylmalate isomerase 41, 3-isopropylmalate dehydrogenase 39, 2-keto acid decarboxylase 42, and short-chain alcohol dehydrogenase 43 so that they can be used also as: designer isopropylmalate synthase 40′, designer isopropylmalate isomerase 41′, designer 3-isopropylmalate dehydrogenase 39′, designer 2-keto acid decarboxylase 42′, and short-chain alcohol dehydrogenase 43′; isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″.

In this case, proper selection of a short-chain alcohol dehydrogenase with certain promiscuity is also essential. SEQ ID NO: 94 presents example 94 of a designer nirA-promoter-controlled Short Chain Alcohol Dehydrogenase DNA construct (1096 bp) that includes a PCR FD primer (sequence 1-20), a 231-bp nirA promoter from Thermosynechococcus elongatus BP1 (21-251), an enzyme-encoding sequence (252-956) selected/modified from the sequences of a Pyrococcus furiosus DSM 3638 Short chain alcohol dehydrogenase (AAC25556), a 120-bp rbcS terminator from BP1 (957-1076), and a PCR RE primer (1077-1096) at the 3′ end.

Therefore, SEQ ID NOS: 58-69 and 94 represent a set of designer genes that can express a designer Calvin-cycle 3-phosphoglycerate-braned photosynthetic NADPH-enhanced pathway for production of 1-pentanol, 1-hexanol, and/or 1-heptanol as shown with numerical labels 34, 35, 03-05, 36-41, 39, 39′-43′, 39′-43′, 39″-43″ in FIG. 8. Similarly, SEQ ID NOS: 58-61, 74-81, 66-69, and 94 represent another set of sample designer genes that can express another Calvin-cycle 3-phophoglycerate-branched NADPH-enhanced pathway for production of 1-pentanol, 1-hexanol, and/or 1-heptanol as numerically labeled as 34, 35, 03, 04, 45-52, 40, 41, 39, 39′-43′, 39′-43′, 39″-43″ in FIG. 8. Note, both of these two pathways produce alcohol mixtures with different chain lengths rather than single alcohols since all 2-keto acids (such as 2-ketohexanoate, 2-ketaheptanoate, and 2-ketooctanoate) can be converted to alcohol because of the use of the promiscuity of designer 2-keto acid decarboxylase 42′ and designer short-chain alcohol dehydrogenase 43′.

To improve product specificity, it is a preferred practice to use substrate specific designer enzymes. For example, use of substrate specific designer 1-hexanol dehydrogenase 12′ (SEQ ID NO: 92) instead of short-chain alcohol dehydrogenase with promiscuity (43′) can improve product specificity more toward 1-hexanol. Consequently, SEQ ID NOS: 58-69 and 92 represent a set of designer genes that can express a designer Calvin-cycle 3-phosphoglycerate-braned photosynthetic NADPH-enhanced pathway for production of 1-hexanol as shown with numerical labels 34, 35, 03-05, 36-41, 39, 39′-40′, 39′-42′ and 12′ in FIG. 8.

### Designer Calvin-Cycle-Channeled Pathways for Production of 3-Methyl-1-Pentanol, 4-Methyl-1-Hexanol, and 5-Methyl-1-Heptanol

According to one of the various embodiments, a designer Calvin-cycle-channeled pathway is created that takes the Calvin-cycle intermediate product, 3-phosphoglycerate, and converts it into 3-methyl-1-pentanol, 4-methyl-1-hexanol, and/or 5-methyl-1-heptanol by using, for example, a set of enzymes consisting of (as shown with the numerical labels 34, 35, 03-05, 36-39, 53-55, 39′-43′, 39′-43′, and 39″-43″ in FIG. 9): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, citramalate synthase 36, 2-methylmalate dehydratase 37, 3-isopropylmalate dehydratase 38, 3-isopropylmalate dehydrogenase 39, acetolactate synthase 53, ketol-acid reductoisomerase 54, dihydroxy-acid dehydratase 55, designer isopropylmalate synthase 40′, designer isopropylmalate isomerase 41′, designer 3-isopropylmalate dehydrogenase 39′, designer 2-keto acid decarboxylase 42′, short-chain alcohol dehydrogenase 43′, designer isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″.

According to another embodiment, a designer Calvin-cycle-channeled pathway is created that takes the intermediate product, 3-phosphoglycerate, and converts it into 3-methyl-1-pentanol, 4-methyl-1-hexanol, and/or 5-methyl-1-heptanol by using, for example, a set of enzymes consisting of (as shown with the numerical labels 34, 35, 03, 04, 45-55, 39′-43′, 39′-43′, and 39″-43″ in FIG. 9): NADPH-dependent glyceraldehyde-3-phosphate dehydrogenase 34, NAD-dependent glyceraldehyde-3-phosphate dehydrogenase 35, phosphoglycerate mutase 03, enolase 04, phosphoenolpyruvate carboxylase 45, aspartate aminotransferase 46, aspartokinase 47, aspartate-semialdehyde dehydrogenase 48, homoserine dehydrogenase 49, homoserine kinase 50, threonine synthase 51, threonine ammonia-lyase 52, acetolactate synthase 53, ketol-acid reductoisomerase 54, dihydroxy-acid dehydratase 55, designer isopropylmalate synthase 40′, designer isopropylmalate isomerase 41′, designer 3-isopropylmalate dehydrogenase 39′, designer 2-keto acid decarboxylase 42′, short-chain alcohol dehydrogenase 43′, designer isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″.

These pathways (FIG. 9) are similar to those of FIG. 8, except they use acetolactate synthase 53, ketol-acid reductoisomerase 54, dihydroxy-acid dehydratase 55 as part of the pathways for production of 3-methyl-1-pentanol, 4-methyl-1-hexanol, and/or 5-methyl-1-heptanol. They all share a common feature of using an NADPH-dependent glyceraldehyde-3-phosphate dehydrogenase 34 and an NAD-dependent glyceraldehyde-3-phosphate dehydrogenase 35 as a mechanism for NADPH/NADH conversion to drive production of 3-methyl-1-pentanol, 4-methyl-1-hexanol, and/or 5-methyl-1-heptanol through a designer Calvin-cycle-channeled pathway in combination with a hydrocarbon chain elongation pathway (40′, 41′, 39′). This embodiment also takes the advantage of the broad substrate specificity (promiscuity) of 2-isopropylmalate synthase 40, isopropylmalate isomerase 41, 3-isopropylmalate dehydrogenase 39, 2-keto acid decarboxylase 42, and short-chain alcohol dehydrogenase 43 so that they can also serve as: designer isopropylmalate synthase 40′, designer isopropylmalate isomerase 41′, designer 3-isopropylmalate dehydrogenase 39′, designer 2-keto acid decarboxylase 42′, and short-chain alcohol dehydrogenase 43′; designer isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″.

Therefore, SEQ ID NOS: 58-69, 82-84, and 94 represent a set of designer genes that can express a designer Calvin-cycle 3-phosphoglycerate-braned photosynthetic NADPH-enhanced pathway for production of 3-methyl-1-pentanol, 4-methyl-1-hexanol, and 5-methyl-1-heptanol as shown with numerical labels 34, 35, 03-05, 36-39, 53-55, 39′-43′, 39′-43′, and 39″-43″ in FIG. 9. Similarly, SEQ ID NOS: 58-61, 74-81, 82-84, 66-69, and 94 represent another set of sample designer genes that can express another Calvin-cycle 3-phophoglycerate-branched NADPH-enhanced pathway for production of 3-methyl-1-pentanol, 4-methyl-1-hexanol, and/or 5-methyl-1-heptanol as numerically labeled as 34, 35, 03, 04, 45-55, 39′-43′, 39′-43′, 39″-43″ in FIG. 9. The net results of the designer photosynthetic NADPH-enhanced pathways in working with the Calvin cycle are production of 3-methyl-1-pentanol (CH₃CH₂CH(CH₃)CH₂CH₂OH), 4-methyl-1-hexanol (CH₃CH₂CH(CH₃)CH₂CH₂CH₂OH), and 5-methyl-1-heptanol(CH₃CH₂CH(CH₃)CH₂CH₂CH₂CH₂OH) from carbon dioxide (CO₂) and water (H₂O) using photosynthetically generated ATP and NADPH according to the following process reactions:

6CO₂+7H₂O→CH₃CH₂CH(CH₃)CH₂CH₂OH+9O₂ [14]

14CO₂+16H₂O→2CH₃CH₂CH(CH₃)CH₂CH₂CH₂OH+21O₂ [15]

8CO₂+9H₂O→CH₃CH₂CH(CH₃)CH₂CH₂CH₂CH₂OH+12O₂ [16]

### Designer Calvin-Cycle-Channeled Pathways for Production of 4-Methyl-1-Pentanol, 5-Methyl-1-Hexanol, and 6-Methyl-1-Heptanol

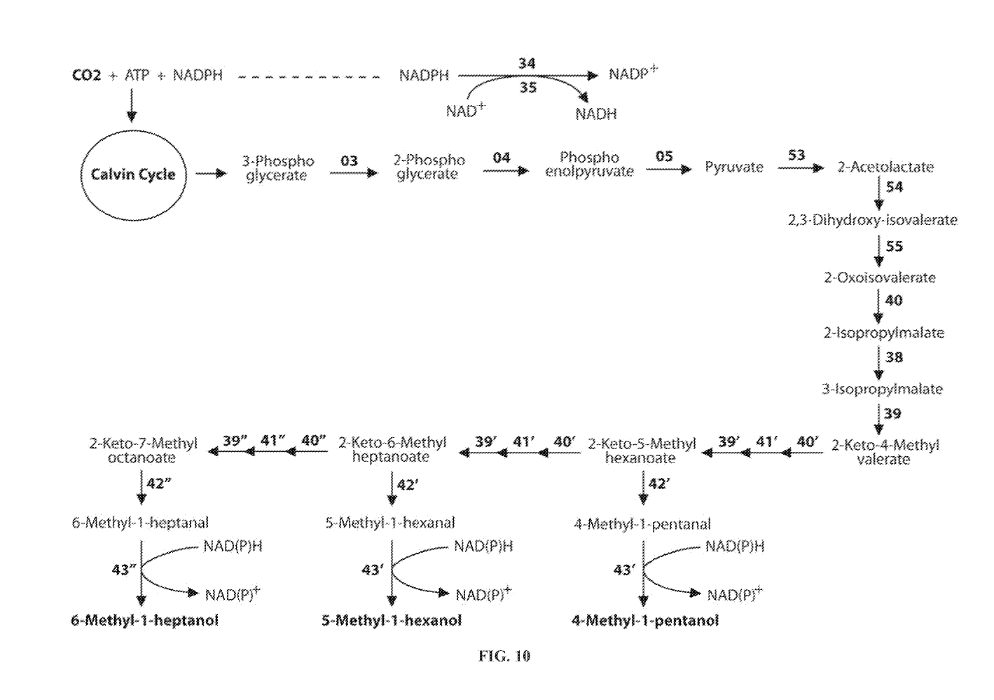

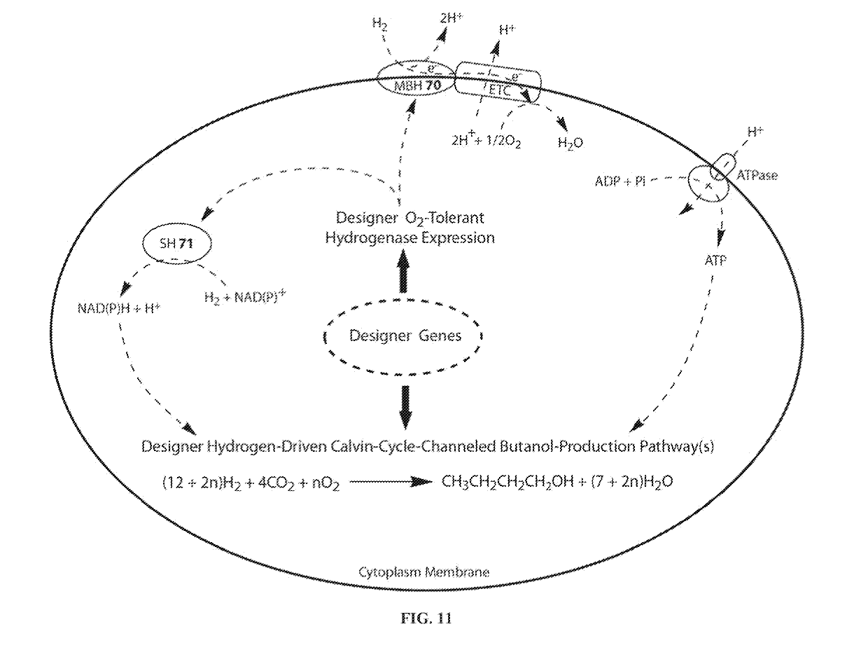

According to one of the various embodiments, a designer Calvin-cycle-channeled pathway is created that takes the Calvin-cycle intermediate product, 3-phosphoglycerate, and converts it into 4-methyl-1-pentanol, 5-methyl-1-hexanol, and 6-methyl-1-heptanol by using, for example, a set of enzymes consisting of (as shown with the numerical labels 34, 35, 03-05, 53-55, 40, 38, 39, 39′-43′, 39′-43′, and 39″-43″ in FIG. 10): NADPH-dependent glyceraldehyde-3-phosphate dehydrogenase 34, NAD-dependent glyceraldehyde-3-phosphate dehydrogenase 35, phosphoglycerate mutase 03, enolase 04, pyruvate kinase 05, acetolactate synthase 53, ketol-acid reductoisomerase 54, dihydroxy-acid dehydratase 55, isopropylmalate synthase 40, dehydratase 38, 3-isopropylmalate dehydrogenase 39, designer isopropylmalate synthase 40′, designer isopropylmalate isomerase 41′, designer 3-isopropylmalate dehydrogenase 39′, designer 2-keto acid decarboxylase 42′, short-chain alcohol dehydrogenase 43′, designer isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″.

This pathway (FIG. 10) is similar to those of FIG. 8, except that it does not use citramalate synthase 36 and 2-methylmalate dehydratase 37, but uses acetolactate synthase 53, ketol-acid reductoisomerase 54, dihydroxy-acid dehydratase 55 as part of the pathways for production of 4-methyl-1-pentano-1,5-methyl-1-hexanol, and/or 6-methyl-1-heptanol. They all share a common feature of using an NADPH-dependent glyceraldehyde-3-phosphate dehydrogenase 34 and an NAD-dependent glyceraldehyde-3-phosphate dehydrogenase 35 as a mechanism for NADPH/NADH conversion to drive production of 3-methyl-1-butanol, 4-methyl-1-butanol, and 5-methyl-1-butanol through a Calvin-cycle-channeled pathway in combination with a designer hydrocarbon chain elongation pathway (40′, 41′, 39′). This embodiment also takes the advantage of the broad substrate specificity (promiscuity) of 2-isopropylmalate synthase 40, isopropylmalate isomerase 41, 3-isopropylmalate dehydrogenase 39, 2-keto acid decarboxylase 42, and short-chain alcohol dehydrogenase 43 so that they may also serve as: designer isopropylmalate synthase 40′, designer isopropylmalate isomerase 41′, designer 3-isopropylmalate dehydrogenase 39′, designer 2-keto acid decarboxylase 42′, and short-chain alcohol dehydrogenase 43′, designer isopropylmalate synthase 40″, designer isopropylmalate isomerase 41″, designer 3-isopropylmalate dehydrogenase 39″, designer 2-keto acid decarboxylase 42″, and designer short-chain alcohol dehydrogenase 43″.

Therefore, SEQ ID NOS: 58-62, 82-84, 65-69 and 94 represent a set of sample designer genes that can be used to express a designer Calvin-cycle 3-phosphoglycerate-braned photosynthetic NADPH-enhanced pathway for production of 4-methyl-1-pentanol, 5-methyl-1-hexanol, and/or 6-methyl-1-heptanol as shown with numerical labels 34, 35, 03-05, 53-55, 40, 38, 39, 39′-43′, 39′-43′, and 39″-43″ in FIG. 10. The net results of the designer photosynthetic NADPH-enhanced pathway in working with the Calvin cycle are production of 4-methyl-1-pentanol (CH₃CH(CH₃)CH₂CH₂CH₂OH), 5-methyl-1-hexanol (CH₃CH(CH₃)CH₂CH₂CH₂CH₂OH), and 6-methyl-1-heptanol (CH₃CH(CH₃)CH₂CH₂CH₂CH₂CH₂OH) from carbon dioxide (CO₂) and water (H₂O) using photosynthetically generated ATP and NADPH according to the following process reactions:

6CO₂+7H₂O→CH₃CH(CH₃)CH₂CH₂CH₂OH+9O₂ [17]

14CO₂+16H₂O→2CH₃CH(CH₃)CH₂CH₂CH₂CH₂OH+21O₂ [18]

8CO₂+9H₂O→CH₃CH(CH₃)CH₂CH₂CH₂CH₂CH₂OH+12O₂ [19]

Designer Oxyphotobacteria with Calvin-Cycle-Channeled Pathways for Production of Butanol and Related Higher Alcohols

According to one of the various embodiments, use of designer DNA constructs in genetic transform of certain oxyphotobacteria hosts can create various designer transgenic oxyphotobacteria with Calvin-cycle-channeled pathways for photobiological production of butanol and related higher alcohols from carbon dioxide and water. To ensure biosafety for use of the designer transgenic photosynthetic organism-based biofuels production technology, it is a preferred practice to incorporate biosafety-guarded features into the designer transgenic photosynthetic organisms as well. Therefore, in accordance with the present invention, various designer photosynthetic organisms including designer transgenic oxyphotobacteria are created with a biosafety-guarded photobiological biofuel-production technology based on cell-division-controllable designer transgenic photosynthetic organisms. The cell-division-controllable designer photosynthetic organisms contain two key functions: a designer biosafety mechanism(s) and a designer biofuel-production pathway(s). The designer biosafety feature(s) is conferred by a number of mechanisms including: a) the inducible insertion of designer proton-channels into cytoplasm membrane to permanently disable any cell division and/or mating capability, b) the selective application of designer cell-division-cycle regulatory protein or interference RNA (iRNA) to permanently inhibit the cell division cycle and preferably keep the cell at the G₁ phase or G₀ state, and c) the innovative use of a high-CO₂-requiring host photosynthetic organism for expression of the designer biofuel-production pathway(s). The designer cell-division-control technology can help ensure biosafety in using the designer organisms for biofuel production.

Oxyphotobacteria (including cyanobacteria and oxychlorobacteria) that can be selected for use as host organisms to create designer transgenic oxyphotobacteria for photobiological production of butanol and related higher alcohols include (but not limited to): Thermosynechococcus elongatus BP-1, Nostoc sp. PCC 7120, Synechococcus elongatus PCC 6301, Syncechococcus sp. strain PCC 7942, Syncechococcus sp. strain PCC 7002, Syncechocystis sp. strain PCC 6803, Prochlorococcus marinus MED4, Prochlorococcus marinus MIT 9313, Prochlorococcus marinus NATL1A, Prochlorococcus SS120, Spirulina platensis (Arthrospira platensis), Spirulina pacifica, Lyngbya majuscule, Anabaena sp., Synechocystis sp., Synechococcus elongates, Synechococcus (MC-A), Trichodesmium sp., Richelia intracellularis, Synechococcus WH7803, Synechococcus WH8102, Nostoc punctiforme, Syncechococcus sp. strain PCC 7943, Synechocyitis PCC 6714 phycocyanin-deficient mutant PD-1, Cyanothece strain 51142, Cyanothece sp. CCY0110, Oscillatoria limosa, Lyngbya majuscula, Symploca muscorum, Gloeobacter violaceus, Prochloron didemni, Prochlorothrix hollandica, Prochlorococcus marinus, Prochlorococcus SS120, Synechococcus WH8102, Lyngbya majuscula, Symploca muscorum, Synechococcus bigranulatus, cryophilic Oscillatoria sp., Phormidium sp., Nostoc sp.-1, Calothrix parietina, thermophilic Synechococcus bigranulatus, Synechococcus lividus, thermophilic Mastigocladus laminosus, Chlorogloeopsis fritschii PCC 6912, Synechococcus vulcanus, Synechococcus sp. strain MA4, Synechococcus sp. strain MA19, and Thermosynechococcus elongatus.

According to one of the examples, use of designer DNA constructs such as SEQ ID NOS: 58-94 in genetic transform of certain oxyphotobacteria hosts such as Thermosynechococcus elongatus BP1 can create a series of designer transgenic oxyphotobacteria with Calvin-cycle-channeled pathways for production of butanol and related higher alcohols. Consequently, SEQ ID NOS: 58-61, 74-81, 66-69, and 72 (and/or 73) represent a designer transgenic oxyphotobacterium such as a designer transgenic Thermosynechococcus that comprises the designer genes of a Calvin-cycle 3-phophoglycerate-branched photosynthetic NADPH-enhanced pathway (numerically labeled as 34, 35, 03, 04, 45-52, 39-42, and 12 in FIG. 4) for photobiological production of 1-butanol from carbon dioxide and water. SEQ ID NOS: 58-69 and 72 (and/or 73) represent another designer transgenic oxyphotobacterium such as designer transgenic Thermosynechococcus that comprises the designer genes of a Calvin-cycle 3-phophoglycerate-branched photosynthetic NADPH-enhanced pathway (numerically labeled as 34, 35, 03-05, 36-42, and 12 in FIG. 4) for photobiological production of 1-butanol from carbon dioxide and water as well.

Similarly, SEQ ID NOS: 58-66, 82-84, 69 and 85 represent another designer transgenic oxyphotobacterium such as designer transgenic Thermosynechococcus with a Calvin-cycle 3-phophoglycerate-branched photosynthetic NADPH-enhanced pathway (numerically labeled as 34, 35, 03-05, 36-39, 53-55, 42 and 56 in FIG. 5) for photobiological production of 2-methyl-1-butanol production from carbon dioxide and water; while SEQ ID NOS: 58-61, 74-84, 69 and 85 represent another designer transgenic Thermosynechococcus with a Calvin-cycle 3-phophoglycerate-branched photosynthetic NADPH-enhanced 2-methyl-1-butanol production pathway (34, 35, 03, 04, 45-55, 42 and 56 in FIG. 5) for photobiological production of 2-methyl-1-butanol production from carbon dioxide and water.

SEQ ID NOS: 58-63, 82-84, 69, 70 (or 71) represent another designer transgenic oxyphotobacterium such as designer transgenic Thermosynechococcus with a Calvin-cycle 3-phosphoglycerate-branched photosynthetic NADPH-enhanced isobutanol production pathway (34, 35, 03-05, 53-5, 42, 43 or 44); while SEQ ID NOS: 58-62, 81-83, 65-67, 69 and 86 represent another designer transgenic Thermosynechococcus with a Calvin-cycle 3-phosphoglycerate-branched photosynthetic NADPH-enhanced 3-methyl-1-butanol production pathway (numerical labels 34, 35, 03-05, 53-55, 40, 38, 39, 42, and 57 in FIG. 6).

SEQ ID NOS: 87-92 represent another designer transgenic Thermosynechococcus with a designer hydrocarbon chain elongation pathway (07′-12′ as shown in FIG. 7) for photobiological production of 1-hexanol. SEQ ID NOS: 87-91 and 93 represent another designer transgenic Thermosynechococcus with a designer hydrocarbon chain elongation pathway (07′-10′ and 07″-12″ as shown in FIG. 7) for photobiological production of 1-octanol.

SEQ ID NOS: 58-69 and 92 represent another designer transgenic Thermosynechococcus with a designer Calvin-cycle 3-phosphoglycerate-braned photosynthetic NADPH-enhanced pathway (34, 35, 03-05, 36-41, 39, 39′-40′, 39′-42′ and 12′ in FIG. 8) for photobiological production of 1-hexanol from carbon dioxide and water.

SEQ ID NOS: 58-69, 82-84, and 94 represent a designer transgenic Thermosynechococcus with a designer Calvin-cycle 3-phosphoglycerate-braned photosynthetic NADPH-enhanced pathway (34, 35, 03-05, 36-39, 53-55, 39′-43′, 39′-43′, 39″-43″ in FIG. 9) for production of 3-methyl-1-pentanol, 4-methyl-1-hexanol, and 5-methyl-1-heptanol from carbon dioxide and water. Similarly, SEQ ID NOS: 58-61, 74-81, 82-84, 66-69, and 94 represent another designer transgenic Thermosynechococcus with a Calvin-cycle 3-phophoglycerate-branched NADPH-enhanced pathway (34, 35, 03, 04, 45-55, 39′-43′, 39′-43′, 39″-43″ in FIG. 9) for photobiological production of 3-methyl-1-pentanol, 4-methyl-1-hexanol, and 5-methyl-1-heptanol from carbon dioxide and water as well.

SEQ ID NOS: 58-62, 82-84, 65-69 and 94 represent a designer transgenic Thermosynechococcus with a designer Calvin-cycle 3-phosphoglycerate-braned photosynthetic NADPH-enhanced pathway labels (34, 35, 03-05, 53-55, 40, 38, 39, 39′-43′, 39′-43′, and 39″-43″ in FIG. 10) for photobiological production of 4-methyl-1-pentanol, 5-methyl-1-hexanol, and/or 6-methyl-1-heptanol from carbon dioxide and water.

Use of other host oxyphotobacteria such as Synechococcus sp. strain PCC 7942, Synechocystis sp. strain PCC 6803, Prochlorococcus marinus, Cyanothece sp. ATCC 51142, for genetic transformation with proper designer DNA constructs (genes) can create other designer oxyphotobacteria for photobiological production of butanol and higher alcohols as well. For example, use of Synechococcus sp. strain PCC 7942 as a host organism in genetic transformation with SEQ ID NOS: 95-98 (and/or 99) can create a designer transgenic Synechococcus for photobiological production of 1-butanol. Briefly, SEQ ID NO: 95 presents example 95 of a detailed DNA construct (1438 base pairs (bp)) of a designer NADPH-dependent Glyceraldehyde-3-Phosphate-Dehydrogenase (34) gene that includes a PCR FD primer (sequence by 1-20), a 88-bp nirA promoter (21-108) selected from the Synechococcus sp. strain PCC 7942 (freshwater cyanobacterium) nitrite-reductase-gene promoter sequence, an enzyme-encoding sequence (109-1110) selected and modified from a Staphylococcus NADPH-dependent glyceraldehyde-3-phosphate-dehydrogenase sequence (GenBank accession number: YP_{—}003471459), a 308-bp Synechococcus rbcS terminator (1111-1418), and a PCR RE primer (1419-1438).

SEQ ID NO: 96 presents example 96 of a detailed DNA construct (1447 bp) of a designer NAD-dependent Glyceraldehyde-3-Phosphate-Dehydrogenase (35) gene that includes a PCR FD primer (sequence by 1-20), a 88-bp nirA promoter (21-108) selected from the Synechococcus nitrite-reductase-gene promoter sequence, an enzyme-encoding sequence (109-1119) selected from a Staphylococcus aureus NAD-dependent glyceraldehyde-3-phosphate-dehydrogenase sequence (GenBank accession number: ADC36961), a 308-bp Synechococcus rbcS terminator (1120-1427), and a PCR RE primer (1428-1447).

SEQ ID NO: 97 presents example 97 of a detailed DNA construct (2080 bp) of a designer 2-Keto Acid Decarboxylase (42) gene that includes a PCR FD primer (sequence by 1-20), a 88-bp nirA promoter (21-108) selected from the Synechococcus nitrite-reductase-gene promoter sequence, an enzyme-encoding sequence (109-1752) selected from a Lactococcus lactis branched-chain alpha-ketoacid decarboxylase (GenBank accession number: AAS49166), a 308-bp Synechococcus rbcS terminator (1753-2060), and a PCR RE primer (2061-2080).

SEQ ID NO: 98 presents a detailed DNA construct (1603 bp) of a designer NADH-dependent butanol dehydrogenase (12a) gene that include a PCR FD primer (sequence by 1-20), a 88-bp nirA promoter (21-108) selected from the Synechococcus nitrite-reductase-gene promoter sequence, an enzyme-encoding sequence (109-1275) selected from a Clostridium NADH-dependent butanol dehydrogenase (GenBank accession number: ADO12118), a 308-bp Synechococcus rbcS terminator (1276-1583), and a PCR RE primer (1584-1603).

SEQ ID NO: 99 presents example 99 of a detailed DNA construct (1654 bp) of a designer NADPH-dependent Butanol Dehydrogenase (12b) gene including: a PCR FD primer (sequence by 1-20), a 88-bp nirA promoter (21-108) selected from the Synechococcus nitrite-reductase-gene promoter sequence, an enzyme-encoding sequence (109-1326) selected from a Butyrivibrio NADPH-dependent butanol dehydrogenase (GenBank: EFF67629), a 308-bp Synechococcus rbcS terminator (1327-1634), and a PCR RE primer (1635-1654).

Note, in the designer transgenic Synechococcus that is represented by SEQ ID NOS: 95-98 (and/or 99), Synechococcus's native enzymes of 03-05, 36-41 and 45-52 are used in combination with the designer nirA-promoter-controlled enzymes of 34, 35, 42 and 12 [encoded by SEQ ID NOS: 95-98 (and/or 99)] to confer the Calvin-cycle 3-phophoglycerate-branched photosynthetic NADPH-enhanced pathways for photobiological production of 1-butanol from carbon dioxide and water (FIG. 4).

Similarly, use of Synechocystis sp. strain PCC 6803 as a host organism in genetic transformation with SEQ ID NOS: 100-102 (and/or 103) creates a designer transgenic Synechocystis for photobiological production of 1-butanol. Briefly, SEQ ID NO: 100 presents example 100 of a designer nirA-promoter-controlled NAD-dependent Glyceraldehyde-3-Phosphate Dehydrogenase (35) DNA construct (1440 bp) that includes a PCR FD primer (sequence 1-20), a 89-bp Synechocystis sp. strain PCC 6803 nitrite-reductase nirA promoter (21-109), an enzyme-encoding sequence (110-1011) selected from a Streptococcus pyogenes NAD-dependent Glyceraldehyde-3-phosphate dehydrogenase (GenBank: YP_{—}002285269), a 409-bp Synechocystis sp. PCC 6803 rbcS terminator (1012-1420), and a PCR RE primer (1421-1440).

SEQ ID NO: 101 presents example 101 of a designer nirA-promoter-controlled 2-Keto Acid Decarboxylase (42) DNA construct (2182 bp) that includes a PCR FD primer (sequence 1-20), a 89-bp Synechocystis sp. strain PCC 6803 nitrite-reductase nirA promoter (21-109), an enzyme-encoding sequence (110-1753) selected from a Lactococcus lactis branched-chain alpha-ketoacid decarboxylase (GenBank: AAS49166), a 409-bp Synechocystis sp. PCC 6803 rbcS terminator (1754-2162), and a PCR RE primer (2163-2182).

SEQ ID NO: 102 presents example 102 of a designer nirA-promoter-controlled NADH-dependent Butanol Dehydrogenase (12a) DNA construct (1705 bp) that includes a PCR FD primer (sequence 1-20), a 89-bp Synechocystis sp. strain PCC 6803 nitrite-reductase nirA promoter (21-109), an enzyme-encoding sequence (110-1276) selected from a Clostridium carboxidivorans P7 NADH-dependent butanol dehydrogenase (GenBank: ADO12118), a 409-bp Synechocystis sp. PCC 6803 rbcS terminator (1277-1685), and a PCR RE primer (1686-1705).

SEQ ID NO: 103 presents example 103 of a designer nirA-promoter-controlled NADPH-dependent butanol dehydrogenase (12b) DNA construct (1756 bp) that includes a PCR FD primer (sequence 1-20), a 89-bp Synechocystis sp. strain PCC 6803 nitrite-reductase nirA promoter (21-109), an enzyme-encoding sequence (110-1327) selected from a Butyrivibrio crossotus NADPH-dependent butanol dehydrogenase (GenBank: EFF67629), a 409-bp Synechocystis sp. PCC 6803 rbcS terminator (1328-1736), and a PCR RE primer (1737-1756).

Note, in the designer transgenic Synechocystis that contains the designer genes of SEQ ID NOS: 100-102 (and/or 103), Synechocystis's native enzymes of 34, 03-05, 36-41 and 45-52 are used in conjunction with the designer nirA-promoter-controlled enzymes of 35, 42 and 12 [encoded by SEQ ID NOS: 100-102 (and/or 103)] to confer the Calvin-cycle 3-phophoglycerate-branched photosynthetic NADPH-enhanced pathways for photobiological production of 1-butanol from carbon dioxide and water (FIG. 4).

Use of Nostoc sp. strain PCC 7120 as a host organism in genetic transformation with SEQ ID NOS: 104-109 can create a designer transgenic Nostoc for photobiological production of 2-methyl-1-butanol (FIG. 5). Briefly, SEQ ID NO: 104 presents example 104 of a designer hox-promoter-controlled NAD-dependent Glyceraldehyde-3-Phosphate Dehydrogenase (35) DNA construct (1655 bp) that includes a PCR FD primer (sequence 1-20), a 172-bp Nostoc sp. strain PCC 7120 (Anabaena PCC 7120) hox promoter (21-192), an enzyme-encoding sequence (193-1203) selected/modified from the sequence of a Streptococcus pyogenes NZ131 NAD-dependent glyceraldehyde-3-phosphate dehydrogenase (GenBank: YP_{—}002285269), a 432-bp Nostoc sp. strain PCC 7120 gor terminator (1204-1635), and a PCR RE primer (1636-1655).

SEQ ID NO: 105 presents example 105 of a designer hox-promoter-controlled Acetolactate Synthase (53) DNA construct (2303 bp) that includes a PCR FD primer (sequence 1-20), a 172-bp Nostoc sp. strain PCC 7120 (Anabaena PCC 7120) hox promoter (21-192), an enzyme-encoding sequence (193-1851) selected/modified from the sequence of a Thermosynechococcus elongatus BP-1 acetolactate synthase (GenBank: NP_{—}682614), a 432-bp Nostoc sp. strain PCC 7120 gor terminator (1852-2283), and a PCR RE primer (2284-2303).

SEQ ID NO: 106 presents example 106 of a designer hox-promoter-controlled Ketol-Acid Reductoisomerase (54) DNA construct (1661 bp) that includes a PCR FD primer (sequence 1-20), a 172-bp Nostoc sp. strain PCC 7120 (Anabaena PCC 7120) hox promoter (21-192), an enzyme-encoding sequence (193-1209) selected/modified from the sequence of a Calditerrivibrio nitroreducens ketol-acid reductoisomerase (GenBank: YP_{—}004050904), a 432-bp Nostoc sp. gor terminator (1210-1641), and a PCR RE primer (1642-1661).

SEQ ID NO: 107 presents example 107 of a designer hox-promoter-controlled Dihydroxy-Acid Dehydratase (55) DNA construct (2324 bp) that includes a PCR FD primer (sequence 1-20), a 172-bp Nostoc sp. strain PCC 7120 (Anabaena PCC 7120) hox promoter (21-192), an enzyme-encoding sequence (193-1872) selected/modified from the sequence of a Marivirga tractuosa DSM 4126 dihydroxy-acid dehydratase (GenBank: YP_{—}004053736), a 432-bp Nostoc sp. gor terminator (1873-2304), and a PCR RE primer (2305-2324).

SEQ ID NO: 108 presents example 108 of a designer hox-promoter-controlled branched-chain alpha-Ketoacid Decarboxylase (42) DNA construct (2288 bp) that includes a PCR FD primer (sequence 1-20), a 172-bp Nostoc sp. (Anabaena PCC 7120) hox promoter (21-192), an enzyme-encoding sequence (193-1836) selected/modified from the sequence of a Lactococcus lactis branched-chain alpha-ketoacid decarboxylase (GenBank: AAS49166), a 432-bp Nostoc sp. gor terminator (1837-2268), and a PCR RE primer (2269-2288).

SEQ ID NO: 109 presents example 109 of a designer hox-promoter-controlled 2-Methylbutyraldehyde Reductase (56) DNA construct (1613 bp) that includes a PCR FD primer (sequence 1-20), a 172-bp Nostoc sp. (Anabaena PCC 7120) hox promoter (21-192), an enzyme-encoding sequence (193-1461) selected/modified from the sequence of a Schizosaccharomyces japonicus y 2-methylbutyraldehyde reductase (GenBank: XP_{—}002173231), a 432-bp Nostoc sp. strain PCC 7120 gor terminator (1462-1893), and a PCR RE primer (1894-1613).

Note, in the designer transgenic Nostoc that contains designer hox-promoter-controlled genes of SEQ ID NOS: 104-109, Nostoc's native enzymes (genes) of 34, 03-05, 36-39 and 45-52 are used in combination with the designer hox-promoter-controlled enzymes of 35, 53-55, 42 and 56 (encoded by DNA constructs of SEQ ID NOS: 104-109) to confer the Calvin-cycle 3-phophoglycerate-branched photosynthetic NADPH-enhanced pathways for photobiological production of 2-methyl-1-butanol from carbon dioxide and water (FIG. 5).

Use of Prochlorococcus marinus MIT 9313 as a host organism in genetic transformation with SEQ ID NOS: 110-122 can create a designer transgenic Prochlorococcus marinus for photobiological production of isobutanol and/or 3-methyl-1-butanol (FIG. 6). Briefly, SEQ ID NO:110 presents example 110 for a designer groE-promoter-controlled NAD-dependent Glyceraldehyde-3-Phosphate Dehydrogenase (35) DNA construct (1300 bp) that includes a PCR FD primer (sequence 1-20), a 137-bp Prochlorococcus marinus MIT 9313 heat- and light-responsive groE promoter (21-157), an enzyme-encoding sequence (158-1159) selected from a Vibrio cholerae MJ-1236 NAD-dependent Glyceraldehyde-3-phosphate dehydrogenase (GenBank: ACQ61431), a 121-bp Prochlorococcus marinus MIT9313 rbcS terminator (1160-1280), and a PCR RE primer (1281-1300).

SEQ ID NO:111 presents example 111 for a designer groE-promoter-controlled Phosphoglycerate Mutase (03) DNA construct (1498 bp) that includes a PCR FD primer (sequence 1-20), a 137-bp Prochlorococcus marinus MIT9313 heat- and light-responsive groE promoter (21-157), an enzyme-encoding sequence (158-1357) selected from a Pelotomaculum thermopropionicum SI phosphoglycerate mutase (GenBank: YP_{—}001212148), a 121-bp Prochlorococcus marinus rbcS terminator (1358-1478), and a PCR RE primer (1479-1498).

SEQ ID NO:112 presents example 112 for a designer groE-promoter-controlled Enolase (04) DNA construct (1588 bp) that includes a PCR FD primer (sequence 1-20), a 137-bp Prochlorococcus heat- and light-responsive groE promoter (21-157), an enzyme-encoding sequence (158-1447) selected from a Thermotoga enolase (GenBank: ABQ46079), a 121-bp Prochlorococcus marinus rbcS terminator (1448-1568), and a PCR RE primer (1569-1588).

SEQ ID NO:113 presents example 113 for a designer groE-promoter-controlled Pyruvate Kinase (05) DNA construct (1717 bp) that includes a PCR FD primer (sequence 1-20), a 137-bp Prochlorococcus marinus MIT9313 heat- and light-responsive groE promoter (21-157), an enzyme-encoding sequence (158-1576) selected from a Thermotoga lettingae TMO pyruvate kinase (GenBank: YP_{—}001471580), a 121-bp Prochlorococcus marinus MIT9313 rbcS terminator (1577-1697), and a PCR RE primer (1698-1717).

SEQ ID NO:114 presents example 114 for a designer groE-promoter-controlled Acetolactate Synthase (53) DNA construct (2017 bp) that includes a PCR FD primer (sequence 1-20), a 137-bp Prochlorococcus marinus MIT 9313 heat- and light-responsive groE promoter (21-157), an enzyme-encoding sequence (158-1876) selected from a Bacillus licheniformis ATCC 14580 acetolactate synthase (GenBank: AAU42663), a 121-bp Prochlorococcus marinus MIT 9313 rbcS terminator (1877-1997), and a PCR RE primer (1998-2017).

SEQ ID NO:115 presents example 115 for a designer groE-promoter-controlled Ketol-Acid Reductoisomerase (54) DNA construct (1588 bp) that includes a PCR FD primer (sequence 1-20), a 137-bp Prochlorococcus marinus MIT9313 heat- and light-responsive groE promoter (21-157), an enzyme-encoding sequence (158-1168) selected from a Thermotoga petrophila RKU-1 ketol-acid reductoisomerase (GenBank: ABQ46398), a 400-bp Prochlorococcus marinus MIT9313 rbcS terminator (1169-1568), and a PCR RE primer (1569-1588).

SEQ ID NO:116 presents example 116 for a designer groE-promoter-controlled Dihydroxy-Acid Dehydratase (55) DNA construct (1960 bp) that includes a PCR FD primer (sequence 1-20), a 137-bp Prochlorococcus marinus heat- and light-responsive groE promoter (21-157), an enzyme-encoding sequence (158-1819) selected from a Syntrophothermus lipocalidus DSM 12680 dihydroxy-acid dehydratase (GenBank: ADI02905), a 121-bp Prochlorococcus marinus rbcS terminator (1820-1940), and a PCR RE primer (1941-1960).

SEQ ID NO:117 presents example 117 for a designer groE-promoter-controlled 2-Keto Acid Decarboxylase (42) DNA construct (1945 bp) that includes a PCR FD primer (sequence 1-20), a 137-bp Prochlorococcus heat- and light-responsive groE promoter (21-157), an enzyme-encoding sequence (158-1804) selected from a Lactococcus lactis Alpha-ketoisovalerate decarboxylase (GenBank: ADA65057), a 121-bp Prochlorococcus rbcS terminator (1805-1925), and a PCR RE primer (1926-1945).

SEQ ID NO:118 presents example 118 for a designer nirA-promoter-controlled Alcohol Dehydrogenase (43/44) DNA construct (1138 bp) that includes a PCR FD primer (sequence 1-20), a 251-bp Prochlorococcus nirA promoter (21-271), an enzyme-encoding sequence (272-997) selected from a Geobacillus short chain alcohol dehydrogenase (GenBank: YP_{—}146837), a 121-bp Prochlorococcus rbcS terminator (998-1118), and a PCR RE primer (1119-1138).

Note, in the designer transgenic Prochlorococcus that contains the designer genes of SEQ ID NOS: 110-118, Prochlorococcus's native gene (enzyme) of 34 is used in combination with the designer groE and nirA-promoters-controlled genes (enzymes) of 35, 03-05, 53-55, 42 and 43/44 (encoded by DNA constructs of SEQ ID NOS: 110-118) to confer the Calvin-cycle 3-phophoglycerate-branched photosynthetic NADPH-enhanced pathways for photobiological production of isobutanol from carbon dioxide and water (FIG. 6). Addition of the following four designer groE promoter-controlled genes (SEQ ID NO:119-122) results in another designer transgenic Prochlorococcus that can produce both isobutanol and 3-methyl-1-butanol from carbon dioxide and water (35, 03-05, 53-55, 42, 43/44, plus 38-40 and 57 as shown in FIG. 6).

Briefly, SEQ ID NO:119 presents example 119 for a designer groE-promoter-controlled 2-Isopropylmalate Synthase (40) DNA construct (1816 bp) that includes a PCR FD primer (sequence 1-20), a 137-bp Prochlorococcus marinus MIT9313 heat- and light-responsive groE promoter (21-157), an enzyme-encoding sequence (158-1675) selected from a Pelotomaculum thermopropionicum S12-isopropylmalate synthase (GenBank: YP_{—}001211081), a 121-bp Prochlorococcus marinus rbcS terminator (1676-1796), and a PCR RE primer (1797-1816).

SEQ ID NO:120 presents example 120 for a designer groE-promoter-controlled 3-Isopropylmalate Dehydratase (38) DNA construct (2199 bp) that includes a PCR FD primer (sequence 1-20), a 137-bp Prochlorococcus marinus MIT9313 heat- and light-responsive groE promoter (21-157), a 3-isopropylmalate dehydratase large subunit-encoding sequence (158-1420) selected from a Pelotomaculum thermopropionicum S13-isopropylmalate dehydratase large subunit (GenBank: YP_{—}001211082), a 137-bp Prochlorococcus marinus MIT9313 heat- and light-responsive groE promoter (1421-1557), a 3-isopropylmalate dehydratase small subunit-encoding sequence (1558-2058) selected from a Pelotomaculum thermopropionicum SI 3-isopropylmalate dehydratase small subunit (GenBank: YP_{—}001211083), a 121-bp Prochlorococcus marinus rbcS terminator (2059-2179), and a PCR RE primer (2180-2199).

SEQ ID NO:121 presents example 121 for a designer groE-promoter-controlled 3-Isopropylmalate Dehydrogenase (39) DNA construct (1378 bp) that includes a PCR FD primer (sequence 1-20), a 137-bp Prochlorococcus marinus MIT9313 heat- and light-responsive groE promoter (21-157), an enzyme-encoding sequence (158-1237) selected from a Syntrophothermus lipocalidus DSM 12680 3-isopropylmalate dehydrogenase (GenBank: ADI02898), a 121-bp Prochlorococcus marinus rbcS terminator (1238-1358), and a PCR RE primer (1359-1378).

SEQ ID NO:122 presents example 122 for a designer groE-promoter-controlled 3-Methylbutanal Reductase (57) DNA construct (1327 bp) that includes a PCR FD primer (sequence 1-20), a 137-bp Prochlorococcus marinus MIT9313 heat- and light-responsive groE promoter (21-157), an enzyme-encoding sequence (158-1186) selected from a Saccharomyces cerevisiae S288c 3-Methylbutanal reductase (GenBank: DAA10635), a 121-bp Prochlorococcus marinus MIT9313 rbcS terminator (1187-1307), and a PCR RE primer (1308-1327).

Note, the use of SEQ ID NOS: 110-117 and 119-122 in genetic transformation of Prochlorococcus marinus MIT 9313 creates another designer transgenic Prochlorococcus marinus with a groE promoter-controlled designer Calvin-cycle-channeled pathway (identified as 34 (native), 35, 03-05, 53-55, 38-40, 42 and 57 in FIG. 6) for photobiological production of 3-methyl-1-butanol from carbon dioxide and water.

Use of Cyanothece sp. ATCC 51142 as a host organism in genetic transformation with SEQ ID NOS: 123-128 can create a designer transgenic Cyanothece for photobiological production of 1-pentanol, 1-hexanol, and/or 1-heptanol (FIG. 8). Briefly, SEQ ID NO:123 presents example 123 for a designer nirA-promoter-controlled 2-Isopropylmalate Synthase (40) DNA construct (2004 bp) that includes a PCR FD primer (sequence 1-20), a 203-bp Cyanothece sp. nirA promoter (21-223), an enzyme-encoding sequence (224-1783) selected from a Hydrogenobacter thermophilus 2-isopropylmalate synthase sequence (GenBank: BAI69273), a 201-bp Cyanothece sp. rbcS terminator (1784-1984), and a PCR RE primer (1985-2004).

SEQ ID NO:124 presents example 124 for a designer nirA-promoter-controlled Isopropylmalate Isomerase (41) large/small subunits DNA construct (2648 bp) that includes a PCR FD primer (sequence 1-20), a 203-bp Cyanothece sp. ATCC 51142 nirA promoter (21-223), an enzyme-large-subunit-encoding sequence (224-1639) selected from a Anoxybacillus flavithermus WK1 isopropylmalate isomerase large subunit sequence (GenBank: YP_{—}002314962), a 203-bp Cyanothece sp. ATCC 51142 nirA promoter (1640-1842), an enzyme-small-subunit-encoding sequence (1843-2427) selected from a Anoxybacillus flavithermus WK1 isopropylmalate isomerase small subunit sequence (GenBank: YP_{—}002314963), a 201-bp Cyanothece sp. ATCC 51142 rbcS terminator (2428-1628), and a PCR RE primer (2629-2648).

SEQ ID NO:125 presents example 125 for a designer g nirA-promoter-controlled 3-Isopropylmalate Dehydrogenase (39) DNA construct (1530 bp) that includes a PCR FD primer (sequence 1-20), a 203-bp Cyanothece sp. ATCC 51142 nirA promoter (21-223), an enzyme-encoding sequence (224-1309) selected from a Thermosynechococcus elongatus BP-1 3-isopropylmalate dehydrogenase sequence (GenBank: BAC09152), a 201-bp Cyanothece sp. ATCC 51142 rbcS terminator (1310-1310), and a PCR RE primer (1311-1530).

SEQ ID NO:126 presents example 126 for a designer nirA-promoter-controlled 2-Keto Acid Decarboxylase (42′) DNA construct (2088 bp) that includes a PCR FD primer (sequence 1-20), a 203-bp Cyanothece nirA promoter (21-223), an enzyme-encoding sequence (224-1867) selected from a Lactococcus lactis 2-keto acid decarboxylase (GenBank: AAS49166), a 201-bp Cyanothece rbcS terminator (1868-2068), and a PCR RE primer (2069-2088).

SEQ ID NO:127 presents example 127 for a designer nirA-promoter-controlled Hexanol Dehydrogenase (12′) DNA construct (1503 bp) that includes a PCR FD primer (sequence 1-20), a 203-bp Cyanothece nirA promoter (21-223), an enzyme-encoding sequence (224-1282) selected from a Mycobacterium chubuense hexanol dehydrogenase (GenBank: ACZ56328), a 201-bp Cyanothece rbcS terminator (1283-1483), and a PCR RE primer (1484-1503).

SEQ ID NO:128 presents example 128 for a designer nirA-promoter-controlled short-chain Alcohol Dehydrogenase (43′, 43″) DNA construct (1149 bp) that includes a PCR FD primer (sequence 1-20), a 203-bp Cyanothece sp. ATCC 51142 nirA promoter (21-223), an enzyme-encoding sequence (224-928) selected from a Pyrococcus furiosus DSM 3638 Short chain alcohol dehydrogenase (GenBank: AAC25556), a 201-bp Cyanothece sp. ATCC 51142 rbcS terminator (929-1129), and a PCR RE primer (1130-1149).

Note, in the designer transgenic Cyanothece that contains designer nirA promoter-controlled genes of SEQ ID NOS: 123-127, Cyanothece's native enzymes of 34,03-05, 36-38, and 45-52 are used in combination with the designer nirA-promoters-controlled enzymes of 35, 39-41 (39′-41′, 39′-41′), 42′ and 12′ (encoded by DNA constructs of SEQ ID NOS: 123-127) to confer the Calvin-cycle 3-phophoglycerate-branched photosynthetic NADPH-enhanced pathways for photobiological production of 1-hexanol from carbon dioxide and water (FIG. 8). Addition of a designer nirA-promoters-controlled gene (SEQ ID NO: 128) of a short chain alcohol dehydrogenase 43′ (43″) with promiscuity results in another designer transgenic Cyanothece containing a Calvin-cycle-channeled pathway (35, 39-41, 39′-43′, 39′-43′, and 39″-43″ as shown in FIG. 8) that can produce 1-pentanol, 1-hexanol, and 1-hexanol from carbon dioxide and water.

Designer Advanced Photosynthetic Organisms with Calvin-Cycle-Channeled Pathways for Production of Butanol and Related Higher Alcohols

According to one of the various embodiments, use of certain designer DNA constructs in genetic transformation of eukaryotic photosynthetic organisms such as plant cells, eukaryotic aquatic plants (including, for example, eukaryotic algae, submersed aquatic herbs, duckweeds, water cabbage, water lily, water hyacinth, Bolbitis heudelotii, Cabomba sp., and seagrasses) can create designer transgenic eukaryotic photosynthetic organisms for production of butanol and related higher alcohols from carbon dioxide and water. Eukaryotic algae that can be selected for use as host organisms to create designer algae for photobiological production of butanol and related higher alcohols include (but not limited to): Dunaliella salina, Dunaliella viridis, Dunaliella bardowil, Crypthecodinium cohnii, Schizochytrium sp., Chlamydomonas reinhardtii, Platymonas subcordiformis, Chlorella fusca, Chlorella sorokiniana, Chlorella vulgaris, ‘Chlorella’ ellipsoidea, Chlorella spp., Haematococcus pluvialis; Parachlorella kessleri, Betaphycus gelatinum, Chondrus crispus, Cyanidioschyzon merolae, Cyanidium caldarium, Galdieria sulphuraria, Gelidiella acerosa, Gracilaria changii, Kappaphycus alvarezii, Porphyra miniata, Ostreococcus tauri, Porphyra yezoensis, Porphyridium sp., Palmaria palmata, Gracilaria spp., Isochrysis galbana, Kappaphycus spp., Laminaria japonica, Laminaria spp., Monostroma spp., Nannochloropsis oculata, Porphyra spp., Porphyridium spp., Undaria pinnatifida, Ulva lactuca, Ulva spp., Undaria spp., Phaeodactylum Tricornutum, Navicula saprophila, Cylindrotheca fusiformis, Cyclotella cryptica, Euglena gracilis, Amphidinium sp., Symbiodinium microadriaticum, Macrocystis pyrifera, Ankistrodesmus braunii, Scenedesmus obliquus, Stichococcus sp., Platymonas sp., Dunalielki sauna, and Stephanoptera gracilis.

According to another embodiment, the transgenic photosynthetic organism comprises a designer transgenic plant or plant cells selected from the group consisting of aquatic plants, plant cells, green algae, red algae, brown algae, blue-green algae (oxyphotobacteria including cyanobacteria and oxychlorobacteria), diatoms, marine algae, freshwater algae, salt-tolerant algal strains, cold-tolerant algal strains, heat-tolerant algal strains, antenna-pigment-deficient mutants, butanol-tolerant algal strains, higher-alcohols-tolerant algal strains, butanol-tolerant oxyphotobacteria, higher-alcohols-tolerant oxyphotobacteria, and combinations thereof.

According to another embodiment, said transgenic photosynthetic organism comprises a biosafety-guarded feature selected from the group consisting of: a designer proton-channel gene inducible under pre-determined inducing conditions, a designer cell-division-cycle iRNA gene inducible under pre-determined inducing conditions, a high-CO₂-requiring mutant as a host organism for transformation with designer biofuel-production-pathway genes in creating designer cell-division-controllable photosynthetic organisms, and combinations thereof.

The greater complexity and compartmentalization of eukaryotic plant cells allow for creation of a wider range of photobiologically active designer organisms and novel metabolic pathways compartmentally segregated for production of butanol and/or higher alcohols from water and carbon dioxide. In a eukaryotic algal cell, for example, the translation of designer nuclear genes occurs in cytosol whereas the photosynthesis/Calvin cycle is located inside an algal chloroplast. This clear separation of algal chloroplast photosynthesis from other subcellular functions such as the functions of cytoplasm membrane, cytosol and mitochondria can be used as an advantage in creation of a biosafety-guarded designer algae through an inducible insertion of designer proton-channels into cytoplasm membrane to permanently disable any cell division and/or mating capability while keeping the algal chloroplast functional work with the designer biofuel production, pathways to produce butanol and related higher alcohols. However, it is essential to genetically deliver designer enzyme(s) into the chloroplast to tame the Calvin cycle and funnel metabolism toward butanol directly from CO₂ and H₂O. This requires more complicated gene design to achieve desirable results.

According to one of various embodiments, designer Calvin-cycle-channeled pathway enzymes encoded with designer unclear genes are targetedly expressed into algal chloroplast through use of a transit signal peptide sequence. The said signal peptide is selected from the group consisting of the hydrogenase transit-peptide sequences (HydA1 and HydA2), ferredoxin transit-peptide sequence (Frx1), thioredoxin-m transit-peptide sequence (Trx2), glutamine synthase transit-peptide sequence (Gs2), LhcII transit-peptide sequences, PSII-T transit-peptide sequence (PsbT), PSII-S transit-peptide sequence (PsbS), PSII-W transit-peptide sequence (PsbW), CF₀CF₁ subunit-γ transit-peptide sequence (AtpC), CF₀CF₁ subunit-δ transit-peptide sequence (AtpD), CFoCF₁ subunit-II transit-peptide sequence (AtpG), photosystem I (PSI) transit-peptide sequences, Rubisco SSU transit-peptide sequences, and combinations thereof. Preferred transit peptide sequences include the Hyd1 transit peptide, the Frx1 transit peptide, and the Rubisco SSU transit peptides (such as RbcS2).

SEQ ID NOS. 129-165 present examples for designer DNA constructs of designer chloroplast-targeted enzymes for creation of designer eukaryotic photosynthetic organisms such as designer algae with Calvin-cycle-channeled photosynthetic NADPH-enhanced pathways for photobiological production of butanol and related higher alcohols. Briefly, SEQ ID NO. 129 presents example 129 for a designer Nia1-promoter-controlled chloroplast-targeted Phosphoglycerate Mutase (03) DNA construct (1910 bp) that includes a PCR FD primer (sequence 1-20), a 2×84-bp Chlamydomonas Nia1 (nitrate reductase) promoter (21-188), a 135-bp Chlamydomonas RbcS2 transit peptide (189-323), a Phosphoglycerate Mutase-encoding sequence (324-1667) selected from Nostoc azollae Phosphoglycerate Mutase (ADI65627), a 223-bp Chlamydomonas RbcS2 terminator (1668-1890), and a PCR RE primer (1891-1910).

SEQ ID NO. 130 presents example 130 for a designer Nia1-promoter-controlled chloroplast-targeted Enolase (04) DNA construct (1856 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), an Enolase-encoding sequence (324-1613) selected/modified from Nostoc azollae Enolase (ADI63801), a 223-bp Chlamydomonas RbcS2 terminator (1614-1836), and a PCR RE primer (18837-1856).

SEQ ID NO. 131 presents example 131 for a designer Nia1-promoter-controlled chloroplast-targeted Pyruvate-Kinase (05) DNA construct (1985 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), an enzyme-encoding sequence (324-1742) selected/modified from Cyanothece sp. PCC 8802 pyruvate-kinase (YP_{—}003138017), a 223-bp Chlamydomonas RbcS2 terminator (1743-1965), and a PCR RE primer (1966-1985).

SEQ ID NO. 132 presents example 132 for a designer Nia1-promoter-controlled chloroplast-targeted NADPH-dependent Glyceraldehyde-3-Phosphate Dehydrogenase (34) DNA construct (1568 bp) that includes a PCR FD primer (sequence 1-20), a 2×84-bp Chlamydomonas reinhardtii Nia1 promoter (21-188), a 135-bp Chlamydomonas RbcS2 transit peptide (189-323), a NADPH-dependent Glyceraldehyde-3-phosphate dehydrogenase-encoding sequence (324-1325) selected/modified from Staphylococcus lugdunensis NADPH-dependent glyceraldehyde-3-phosphate dehydrogenase (ADC87332), a 223-bp Chlamydomonas RbcS2 terminator (1326-1548), and a PCR RE primer (1549-1568).

SEQ ID NO. 133 presents example 133 for a designer Nia1-promoter-controlled chloroplast-targeted NAD-dependent Glyceraldehyde-3-phosphate dehydrogenase (35) DNA construct (1571 bp) that includes a PCR FD primer (sequence 1-20), a 2×84-bp Chlamydomonas Nia1 (nitrate reductase) promoter (21-188), a 135-bp Chlamydomonas RbcS2 transit peptide (189-323), a NAD-dependent Glyceraldehyde-3-phosphate dehydrogenase-encoding sequence (324-1328) selected/modified from Flavobacteriaceae bacterium NAD-dependent Glyceraldehyde-3-phosphate dehydrogenase (YP_{—}003095198), a 223-bp Chlamydomonas RbcS2 terminator (1329-1551), and a PCR RE primer (1552-1571).

SEQ ID NO. 134 presents example 134 for a designer Nia1-promoter-controlled chloroplast-targeted Citramalate Synthase (36) DNA construct (2150 bp) that includes a PCR FD primer (sequence 1-20), a 2×84-bp Chlamydomonas Nia1 (nitrate reductase) promoter (21-188), a 135-bp Chlamydomonas RbcS2 transit peptide (189-323), a Citramalate Synthase-encoding sequence (324-1907) selected from Hydrogenobacter Citramalate Synthase (ADO45737), a 223-bp Chlamydomonas RbcS2 terminator (1908-2130), and a PCR RE primer (2131-2150).

SEQ ID NO. 135 presents example 135 for a designer Nia1-promoter-controlled chloroplast-targeted 3-Isopropylmalate/(R)-2-Methylmalate Dehydratase (37) large/small subunits DNA construct (3125 bp) that includes a PCR FD primer (sequence 1-20), a 2×84-bp Chlamydomonas reinhardtii Nia1 promoter (21-188), a 135-bp Chlamydomonas RbcS2 transit peptide (189-323), a 3-isopropylmalate/(R)-2-methylmalate dehydratase large subunit-encoding sequence (324-2084) selected/modified from Eubacterium eligens 3-isopropylmalate/(R)-2-methylmalate dehydratase large subunit (YP_{—}002930810), a 2×84-bp Chlamydomonas Nia1 promoter (2085-2252), a 135-bp Chlamydomonas RbcS2 transit peptide (2253-2387), a 3-isopropylmalate/(R)-2-methylmalate dehydratase small subunit-encoding sequence (2388-2882) selected/modified from Eubacterium eligens 3-isopropylmalate/(R)-2-methylmalate dehydratase small subunit (YP_{—}002930809), a 223-bp Chlamydomonas RbcS2 terminator (2883-3105), and a PCR RE primer (3106-3125).

SEQ ID NO. 136 presents example 136 for a designer Nia1-promoter-controlled chloroplast-targeted 3-Isopropylmalate Dehydratase (38) large/small subunits DNA construct (2879 bp) that includes a PCR FD primer (sequence 1-20), a 2×84-bp Chlamydomonas Nia1 promoter (21-188), a 135-bp Chlamydomonas RbcS2 transit peptide (189-323), a 3-isopropylmalate dehydratase large subunit-encoding sequence (324-1727) selected/modified from Cyanothece 3-isopropylmalate dehydratase large subunit (YP_{—}003886427), a 2×84-bp Chlamydomonas Nia1 promoter (1727-1894), a 135-bp Chlamydomonas RbcS2 transit peptide (1895-2029), a 3-isopropylmalate dehydratase small subunit-encoding sequence (2030-2636) selected from Cyanothece 3-isopropylmalate dehydratase small subunit (YP_{—}003889452), a 223-bp Chlamydomonas r RbcS2 terminator (2637-2859), and a PCR RE primer (2860-2879).

SEQ ID NO. 137 presents example 137 for a designer Nia1-promoter-controlled chloroplast-targeted 3-Isopropylmalate Dehydrogenase (39) DNA construct (1661 bp) that includes a PCR FD primer (sequence 1-20), a 2×84-bp Chlamydomonas Nia1 (nitrate reductase) promoter (21-188), a 135-bp Chlamydomonas RbcS2 transit peptide (189-323), a 3-isopropylmalate dehydrogenase-encoding sequence (324-1418) selected/modified from Cyanothece 3-isopropylmalate dehydrogenase (YP_{—}003888480), a 223-bp Chlamydomonas RbcS2 terminator (1419-1641), and a PCR RE primer (1642-1661).

SEQ ID NO. 138 presents example 138 for a designer Nia1-promoter-controlled chloroplast-targeted 2-Isopropylmalate Synthase (40) DNA construct (2174 bp) that includes a PCR FD primer (sequence 1-20), a 2×84-bp Chlamydomonas Nia1 promoter (21-188), a 135-bp Chlamydomonas RbcS2 transit peptide (189-323), a 2-isopropylmalate synthase-encoding sequence (324-1931) selected/modified from Cyanothece 2-isopropylmalate synthase (YP_{—}003890122), a 223-bp Chlamydomonas RbcS2 terminator (1932-2154), and a PCR RE primer (2155-2174).

SEQ ID NO. 139 presents example 139 for a designer Nia1-promoter-controlled chloroplast-targeted Isopropylmalate Isomerase (41) large/small subunit DNA construct (2882 bp) that includes a PCR FD primer (sequence 1-20), a 2×84-bp Chlamydomonas Nia1 promoter (21-188), a 135-bp Chlamydomonas RbcS2 transit peptide (189-323), an isopropylmalate isomerase large subunit-encoding sequence (324-1727) selected/modified from Anabaena variabilis isopropylmalate isomerase large subunit (YP_{—}324467), a 2×84-bp Chlamydomonas reinhardtii Nia1 promoter (1728-1895), a 135-bp Chlamydomonas RbcS2 transit peptide (1896-2030), an isopropylmalate isomerase small subunit-encoding sequence (2031-2639) selected/modified from Anabaena isopropylmalate isomerase small subunit (YP_{—}324466), a 223-bp Chlamydomonas RbcS2 terminator (2640-2862), and a PCR RE primer (2863-2882).

SEQ ID NO. 140 presents example 140 for a designer Nia1-promoter-controlled chloroplast-targeted 2-Keto Acid Decarboxylase (42) DNA construct (2210 bp) that includes a PCR FD primer (1-20), a 2×84-bp Chlamydomonas Nia1 promoter (21-188), a 135-bp Chlamydomonas RbcS2 transit peptide (189-323), a 2-keto acid decarboxylase-encoding sequence (324-1967) selected from Lactococcus 2-keto acid decarboxylase (AAS49166), a 223-bp Chlamydomonas RbcS2 terminator (1968-2190), and a PCR RE primer (2191-2210).

SEQ ID NO. 141 presents example 141 for a designer Nia1-promoter-controlled chloroplast-targeted NADH-dependent Alcohol Dehydrogenase (43) DNA construct (1724 bp) that includes a PCR FD primer (sequence 1-20), a 2×84-bp Chlamydomonas Nia1 promoter (21-188), a 135-bp Chlamydomonas RbcS2 transit peptide (189-323), a NADH-dependent alcohol dehydrogenase-encoding sequence (324-1481) selected/modified from Gluconacetobacter hansenii NADH-dependent alcohol dehydrogenase (ZP_{—}06834544), a 223-bp Chlamydomonas RbcS2 terminator (1482-1704), and a PCR RE primer (1705-1724).

SEQ ID NO. 142 presents example 142 for a designer Nia1-promoter-controlled chloroplast-targeted NADPH-dependent Alcohol Dehydrogenase (44) DNA construct (1676 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), a NADPH-dependent alcohol dehydrogenase-encoding sequence (324-1433) selected/modified from Fusobacterium NADPH-dependent alcohol dehydrogenase (ZP_{—}04573952), a 223-bp Chlamydomonas reinhardtii RbcS2 terminator (1434-1656), and a PCR RE primer (1657-1676).

Note, use of SEQ ID NOS. 129-141 (and/or 142) in genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced pathway (03-05, 34-43/44 in FIG. 4) for photobiological production of 1-butanol from carbon dioxide and water.

SEQ ID NO. 143 presents example 143 for a designer Nia1-promoter-controlled chloroplast-targeted Phosphoenolpyruvate Carboxylase (45) DNA construct (3629 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), a Phosphoenolpyruvate Carboxylase-encoding sequence (324-3386) selected/modified from Cyanothece sp. PCC 7822 Phosphoenolpyruvate Carboxylase (YP_{—}003887888), a 223-bp Chlamydomonas reinhardtii RbcS2 terminator (3387-3609), and a PCR RE primer (3610-3629).

SEQ ID NO. 144 presents example 144 for a designer Nia1-promoter-controlled chloroplast-targeted Aspartate Aminotransferase (46) DNA construct (1745 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), a Aspartate Aminotransferase-encoding sequence (324-1502) selected/modified from Synechococcus elongatus PCC 6301 Aspartate Aminotransferase (YP_{—}172275), a 223-bp Chlamydomonas reinhardtii RbcS2 terminator (1503-1525), and a PCR RE primer (1526-1745).

SEQ ID NO. 145 presents example 145 for a designer Nia1-promoter-controlled chloroplast-targeted Aspartokinase (47) DNA construct (2366 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), an Aspartokinase-encoding sequence (324-2123) selected/modified from Cyanothece Aspartokinase (YP_{—}003136939), a 223-bp Chlamydomonas RbcS2 terminator (2124-2346), and a PCR RE primer (2347-2366).

SEQ ID NO. 146 presents example 146 for a designer Nia1-promoter-controlled chloroplast-targeted Aspartate-Semialdehyde Dehydrogenase (48) DNA construct (1604 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), an Aspartate-semialdehyde dehydrogenase-encoding sequence (324-1361) selected/modified from Trichodesmium erythraeum IMS101 Aspartate-semialdehyde dehydrogenase (ABG50031), a 223-bp Chlamydomonas RbcS2 terminator (1362-1584), and a PCR RE primer (1585-1604).

SEQ ID NO. 147 presents example 147 for a designer Nia1-promoter-controlled chloroplast-targeted Homoserine Dehydrogenase (49) DNA construct (1868 bp) that includes a PCR FD primer (sequence 1-20), a 2×84-bp Chlamydomonas Nia1 promoter (21-188), a 135-bp Chlamydomonas RbcS2 transit peptide (189-323), a homoserine dehydrogenase-encoding sequence (324-1625) selected from Cyanothece homoserine dehydrogenase (YP_{—}003887242), a 223-bp Chlamydomonas RbcS2 terminator (1626-1848), and a PCR RE primer (1849-1868).

SEQ ID NO. 148 presents example 148 for a designer Nia1-promoter-controlled chloroplast-targeted Homoserine Kinase (50) DNA construct (1472 bp) that includes a PCR FD primer (sequence 1-20), a 2×84-bp Chlamydomonas Nia1 promoter (21-188), a 135-bp Chlamydomonas RbcS2 transit peptide (189-323), a Homoserine kinase-encoding sequence (324-1229) selected/modified from Cyanothece Homoserine kinase (YP_{—}003886645), a 223-bp Chlamydomonas RbcS2 terminator (1230-1452), and a PCR RE primer (1453-1472).

SEQ ID NO. 149 presents example 149 for a designer Nia1-promoter-controlled chloroplast-targeted Threonine Synthase (51) DNA construct (1655 bp) that includes a PCR FD primer (sequence 1-20), a 2×84-bp Chlamydomonas Nia1 promoter (21-188), a 135-bp Chlamydomonas RbcS2 transit peptide (189-323), a Threonine synthase-encoding sequence (324-1412) selected/modified from Cyanothece Threonine synthase (YP_{—}002485009), a 223-bp Chlamydomonas RbcS2 terminator (1413-1635), and a PCR RE primer (1636-1655).

SEQ ID NO. 150 presents example 150 for a designer Nia1-promoter-controlled chloroplast-targeted Threonine Ammonia-Lyase (52) DNA construct (2078 bp) that includes a PCR FD primer (sequence 1-20), a 2×84-bp Chlamydomonas Nia1 promoter (21-188), a 135-bp Chlamydomonas RbcS2 transit peptide (189-323), a threonine ammonia-lyase-encoding sequence (324-1835) selected/modified from Synechococcus threonine ammonia-lyase (ZP_{—}05035047), a 223-bp Chlamydomonas RbcS2 terminator (1836-2058), and a PCR RE primer (2059-2078).

Note, use of SEQ ID NOS. 129, 130, 132, 133, 143-150, 137-141 (and/or 141) through genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced pathway (03, 04, 34, 35, 45-52, 39-43/44 in FIG. 4) for photobiological production of 1-butanol from carbon dioxide and water.

SEQ ID NO. 151 presents example 151 for a designer Nia1-promoter-controlled chloroplast-targeted Acetolactate Synthase (53) DNA construct (2282 bp) that includes a PCR FD primer (sequence 1-20), a 2×84-bp Chlamydomonas reinhardtii Nia1 promoter (21-188), a 135-bp Chlamydomonas RbcS2 transit peptide (189-323), an acetolactate synthase-encoding sequence (324-2039) selected from Bacillus subtilis acetolactate synthase (CAB07802), a 223-bp Chlamydomonas RbcS2 terminator (2040-2262), and a PCR RE primer (2263-2282).

SEQ ID NO. 152 presents example 152 for a designer Nia1-promoter-controlled chloroplast-targeted Ketol-Acid Reductoisomerase (54) DNA construct (1562 bp) that includes a PCR FD primer (sequence 1-20), a 2×84-bp Chlamydomonas Nia1 promoter (21-188), a 135-bp Chlamydomonas RbcS2 transit peptide (189-323), an enzyme-encoding sequence (324-1319) selected/modified from Cyanothece ketol-acid reductoisomerase (YP_{—}003885458), a 223-bp Chlamydomonas RbcS2 terminator (1320-1542), and a PCR RE primer (1543-1562).

SEQ ID NO. 153 presents example 153 for a designer Nia1-promoter-controlled chloroplast-targeted Dihydroxy-Acid Dehydratase (55) DNA construct (2252 bp) that includes a PCR FD primer (sequence 1-20), a 2×84-bp Chlamydomonas Nia1 promoter (21-188), a 135-bp Chlamydomonas RbcS2 transit peptide (189-323), a dihydroxy-acid dehydratase-encoding sequence (324-2009) selected from Cyanothece dihydroxy-acid dehydratase (YP_{—}003887466), a 223-bp Chlamydomonas RbcS2 terminator (2010-2232), and a PCR RE primer (2233-2252).

SEQ ID NO. 154 presents example 154 for a designer Nia1-promoter-controlled chloroplast-targeted 2-Methylbutyraldehyde Reductase (56) DNA construct (1496 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), an enzyme-encoding sequence (324-1253) selected/modified from Pichia pastoris GS115 2-methylbutyraldehyde reductase (XP_{—}002490018), a 223-bp Chlamydomonas reinhardtii RbcS2 terminator (1254-1476), and a PCR RE primer (1477-1496).

Note, use of SEQ ID NOS. 129-137, 140, and 151-154 in genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced pathway (03-05, 34-39, 53-55, 42, and 56 in FIG. 5) for photobiological production of 2-methyl-1-butanol from carbon dioxide and water.

SEQ ID NO. 155 presents example 155 for a designer Nia1-promoter-controlled chloroplast-targeted 3-Methylbutanal Reductase (57) DNA construct (1595 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), a 3-methylbutanal reductase-encoding sequence (324-1352) selected/modified from Saccharomyces cerevisiae S288c 3-methylbutanal reductase (DAA10635), a 223-bp Chlamydomonas reinhardtii RbcS2 terminator (1353-1575), and a PCR RE primer (1576-1595).

Note, use of SEQ ID NOS. 129-133, 151-153, 140 and 141 (or 142) in genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced pathway (03-05, 34, 35, 53-55, 42, and 43 (44) in FIG. 6) for photobiological production of isobutanol from carbon dioxide and water. Whereas, SEQ ID NOS. 129-133, 151-153, 136-138, 140 and 155 represent a designer eukaryotic photosynthetic organism such as designer Chlamydomonas with a Calvin-cycle 3-phosphogylcerate-branched NADPH-enhanced pathway (03-05, 34, 35, 53-55, 40, 38, 39, 42, and 57 in FIG. 6) that can photobiologically produce 3-methyl-1-butanol from carbon dioxide and water.

SEQ ID NO. 156 presents example 156 for a designer Nia1-promoter-controlled chloroplast-targeted NADH-dependent Butanol Dehydrogenase (12a) DNA construct (1739 bp) that includes a PCR FD primer (sequence 1-20), a 2×84-bp Chlamydomonas reinhardtii Nia1 (nitrate reductase) promoter (21-188), a 135-bp Chlamydomonas reinhardtii RbcS2 transit peptide (189-323), an enzyme-encoding sequence (324-1496) selected/modified from Clostridium perfringens NADH-dependent butanol dehydrogenase (NP_{—}561774), a 223-bp Chlamydomonas RbcS2 terminator (1497-1719), and a PCR RE primer (1720-1739).

SEQ ID NO. 157 presents example 157 for a designer Nia1-promoter-controlled chloroplast-targeted NADPH-dependent Butanol Dehydrogenase (12b) DNA construct (1733 bp) that includes a PCR FD primer (sequence 1-20), a 2×84-bp Chlamydomonas reinhardtii Nia1 promoter (21-188), a 135-bp Chlamydomonas reinhardtii RbcS2 transit peptide (189-323), an enzyme-encoding sequence (324-1490) selected/modified from Clostridium saccharobutylicum NADPH-dependent butanol dehydrogenase (AAA83520), a 223-bp Chlamydomonas reinhardtii RbcS2 terminator (1491-1713), and a PCR RE primer (1714-1733).

Note, use of SEQ ID NOS. 129-140 and 156 (and/or 157) in genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced butanol production pathway (03-05, 34-42 and 12 in FIG. 4) for more specific photobiological production of 1-butanol from carbon dioxide and water. Similarly, SEQ ID NOS. 129, 130, 132, 133, 143-150, 137-140, and 156 (and/or 157) represent another designer eukaryotic photosynthetic organism such as designer Chlamydomonas with a Calvin-cycle 3-phosphogylcerate-branched NADPH-enhanced butanol-production pathway (03, 04, 34, 35, 45-52, 39-42 and 12 in FIG. 4) for photobiological production of 1-butanol from carbon dioxide and water.

SEQ ID NO. 158 presents example 158 for a designer Nia1-promoter-controlled chloroplast-targeted 3-Ketothiolase (07′) DNA construct (1745 bp) that includes a PCR FD primer (sequence 1-20), a 2×84-bp Chlamydomonas Nia1 (nitrate reductase) promoter (21-188), a 135-bp Chlamydomonas RbcS2 transit peptide (189-323), a 3-Ketothiolase-encoding sequence (324-1502) selected/modified from Azohydromonas lata 3-Ketothiolase (AAD10275), a 223-bp Chlamydomonas RbcS2 terminator (1503-1725), and a PCR RE primer (1726-1745).

SEQ ID NO. 159 presents a designer Nia1-promoter-controlled chloroplast-targeted 3-Hydroxyacyl-CoA dehydrogenase (08′) DNA construct (1439 bp) that includes a PCR FD primer (sequence 1-20), a 2×84-bp Chlamydomonas Nia1 promoter (21-188), a 135-bp Chlamydomonas RbcS2 transit peptide (189-323), an enzyme-encoding sequence (324-1196) selected/modified from Oceanithermus 3-Hydroxyacyl-CoA dehydrogenase (ADR36325), a 223-bp Chlamydomonas RbcS2 terminator (1197-1419), and a PCR RE primer (1420-1439).

SEQ ID NO. 160 presents example 160 for a designer Nia1-promoter-controlled chloroplast-targeted Enoyl-CoA dehydratase (09′) DNA construct (1337 bp) that includes a PCR FD primer (sequence 1-20), a 2×84-bp Chlamydomonas Nia1 promoter (21-188), a 135-bp Chlamydomonas RbcS2 transit peptide (189-323), an enzyme-encoding sequence (324-1094) selected/modified from Bordetella petrii Enoyl-CoA dehydratase (YP_{—}001629844), a 223-bp Chlamydomonas RbcS2 terminator (1095-1317), and a PCR RE primer (1318-1337).

SEQ ID NO. 161 presents example 161 for a designer Nia1-promoter-controlled 2-Enoyl-CoA reductase (10′) DNA construct (1736 bp) that includes a PCR FD primer (sequence 1-20), a 2×84-bp Chlamydomonas Nia1 promoter (21-188), a 135-bp Chlamydomonas RbcS2 transit peptide (189-323), an enzyme-encoding sequence (324-1493) selected/modified from Xanthomonas campestris 2-Enoyl-CoA reductase (YP_{—}001905744), a 223-bp Chlamydomonas RbcS2 terminator (1494-1716), and a PCR RE primer (1717-1736).

SEQ ID NO. 162 presents example 162 for a designer Nia1-promoter-controlled chloroplast-targeted Acyl-CoA reductase (11′) DNA construct (2036 bp) that includes a PCR FD primer (sequence 1-20), a 2×84-bp Chlamydomonas reinhardtii Nia1 promoter (21-188), a 135-bp Chlamydomonas RbcS2 transit peptide (189-323), an enzyme-encoding sequence (324-1793) selected/modified from Thermosphaera aggregans Acyl-CoA reductase (YP_{—}003649571), a 223-bp Chlamydomonas RbcS2 terminator (1794-2016), and a PCR RE primer (2017-2036).

SEQ ID NO. 163 presents example 163 for a designer Nia1-promoter-controlled chloroplast-targeted Hexanol Dehydrogenase (12′) DNA construct (1625 bp) that includes a PCR FD primer (sequence 1-20), a 2×84-bp Chlamydomonas Nia1 promoter (21-188), a 135-bp Chlamydomonas RbcS2 transit peptide (189-323), an enzyme-encoding sequence (324-1382) selected/modified from Mycobacterium chubuense hexanol dehydrogenase (ACZ56328), a 223-bp Chlamydomonas RbcS2 terminator (1383-1605), and a PCR RE primer (1606-1625).

Note, use of SEQ ID NOS. 158-163 with other proper DNA constructs such as SEQ ID NOS. 132 and 133 in genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced hexanol production pathway (34, 35, 03-10, and 07′-12′ in FIG. 7) for photobiological production of 1-hexanol from carbon dioxide and water.

SEQ ID NO. 164 presents example 164 for a designer Nia1-promoter-controlled chloroplast-targeted Octanol Dehydrogenase (12″) DNA construct (1249 bp) that includes a PCR FD primer (sequence 1-20), a 2×84-bp Chlamydomonas Nia1 promoter (21-188), a 135-bp Chlamydomonas RbcS2 transit peptide (189-323), an enzyme-encoding sequence (324-1006) selected/modified from Drosophila subobscura Octanol dehydrogenase (ABO65263), a 223-bp Chlamydomonas RbcS2 terminator (1007-1229), and a PCR RE primer (1230-1249).

Note, SEQ ID NOS. 132, 133, and 158-163 represent a designer eukaryotic photosynthetic organism such as a designer Chlamydomonas with a designer hydrocarbon chain elongation pathway (34, 35, 07′-12′ as shown in FIG. 7) for photobiological production of 1-hexanol. SEQ ID NOS: 132, 133, 158-162 and 164 represent another designer eukaryotic photosynthetic organism such as a designer Chlamydomonas with a designer hydrocarbon chain elongation pathway (34, 35, 07′-10′ and 07″-12″ as shown in FIG. 7) for photobiological production of 1-octanol.

SEQ ID NO. 165: a designer Nia1-promoter-controlled chloroplast-targeted Short Chain Alcohol Dehydrogenase (43′) DNA construct (1769 bp) that includes a PCR FD primer (sequence 1-20), a 2×84-bp Chlamydomonas Nia1 promoter (21-188), a 135-bp Chlamydomonas RbcS2 transit peptide (189-323), an enzyme-encoding sequence (324-1526) selected/modified from Burkholderia Short chain alcohol dehydrogenase (AB056626), a 223-bp Chlamydomonas RbcS2 terminator (1527-1749), and a PCR RE primer (1750-1769).

Note, use of SEQ ID NOS. 129-140 and 165 in genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced pathway (03-05, 34-41, 39′-43′, 39′-43′ and 39″-43″ in FIG. 8) for photobiological production of 1-pentanol, 1-hexanol, and 1-heptanol from carbon dioxide and water. Similarly, SEQ ID NOS. 129-140 and 163 represent another designer eukaryotic photosynthetic organism such as designer Chlamydomonas with a Calvin-cycle 3-phosphogylcerate-branched NADPH-enhanced pathway (03-05, 34-41, 39′-41′, 39′-42′ and 12′ in FIG. 8) for photobiological production of 1-hexanol from carbon dioxide and water.

Likewise, use of SEQ ID NOS. 129-137, 151-153, 138-140 and 165 through genetic transformation of an eukaryotic photosynthetic organism such as Chlamydomonas can create a designer eukaryotic photosynthetic organism such as designer Chlamydomonas with a Calvin-cycle 3-phosphogylcerate-branched NADPH-enhanced pathway (03-05, 34-39, 53-55, 39′-43′, 39′-43′, and 39″-43″ in FIG. 9) for photobiological production of 3-methyl-1-pentanol, 4-methyl-1-hexanol, and 5-methyl-1-heptanol from carbon dioxide and water; The expression of SEQ ID NOS. 129, 130, 132, 133, 143-150, 151-153, 137-140 and 165 in an eukaryotic photosynthetic organism such as a host Chlamydomonas represent another designer eukaryotic photosynthetic organism with a Calvin-cycle 3-phosphogylcerate-branched NADPH-enhanced pathway (03, 05, 34, 35, 42-55, 39′-43′, 39′-43′, and 39″-43″ in FIG. 9) for photobiological production of 3-methyl-1-pentanol, 4-methyl-1-hexanol, and 5-methyl-1-heptanol from carbon dioxide and water; The expression of SEQ ID NOS. 129-133, 151-153, 136-140 and 165 in a host eukaryotic photosynthetic organism such as Chlamydomonas represent yet another designer eukaryotic photosynthetic organism with a Calvin-cycle 3-phosphogylcerate-branched NADPH-enhanced pathway (03-05, 34, 35, 53-55, 40, 38, 39, 39′-43′, 39′-43′, and 39″-43″ in FIG. 10) for photobiological production of 4-methyl-1-pentanol, 5-methyl-1-hexanol, and 6-methyl-1-heptanol from carbon dioxide and water.

Use of Designer Photosynthetic Organisms with Photobioreactor for Production and Harvesting of Butanol and Related Higher Alcohols

The designer photosynthetic organisms with designer Calvin-cycle channeled photosynthetic NADPH-enhanced pathways (FIGS. 1, and 4-10) can be used with photobioreactors for production and harvesting of butanol and/or related higher alcohols. The said butanol and/or related higher alcohols are selected from the group consisting of: 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, 6-methyl-1-heptanol, and combinations thereof.

The said designer photosynthetic organisms such as designer transgenic oxyphotobacteria and algae comprise designer Calvin-cycle-channeled and photosynthetic NADPH-enhanced pathway gene(s) and biosafety-guarding technology for enhanced photobiological production of butanol and related higher alcohols from carbon dioxide and water. According to one of the various embodiments, it is a preferred practice to grow designer photosynthetic organisms photoautotrophically using carbon dioxide (CO₂) and water (H₂O) as the sources of carbon and electrons with a culture medium containing inorganic nutrients. The nutrient elements that are commonly required for oxygenic photosynthetic organism growth are: N, P, and K at the concentrations of about 1-10 mM, and Mg, Ca, S, and Cl at the concentrations of about 0.5 to 1.0 mM, plus some trace elements Mn, Fe, Cu, Zn, B, Co, Mo among others at μM concentration levels. All of the mineral nutrients can be supplied in an aqueous minimal medium that can be made with well-established recipes of oxygenic photosynthetic organism (such as algal) culture media using water (freshwater for the designer freshwater algae; seawater for the salt-tolerant designer marine algae) and relatively small of inexpensive fertilizers and mineral salts such as ammonium bicarbonate (NH₄HCO₃) (or ammonium nitrate, urea, ammonium chloride), potassium phosphates (K₂HPO₄ and KH₂PO₄), magnesium sulfate heptahydrate (MgSO₄.7H₂O), calcium chloride (CaCl₂), zinc sulfate heptahydrate (ZnSO₄.7H₂O), iron (II) sulfate heptahydrate (FeSO₄.7H₂O), and boric acid (H₃BO₃), among others. That is, large amounts of designer algae (or oxyphotobacteria) cells can be inexpensively grown in a short period of time because, under aerobic conditions such as in an open pond, the designer algae can photoautotrophically grow by themselves using air CO₂ as rapidly as their wild-type parental strains. This is a significant feature (benefit) of the invention that could provide a cost-effective solution in generation of photoactive biocatalysts (the designer photosynthetic biofuel-producing organisms such as designer algae or oxyphotobacteria) for renewable solar energy production.

According to one of the various embodiments, when designer photosynthetic organism culture is grown and ready for photobiological production of butanol and/or related higher alcohols, the designer photosynthetic organism cells are then induced to express the designer Calvin-cycle channeled photosynthetic NADPH-enhanced pathway(s) to photobiologically produce butanol and/or related higher alcohols from carbon dioxide and water. The method of induction is designer pathway gene(s) specific. For example, if/when a nirA promoter is used to control the designer Calvin-cycle channeled pathway gene(s) such as those of SEQ ID NOS: 58-69 and 72 (and/or 73) which represent a designer transgenic Thermosynechococcus that comprises the designer genes of a Calvin-cycle 3-phophoglycerate-branched photosynthetic NADPH-enhanced pathway (numerically labeled as 34, 35, 03-05, 36-42, and 12 in FIG. 4) for photobiological production of 1-butanol from carbon dioxide and water, the designer transgenic Thermosynechococcus is grown in a minimal liquid culture medium containing ammonium (but no nitrate) and other inorganic nutrients. When the designer transgenic Thermosynechococcus culture is grown and ready for photobiological production of biofuel 1-butanol, nitrate fertilizer will then be added into the culture medium to induce the expression of the designer nirA-controlled Calvin-cycle-channeled pathway to photobiologically produce 1-butanol from carbon dioxide and water in this example.

For the designer photosynthetic organism(s) with anaerobic promoter-controlled pathway(s) such as the designer transgenic Nostoc that contains designer hox-promoter-controlled Calvin-cycle 3-phophoglycerate-branched pathway genes of SEQ ID NOS. 104-109, anaerobic conditions can be used to induce the expression of the designer pathway gene(s) for photobiological production of 2-methyl-1-butanol from carbon dioxide and water (FIG. 5). That is, when the designer transgenic Nostoc culture is grown and ready for photobiological biofuel production, its cells will then be placed (or sealed) into certain anaerobic conditions to induce the expression of the designer hox-controlled pathway gene(s) to photobiologically produce 2-methyl-1-butanol from carbon dioxide and water.

For those designer photosynthetic organism(s) that contains a heat- and light-responsive promoter-controlled and nirA-promoter-controlled pathway(s) such as the designer transgenic Prochlorococcus that contains a set of designer groE-promoter-controlled and nirA-promoter-controlled Calvin-cycle 3-phophoglycerate-branched pathway genes of SEQ ID NOS. 110-118, light and heat are used in conjunction of nitrate addition to induce the expression of the designer pathway genes for photobiological production of isobutanol from carbon dioxide and water (FIG. 6).

According to another embodiment, use of designer marine algae or marine oxyphotobacteria enables the use of seawater and/or groundwater for photobiological production of biofuels without requiring freshwater or agricultural soil. For example, designer Prochlorococcus marinus that contains the designer genes of SEQ ID NOS: 110-117 and 119-122 can use seawater and/or certain groundwater for photoautotrophic growth and synthesis of 3-methyl-1-butanol from carbon dioxide and water with its groE promoter-controlled designer Calvin-cycle-channeled pathway (identified as 34 (native), 35, 03-05, 53-55, 38-40, 42 and 57 in FIG. 6). The designer photosynthetic organisms can be used also in a sealed photobioreactor that is operated on a desert for production of isobutanol with highly efficient use of water since there will be little or no water loss by evaporation and/or transpiration that a common crop system would suffer. That is, this embodiment may represent a new generation of renewable energy (butanol and related higher alcohols) production technology without requiring arable land or freshwater resources.

According to another embodiment, use of nitrogen-fixing designer oxyphotobacteria enables photobiological production of biofuels without requiring nitrogen fertilizer. For example, the designer transgenic Nostoc that contains designer hox-promoter-controlled genes of SEQ ID NOS.104-109 is capable of both fixing nitrogen (N₂) and photobiologically producing 2-methyl-1-butanol from carbon dioxide and water (FIG. 6). Therefore, use of the designer transgenic Nostoc enables photoautotrophic growth and 2-methyl-1-butanol synthesis from carbon dioxide and water.

Certain designer oxyphotobacteria are designed to perform multiple functions. For example, the designer transgenic Cyanothece that contains designer nirA promoter-controlled genes of SEQ ID NOS. 123-127 is capable of (1) using seawater, (2) N₂ fixing nitrogen, and photobiological producing 1-hexanol from carbon dioxide and water (FIG. 8). Use of this type of designer oxyphotobacteria enables photobiological production of advanced biofuels such as 1-hexanol using seawater without requiring nitrogen fertilizer

According to one of various embodiments, a method for photobiological production and harvesting of butanol and related higher alcohols comprises: a) introducing a transgenic photosynthetic organism into a photobiological reactor system, the transgenic photosynthetic organism comprising transgenes coding for a set of enzymes configured to act on an intermediate product of a Calvin cycle and to convert the intermediate product into butanol and related higher alcohols; b) using reducing power and energy associated with the transgenic photosynthetic organism acquired from photosynthetic water splitting and proton gradient coupled electron transport process in the photobioreactor to synthesize butanol and related higher alcohols from carbon dioxide and water; and c) using a product separation process to harvest the synthesized butanol and/or related higher alcohols from the photobioreactor.

In summary, there are a number of embodiments on how the designer organisms may be used for photobiological butanol (and/or related higher alcohols) production. One of the preferred embodiments is to use the designer organisms for direct photosynthetic butanol production from CO₂ and H₂O with a photobiological reactor and butanol-harvesting (filtration and distillation/evaporation) system, which includes a specific operational process described as a series of the following steps: a) Growing a designer transgenic organism photoautotrophically in minimal culture medium using air CO₂ as the carbon source under aerobic (normal) conditions before inducing the expression of the designer butanol-production-pathway genes; b) When the designer organism culture is grown and ready for butanol production, sealing or placing the culture into a specific condition to induce the expression of designer Calvin-cycle-channeled pathway genes; c) When the designer pathway enzymes are expressed, supplying visible light energy such as sunlight for the designer-genes-expressed cells to work as the catalysts for photosynthetic production of butanol and/or related higher alcohols from CO₂ and H₂O; d) Harvesting the product butanol and/or related higher alcohols by any method known to those skilled in the art. For example, harvesting the butanol and/or related higher alcohols from the photobiological reactor can be achieved by a combination of membrane filtration and distillation/evaporation butanol-harvesting techniques.

The above process to use the designer organisms for photosynthetic production and harvesting of butanol and related higher alcohols can be repeated for a plurality of operational cycles to achieve more desirable results. Any of the steps a) through d) of this process described above can also be adjusted in accordance of the invention to suit for certain specific conditions. In practice, any of the steps a) through d) of the process can be applied in full or in part, and/or in any adjusted combination as well for enhanced photobiological production of butanol and higher alcohol in accordance of this invention.

In addition to butanol and/or related higher alcohols production, it is also possible to use a designer organism or part of its designer butanol-production pathway(s) to produce certain intermediate products of the designer Calvin-cycle-channeled pathways (FIGS. 1 and 4-10) including (but not limited to): butyraldehyde, butyryl-CoA, crotonyl-CoA, 3-hydroxybutyryl-CoA, acetoacetyl-CoA, acetyl-CoA, pyruvate, phosphoenolpyruvate, 2-phosphoglycerate, 1,3-diphosphoglycerate, glyceraldehye-3-phosphate, dihydroxyacetone phosphate, fructose-1,6-diphosphate, fructose-6-phosphate, glucose-6-phosphate, glucose, glucose-1-phosphate, citramalate, citraconate, methyl-D-malate, 2-ketobutyrate, 2-ketovalerate, oxaloacetate, aspartate, homoserine, threonine, 2-keto-3-methylvalerate, 2-methylbutyraldehyde, 3-methylbutyraldehyde, 4-methyl-2-oxopentanoate, 3-isopropylmalate, 2-isopropylmalate, 2-oxoisovalerate, 2,3-dihydroxy-isovalerate, 2-acetolactate, isobutyraldehyde, 3-keto-C6-acyl-CoA, 3-hydroxy-C6-acyl-CoA, C6-enoyl-CoA, C6-acyl-CoA, 3-keto-C8-acyl-CoA, 3-hydroxy-C8-acyl-CoA, C8-enoyl-CoA, C8-acyl-CoA, octanal, 1-pentanol, 1-hexanal, 1-heptanal, 2-ketohexanoate, 2-ketoheptanoate, 2-ketooctanoate, 2-ethylmalate, 3-ethylmalate, 3-methyl-1-pentanal, 4-methyl-1-hexanal, 5-methyl-1-heptanal, 2-hydroxy-2-ethyl-3-oxobutanoate, 2,3-dihydroxy-3-methyl-pentanoate, 2-keto-4-methyl-hexanoate, 2-keto-5-methyl-heptnoate, 2-keto-6-methyl-octanoate, 4-methyl-1-pentanal, 5-methyl-1-hexanal, 6-methyl-1-heptanal, 2-keto-7-methyl-octanoate, 2-keto-6-methyl-heptanoate, and 2-keto-5-methyl-hexanoate. According to one of various embodiments, therefore, a further embodiment comprises an additional step of harvesting the intermediate products that can be produced also from an induced transgenic designer organism. The production of an intermediate product can be selectively enhanced by switching off a designer-enzyme activity that catalyzes its consumption in the designer pathways. The production of a said intermediate product can be enhanced also by using a designer organism with one or some of designer enzymes omitted from the designer butanol-production pathways. For example, a designer organism with the butanol dehydrogenase or butyraldehyde dehydrogenase omitted from the designer pathway(s) of FIG. 1 may be used to produce butyraldehyde or butyryl-CoA, respectively.

### Designer Calvin-Cycle-Channeled Aerobic Hydrogenotrophic Biofuel Pathways

According to one of the various embodiments, a designer hydrogenotrophic Calvin-cycle-channeled pathway technology (FIG. 11) is created that takes hydrogen (H₂), oxygen (O₂) and carbon dioxide (CO₂) to produce advanced biofuels including butanol and related higher alcohols through the designer Calvin-cycle-channeled pathways (FIGS. 1 and 4-10). As illustrated in FIG. 11, one of the various embodiments here is the expression of designer oxygen (O₂)-tolerant hydrogenases in a designer microbial cell such as cyanobacteria to generate NAD(P)H and ATP from consumption of hydrogen. The expression of a membrane bound hydrogenase (MBH, 70 and its accessory proteins 72 as listed in Table 1) enables oxidation of H₂ through the respiratory electron transport chain (ETC) system to pump protons (H⁺) across the cytoplasm membrane to create transmembrane electrochemical potential for ATP synthesis; whereas the use of a soluble hydrogenase (SH, 71 and its accessory proteins 72) enables generation of NAD(P)H through SH-mediated reduction of NAD(P)⁺ by H₂. Use of ATP and NAD(P)H drives the designer Calvin-cycle-channeled pathways (FIGS. 1 and 4-10) for CO₂ fixation and biofuel butanol and related higher alcohol production. Therefore, this represents an innovative application of the designer Calvin-cycle-channeled biofuel-production pathways.

For example, the expression of a membrane bound hydrogenase (MBH, 70 and its accessory proteins 72) and a soluble hydrogenase (SH, 71 and its accessory proteins 72) in a designer transgenic cyanobacterium that already contains the designer butanol-production-pathway genes of SEQ ID NOS: 58-69 and 72 (and/or 73) can create a hydrogenotrophic Calvin-cycle 3-phophoglycerate-branched 1-butanol production pathway as numerically labeled as 34, 35, 03-05, 36-42, and 12 in FIG. 4. The net result of the designer hydrogenotrophic pathway is the production of 1-butanol(CH₃CH₂CH₂CH₂OH) from hydrogen (H₂), carbon dioxide (CO₂) and oxygen (O₂) according to the following process reaction:

(12+2n)H₂+4CO₂+nO₂→CH₃CH₂CH₂CH₂OH+(7+n)H₂O [20]

The number (n) of oxygen (O₂) molecules used to oxidize hydrogen (H₂) by the respiratory electron-transport-coupled phosphorylation to support the synthesis of a 1-butuanol was estimated to be about 5 in this example.

Note, before the designer genes are turned on, the transgenic cyanobacteria (FIG. 11) can grow photoautotrophically using CO₂, H₂O and sunlight just like their wild-type parental strains. When they are grown and ready for use, they can then be placed into a bioreactor supplied with H₂ (about 85%) and CO₂ (about 10%) with limiting amount of O₂ (about 5%) for hydrogenotrophic synthesis of higher alcohols such as 1-butanol, for example, through the Calvin-cycle-channeled butanol-production pathway of FIG. 1 without requiring any photosynthesis or sunlight. Since hydrogen (H₂) can be made from a number of sources including the electrolysis of water, the designer hydrogenotrophic Calvin-cycle-channeled pathway technology (FIG. 11) enables utilization of inexpensive industrial CO₂ and electricity from solar photovoltaic, wind and nuclear power stations to produce “drop-in-ready” liquid transportation fuel such as butanol without requiring any arable lands or photosynthesis.

### Designer Anaerobic Hydrogenotrophic Reductive-Acetyl-CoA Biofuel-Production Pathways

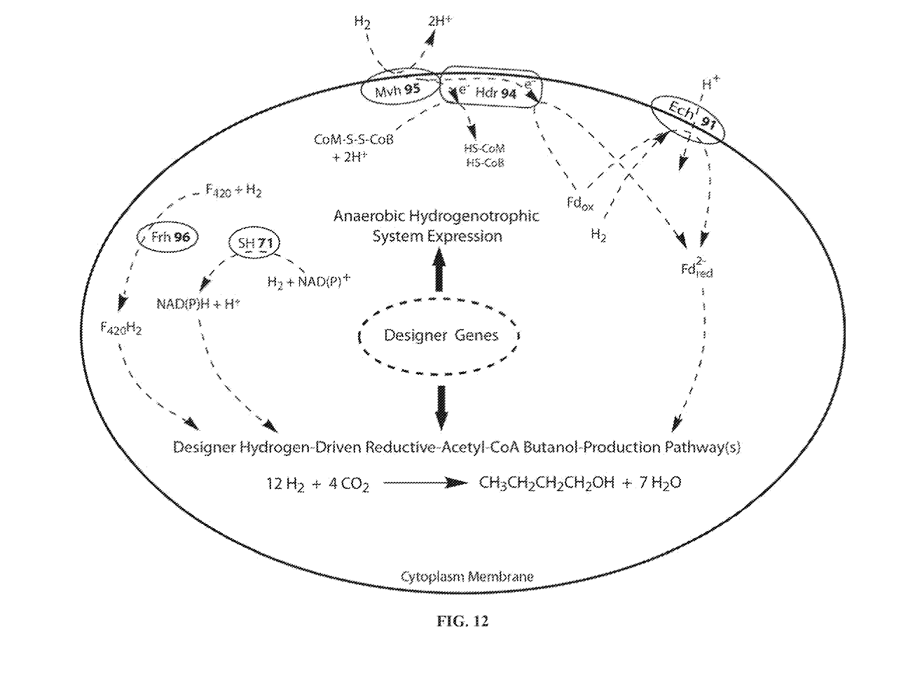

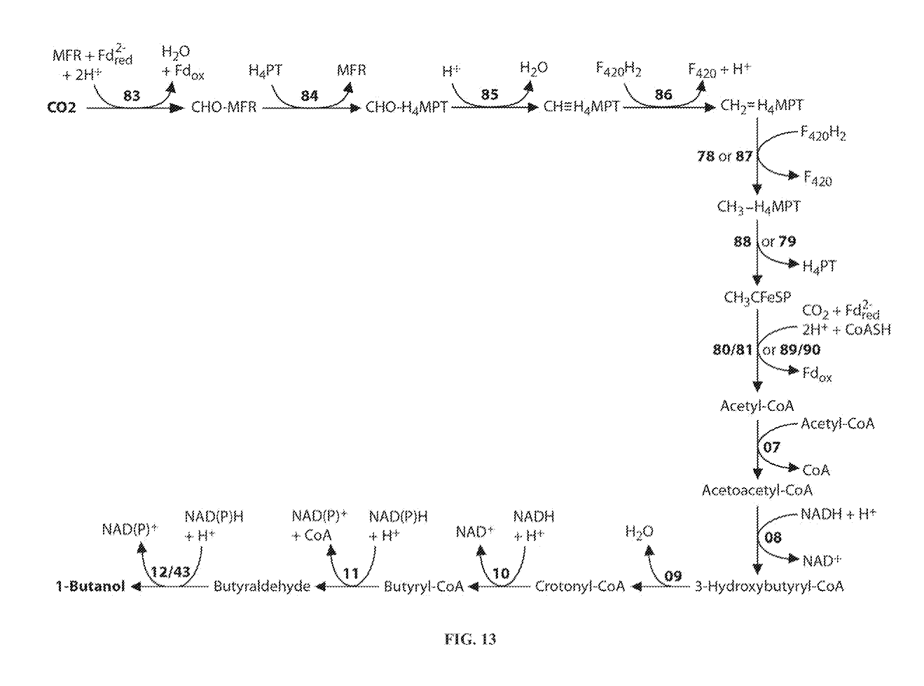

According to one of the various embodiments, a designer hydrogenotrophic reductive-acetyl-CoA biofuel-production pathway technology (FIG. 12) is created that takes hydrogen (H₂) and carbon dioxide (CO₂) to produce advanced biofuels such as butanol and related higher alcohols under anaerobic conditions. As illustrated in FIG. 12, one of the various embodiments here is the expression of a set of designer genes that confer a designer anaerobic hydrogenotrophic system and a reductive-acetyl-CoA butanol-producing pathway (FIG. 13) in a microbial host cell such as a cyanobacterium. Designer anaerobic hydrogenotrophic system includes, for example, energy converting hydrogenase (Ech, 91 in Table 1), [NiFe]-hydrogenase Mvh (95), Coenzyme F₄₂₀-reducing hydrogenase (Frh, 96), native (or heterologous) soluble hydrogenase (SH, 71), NAD(P)H, reduced ferredoxin (Fd_{red}²⁻), HS-CoM, HS-CoB, and heterodissulfide reductase (Hdr; 94); while designer reductive-acetyl-CoA butanol-producing pathway (as shown with the numerical labels 83-90 and 07-12/43 in FIG. 13) comprises formylmethanofuran dehydroganse 83, formyl transferase 84, 10-methenyl-tetrahydromethanopterin cyclohydrolase 85, 10-methylene-H₄ methanopterin dehydrogenase 86, 10-methylene-H₄-methanopterin reductase 87, methyl-H₄-methanopterin: corrinoid iron-sulfur protein methyltransferase 88, corrinoid iron-sulfur protein 89, CO dehydrogenase/acetyl-CoA synthase 90, thiolase 07, 3-hydroxybutyryl-CoA dehydrogenase 08, crotonase 09, butyryl-CoA dehydrogenase 10, butyaldehyde dehydrogenase 11, butanol dehydrogenase 12, and/or alcohol dehydrogenase 43. In this example, the net result of the designer anaerobic hydrogenotrophic reductive-acetyl-CoA butanol-production pathway technology (FIGS. 12 and 13) is the production of 1-butanol(CH₃CH₂CH₂CH₂OH) from hydrogen (H₂) and carbon dioxide (CO₂) according to the following process reaction:

12H₂+4CO₂→CH₃CH₂CH₂CH₂OH+7H₂O [21]

The standard free energy change) (ΔᵣG^{o}) for this overall reaction is −244.7 kJ/mol 1-butanol, which demonstrates that this hydrogen-driven butanol-production technology is not in violation of thermodynamic laws. This equation shows that the use of 12 molecules (24 electrons) of hydrogen (H₂) can produce one molecule of 1-butanol from 4 molecules of carbon dioxide (CO₂). To produce 12 molecules of H₂ by electrolysis of water, it uses 24 electrons from electricity. Therefore, if electrolysis of water is used as a hydrogen source, then 24 electrons (from electricity) are sufficient to generate one molecule of 1-butanol from 4 molecules of CO₂ through the designer anaerobic hydrogenotrophic reductive-acetyl-CoA butanol-production pathway technology (FIGS. 12 and 13).

Therefore, in one of the various embodiments, a designer autotrophic organism comprises a set of designer genes (e.g., designer DNA constructs) that express a set of enzymes conferring the designer anaerobic hydrogenotrophic butanol-production-pathway system (as shown in FIGS. 12 and 13) that comprises: energy converting hydrogenase (Ech) 91, [NiFe]-hydrogenase (Mvh) 95, Coenzyme F₄₂₀-reducing hydrogenase (Frh) 96, native (or heterologous) soluble hydrogenase (SH) 71, heterodissulfide reductase (Hdr) 94, formylmethanofuran dehydroganse 83, formyl transferase 84, 10-methenyl-tetrahydromethanopterin cyclohydrolase 85, 10-methylene-H₄ methanopterin dehydrogenase 86, 10-methylene-H₄-methanopterin reductase 87, methyl-H₄-methanopterin: corrinoid iron-sulfur protein methyltransferase 88, corrinoid iron-sulfur protein 89, CO dehydrogenase/acetyl-CoA synthase 90, thiolase 07, 3-hydroxybutyryl-CoA dehydrogenase 08, crotonase 09, butyryl-CoA dehydrogenase 10, butyaldehyde dehydrogenase 11, butanol dehydrogenase 12 and/or alcohol dehydrogenase 43.

Before the designer genes are turned on, the designer transgenic cyanobacteria (FIG. 12) can grow photoautotrophically using CO₂, H₂O and sunlight just like their wild-type parental strains. When they are grown and ready for use, they can then be placed into a bioreactor for butanol production from H₂ and CO₂ under anaerobic conditions without requiring any photosynthesis or any respiratory oxidation of H₂ by molecular oxygen (O₂). A unique feature of this designer reductive-acetyl-CoA butanol-production pathway (FIG. 13) is that it does not require any ATP; this pathway uses reduced ferredoxin (Fd_{red}²⁻), F₄₂₀H₂ and NAD(P)H that the designer anaerobic hydrogenotrophic system (FIG. 12) can supply from H₂ employing certain electro-proton-coupled bioenergetics bifurcating mechanism. In accordance with one of the various embodiments, this designer pathway (FIG. 13) represents one of the most energy-efficient butanol-production processes identified so for. The standard free energy change (ΔG^{o}) of this specific anaerobic hydrogenotrophic butanol-production process [Eq. 21] is −20.4 kJ/mol per H₂ used. Its maximum hydrogen (H₂)-to-butanol energy conversion efficiency was estimated to be about 91.4%.

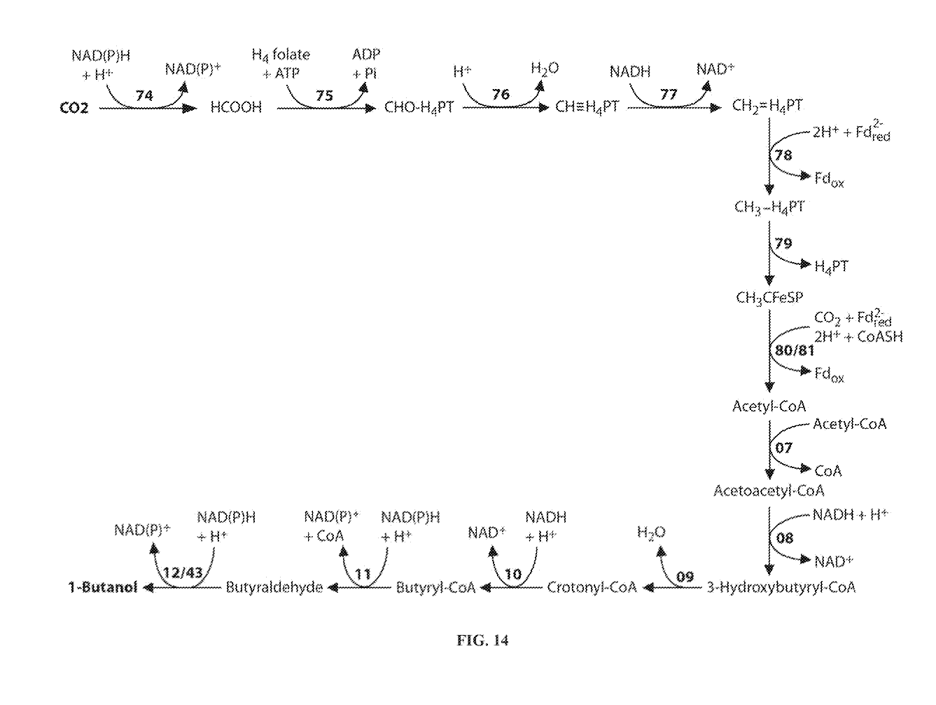

According to one of the various embodiments, another designer anaerobic reductive-acetyl-CoA butanol-production pathway (as shown with the numerical labels 74-81 and 07-12/43 in FIG. 14) is created that can produce 1-butanol from H₂ and CO₂ through use of a set of enzymes comprising: formate dehydroganse 74, 10-formyl-H₄ folate synthetase 75, methenyltetrahydrofolate cyclohydrolase 76, 10-methylene-H₄ folate dehydrogenase 77, 10-methylene-H₄ folate reductase 78, methyl-H₄ folate: corrinoid iron-sulfur protein methyltransferase 79, corrinoid iron-sulfur protein 80, CO dehydrogenase/acetyl-CoA synthase 81, thiolase 07, 3-hydroxybutyryl-CoA dehydrogenase 08, crotonase 09, butyryl-CoA dehydrogenase 10, butyaldehyde dehydrogenase 11, butanol dehydrogenase 12, and/or alcohol dehydrogenase 43.

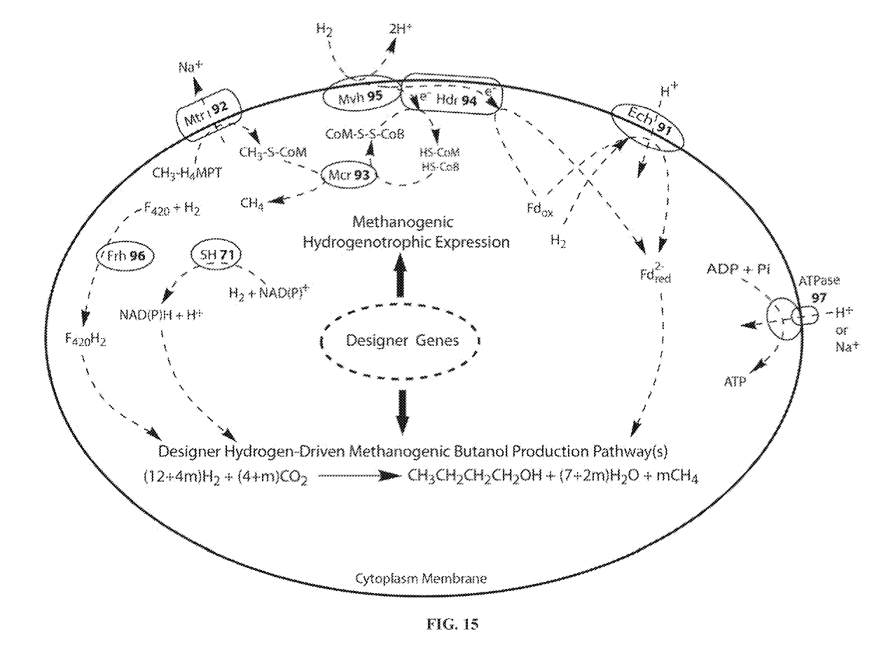

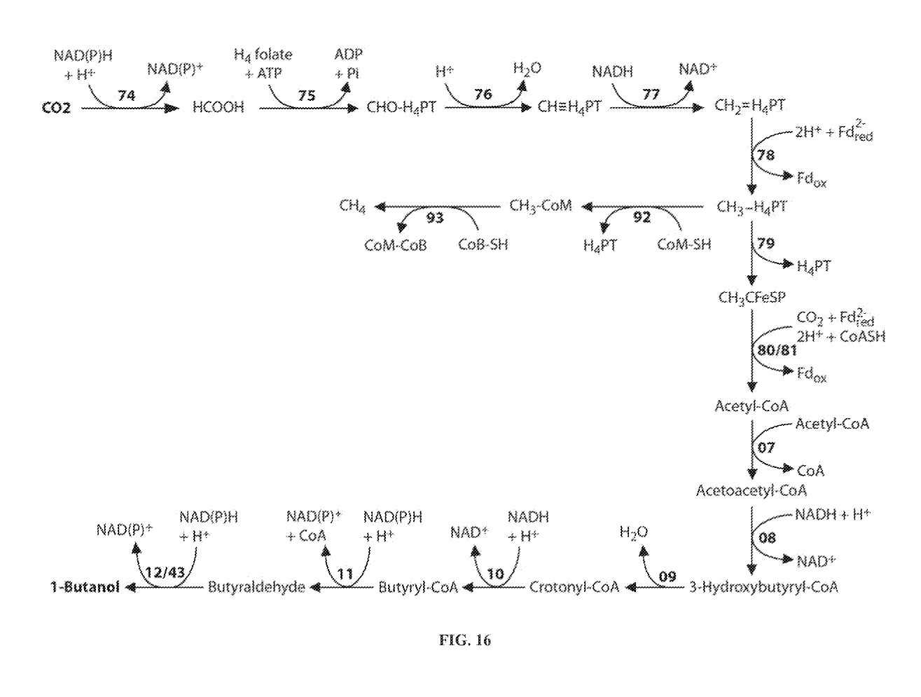

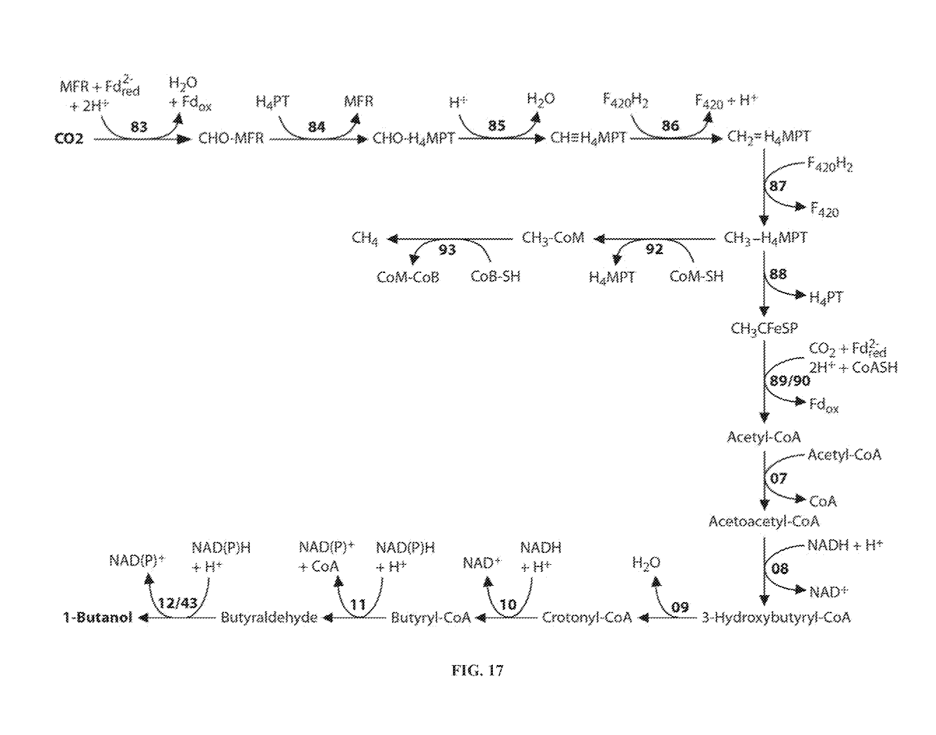

This designer pathway is similar to that of FIG. 13, except that it requires consumption of ATP at the step of 10-formyl-H₄ folate synthetase 75 (FIG. 14). Therefore, it requires ATP supply from other cellular processes in order to operate. According to one of the various embodiments, this pathway (FIG. 14) can be supported by a designer methanogenic hydrogenotrophic cell system (FIG. 15) that produces ATP, Fd_{red}²⁻, F₄₂₀H₂, and NAD(P)H. This designer autotrophic organism comprises a set of designer genes (e.g., designer DNA constructs) that express the designer methanogenic hydrogenotrophic butanol-production-pathway system (as shown in FIGS. 14 and 16) comprising: methyl-H4MPT: coenzyme-M methyltransferase Mtr 92, native (or heterologous) A₁Aₒ-ATP synthase 97, methyl-coenzyme M reductase Mcr 93, energy converting hydrogenase (Ech) 91, [NiFe]-hydrogenase (Mvh) 95, Coenzyme F₄₂₀-reducing hydrogenase (Frh) 96, native (or heterologous) soluble hydrogenase (SH) 71, heterodissulfide reductase (Hdr) 94, formylmethanofuran dehydroganse 83, formyl transferase 84, 10-methenyl-tetrahydromethanopterin cyclohydrolase 85, 10-methylene-H₄ methanopterin dehydrogenase 86, 10-methylene-H₄-methanopterin reductase 87, methyl-H₄-methanopterin: corrinoid iron-sulfur protein methyltransferase 88, corrinoid iron-sulfur protein 89, CO dehydrogenase/acetyl-CoA synthase 90, thiolase 07, 3-hydroxybutyryl-CoA dehydrogenase 08, crotonase 09, butyryl-CoA dehydrogenase 10, butyaldehyde dehydrogenase 11, butanol dehydrogenase 12 and/or alcohol dehydrogenase 43.

For example, the designer methanogenic hydrogenotrophic system (FIG. 15) comprises methyl-H4MPT: coenzyme-M methyltransferase Mtr 92, A₁Aₒ-ATP synthase 97, energy converting hydrogenase (Ech; 91 in Table 1), [NiFe]-hydrogenase Mvh (95), Coenzyme F₄₂₀-reducing hydrogenase (Frh, 96), native (or heterologous) soluble hydrogenase (SH, 71), NAD(P)H, reduced ferredoxin (Fd_{red}²⁻), HS-CoM, HS-CoM, methyl-coenzyme M reductase Mcr 93, and heterodissulfide reductase (Hdr, 94). The Mtr 92 in this system can take a fraction of the CH₃—H₄ MPT intermediate to produce methane and generate a transmembrane electrochemical potential for synthesis of ATP, which can support the ATP-requiring anaerobic reductive-acetyl-CoA butanol-production pathway of FIG. 14. Therefore, the combination of the methanogenic hydrogenotrophic system (FIG. 15) and the ATP-requiring anaerobic reductive-acetyl-CoA butanol-production pathway (FIG. 14) results in a combined pathway (FIG. 16) for production of both butanol and methane. The net result is the production of both butanol and methane (CH₄) from hydrogen (H₂) and carbon dioxide (CO₂) according to the following process reaction where m is the number of CH₄ molecules co-generated per 1-butanol produced:

(12+4m)H₂+(4+m)CO₂→CH₃CH₂CH₂CH₂OH+(7+m)H₂O+mCH₄ [22]

The non-ATP-requiring anaerobic reductive-acetyl-CoA butanol-production pathway (FIG. 13) can, of course, operate with this designer methanogenic hydrogenotrophic system (FIG. 15) as well, resulting in another combined pathway for production of both butanol and methane (FIG. 17). Therefore, in one of the various embodiments, a designer autotrophic organism comprises a set of designer genes (e.g., designer DNA constructs) that express a designer methanogenic hydrogenotrophic butanol-production-pathway system (as shown in FIGS. 15,13, and 17) comprising: methyl-H4MPT: coenzyme-M methyltransferase Mtr 92, native (or heterologous) A₁Aₒ-ATP synthase 97, methyl-coenzyme M reductase Mcr 93, energy converting hydrogenase (Ech) 91, [NiFe]-hydrogenase (Mvh) 95, Coenzyme F₄₂₀-reducing hydrogenase (Frh) 96, native (or heterologous) soluble hydrogenase (SH) 71, heterodissulfide reductase (Hdr) 94, formate dehydroganse 74, 10-formyl-H₄ folate synthetase 75, methenyltetrahydrofolate cyclohydrolase 76, 10-methylene-H₄ folate dehydrogenase 77, 10-methylene-H₄ folate reductase 78, methyl-H₄ folate: corrinoid iron-sulfur protein methyltransferase 79, corrinoid iron-sulfur protein 80, CO dehydrogenase/acetyl-CoA synthase 81, thiolase 07, 3-hydroxybutyryl-CoA dehydrogenase 08, crotonase 09, butyryl-CoA dehydrogenase 10, butyaldehyde dehydrogenase 11, butanol dehydrogenase 12, and/or alcohol dehydrogenase 43.

Some of these enzymes may naturally exist in some of the host organisms depending on their genetic background; some of these native enzymes may be used in constructing part of the designer pathways (FIGS. 12-17) along with designer genes. Therefore, according to one of the various embodiments, a designer autotrophic organism for production of biofuels such as butanol through anaerobic hydrogenotrophic reductive-acetyl-CoA biofuel-production-pathway(s) comprises designer genes that can express at least one of the enzymes selected from the group consisting of: energy converting hydrogenase (Ech) 91, methyl-H4MPT: coenzyme-M methyltransferase Mtr 92, methyl-coenzyme M reductase Mcr 93, heterodissulfide reductase (Hdr) 94, [NiFe]-hydrogenase (Mvh) 95, Coenzyme F₄₂₀-reducing hydrogenase (Frh) 96, soluble hydrogenase (SH) 71, A₁Aₒ-ATP synthase 97, formate dehydroganse 74, 10-formyl-H₄ folate synthetase 75, methenyltetrahydrofolate cyclohydrolase 76, 10-methylene-H₄ folate dehydrogenase 77, 10-methylene-H₄ folate reductase 78, methyl-H₄ folate: corrinoid iron-sulfur protein methyltransferase 79, corrinoid iron-sulfur protein 80, CO dehydrogenase/acetyl-CoA synthase 81, formylmethanofuran dehydroganse 83, formyl transferase 84, 10-methenyl-tetrahydromethanopterin cyclohydrolase 85, 10-methylene-H₄ methanopterin dehydrogenase 86, 10-methylene-H₄-methanopterin reductase 87, methyl-H₄-methanopterin: corrinoid iron-sulfur protein methyltransferase 88, corrinoid iron-sulfur protein 89, CO dehydrogenase/acetyl-CoA synthase 90, thiolase 07, 3-hydroxybutyryl-CoA dehydrogenase 08, crotonase 09, butyryl-CoA dehydrogenase 10, butyaldehyde dehydrogenase 11, butanol dehydrogenase 12 and/or alcohol dehydrogenase 43.

SEQ ID NOS. 166-198 present examples for designer DNA constructs of designer enzymes for creation of designer hydrogenotrophic biofuel-producing organisms such as designer cyanobacteria with reductive-acetyl-CoA biofuel-production pathways. Briefly, SEQ ID NO: 166 presents example 166 of a designer hox-promoter-controlled Formylmethanofuran dehydrogenase (Fmd; 83) DNA construct (6110 bp) that includes a PCR FD primer (sequence 1-20), a 172-bp Nostoc (Anabaena PCC 7120) hox promoter (21-192), an enzyme-encoding sequence (193-5659) selected/modified from the sequence of formylmethanofuran dehydrogenase subunits B, C, E (GenBank: ADL58895, ADL58894, ADL58893) of Methanothermobacter marburgensis and formylmethanofuran dehydrogenase subunits subunits A, D, and G (GenBank: ABC56660, ABC56658, ABC56657) of Methanosphaera stadtmanae, a 432-bp Nostoc sp. strain PCC 7120 gor terminator (5659-6090), and a PCR RE primer (6091-6110) at the 3′ end.

SEQ ID NO: 167 presents example 167 of a designer hox-promoter-controlled Formyl transferase (84) DNA construct (1538 bp) that includes a PCR FD primer (sequence 1-20), a 172-bp Nostoc (Anabaena PCC 7120) hox promoter (21-192), an enzyme-encoding sequence (193-1086) selected/modified from the sequence of a formylmethanofuran-tetrahydromethanopterin formyltransferase (GenBank: ADL59225) of Methanothermobacter marburgensis, a 432-bp Nostoc gor terminator (1087-1518), and a PCR RE primer (1519-1538).

SEQ ID NO: 168 presents example 168 of a designer hox-promoter-controlled 5,10-Methenyl-tetrahydromethanopterin (H₄ methanopterin) cyclohydrolase (85) DNA construct (1631 bp) that includes a PCR FD primer (sequence 1-20), a 172-bp Anabaena PCC 7120 hox promoter (21-192), an enzyme-encoding sequence (193-1179) selected from the sequence of a N(5),N(10)-methenyltetrahydromethanopterin cyclohydrolase (GenBank: ABC57615) of Methanosphaera stadtmanae, a 432-bp Nostoc gor terminator (1180-1161), and a PCR RE primer (1162-1631).

SEQ ID NO: 169 presents example 169 of a designer hox-promoter-controlled 5,10-Methylene-H₄-methanopterin dehydrogenase (86) DNA construct (1475 bp) that includes a PCR FD primer (sequence 1-20), a 172-bp Anabaena PCC 7120 hox promoter (21-192), an enzyme-encoding sequence (193-1023) selected from the sequence of a F₄₂₀-dependent methylene-5,6,7,8-tetrahydromethanopterin dehydrogenase (GenBank: ADL57660) of Methanothermobacter marburgensis, a 432-bp Nostoc gor terminator (1023-1455), and a PCR RE primer (1456-1475).

SEQ ID NO: 170 presents example 170 of a designer hox-promoter-controlled Methylenetetrahydrofolate reductase and/or Methylene-H₄-methanopterin reductase (78, 87) DNA construct (2594 bp) that includes a PCR FD primer (sequence 1-20), a 172-bp Nostoc sp. strain PCC 7120 (Anabaena PCC 7120) hox promoter (21-192), an enzyme-encoding sequence (193-2142) selected/modified from the sequence of a methylenetetrahydrofolate reductase (GenBank: YP_{—}430048) of Moorella thermoacetica and a coenzyme F₄₂₀-dependent N(5),N(10)-methenyltetrahydromethanopterin reductase (GenBank: ADN36752) of Methanoplanus petrolearius, a 432-bp Nostoc gor terminator (2143-2574), and a PCR RE primer (2575-2594).

SEQ ID NO: 171 presents example 171 of a designer hox-promoter-controlled Methyltetrahydrofolate:corrinoid/iron-sulfur protein methyltransferase (79, 88) DNA construct (2819 bp) that includes a PCR FD primer (sequence 1-20), a 172-bp Nostoc (Anabaena PCC 7120) hox promoter (21-192), an enzyme-encoding sequence (193-2467) selected/modified from the sequence of a methyltetrahydrofolate:corrinoid/iron-sulfur protein methyltransferase (GenBank: YP_{—}430950) of Moorella thermoacetica, and acetyl-CoA decarbonylase/synthase, subunit gamma (GenBank: ADL57900) of Methanothermobacter marburgensis, a 432-bp Nostoc sp. strain PCC 7120 gor terminator (2468-2899), and a PCR RE primer (2900-2819).

SEQ ID NO: 172 presents example 172 of a designer hox-promoter-controlled Corrinoid iron-sulfur protein (80, 89) DNA construct (2771 bp) that includes a PCR FD primer (sequence 1-20), a 172-bp Nostoc (Anabaena PCC 7120) hox promoter (21-192), an enzyme-encoding sequence (193-2319) selected/modified from the sequence of a small subunit corrinoid iron-sulfur protein (GenBank: AAA23255) of Moorella thermoacetica, and acetyl-CoA decarbonylase/synthase subunit delta (GenBank: ADL57899) of Methanothermobacter marburgensis, a 432-bp Nostoc gor terminator (2319-2751), and a PCR RE primer (2752-2771).

SEQ ID NO: 173 presents example 173 of a designer hox-promoter-controlled CO dehydrogenase/acetyl-CoA synthase (81, 90) DNA construct (7061 bp) that includes a PCR FD primer (sequence 1-20), a 172-bp Nostoc (Anabaena PCC 7120) hox promoter (21-192), an enzyme-encoding sequence (193-6609) selected/modified from the sequence of acetyl-CoA decarbonylase/synthase beta subunit/acetyl-CoA decarbonylase/synthase alpha subunit (GenBank: ABC19516) of Moorella thermoacetica, and acetyl-CoA decarbonylase/synthase subunits alpha, beta, epsilon (GenBank: ADL57895, ADL59006, ADL57897) of Methanothermobacter marburgensis, a 432-bp Nostoc sp. strain PCC 7120 gor terminator (6610-7041), and a PCR RE primer (7042-7061).

SEQ ID NO: 174 presents example 174 of a designer hox-promoter-controlled Thiolase (07) DNA construct (1847 bp) that includes a PCR FD primer (sequence 1-20), a 172-bp Nostoc (Anabaena PCC 7120) hox promoter (21-192), an enzyme-encoding sequence (193-1395) selected/modified from the sequence of thiolase (GenBank: AB190764) of Butyrivibrio fibrisolvens, a 432-bp Nostoc gor terminator (1396-1827), and a PCR RE primer (1828-1847).

SEQ ID NO: 175 presents example 175 of a designer hox-promoter-controlled 3-Hydroxybutyryl-CoA dehydrogenase (08) DNA construct (1514 bp) that includes a PCR FD primer (sequence 1-20), a 172-bp Nostoc (Anabaena PCC 7120) hox promoter (21-192), an enzyme-encoding sequence (193-1062) selected/modified from the sequence of 3-hydroxybutyryl coenzyme A dehydrogenase (GenBank: Z92974) of Thermoanaerobacterium, a 432-bp Nostoc gor terminator (1063-1494), and a PCR RE primer (1495-1514).

SEQ ID NO: 176 presents example 176 of a designer hox-promoter-controlled Crotonase (09) DNA construct (1430 bp) that includes a PCR FD primer (sequence 1-20), a 172-bp Nostoc (Anabaena PCC 7120) hox promoter (21-192), an enzyme-encoding sequence (193-978) selected from the sequence of crotonase (GenBank: AF494018) of Clostridium beijerinckii, a 432-bp Nostoc gor terminator (979-1410), and a PCR RE primer (1411-1430).

SEQ ID NO: 177 presents example 177 of a designer hox-promoter-controlled Butyryl-CoA dehydrogenase (10) DNA construct (1784 bp) that includes a PCR FD primer (sequence 1-20), a 172-bp Nostoc (Anabaena PCC 7120) hox promoter (21-192), an enzyme-encoding sequence (193-1332) selected/modified from the sequence of butyryl-CoA dehydrogenase (GenBank: AF494018) of Clostridium beijerinckii, a 432-bp Nostoc gor terminator (1333-1764), and a PCR RE primer (1765-1784).

SEQ ID NO: 178 presents example 178 of a designer hox-promoter-controlled Butyraldehyde dehydrogenase (11) DNA construct (2051 bp) that includes a PCR FD primer (sequence 1-20), a 172-bp Nostoc (Anabaena PCC 7120) hox promoter (21-192), an enzyme-encoding sequence (193-1599) selected/modified from the sequence of butyraldehyde dehydrogenase (GenBank: AY251646) of Clostridium saccharoperbutylacetonicum, a 432-bp Nostoc gor terminator (1600-2031), and a PCR RE primer (2032-2051).

SEQ ID NO: 179 presents example 179 of a designer hox-promoter-controlled NADH-dependent Butanol dehydrogenase (12) DNA construct (1808 bp) that includes a PCR FD primer (sequence 1-20), a 172-bp Nostoc (Anabaena PCC 7120) hox promoter (21-192), an enzyme-encoding sequence (193-1356) selected/modified from the sequence of NADH-dependent butanol dehydrogenase (GenBank: YP_{—}148778) of Geobacillus kaustophilus, a 432-bp Nostoc sp. strain PCC 7120 gor terminator (1367-1788), and a PCR RE primer (1789-1808) at the 3′ end.

Note, use of SEQ ID NOS. 166-179 in genetic transformation of a microbial host cell including (but not limited to) bacterial cells such as a cyanobacterium Anabaena PCC 7120 can create a designer cyanobacterium such as designer Anabaena with a designer reductive-acetyl-CoA biofuel-production pathway (numerically labeled as 83-90 and 07-12 in FIG. 13) for production of 1-butanol from hydrogen and carbon dioxide without requiring photosynthesis or sunlight. That is, the expression of SEQ ID NOS. 166-179 in a bacterium such as Anabaena PCC 7120 represents a designer organism with the designer hydrogenotrophic reductive-acetyl-CoA biofuel-production pathway (83-90 and 07-12 in FIG. 13) that can operate for anaerobic chemolithoautotrophic production of butanol from hydrogen and carbon dioxide even if it is in complete darkness.

SEQ ID NO: 180 presents example 180 of a designer hox-promoter-controlled Energy converting hydrogenase (Ech) (91) DNA construct (10538 bp) that includes a PCR FD primer (sequence 1-20), a 172-bp Nostoc (Anabaena PCC 7120) hox promoter (21-192), an enzyme-encoding sequence (193-10086) selected/modified from the sequence of Energy converting hydrogenase subunits (EchA, B, C, D, E. F, G, H, I, J, K, L, M, N, O, P, Q) (GenBank: ABC57807, and ABC57812-ABC57827) of Methanosphaera stadtmanae DSM 3091, a 432-bp Nostoc gor terminator (10087-10518), and a PCR RE primer (10519-10538).

SEQ ID NO: 181 presents example 181 of a designer hox-promoter-controlled [NiFe]-hydrogenase MvhADG (95) DNA construct (3416 bp) that includes a PCR FD primer (sequence 1-20), a 172-bp Nostoc (Anabaena PCC 7120) hox promoter (21-192), an enzyme-encoding sequence (193-2964) selected/modified from the sequence of [NiFe]-hydrogenase MvhADG (GenBank: ADL59096, ADL59098, ADL59097) of Methanothermobacter marburgensis, a 432-bp Nostoc sp. strain PCC 7120 gor terminator (2965-3396), and a PCR RE primer (3397-3416).

SEQ ID NO: 182 presents example 182 of a designer hox-promoter-controlled Heterodisulfide reductases (HdrABC, HdrDE) (94) DNA construct (6695 bp) that includes a PCR FD primer (sequence 1-20), a 172-bp Anabaena PCC 7120 hox promoter (21-192), an enzyme-encoding sequence (193-6243) selected/modified from the sequence of Heterodisulfide reductases (HdrABC, HdrDE) (GenBank: AET63985, AET63982, AET63983, AET64166, AET64165) of Methanosaeta harundinacea, a 432-bp Nostoc gor terminator (6244-6675), and a PCR RE primer (6676-6695).

SEQ ID NO: 183 presents example 183 of a designer hox-promoter-controlled Coenzyme F₄₂₀-reducing hydrogenase (Frh) (96) DNA construct (3407 bp) that includes a PCR FD primer (sequence 1-20), a 172-bp Nostoc sp. strain PCC 7120 (Anabaena PCC 7120) hox promoter (21-192), an enzyme-encoding sequence (193-2955) selected/modified from the sequence of Coenzyme F₄₂₀-reducing hydrogenase (FrhB1-3) (GenBank: YP_{—}003357229, YP_{—}003357467, YP_{—}003357509) of Methanocella paludicola SANAE, a 432-bp Nostoc sp. strain PCC 7120 gor terminator (2956-3387), and a PCR RE primer (3388-3407) at the 3′ end.

Note, use of SEQ ID NOS. 180-183 in genetic transformation of a microbial host cell including (but not limited to) bacterial cells such as a cyanobacterium Anabaena PCC 7120 can confer an anaerobic chemolithoautotrophic hydrogen (H₂) utilization system [which, as shown in FIG. 12, comprises Energy converting hydrogenase (Ech) (91), [NiFe]-hydrogenase MvhADG (95), Coenzyme F₄₂₀-reducing hydrogenase (Frh) (96), and Coenzyme F₄₂₀-reducing hydrogenase (Frh) (96)] that can produce reducing power (Fd_{red}²⁻ and F₄₂₀H₂) from H₂ in support of the designer reductive-acetyl-CoA butanol-production pathway (83-90 and 07-12 in FIG. 13). Therefore, the expression of SEQ ID NOS. 180-183 along with SEQ ID NOS. 166-179 in a bacterium such as Anabaena PCC 7120 represents a designer organism (such as designer Anabaena) with a full designer reductive-acetyl-CoA biofuel-production pathway system (FIGS. 12 and 13) that can operate for anaerobic chemolithoautotrophic production of butanol from hydrogen and carbon dioxide without requiring photosynthesis or aerobic respiration. The net result in this example is the anaerobic chemolithoautotrophic production of butanol from hydrogen and carbon dioxide as shown in the process equation [21].

Also note, these designer genes (SEQ ID NOS. 166-183) are controlled by a designer hox anaerobic promoter. Therefore, under aerobic conditions such as in an open pond mass culture, the designer Anabaena in this example can quickly grow photoautotrophically using air carbon dioxide and water as the sources of carbon and electrons just like the wild-type parental strain. When the designer Anabaena cells cultures are grown and ready for use (as catalysts in this application), they can then be placed into an anaerobic reactor supplied with industrial CO₂ and H₂ gas for induction of the designer genes expression for anaerobic chemolithoautotrophic production of butanol (as shown in FIGS. 12 and 13) in dark.

SEQ ID NO: 184 presents example 184 of a designer hox-promoter-controlled Methyl-H4MPT: coenzyme M methyltransferase (MtrA-H) (92) DNA construct (5417 bp) that includes a PCR FD primer (sequence 1-20), a 172-bp Anabaena PCC 7120 hox promoter (21-192), an enzyme-encoding sequence (193-4965) selected/modified from the sequence of Methyl-H4MPT: coenzyme M methyltransferase (MtrA-H) (GenBank: ABC56714, ABC56713,YP_{—}447360, YP_{—}447354, YP_{—}447359,YP_{—}447355) of Methanosphaera stadtmanae, and mtrEF (AET65445, NC_{—}009051) of Methanosaeta harundinacea and Methanoculleus marisnigri, a 432-bp Nostoc sp. strain PCC 7120 gor terminator (4966-5397), and a PCR RE primer (5398-5417).

SEQ ID NO: 185 presents example 185 of a designer hox-promoter-controlled Methyl-coenzyme M reductase (Mcr) (93) DNA construct (5042 bp) that includes a PCR FD primer (sequence 1-20), a 172-bp Nostoc sp. strain PCC 7120 (Anabaena PCC 7120) hox promoter (21-192), an enzyme-encoding sequence (193-4590) selected/modified from the sequence of methylcoenzyme M reductase subunits A, B. C, G (GenBank: CAE48306, CAE48303, ABC56709, CAE48305) of Methanosphaera stadtmanae, a 432-bp Nostoc sp. strain PCC 7120 gor terminator (4591-5022), and a PCR RE primer (5023-5042).

Note, use of SEQ ID NOS. 184 and 185 along with SEQ ID NOS. 180-183 in genetic transformation of a microbial host cell including bacterial cells such as a cyanobacterium Anabaena PCC 7120 can confer a methanogenic hydrogenotrophic system which, as shown in FIG. 15, comprises Methyl-H4MPT: coenzyme M methyltransferase (MtrA-H) (92), Methyl-coenzyme M reductase (Mcr) (93), Energy converting hydrogenase (Ech) (91), [NiFe]-hydrogenase MvhADG (95), Coenzyme F₄₂₀-reducing hydrogenase (Frh) (96), Coenzyme F₄₂₀-reducing hydrogenase (Frh) (96). These enzymes along with a native ATPase 97 can produce ATP and reducing power (Fd_{red}²⁻ and F₄₂₀H₂) from H₂ in support of the designer reductive-acetyl-CoA methanogenic butanol-production pathways (FIGS. 16 and 17). Therefore, the expression of SEQ ID NOS. 180-185 along with SEQ ID NOS. 166-179 in a bacterium such as Anabaena PCC 7120 represents a designer organism (such as designer Anabaena) with a designer hydrogenotrophic reductive-acetyl-CoA methanogenic biofuel-production pathway system (FIGS. 15 and 17) that can operate for anaerobic production of both butanol and methane from hydrogen and carbon dioxide without requiring any photosynthesis. The net result in this example is the anaerobic chemolithoautotrophic production of butanol and methane from hydrogen and carbon dioxide as shown in the process equation [22].

SEQ ID NO: 186 presents example 186 of a designer hox-promoter-controlled Formate dehydrogenase (74) DNA construct (5450 bp) that includes a PCR FD primer (sequence 1-20), a 172-bp Nostoc sp. strain PCC 7120 (Anabaena PCC 7120) hox promoter (21-192), an enzyme-encoding sequence (193-4998) selected/modified from the sequence of formate dehydrogenase alpha and beta subunits (GenBank: AAB18330, AAB18329) of Moorella thermoacetica, a 432-bp Nostoc sp. strain PCC 7120 gor terminator (4999-5430), and a PCR RE primer (5431-5450).

SEQ ID NO: 187 presents example 187 of a designer hox-promoter-controlled 10-Formyl-H₄ folate synthetase (75) DNA construct (2324 bp) that includes a PCR FD primer (sequence 1-20), a 172-bp Nostoc (Anabaena PCC 7120) hox promoter (21-192), an enzyme-encoding sequence (193-1872) selected/modified from the sequence of 10-formyltetrahydrofolate synthetase (GenBank: YP_{—}428991) of Moorella thermoacetica, a 432-bp Nostoc sp. strain PCC 7120 gor terminator (1873-2304), and a PCR RE primer (2305-2324).

SEQ ID NO: 188 presents example 188 of a designer hox-promoter-controlled 10-Methenyl-H₄ folate cyclohydrolase (76) DNA construct (1487 bp) that includes a PCR FD primer (sequence 1-20), a 172-bp Nostoc (Anabaena PCC 7120) hox promoter (21-192), an enzyme-encoding sequence (193-1035) selected/modified from the sequence of methenyltetrahydrofolate cyclohydrolase (GenBank: YP_{—}430368) of Moorella thermoacetica ATCC 39073, a 432-bp Nostoc gor terminator (1036-1467), and a PCR RE primer (1468-1487).

SEQ ID NO: 189 presents example 189 of a designer hox-promoter-controlled 10-Methylene-H₄ folate dehydrogenase (77) DNA construct (1487 bp) that includes a PCR FD primer (sequence 1-20), a 172-bp Nostoc (Anabaena PCC 7120) hox promoter (21-192), an enzyme-encoding sequence (193-1035) selected/modified from the sequence of methenyltetrahydrofolate cyclohydrolase/5,10-methylenetetrahydrofolate dehydrogenase (GenBank: ABC19825) of Moorella thermoacetica, a 432-bp Nostoc sp. strain PCC 7120 gor terminator (1036-1467), and a PCR RE primer (1468-1487).

SEQ ID NO: 190 presents example 190 of a designer hox-promoter-controlled 10-Methylene-H₄ folate reductase (78) DNA construct (1565 bp) that includes a PCR FD primer (sequence 1-20), a 172-bp Nostoc (Anabaena PCC 7120) hox promoter (21-192), an enzyme-encoding sequence (193-1113) selected/modified from the sequence of methylenetetrahydrofolate reductase (GenBank: ABC19505) of Moorella thermoacetica, a 432-bp Nostoc gor terminator (1114-1545), and a PCR RE primer (1546-1565).

SEQ ID NO: 191 presents example 191 of a designer hox-promoter-controlled Methyl-H₄ folate: corrinoid iron-sulfur protein Methyltransferase (79) DNA construct (1442 bp) that includes a PCR FD primer (sequence 1-20), a 172-bp Anabaena PCC 7120 hox promoter (21-192), an enzyme-encoding sequence (193-690) selected/modified from the sequence of methyltetrahydrofolate:corrinoid/iron-sulfur protein methyltransferase (GenBank: YP_{—}430174) of Moorella thermoacetica, a 432-bp Nostoc gor terminator (691-1122), and a PCR RE primer (1123-1442).

SEQ ID NO: 192 presents example 192 of a designer hox-promoter-controlled Corrinoid iron-sulfur protein (80) DNA construct (2942 bp) that includes a PCR FD primer (sequence 1-20), a 172-bp Anabaena hox promoter (21-192), an enzyme-encoding sequence (193-2490) selected/modified from the sequence of corrinoid iron-sulfur protein large and small subunits (GenBank: AEI90745, AEI90746) of Clostridium autoethanogenum, a 432-bp Nostoc sp. strain PCC 7120 gor terminator (2491-2922), and a PCR RE primer (2923-2942).

SEQ ID NO: 193 presents example 193 of a designer hox-promoter-controlled CO dehydrogenase/acetyl-CoA synthase (81) DNA construct (4859 bp) that includes a PCR FD primer (sequence 1-20), a 172-bp Anabaena PCC 7120 hox promoter (21-192), an enzyme-encoding sequence (193-4407) selected/modified from the sequence of carbon monoxide dehydrogenase alpha subunit alpha and beta subunits (GenBank: AAA23229, AAA23228) of Moorella thermoacetica, a 432-bp Nostoc gor terminator (4408-4839), and a PCR RE primer (4840-4859).

Note, use of SEQ ID NOS. 186-193 along with SEQ ID NOS. 174-179 in genetic transformation of a microbial host cell such as a cyanobacterium Anabaena PCC 7120 confers an ATP-requiring reductive-acetyl-CoA butanol-production pathway (74-81 and 07-12/42 in FIG. 14). Similarly, the expression of SEQ ID NOS. 186-193 and SEQ ID NOS. 180-185 along with SEQ ID NOS. 174-179 in a bacterium such as Anabaena PCC 7120 represents a designer organism (such as designer Anabaena) with a designer ATP-requiring reductive-acetyl-CoA methanogenic biofuel-production pathway and a hydrogenotrophic methanogenesis-coupled ATP-generating system (FIGS. 15 and 16) that can operate for production of both butanol and methane from hydrogen and carbon dioxide. The net result in this example is the anaerobic chemolithotrophic production of both butanol and methane from hydrogen and carbon dioxide as shown in the process equation [22].

SEQ ID NO: 194 presents example 194 of a designer hox-promoter-controlled F₄₂₀ synthesis enzymes (99) DNA construct (6428 bp) that includes a PCR FD primer (sequence 1-20), a 172-bp Anabaena PCC 7120 hox promoter (21-192), enzymes-encoding sequence (193-4976) selected/modified from the sequence of lactaldehyde dehydrogenase CofA (GenBank: ADC46523,) of Methanobrevibacter ruminantium, 2-phospho-l-lactate guanylyltransferase (GenBank: ADL58588) of Methanothermobacter Marburgensis, 2-phospho-L-lactate transferase (GenBank: NP_{—}987524) of Methanococcus maripaludis, coenzyme F420-0 gamma-glutamyl ligase (YP_{—}001030766) of Methanocorpusculum labreanum, FO synthase subunits 1 and 2 (YP_{—}003357513, YP_{—}003357511) of Methanocella paludicolam, a 432-bp Nostoc sp. strain PCC 7120 gor terminator (4977-6408), and a PCR RE primer (6409-6428).

SEQ ID NO: 195 presents example 195 of a designer hox-promoter-controlled Pyridoxal phosphate-dependent L-tyrosine decarboxylase(mfnA for methanofuran synthesis) (100) DNA construct (1778 bp) that includes a PCR FD primer (sequence 1-20), a 172-bp Anabaena PCC 7120 hox promoter (21-192), an enzyme-encoding sequence (193-1326) selected/modified from the sequence of L-tyrosine decarboxylase (GenBank: YP_{—}003355454) of Methanocella paludicola, a 432-bp Nostoc gor terminator (1327-1758), and a PCR RE primer (1759-1778).

SEQ ID NO: 196 presents example 196 of a designer hox-promoter-controlled Methanopterin synthesis enzymes (101) DNA construct (3215 bp) that includes a PCR FD primer (sequence 1-20), a 172-bp Anabaena PCC 7120 hox promoter (21-192), an enzymes-encoding sequence (193-2763) selected/modified from the sequence of GTP cyclohydrolase (GenBank: YP_{—}447347) of Methanosphaera stadtmanae DSM 3091, cyclic phosphodiesterase MptB (AB035741) of Methanococcus maripaludis C5, beta-ribofuranosylaminobenzene 5′-phosphate synthase (YP_{—}003356610) of Methanocella paludicola SANAE, a 432-bp Nostoc sp. strain PCC 7120 gor terminator (2764-3195), and a PCR RE primer (3195-3215).

SEQ ID NO: 197 presents example 197 of a designer hox-promoter-controlled Coenzyme M synthesis enzymes (102) DNA construct (4226 bp) that includes a PCR FD primer (sequence 1-20), a 172-bp Nostoc sp. strain PCC 7120 (Anabaena PCC 7120) hox promoter (21-192), an enzymes-encoding sequence (193-3774) selected/modified from the sequence of phosphosulfolactate synthase, 2-phosphosulfolactate phosphatase and sulfolactate dehydrogenase (GenBank: ADL57861, YP_{—}003850451, ADL59162) of Methanothermobacter marburgensis, and sulfopyruvate decarboxylase (YP_{—}003357048) of Methanocella paludicola SANAE, a 432-bp Nostoc sp. strain PCC 7120 gor terminator (3775-4026), and a PCR RE primer (4027-4226).

SEQ ID NO: 198 presents example 198 of a designer hox-promoter-controlled Coenzyme B synthesis enzymes (103) DNA construct (5198 bp) that includes a PCR FD primer (sequence 1-20), a 172-bp Anabaena PCC 7120 hox promoter (21-192), an enzymes-encoding sequence (193-4746) selected/modified from the sequence of isopropylmalate synthase, isopropylmalate dehydrogenase (GenBank: AAM01606, NP_{—}614498) of Methanopyrus kandleri, isopropylmalate isomerase large and small subunits (ADP98363, ADP98362) of Marinobacter adhaerens, a 432-bp Nostoc gor terminator (4747-5178), and a PCR RE primer (5179-5198).

Note, the expression of SEQ ID NOS. 194-198 in a microbial host cell such as cyanobacterium Anabaena PCC 7120 provides the ability of synthesizing some of the cofactors such as F₄₂₀, methanofuran, methanopterin, Coenzyme M, and Coenzyme B that are needed for the designer hydrogenotrophic reductive-acetyl-CoA biofuel-production pathways (of FIGS. 13,14,16 and 17) to properly operate. Depending on the genetic backgrounds of various host cells such as cyanobacteria, many of them may or may not possess some of these enzymes to synthesize this type of special cofactors. Therefore, in one of the various embodiments, it is a preferred practice to express this type of designer cofactor-synthesis enzymes (e.g., SEQ ID NOS. 194-198) along with the hydrogenotrophic designer reductive-acetyl-CoA biofuel-production pathway genes (e.g., SEQ ID NOS. 166-193) as shown in these examples.

Note, many of the hydrogenotrophic bacteria and methanogens such as Methanocella paludicola SANAE naturally possess certain hydrogenotrophic and/or reductive acetyl-CoA pathway(s) and the ability of synthesizing the associated cofactors including F420, methanofuran, methanopterin, Coenzyme M, and Coenzyme B. Therefore, in one of the various embodiments, it is also a preferred practice to express certain designer genes of biofuel-production-pathways (FIGS. 1, 4, 5, 6, 7, 8, 10, 13, and 14) such as SEQ ID NOS. 174-179 in a hydrogenotrophic and/or methanogenic host cell for chemolithotrophic production of advanced biofuels such as 1-buatanol from hydrogen (H₂) and carbon dioxide (CO₂). According to one of the various embodiments, a hydrogenotrophic and/or methanogenic host organism for this specific application is selected from the group consisting of: Methanocella paludicola SANAE, Acinetobacter baumannii ABNIH3, Acinetobacter baumannii ABNIH4, Acinetobacter sp. DR1, Agrobacterium sp. H13-3; Agrobacterium vitis S4, Alcaligenes sp., Allochromatium vinosum DSM 180, Amycolatopsis mediterranei S699, Anoxybacillus flavithermus WK1, Aquifex aeolicus VF5, Archaeoglobus fulgidus DSM 4304, Archaeoglobus veneficus SNP6, Azospirillum sp. B510, Burkholderia cenocepacia HI2424, Caldicellulosiruptor bescii DSM 6725, Carboxydothermus hydrogenoformans, Centipeda periodontii DSM 2778, Clostridium autoethanogenum, Clostridium ragsdalei, Clostridium sticklandii DSM 519, Clostridium sticklandii, Corynebacterium glutamicum, Cupriavidus metallidurans CH34, Cupriavidus necator N-1, Desulfobacca acetoxidans DSM 11109, Exiguobacterium sp. AT1b, Ferrimonas balearica DSM 9799, Ferroglobus placidus DSM 10642, Geobacillus kaustophilus HTA426, Helicobacter bilis ATCC 43879, Herbaspirillum seropedicae SmR1, Hydrogenobacter thermophilus TK-6,

Hydrogenovibrio marinus, Klebsiella variicola At-22, Methanobacterium sp. SWAN-1, Methanobrevibacter ruminantium M1, Methanocaldococcus fervens AG86, Methanocaldococcus infernus ME, Methanocaldococcus jannaschii, Methanocaldococcus sp. FS406-22, Methanocaldococcus vulcanius M7, Methanococcus aeolicus Nankai-3, Methanococcus maripaludis C6, Methanococcus maripaludis S2, Methanococcus voltae A3, Methanocorpusculum labreanum Z, Methanoculleus marisnigri JR1, Methanohalophilus mahii DSM 5219, Methanolinea tarda NOBI-1, Methanoplanus petrolearius DSM 11571, Methanoplanus petrolearius, Methanopyrus kandleri AV19, Methanoregula boonei 6A8, Methanosaeta harundinacea 6Ac, Methanosalsum zhilinae DSM 4017, Methanosarcina acetivorans C2A, Methanosarcina barkeri str. Fusaro, Methanosarcina mazei Go1, Methanosphaera stadtmanae, Methanospirillum hungatei JF-1, Methanothermobacter marburgensis str. Marburg, Methanothermobacter marburgensis, Methanothermobacter thermautotrophicus, Methanothermococcus okinawensis IH1, Methanothermus fervidus DSM 2088, Methylobacillus flagellates, Methylobacterium organophilum, Methylococcus capsulatus, Methylomicrobium kenyense, Methylomonas methanica MC09, Methylomonas sp. LW13, Methylosinus sp. LW2, Methylosinus trichosporium OB3b, Methylotenera mobilis JLW8, Methylotenera versatilis 301, Methylovorus glucosetrophus SIP3-4, Moorella thermoacetica ATCC 39073, Moorella thermoacetica, Oligotropha carboxidovorans OM5, Paenibacillus terse HPL-003, Pelotomaculum thermopropionicum SI Planctomyces brasiliensis DSM 5305, Pyrococcus furiosus DSM 3638, Pyrococcus horikoshii OT3, Pyrococcus yayanosii CH1, Ralstonia eutropha H16, Rubrivivax sp., Selenomonas noxia ATCC 43541, Shewanella baltica BA175, Stenotrophomonas sp. SKA14, Synechococcus sp. JA-2-3B′ a(2-13), Synechococcus sp. JA-3-3Ab, Thermococcus gammatolerans EJ3, Thermococcus kodakarensis KOD1, Thermococcus onnurineus NA1, Thermococcus sp. 4557, Thermodesulfatator indicus DSM 15286, Thermofilum pendens Hrk 5, Thermotoga lettingae TMO, Thermotoga petrophila RKU-1, Thiocapsa roseopersicina, Thiomonas intermedia K12, Xanthobacter autotrophicus, Yersinia pestis Antigua, and combinations thereof.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

1. A method for autotrophic production of butanol and related higher alcohols comprising:
introducing a transgenic autotrophic organism into a reactor system, the transgenic autotrophic organism comprising transgenes coding for a set of enzymes configured to confer a hydrogenotrophic pathway for production of a higher alcohol comprising at least four carbon atoms;
using reducing power such as NADPH, reduced ferredoxin, and energy ATP associated with the transgenic autotrophic organism acquired from hydrogenotrophic process in the biological reactor to synthesize the higher alcohol from carbon dioxide and water; and
using a product separation process to harvest the synthesized alcohol from the photobioreactor.

2. The method of claim 1, wherein:
said designer transgenic autotrophic organism comprises at least one of designer Calvin-cycle-channeled pathways and designer hydrogenotrophic pathways for producing at least one of the higher alcohols selected from the group consisting of: 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, 6-methyl-1-heptanol and combinations thereof.

3. The method of claim 1, wherein the transgenic autotrophic organism comprises at least one of a transgenic designer plant or transgenic designer plant cell, or bacterial cell selected from the group consisting of blue-green algae (oxyphotobacteria including cyanobacteria and oxychlorobacteria), hydrogenotrophic bacteria, methanogens, aquatic plants, plant cells, green algae, red algae, brown algae, diatoms, marine algae, freshwater algae, salt-tolerant algal strains, cold-tolerant algal strains, heat-tolerant algal strains, antenna-pigment-deficient mutants, butanol-tolerant algal strains, higher-alcohols-tolerant algal strains, butanol-tolerant oxyphotobacteria, butanol-tolerant hydrogenotrophic bacteria and methanogens, higher-alcohols-tolerant oxyphotobacteria and hydrogenotrophic bacteria or methanogens.

4. The method of claim 1, wherein said transgenic autotrophic organism comprises a set of designer genes that express a designer anaerobic hydrogenotrophic butanol-production-pathway system comprising: energy converting hydrogenase (Ech), [NiFe]-hydrogenase (Mvh), Coenzyme F₄₂₀-reducing hydrogenase (Frh), native (or heterologous) soluble hydrogenase (SH), heterodissulfide reductase (Hdr), formylmethanofuran dehydroganse, formyl transferase, 10-methenyl-tetrahydromethanopterin cyclohydrolase, 10-methylene-H₄ methanopterin dehydrogenase, 10-methylene-H₄-methanopterin reductase, methyl-H₄-methanopterin: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, and butanol dehydrogenase.

5. The method of claim 1, wherein the transgenic autotrophic organism comprises bacteria selected from the group consisting of Thermosynechococcus elongatus BP-1, Nostoc sp. PCC 7120, Synechococcus elongatus PCC 6301, Syncechococcus sp. strain PCC 7942, Syncechococcus sp. strain PCC 7002, Syncechocystis sp. strain PCC 6803, Prochlorococcus marinus MED4, Prochlorococcus marinus MIT 9313, Prochlorococcus marinus NATL1A, Prochlorococcus SS120, Spirulina platensis (Arthrospira platensis), Spirulina pacifica, Lyngbya majuscule, Anabaena sp., Synechocystis sp., Synechococcus elongates, Synechococcus (MC-A), Trichodesmium sp., Richelia intracellularis, Synechococcus WH7803, Synechococcus WH8102, Nostoc punctiforme, Syncechococcus sp. strain PCC 7943, Synechocyitis PCC 6714 phycocyanin-deficient mutant PD-1, Cyanothece strain 51142, Cyanothece sp. CCY0110, Oscillatoria limosa, Lyngbya majuscula, Symploca muscorum, Gloeobacter violaceus, Prochloron didemni, Prochlorothrix hollandica, Synechococcus (MC-A), Trichodesmium sp., Richelia intracellularis, Prochlorococcus marinus, Prochlorococcus SS120, Synechococcus WH8102, Lyngbya majuscula, Symploca muscorum, Synechococcus bigranulatus, cryophilic Oscillatoria sp., Phormidium sp., Nostoc sp.-1, Calothrix parietina, thermophilic Synechococcus bigranulatus, Synechococcus lividus, thermophilic Mastigocladus laminosus, Chlorogloeopsis fritschii PCC 6912, Synechococcus vulcanus, Synechococcus sp. strain MA4, Synechococcus sp. strain MA19, Methanocella paludicola SANAE, Acinetobacter baumannii ABNIH3, Acinetobacter baumannii ABNIH4, Acinetobacter sp. DR1, Agrobacterium sp. H13-3; Agrobacterium vitis S4, Alcaligenes sp., Allochromatium vinosum DSM 180, Amycolatopsis mediterranei S699, Anoxybacillus flavithermus WK1, Aquifex aeolicus VF5, Archaeoglobus fulgidus DSM 4304, Archaeoglobus veneficus SNP6, Azospirillum sp. B510, Burkholderia cenocepacia HI2424, Caldicellulosiruptor bescii DSM 6725, Carboxydothermus hydrogenoformans, Centipeda periodontii DSM 2778, Clostridium autoethanogenum, Clostridium ragsdalei, Clostridium sticklandii DSM 519, Clostridium sticklandii, Corynebacterium glutamicum, Cupriavidus metallidurans CH34, Cupriavidus necator N-1, Desulfobacca acetoxidans DSM 11109, Exiguobacterium sp. AT1b, Ferrimonas balearica DSM 9799, Ferroglobus placidus DSM 10642, Geobacillus kaustophilus HTA426, Helicobacter bilis ATCC 43879, Herbaspirillum seropedicae SmR1, Hydrogenobacter thermophilus TK-6, Hydrogenovibrio marinus, Klebsiella variicola At-22, Methanobacterium sp. SWAN-1, Methanobrevibacter ruminantium M1, Methanocaldococcus fervens AG86, Methanocaldococcus infernus ME, Methanocaldococcus jannaschii, Methanocaldococcus sp. FS406-22, Methanocaldococcus vulcanius M7, Methanococcus aeolicus Nankai-3, Methanococcus maripaludis C6, Methanococcus maripaludis S2, Methanococcus voltae A3, Methanocorpusculum labreanum Z, Methanoculleus marisnigri JR1, Methanohalophilus mahii DSM 5219, Methanolinea tarda NOBI-1, Methanoplanus petrolearius DSM 11571, Methanoplanus petrolearius, Methanopyrus kandleri AV19, Methanoregula boonei 6A8, Methanosaeta harundinacea 6Ac, Methanosalsum zhilinae DSM 4017, Methanosarcina acetivorans C2A, Methanosarcina barkeri str. Fusaro, Methanosarcina mazei Go1, Methanosphaera stadtmanae, Methanospirillum hungatei JF-1, Methanothermobacter marburgensis str. Marburg, Methanothermobacter marburgensis, Methanothermobacter thermautotrophicus, Methanothermococcus okinawensis IH1, Methanothermus fervidus DSM 2088, Methylobacillus flagellates, Methylobacterium organophilum, Methylococcus capsulatus, Methylomicrobium kenyense, Methylomonas methanica MC09, Methylomonas sp. LW13, Methylosinus sp. LW2, Methylosinus trichosporium OB3b, Methylotenera mobilis JLW8, Methylotenera versatilis 301, Methylovorus glucosetrophus SIP3-4, Moorella thermoacetica ATCC 39073, Moorella thermoacetica, Oligotropha carboxidovorans OM5, Paenibacillus terse HPL-003, Pelotomaculum thermopropionicum SI, Planctomyces brasiliensis DSM 5305, Pyrococcus furiosus DSM 3638, Pyrococcus horikoshii OT3, Pyrococcus yayanosii CH1, Ralstonia eutropha H16, Rubrivivax sp., Selenomonas noxia ATCC 43541, Shewanella baltica BA175, Stenotrophomonas sp. SKA14, Synechococcus sp. JA-2-3B′ a(2-13), Synechococcus sp. JA-3-3Ab, Thermococcus gammatolerans EJ3, Thermococcus kodakarensis KOD1, Thermococcus onnurineus NA1, Thermococcus sp. 4557, Thermodesulfatator indicus DSM 15286, Thermofilum pendens Hrk 5, Thermotoga lettingae TMO, Thermotoga petrophila RKU-1, Thiocapsa roseopersicina, Thiomonas intermedia K12, Xanthobacter autotrophicus, Yersinia pestis Antigua, and Thermosynechococcus elongatus.

6. The method of claim 1, wherein the transgenic autotrophic organism comprises a biosafety-guarded feature selected from the group consisting of a designer proton-channel gene inducible under pre-determined inducing conditions, a designer cell-division-cycle iRNA gene inducible under pre-determined inducing conditions, a high-CO₂-requiring mutant as a host organism for transformation with designer biofuel-production-pathway genes in creating designer cell-division-controllable autotrophic organisms, and combinations thereof; and wherein said transgenic autotrophic organism comprises a set of designer genes exemplified with exemplary designer DNA constructs of SEQ ID NOS. 1-198 shown in the sequence listings for expressing at least one of the enzymes selected from the group consisting of oxygen-tolerant soluble hydrogenase (SH), oxygen-tolerant membrane bound hydrogenase (MBH), energy converting hydrogenase (Ech), methyl-H4MPT: coenzyme-M methyltransferase (Mtr), methyl-coenzyme M reductase (Mcr), heterodissulfide reductase (Hdr), [NiFe]-hydrogenase (Mvh), Coenzyme F₄₂₀-reducing hydrogenase (Frh), A₁Aₒ-ATP synthase, formate dehydroganse, 10-formyl-H₄ folate synthetase, methenyltetrahydrofolate cyclohydrolase, 10-methylene-H₄ folate dehydrogenase, 10-methylene-H₄ folate reductase, methyl-H₄ folate: corrinoid iron-sulfur protein methyltransferase, corrinoid iron-sulfur protein, CO dehydrogenase/acetyl-CoA synthase, formylmethanofuran dehydroganse, formyl transferase, 10-methenyl-tetrahydromethanopterin cyclohydrolase, 10-methylene-H₄ methanopterin dehydrogenase, 10-methylene-H₄-methanopterin reductase, methyl-H₄-methanopterin: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, butanol dehydrogenase, 2-keto acid decarboxylase, alcohol dehydrogenase, 2-methylbutyraldehyde reductase, 3-methylbutanal reductase, hexanol dehydrogenase, octanol dehydrogenase, and short-chain alcohol dehydrogenase.

7. The method of claim 1, wherein the set of enzymes comprises at least one of the enzymes selected from the group consisting of NADPH-dependent glyceraldehyde-3-phosphate dehydrogenase, NAD-dependent glyceraldehyde-3-phosphate dehydrogenase, phosphoglycerate mutase, enolase, pyruvate kinase, citramalate synthase, 2-methylmalate dehydratase, 3-isopropylmalate dehydratase, 3-isopropylmalate dehydrogenase, 2-isopropylmalate synthase, isopropylmalate isomerase, 2-keto acid decarboxylase, alcohol dehydrogenase, NADPH-dependent alcohol dehydrogenase, and butanol dehydrogenase.

8. The method of claim 1, wherein the set of enzymes comprises at least one of the enzymes conferring a designer anaerobic hydrogenotrophic system and butanol-production pathway selected from the group consisting of energy converting hydrogenase (Ech), [NiFe]-hydrogenase Mvh, Coenzyme F₄₂₀-reducing hydrogenase (Frh), soluble hydrogenase (SH), reduced ferredoxin (Fd_{red}²⁻), and heterodissulfide reductase (Hdr), NADPH-dependent glyceraldehyde-3-phosphate dehydrogenase, NAD-dependent glyceraldehyde-3-phosphate dehydrogenase, phosphoglycerate mutase, enolase, phosphoenolpyruvate carboxylase, aspartate aminotransferase, aspartokinase, aspartate-semialdehyde dehydrogenase, homoserine dehydrogenase, homoserine kinase, threonine synthase, threonine ammonia-lyase, 2-isopropylmalate synthase, isopropylmalate isomerase, 3-isopropylmalate dehydrogenase, 2-keto acid decarboxylase, and NAD-dependent alcohol dehydrogenase, NADPH-dependent alcohol dehydrogenase, butanol dehydrogenase and combinations thereof.

9. The method of claim 1, wherein the set of enzymes comprises at least one of the enzymes conferring a designer hydrogenotrophic methanogenic 2-methylbutanol-production pathway selected from the group consisting of methyl-H4MPT: coenzyme-M methyltransferase Mtr, A₁Aₒ-ATP synthase, methyl-coenzyme M reductase Mcr, energy converting hydrogenase (Ech), [NiFe]-hydrogenase (Mvh), Coenzyme F₄₂₀-reducing hydrogenase (Frh), soluble hydrogenase (SH), heterodissulfide reductase (Hdr), NADPH-dependent glyceraldehyde-3-phosphate dehydrogenase, NAD-dependent glyceraldehyde-3-phosphate dehydrogenase, phosphoglycerate mutase, enolase, pyruvate kinase, citramalate synthase, 2-methylmalate dehydratase, 3-isopropylmalate dehydratase, 3-isopropylmalate dehydrogenase, acetolactate synthase, ketol-acid reductoisomerase, dihydroxy-acid dehydratase, 2-keto acid decarboxylase, NAD-dependent alcohol dehydrogenase, NADPH-dependent alcohol dehydrogenase, and 2-methylbutyraldehyde reductase.

10. The method of claim 1, wherein the set of enzymes comprises at least one of the enzymes selected from the group consisting of membrane bound hydrogenase (MBH), soluble hydrogenase (SH), NADPH-dependent glyceraldehyde-3-phosphate dehydrogenase, NAD-dependent glyceraldehyde-3-phosphate dehydrogenase, phosphoglycerate mutase, enolase, phosphoenolpyruvate carboxylase, aspartate aminotransferase, aspartokinase, aspartate-semialdehyde dehydrogenase, homoserine dehydrogenase, homoserine kinase, threonine synthase, threonine ammonia-lyase, acetolactate synthase, ketol-acid reductoisomerase, dihydroxy-acid dehydratase, 2-keto acid decarboxylase, and NAD dependent alcohol dehydrogenase, NADPH dependent alcohol dehydrogenase, and 2-methylbutyraldehyde reductase.

11. The method of claim 1, wherein the set of enzymes comprises at least one of the enzymes selected from the group consisting of methyl-H4MPT: coenzyme-M methyltransferase Mtr, A₁Aₒ-ATP synthase, energy converting hydrogenase (Ech), [NiFe]-hydrogenase Mvh, Coenzyme F₄₂₀-reducing hydrogenase (Frh), native (or heterologous) soluble hydrogenase (SH), reduced ferredoxin (Fd_{red}²⁻), methyl-coenzyme M reductase Mcr, heterodissulfide reductase (Hdr), NADPH-dependent glyceraldehyde-3-phosphate dehydrogenase, NAD-dependent glyceraldehyde-3-phosphate dehydrogenase, phosphoglycerate mutase, enolase, pyruvate kinase, acetolactate synthase, ketol-acid reductoisomerase, dihydroxy-acid dehydratase, 2-keto acid decarboxylase, and NAD-dependent alcohol dehydrogenase, and NADPH-dependent alcohol dehydrogenase.

12. The method of claim 1, wherein the set of enzymes comprises at least one of the enzymes selected from the group consisting of membrane bound hydrogenase (MBH), soluble hydrogenase (SH), NADPH-dependent glyceraldehyde-3-phosphate dehydrogenase, NAD-dependent glyceraldehyde-3-phosphate dehydrogenase, phosphoglycerate mutase, enolase, pyruvate kinase, acetolactate synthase, ketol-acid reductoisomerase, dihydroxy-acid dehydratase, 2-isopropylmalate synthase, 3-isopropylmalate dehydratase, 3-isopropylmalate dehydrogenase, 2-keto acid decarboxylase, and NAD-dependent alcohol dehydrogenase, NADPH-dependent alcohol dehydrogenase, and 3-methylbutanal reductase.

13. The method of claim 1, wherein the set of enzymes comprises at least one of the enzymes conferring a designer anaerobic reductive-acetyl-CoA butanol-production pathway selected from the group consisting of: formate dehydroganse, 10-formyl-H₄ folate synthetase, methenyltetrahydrofolate cyclohydrolase, 10-methylene-H₄ folate dehydrogenase, 10-methylene-H₄ folate reductase, methyl-H₄ folate: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, butanol dehydrogenase, and alcohol dehydrogenase.

14. The method of claim 1, wherein the set of enzymes comprises at least one of the enzymes selected from the group consisting of membrane bound hydrogenase (MBH), soluble hydrogenase (SH), NADPH-dependent glyceraldehyde-3-phosphate dehydrogenase, NAD-dependent glyceraldehyde-3-phosphate dehydrogenase, phosphoglycerate mutase, enolase, pyruvate kinase, citramalate synthase, 2-methylmalate dehydratase, 3-isopropylmalate dehydratase, 3-isopropylmalate dehydrogenase, 2-isopropylmalate synthase, isopropylmalate isomerase, 3-isopropylmalate dehydrogenase, designer isopropylmalate synthase, designer isopropylmalate isomerase, designer 3-isopropylmalate dehydrogenase, designer 2-keto acid decarboxylase, short-chain alcohol dehydrogenase, hexanol dehydrogenase, designer isopropylmalate synthase, designer isopropylmalate isomerase, designer 3-isopropylmalate dehydrogenase, designer 2-keto acid decarboxylase, and designer short-chain alcohol dehydrogenase.

15. The method of claim 1, wherein the set of enzymes comprises at least one of the enzymes selected from the group consisting of membrane bound hydrogenase (MBH), soluble hydrogenase (SH), NADPH-dependent glyceraldehyde-3-phosphate dehydrogenase, NAD-dependent glyceraldehyde-3-phosphate dehydrogenase, phosphoglycerate mutase, enolase, phosphoenolpyruvate carboxylase, aspartate aminotransferase, aspartokinase, aspartate-semialdehyde dehydrogenase, homoserine dehydrogenase, homoserine kinase, threonine synthase, threonine ammonia-lyase, 2-isopropylmalate synthase, isopropylmalate isomerase, 3-isopropylmalate dehydrogenase, designer isopropylmalate synthase, designer isopropylmalate isomerase, designer 3-isopropylmalate dehydrogenase, designer 2-keto acid decarboxylase, short-chain alcohol dehydrogenase, hexanol dehydrogenase, designer isopropylmalate synthase, designer isopropylmalate isomerase, designer 3-isopropylmalate dehydrogenase, designer 2-keto acid decarboxylase, and designer short-chain alcohol dehydrogenase.

16. The method of claim 1, wherein the set of enzymes comprises at least one of the enzymes conferring a designer hydrogenotrophic Calvin-cycle-channeled pathway selected from the group consisting of membrane bound hydrogenase (MBH), soluble hydrogenase (SH), NADPH-dependent glyceraldehyde-3-phosphate dehydrogenase, NAD-dependent glyceraldehyde-3-phosphate dehydrogenase, phosphoglycerate mutase, enolase, pyruvate kinase, citramalate synthase, 2-methylmalate dehydratase, 3-isopropylmalate dehydratase, 3-isopropylmalate dehydrogenase, acetolactate synthase, ketol-acid reductoisomerase, dihydroxy-acid dehydratase, designer isopropylmalate synthase, designer isopropylmalate isomerase, designer 3-isopropylmalate dehydrogenase, designer 2-keto acid decarboxylase, short-chain alcohol dehydrogenase, designer isopropylmalate synthase, designer isopropylmalate isomerase, designer 3-isopropylmalate dehydrogenase, designer 2-keto acid decarboxylase, and designer short-chain alcohol dehydrogenase.

17. The method of claim 1, wherein the set of enzymes comprises at least one of the enzymes conferring a designer hydrogenotrophic Calvin-cycle-channeled pathway selected from the group consisting of membrane bound hydrogenase (MBH), soluble hydrogenase (SH), NADPH-dependent glyceraldehyde-3-phosphate dehydrogenase, NAD-dependent glyceraldehyde-3-phosphate dehydrogenase, phosphoglycerate mutase, enolase, phosphoenolpyruvate carboxylase, aspartate aminotransferase, aspartokinase, aspartate-semialdehyde dehydrogenase, homoserine dehydrogenase, homoserine kinase, threonine synthase, threonine ammonia-lyase, acetolactate synthase, ketol-acid reductoisomerase, dihydroxy-acid dehydratase, designer isopropylmalate synthase, designer isopropylmalate isomerase, designer 3-isopropylmalate dehydrogenase, designer 2-keto acid decarboxylase, short-chain alcohol dehydrogenase, designer isopropylmalate synthase, designer isopropylmalate isomerase, designer 3-isopropylmalate dehydrogenase, designer 2-keto acid decarboxylase, and designer short-chain alcohol dehydrogenase.

18. The method of claim 1, wherein the set of enzymes comprises at least one of the enzymes conferring a designer hydrogenotrophic Calvin-cycle-channeled pathway selected from the group consisting of membrane bound hydrogenase (MBH), soluble hydrogenase (SH), NADPH-dependent glyceraldehyde-3-phosphate dehydrogenase, NAD-dependent glyceraldehyde-3-phosphate dehydrogenase, phosphoglycerate mutase, enolase, pyruvate kinase, acetolactate synthase, ketol-acid reductoisomerase, dihydroxy-acid dehydratase, isopropylmalate synthase, dehydratase, 3-isopropylmalate dehydrogenase, designer isopropylmalate synthase, designer isopropylmalate isomerase, designer 3-isopropylmalate dehydrogenase, designer 2-keto acid decarboxylase, short-chain alcohol dehydrogenase, designer isopropylmalate synthase, designer isopropylmalate isomerase, designer 3-isopropylmalate dehydrogenase, designer 2-keto acid decarboxylase, and designer short-chain alcohol dehydrogenase.

19. The method of claim 1, wherein the set of enzymes comprises at least one of the enzymes conferring a designer methanogenic hydrogenotrophic butanol-production-pathway selected from the group consisting of: methyl-H4MPT: coenzyme-M methyltransferase Mtr, A₁Aₒ-ATP synthase, methyl-coenzyme M reductase Mcr, energy converting hydrogenase (Ech), [NiFe]-hydrogenase (Mvh), Coenzyme F₄₂₀-reducing hydrogenase (Frh), soluble hydrogenase (SH), heterodissulfide reductase (Hdr), formate dehydroganse, 10-formyl-H₄ folate synthetase, methenyltetrahydrofolate cyclohydrolase, 10-methylene-H₄ folate dehydrogenase, 10-methylene-H₄ folate reductase, methyl-H₄ folate: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, butanol dehydrogenase, and alcohol dehydrogenase.

20. The method of claim 1, wherein the designer transgenic organism a designer autotrophic organism comprises a set of designer genes that express a designer methanogenic hydrogenotrophic butanol-production-pathway system comprising: methyl-H4MPT: coenzyme-M methyltransferase Mtr, A₁Aₒ-ATP synthase, methyl-coenzyme M reductase Mcr, energy converting hydrogenase (Ech), [NiFe]-hydrogenase (Mvh), Coenzyme F₄₂₀-reducing hydrogenase (Frh), soluble hydrogenase (SH), heterodissulfide reductase (Hdr), formate dehydroganse, 10-formyl-H₄ folate synthetase, methenyltetrahydrofolate cyclohydrolase, 10-methylene-H₄ folate dehydrogenase, 10-methylene-H₄ folate reductase, methyl-H₄ folate: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, and butanol dehydrogenase; and wherein said autotrophic organism comprise a set of designer genes that express a designer methanogenic hydrogenotrophic butanol-production-pathway system comprising: methyl-H4MPT: coenzyme-M methyltransferase Mtr, native (or heterologous) A₁Aₒ-ATP synthase, methyl-coenzyme M reductase Mcr, energy converting hydrogenase (Ech), [NiFe]-hydrogenase (Mvh), Coenzyme F₄₂₀-reducing hydrogenase (Frh), native (or heterologous) soluble hydrogenase (SH), heterodissulfide reductase (Hdr), formylmethanofuran dehydroganse, formyl transferase, 10-methenyl-tetrahydromethanopterin cyclohydrolase, 10-methylene-H₄ methanopterin dehydrogenase, 10-methylene-H₄-methanopterin reductase, methyl-H₄-methanopterin: corrinoid iron-sulfur protein methyltransferase, corrinoid iron-sulfur protein, CO dehydrogenase/acetyl-CoA synthase, thiolase, 3-hydroxybutyryl-CoA dehydrogenase, crotonase, butyryl-CoA dehydrogenase, butyaldehyde dehydrogenase, and butanol dehydrogenase.
